(19) United States
(12) Patent Application Publication
(10) Pub. No.: US 2024/0180963 A1
(43) Pub. Date: Jun. 6, 2024

(54) DIAGNOSIS AND TREATMENT OF MYELOID DISORDERS AND ACUTE LEUKEMIAS USING NOVEL TUMOR SPECIFIC ANTIGENS

(71) Applicant: ALTHEIA SCIENCE S.R.L., Milan (IT)

(72) Inventors: Martina PIGAZZI, Padova (IT); Alessandra BIFFI, Padova (IT)

(21) Appl. No.: 18/285,850

(22) PCT Filed: Apr. 5, 2022
(86) PCT No.: PCT/EP2022/059031

§ 371 Date: Oct. 5, 2023

Related U.S. Application Data
(60) Provisional application No. 63/170,976, filed on Apr. 5, 2021.

(51) Int. Cl. A61K 35/17; A61K 39/00; A61K 45/06; A61K 47/68; A61P 35/02; C07K 16/28; C12N 5/0783; C12N 15/86; C12Q 1/6841; C12Q 1/6874; C12Q 1/6886; G01N 33/574; G01N 33/58

(52) U.S. Cl. CPC A61K 35/17; A61K 39/4611; A61K 39/4631; A61K 39/464429; A61K 45/06; A61K 47/6849; A61P 35/02; C07K 16/2851; C07K 16/2896; C12N 5/0636; C12N 15/86; C12Q 1/6841; C12Q 1/6874; C12Q 1/6886; G01N 33/57426; G01N 33/581; G01N 33/582; A61K 2239/13; A61K 2239/17; A61K 2239/21; A61K 2239/22; C12Q 2600/158; G01N 2333/70503

(57) ABSTRACT

The present disclosure relates to a method of diagnosing or treating myeloid disorders and acute leukemias by using a tumor specific antigen selected from CD63, CD151, CD72, CD84, CD69, and CD109. Further provided are an antigen binding protein (ABP), an ABP-drug conjugate, and a CAR targeting the tumor specific antigen, and methods for their use.

### CROSS-REFERENCE TO RELATED APPLICATIONS

This application claims the benefit of U.S. Provisional Application Nos. 63/170,976, filed Apr. 5, 2021, the disclosure of which is hereby incorporated in its entirety by reference.

### 1. SEQUENCE LISTING

The instant application contains a Sequence Listing, which has been submitted electronically in ASCII format and is hereby incorporated by reference in its entirety. Said ASCII copy, created on Apr. 4, 2022, is named 48002US_CRF_sequencelisting.txt and is 128,977 bytes in size.

### 2. BACKGROUND

Acute myeloid leukemia (AML) is a blood cancer that originates in the bone marrow and accounts for approximately one third of all pediatric malignancies. AIL causes the greatest number of cancer-related deaths in children with an overall survival ranging from 55 to 70% in five years of follow up. Chemotherapy has been the standard AML treatment for more than 40 years, and while it often causes the cancer to go into remission, it rarely eliminates the cancer cells completely. This often leads to disease recurrence and eventually to patients' death, because second line therapies are limited. Aggressive post-remission treatments, like high-dose chemotherapy and hematopoietic cell transplant, are currently adopted in more than 50% of patients after reaching the first remission. There is no therapeutic option available for the many relapsed patients who are not healthy enough to tolerate such aggressive post-remission treatments.

Thus, there have been extensive efforts to develop alternative primary treatments and post-remission treatments of AML, but without notable success. AIL cancer cells display variations in transcription factor occupancy and transcriptional regulation, and AML patients have various subtypes of leukemia associated symptomatic and prognostic differences. It has been suggested that targeting a specific subtype of leukemia could allow more effective personalized therapies. However, the subtype of leukemia within individual patients can change over time or as a result of treatment, and an individual patient can have multiple subclones.

There has also been research into immunotherapy approaches for treating AML. The interleukin-3 receptor a chain (IL3RA, also known as CD123) is one of the first antigens targeted for treatment of AML, because of its over-expression on a vast majority of AML cells as compared to normal bone marrow. Monoclonal antibodies and recombinant immunotoxins targeting CD123 showed promise in preclinical evaluations. CD33 is another antigen of interest because it is expressed on more than 80% of the AML malignant cells; however, it is also expressed on normal myeloid progenitor cell lines. Based on promising preclinical data, immunotherapies, including CAR-T cells, targeting these two surface molecules were tested in early phase clinical trials in relapsed and refractory AML patients. However, preliminary clinical results have been disappointing because these approaches showed only short-term response with no long-term benefit on the disease and caused severe adverse effects due to poor specificity of the employed targets i.e. severe pancytopenia and severe myeloablation due to expression of the targets also by hematopoietic stem and progenitor cells.

Therefore, there is a need for development of safe and effective alternative therapy for treatment of AML.

### 1. SUMMARY

Applicant identified six novel antigens (i.e., CD63, CD151, CD72, CD84, CD69, and CD109) specific to AML cells, with little to no expression within the hematopoietic stem cell compartment, using in silico analysis followed by experimental analysis and validation. The six tumor specific antigens (TSAs) were selected based in part on: stable, specific and high level expression in AML cells, as determined by flow cytometry using AML cell lines (SHI-1, HL-60, KASUMI-1, MOLM-13, MV4-1, and AML).

Based on the discovery, Applicant provides methods of diagnosing and treating myeloid disorders and acute leukemias (e.g., AML) by targeting a TSA selected from CD63, CD151, CD72, CD84, CD69, and CD109. Further provided herein are antigen-binding proteins (ABP), ABP-drug conjugates, and chimeric antigen receptors (CAR) that can be used for the treatment methods.

Accordingly, the present disclosure provides a method of diagnosing myeloid disorders (MD) and acute leukemias (AL) in a subject, the method comprising the step of: detecting the presence or level of a tumor specific antigen in a biological sample of the subject, wherein the tumor specific antigen is selected from the group consisting of CD63, CD151, CD72, CD84, CD69, and CD109.

In some embodiments, the biological sample is a blood sample or a bone marrow sample. In some embodiments, the biological sample comprises blast cells. In some embodiments, the blast cells are selected from myeloid blast cells, lymphoid blast cells, or a combination of myeloid and lymphoid blast cells.

In some embodiments, the step of detecting comprises contacting the biological sample with an antibody, wherein the antibody specifically binds to the tumor specific antigen. In some embodiments, the step of detecting comprises flow cytometry, immunocytochemistry, immunohistochemistry, fluorescence, or enzyme-linked immunosorbent assay (ELISA). In some embodiments, the antibody is labeled. In some embodiments, the antibody is labeled with a fluorophore, or an enzyme. In some embodiments, the target binding protein is labeled. In some embodiments, the target binding protein is labeled with a fluorophore or an enzyme. In some embodiments, the step of detecting comprise measuring mRNA level of the tumor specific antigen in the biological sample.

In some embodiments, the step of detecting comprises measuring mRNA level of the tumor specific antigen in the biological sample. In some embodiments, the mRNA level is measured by in situ hybridization, reverse transcription-polymerase chain reaction (RT-PCR), or by next generation sequencing.

In some embodiments, the myeloid disorders (MD) and acute leukemias (AL) have onset in pediatric or adult age. In some embodiments, the method further comprises the step of determining the presence or absence of cancer cells in the subject based on the detection of the presence or level of the tumor specific antigen in blood and/or bone marrow samples from the subject. In some embodiments, the method further comprises the step of determining the presence or absence of cancer cells in the subject based on the detection of the presence or level of the tumor specific antigen in the biological sample from the subject.

In some embodiments, the method further comprises administering to the subject a therapeutic effective amount of a therapeutic agent that specifically binds to the tumor specific antigen selected from the group consisting of: CD63, CD151, CD72, CD84, CD69, and CD109.

In some embodiments, wherein the therapeutic agent is an antigen-binding protein (ABP), an ABP-drug conjugate, an immunoresponsive cell expressing a chimeric antigen receptor (CAR), or a bispecific T-cell engager (BiTE), wherein the ABP, the ABP-drug conjugate, the CAR, or the BiTE specifically binds to the tumor specific antigen selected from the group consisting of: CD63, CD151, CD72, CD84, CD69, and CD109. In some embodiments, wherein the therapeutic agent is the ABP described in the present disclosure, an ABP-drug conjugate described in the present disclosure, an immunoresponsive cell expressing a chimeric antigen receptor (CAR) described in the present disclosure, or a bispecific T-cell engager (BiTE) described in the present disclosure.

In another aspect, the present disclosure provides a method of treating a subject with myeloid disorders (MD) or acute leukemia (AL), the method comprising: a) detecting the presence or level of a tumor specific antigen in a biological sample of the subject, wherein the tumor specific antigen is selected from the group consisting of CD63, CD151, CD72, CD84, CD69, and CD109; b) administering to the subject a therapeutically effective amount of an antigen-binding protein (ABP), an ABP-drug conjugate, or an immunoresponsive cell expressing a chimeric antigen receptor (CAR), wherein the ABP, the ABP-drug conjugate, or the CAR specifically binds to a target protein selected from the group consisting of CD63, CD151, CD72, CD84, CD69, and CD109.

In some embodiments, the biological sample is a blood sample, a bone marrow sample. In some embodiments, the biological sample comprises myeloid disorder (MD) and acute leukemia (AL) blast cells. In some embodiments, the blast cells are selected from myeloid blast cells, lymphoid blast cells, or a combination of myeloid and lymphoid blast cells.

In some embodiments, the presence or level of the tumor specific antigen is detected by contacting the biological sample with an antibody, wherein the antibody specifically binds to the tumor specific antigen. In some embodiments, the presence or level of the tumor specific antigen is detected by flow cytometry, immunocytochemistry, immunohistochemistry, fluorescence, or enzyme-linked immunosorbent assay (ELISA).

In some embodiments, the presence or level of the tumor specific antigen is detected by measuring mRNA level of the tumor specific antigen in the biological sample. In some embodiments, the mRNA level is measured by in situ hybridization, reverse transcription-polymerase chain reaction (RT-PCR), or next generation sequencing.

In one aspect, the present disclosure provides an antigen-binding protein (ABP) that specifically binds a target protein selected from CD63, CD151, CD72, CD84, CD69 and CD109.

In some embodiments, the ABP specifically binds human CD84.

In some embodiments, the ABP comprises an amino acid sequence having at least 80%, at least 85%, at least 90%, at least 95%, at least 97%, at least 99%, or 100% sequence identity to the amino acid sequence of SEQ ID NO: 97, SEQ ID NO: 98, or SEQ ID NO: 90.

In some embodiments, the ABP comprises:
a. V_{L} CDR1 having a sequence of SEQ ID NO: 51, V_{L} CDR2 having a sequence of SEQ ID NO: 54, V_{L} CDR3 having a sequence of SEQ ID NO: 61, V_{H} CDR1 having a sequence of SEQ ID NO: 63, V_{H} CDR2 having a sequence of SEQ ID NO: 68, and V_{H} CDR3 having a sequence of SEQ ID NO: 72;
b. V_{L} CDR1 having a sequence of SEQ ID NO: 47, V_{L} CDR2 having a sequence of SEQ ID NO: 54, V_{L} CDR3 having a sequence of SEQ ID NO: 55, V_{H} CDR1 having a sequence of SEQ ID NO: 63, V_{H} CDR2 having a sequence of SEQ ID NO: 68, and V_{H} CDR3 having a sequence of SEQ ID NO: 72; or
c. V_{L} CDR1 having a sequence of SEQ ID NO: 45, V_{L} CDR2 having a sequence of SEQ ID NO: 52, V_{L} CDR3 having a sequence of SEQ ID NO: 55, V_{H} CDR1 having a sequence of SEQ ID NO: 63, V_{H} CDR2 having a sequence of SEQ ID NO: 66, and V_{H} CDR3 having a sequence of SEQ ID NO: 71;

In some embodiments, the ABP comprises:
d. a light chain variable domain having at least 80%, at least 85%, at least 90%, at least 95%, at least 95%, at least 97% or 100% sequence identity to the amino acid sequence of SEQ ID NO: 80 and a heavy chain variable domain having at least 80%, at least 85%, at least 90%, at least 95%, at least 95%, at least 97% or 100% sequence identity to the amino acid sequence of SEQ ID NO: 88;
e. a light chain variable domain having at least 80%, at least 85%, at least 90%, at least 95%, at least 95%, at least 97% or 100% sequence identity to the amino acid sequence of SEQ ID NO: 81 and a heavy chain variable domain having at least 80%, at least 85%, at least 90%, at least 95%, at least 95%, at least 97% or 100% sequence identity to the amino acid sequence of SEQ ID NO: 88; or
f. a light chain variable domain having at least 80%, at least 85%, at least 90%, at least 95%, at least 95%, at least 97% or 100% sequence identity to the amino acid sequence of SEQ ID NO: 74 and a heavy chain variable domain having at least 80%, at least 85%, at least 90%, at least 95%, at least 95%, at least 97% or 100% sequence identity to the amino acid sequence of SEQ ID NO: 83.

In some embodiments, the ABP specifically binds human CD69.

In some embodiments, the ABP comprises an amino acid sequence having at least 80%, at least 85%, at least 90%, at least 95%, at least 97%, at least 99%, or 100% sequence identity to the amino acid sequence selected from: SEQ ID NOs: 89-96.

In some embodiments, the ABP comprises:
V_{L} CDR1 having a sequence of SEQ ID NO: 47, V_{L} CDR2 having a sequence of SEQ ID NO: 54, V_{L} CDR3 having a sequence of SEQ ID NO: 57, V_{H} CDR1 having a sequence of SEQ ID NO: 64, V_{H} CDR2 having a sequence of SEQ ID NO: 67, and V_{H} CDR3 having a sequence of SEQ ID NO: 71;
V_{L} CDR1 having a sequence of SEQ ID NO: 50, V_{L} CDR2 having a sequence of SEQ ID NO: 54, V_{L} CDR3 having a sequence of SEQ ID NO: 60, V_{H} CDR1 having a sequence of SEQ ID NO: 62, V_{H} CDR2 having a sequence of SEQ ID NO: 69, and V_{H} CDR3 having a sequence of SEQ ID NO: 73;
V_{L} CDR1 having a sequence of SEQ ID NO: 45, V_{L} CDR2 having a sequence of SEQ ID NO: 52, V_{L} CDR3 having a sequence of SEQ ID NO: 55, V_{H} CDR1 having a sequence of SEQ ID NO: 62, V_{H} CDR2 having a sequence of SEQ ID NO: 65, and V_{H} CDR3 having a sequence of SEQ ID NO: 70;
V_{L} CDR1 having a sequence of SEQ ID NO: 46, V_{L} CDR2 having a sequence of SEQ ID NO: 53, V_{L} CDR3 having a sequence of SEQ ID NO: 56, V_{H} CDR1 having a sequence of SEQ ID NO: 63, V_{H} CDR2 having a sequence of SEQ ID NO: 67, and V_{H} CDR3 having a sequence of SEQ ID NO: 71;
V_{L} CDR1 having a sequence of SEQ ID NO: 48, V_{L} CDR2 having a sequence of SEQ ID NO: 54, V_{L} CDR3 having a sequence of SEQ ID NO: 58, V_{H} CDR1 having a sequence of SEQ ID NO: 63, V_{H} CDR2 having a sequence of SEQ ID NO: 68, and V_{H} CDR3 having a sequence of SEQ ID NO: 72;
V_{L} CDR1 having a sequence of SEQ ID NO: 49, V_{L} CDR2 having a sequence of SEQ ID NO: 52, V_{L} CDR3 having a sequence of SEQ ID NO: 59, V_{H} CDR1 having a sequence of SEQ ID NO: 63, V_{H} CDR2 having a sequence of SEQ ID NO: 67, and V_{H} CDR3 having a sequence of SEQ ID NO: 71;
V_{L} CDR1 having a sequence of SEQ ID NO: 49, V_{L} CDR2 having a sequence of SEQ ID NO: 52, V_{L} CDR3 having a sequence of SEQ ID NO: 59, V_{H} CDR1 having a sequence of SEQ ID NO: 63, V_{H} CDR2 having a sequence of SEQ ID NO: 67, and V_{H} CDR3 having a sequence of SEQ ID NO: 71; or
V_{L} CDR1 having a sequence of SEQ ID NO: 45, V_{L} CDR2 having a sequence of SEQ ID NO: 52, V_{L} CDR3 having a sequence of SEQ ID NO: 55, V_{H} CDR1 having a sequence of SEQ ID NO: 63, V_{H} CDR2 having a sequence of SEQ ID NO: 66, and V_{H} CDR3 having a sequence of SEQ ID NO: 71.

In some embodiments, the ABP comprises:
a light chain variable domain having at least 80%, at least 85%, at least 90%, at least 95%, at least 95%, at least 97% or 100% sequence identity to the amino acid sequence of SEQ ID NO: 76 and a heavy chain variable domain having at least 80%, at least 85%, at least 90%, at least 95%, at least 95%, at least 97% or 100% sequence identity to the amino acid sequence of SEQ ID NO: 85;
a light chain variable domain having at least 80%, at least 85%, at least 90%, at least 95%, at least 95%, at least 97% or 100% sequence identity to the amino acid sequence of SEQ ID NO: 79 and a heavy chain variable domain having at least 80%, at least 85%, at least 90%, at least 95%, at least 95%, at least 97% or 100% sequence identity to the amino acid sequence of SEQ ID NO: 87;
a light chain variable domain having at least 80%, at least 85%, at least 90%, at least 95%, at least 95%, at least 97% or 100% sequence identity to the amino acid sequence of SEQ ID NO: 74 and a heavy chain variable domain having at least 80%, at least 85%, at least 90%, at least 95%, at least 95%, at least 97% or 100% sequence identity to the amino acid sequence of SEQ ID NO: 83;
a light chain variable domain having at least 80%, at least 85%, at least 90%, at least 95%, at least 95%, at least 97% or 100% sequence identity to the amino acid sequence of SEQ ID NO: 74 and a heavy chain variable domain having at least 80%, at least 85%, at least 90%, at least 95%, at least 95%, at least 97% or 100% sequence identity to the amino acid sequence of SEQ ID NO: 82;
a light chain variable domain having at least 80%, at least 85%, at least 90%, at least 95%, at least 95%, at least 97% or 100% sequence identity to the amino acid sequence of SEQ ID NO: 75 and a heavy chain variable domain having at least 80%, at least 85%, at least 90%, at least 95%, at least 95%, at least 97% or 100% sequence identity to the amino acid sequence of SEQ ID NO: 84;
a light chain variable domain having at least 80%, at least 85%, at least 90%, at least 95%, at least 95%, at least 97% or 100% sequence identity to the amino acid sequence of SEQ ID NO: 77 and a heavy chain variable domain having at least 80%, at least 85%, at least 90%, at least 95%, at least 95%, at least 97% or 100% sequence identity to the amino acid sequence of SEQ ID NO: 86;
a light chain variable domain having at least 80%, at least 85%, at least 90%, at least 95%, at least 95%, at least 97% or 100% sequence identity to the amino acid sequence of SEQ ID NO: 77 and a heavy chain variable domain having at least 80%, at least 85%, at least 90%, at least 95%, at least 95%, at least 97% or 100% sequence identity to the amino acid sequence of SEQ ID NO: 84;
a light chain variable domain having at least 80%, at least 85%, at least 90%, at least 95%, at least 95%, at least 97% or 100% sequence identity to the amino acid sequence of SEQ ID NO: 78 and a heavy chain variable domain having at least 80%, at least 85%, at least 90%, at least 95%, at least 95%, at least 97% or 100% sequence identity to the amino acid sequence of SEQ ID NO: 84; or a light chain variable domain having at least 80%, at least 85%, at least 90%, at least 95%, at least 95%, at least 97% or 100% sequence identity to the amino acid sequence of SEQ ID NO: 74 and a heavy chain variable domain having at least 80%, at least 85%, at least 90%, at least 95%, at least 95%, at least 97% or 100% sequence identity to the amino acid sequence of SEQ ID NO: 83.

In some embodiments, the ABP specifically binds human CD69 and CD84.

In some embodiments, the ABP comprises an amino acid sequence having at least 80%, at least 85%, at least 90%, at least 95%, at least 97%, at least 99%, or 100% sequence identity to the amino acid sequence of SEQ ID NO: 36.

In some embodiments, the ABP comprises:
V_{L} CDR1 having a sequence of SEQ ID NO: 45, V_{L} CDR2 having a sequence of SEQ ID NO: 52, V_{L} CDR3 having a sequence of SEQ ID NO: 55, V_{H} CDR1 having a sequence of SEQ ID NO: 63, V_{H} CDR2 having a sequence of SEQ ID NO: 66, and V_{H} CDR3 having a sequence of SEQ ID NO: 71.

In some embodiments, the ABP comprises a light chain variable domain having at least 80%, at least 85%, at least 90%, at least 95%, at least 95%, at least 97% or 100% sequence identity to the amino acid sequence of SEQ ID NO: 74 and a heavy chain variable domain having at least 80%, at least 85%, at least 90%, at least 95%, at least 95%, at least 97% or 100% sequence identity to the amino acid sequence of SEQ ID NO: 83.

In some embodiments, the ABP comprises an amino acid sequence selected from SEQ ID NOs: 89-98.

In some embodiments, the ABP comprises a Fab, Fab′, F(ab′)₂, Fv, scFv, (scFv)₂, single chain antibody molecule, dual variable domain antibody, single variable domain antibody, linear antibody, or V domain antibody.

In some embodiments, the ABP is a Fab, Fab′, F(ab′)₂, Fv, scFv, (scFv)₂, single chain antibody molecule, dual variable domain antibody, single variable domain antibody, linear antibody, or V domain antibody.

In some embodiments, the ABP is a monoclonal antibody.

In some embodiments, the ABP is selected from an IgG, IgM, IgA, IgD, and IgE antibody.

In some embodiments, the ABP comprises a heavy chain constant region of the class IgG and a subclass selected from IgG1, IgG2, IgG3, and IgG4.

In some embodiments, the ABP is conjugated to a drug.

In some embodiments, the ABP is capable of inducing antibody-dependent cell-mediated cytotoxicity (ADCC), wherein the ABP specifically binds to a target protein selected from the group consisting of CD63, CD151, CD72, CD84, CD69, and CD109, and wherein the ABP comprises a human Fc.

In some embodiments, the ABP is human, humanized or chimeric.

In some embodiments, the ABP is monoclonal.

In some embodiments, the ABP is bispecific or multispecific.

In some embodiments, the ABP comprises a heavy chain constant region of IgG.

In some embodiments, the ABP is afucosylated.

In some embodiments, the ABP binds to the target protein with a K_{D} of less than or equal to 50 nM, 10 nM, 5 nM, 1 nM, 0.5 nM or 0.1 nM, as measured by surface plasmon resonance (SPR) assay.

In one aspect, the present disclosure provides an isolated polynucleotide or set of polynucleotides encoding the ABP described in the present disclosure. In another aspect, the present disclosure provides a vector or set of vectors comprising the isolated polynucleotide described in the present disclosure. In another aspect, the present disclosure provides a host cell comprising the isolated polynucleotide or vector described in the present disclosure.

In one aspect, the present disclosure provides a method of producing an isolated antigen binding protein (ABP), comprising expressing the ABP in the host cell as described in the present disclosure, and isolating the ABP.

In one aspect, the present disclosure provides an antigen-binding protein (ABP) capable of inducing antibody-dependent cell-mediated cytotoxicity (ADCC), wherein the ABP specifically binds to a target protein selected from the group consisting of CD63, CD151, CD72, CD84, CD69, and CD109, and wherein the ABP comprises a human Fc.

In some embodiments, the ABP is human, humanized, or chimeric. In some embodiments, the ABP is monoclonal. In some embodiments, the ABP is bispecific or multispecific. In some embodiments, the ABP comprises a heavy chain constant region of IgG. In some embodiments, the ABP comprises a heavy chain constant region of IgG1. In some embodiments, the ABP is afucosylated.

In some embodiments, the ABP binds to the target protein with a K_{D} of less than or equal to 50 nM, 10 nM, 5 nM, 1 nM, 0.5 nM or 0.1 nM, as measured by surface plasmon resonance (SPR) assay.

In another aspect, the present disclosure provides a pharmaceutical composition comprising the ABP provided herein and a pharmaceutically acceptable excipient.

In yet another aspect, the present disclosure provides a method of treating a subject with a myeloid disorders (MD) or acute leukemia (AL), the method comprising: administering a therapeutically effective amount of the ABP or the pharmaceutical composition provided herein. In some embodiments, the myeloid disorders (MD) and acute leukemia (AL) are of pediatric or adult onset.

In some embodiments, the ABP or the pharmaceutical composition is administered in combination with an additional agent. In some embodiments, the additional agent is a chemotherapeutic or biological agent. In some embodiments, the chemotherapeutic agent is selected from the group consisting of cytarabine, daunorubicin, idarubicin, cladribine, mitoxantrone, azacitidine, decitabine, and CPX-351 (Vyxeos®). In some embodiments, the additional agent is a hedgehog pathway inhibitor. In some embodiments, the hedgehog pathway inhibitor is a sonic hedgehog pathway inhibitor. In some embodiments, the sonic hedgehog pathway inhibitor is selected from vismodegib, sonidigib, and arsenic trioxide (ATO). In some embodiments, the hedgehog pathway inhibitor is glasdegib (Daurismo™). In some embodiments, the additional agent is an FMS-like tyrosine kinase 3 (FLT3) inhibitor. In some embodiments, the FLT3 inhibitor is selected from the group consisting of midostaurin (Rydapt®), gilteritinib (Xospata®), sorafenib, lestaurtinib, quizartinib, and crenolanib. In some embodiments, the additional agent is an isocitrate dehydrogenase 1 (IDH1) or isocitrate dehydrogenase 2 (IDH2) inhibitor. In some embodiments, the IDH1 or IDH2 inhibitor is ivosidenib (Tibsovo®) or enasidenib (Idhifa®). In some embodiments, the additional agent is a B-cell lymphoma 2 (BCL2) inhibitor. In some embodiments, the BCL2 inhibitor is venetoclax (Venclexta®). In some embodiments, the additional agent is a CD33-targeting agent. In some embodiments, the CD33-targeting agent is gemtuzumab ozogamicin (Mylotarg™) or vadastuximab talirine (SGN-CD33A). In some embodiments, the additional agent is a cell cycle checkpoint inhibitor. In some embodiments, the cell cycle checkpoint inhibitor is a Aurora kinase inhibitor, a Polo-like kinase 1 (PLK1) inhibitor, a cyclin dependent kinase (CDK) inhibitor, or a checkpoint kinase 1 (CHK1) inhibitor. In some embodiments, the additional agent is an immune checkpoint inhibitor. In some embodiments, the immune checkpoint inhibitor is an anti-CTLA-4 antibody, an anti-PD-1 antibody, or an anti-PD-L1 antibody.

In yet another aspect, the present disclosure provides an antigen-binding protein (ABP)-drug conjugate, comprising: an antigen-binding protein (ABP), a cytotoxic agent linked to the ABP, and optionally a linker that links the cytotoxic agent to the ABP, wherein the ABP specifically binds to a target protein selected from the group consisting of CD63, CD151, CD72, CD84, CD69, and CD109.

In some embodiments, the ABP is human, humanized, or chimeric. In some embodiments, the ABP is monoclonal. In some embodiments, the ABP is bispecific or multispecific. In some embodiments, the ABP comprises a Fab, Fab″, F(ab″)2, Fv, scFv, (scFv)2, single chain antibody molecule, dual variable domain antibody, single variable domain antibody, linear antibody, V domain antibody, or bispecific tandem bivalent scFvs, or bispecific T-cell engager (BiTE). In some embodiments, the ABP comprises an Fe, optionally human Fc. In some embodiments, the ABP comprises a heavy chain constant region of a class selected from IgG, IgA, IgD, IgE, and IgM. In some embodiments, the ABP comprises a heavy chain constant region of the class IgG and a subclass selected from IgG1, IgG2, IgG3, and IgG4. In some embodiments, the ABP comprises a heavy chain constant region of IgG1.

In certain embodiments, the ABP is a BiTE. In particular embodiments, the BiTE comprises an antigen-binding domain and a T-cell activating domain. In certain embodiments, the antigen-binding domain specifically binds to a target protein/antigen selected from the group consisting of CD63, CD151, CD72, CD84, CD69, and CD109.

In some embodiments, the antigen-binding domain comprises a single-chain variable fragment (scFv) of an antibody that specifically binds to the target protein (CD63, CD151, CD72, CD84, CD69, or CD109).

In some embodiments, the antigen-binding domain binds to an epitope on a CD63, CD151, CD72, CD84, CD69, or CD109 target antigen. In some embodiments, said antigen-binding domain comprises the CDRs of the CD63, CD151, CD72, CD84, CD69, or CD109 antibody. In some embodiments, said antigen-binding domain comprises the V_{H} and V_{L} domains of the CD63, CD151, CD72, CD84, CD69, or CD109 antibody. In some embodiments, said antigen-binding domain comprises an CD63, CD151, CD72, CD84, CD69, or CD109 single-chain variable fragment (scFv).

In some embodiments, the T-cell activating domain comprises the intracellular domain of CD3ζ. In certain embodiments, the T-cell activation domain binds to CD3.

In some embodiments, the ABP binds to the target protein with a K_{D} of less than or equal to 50 nM, 10 nM, 5 nM, 1 nM, 0.5 nM or 0.1 nM, as measured by surface plasmon resonance (SPR) assay.

In some embodiments, the cytotoxic agent comprises an anti-angiogenic agent, a pro-apoptotic agent, an anti-mitotic agent, an anti-kinase agent, an alkylating agent, a hormone, a hormone agonist, a hormone antagonist, a chemokine, a drug, a prodrug, a toxin, an enzyme, an antimetabolite, an antibiotic, an alkaloid, or a radioactive isotope. In some embodiments, the linker is a cleavable linker. In some embodiments, the linker is a non-cleavable linker.

One aspect of the present disclosure provides a pharmaceutical composition comprising the ABP-drug conjugate described in the present disclosure and a pharmaceutically acceptable excipient.

In one aspect, the present disclosure provides a method of treating a subject with myeloid disorders (MD) or acute leukemia (AL), the method comprising: administering a therapeutically effective amount of the ABP-drug conjugate or the pharmaceutical composition described in the present disclosure.

In some embodiments, the myeloid disorders (MD) and acute leukemias (AL) are of pediatric or adult onset. In some embodiments, the ABP-drug conjugate or the pharmaceutical composition is administered in combination with an additional agent. In some embodiments, the additional agent is a chemotherapeutic or biological agent. In some embodiments, the chemotherapeutic agent is selected from the group consisting of cytarabine, daunorubicin, idarubicin, cladribine, mitoxantrone, azacitidine, decitabine, and CPX-351 (Vyxeos®). In some embodiments, the additional agent is a hedgehog pathway inhibitor. In some embodiments, the hedgehog pathway inhibitor is a sonic hedgehog pathway inhibitor. In some embodiments, the sonic hedgehog pathway inhibitor is selected from vismodegib, sonidigib, and arsenic trioxide (ATO). In some embodiments, the hedgehog pathway inhibitor is glasdegib (Daurismo™). In some embodiments, the additional agent is an FMS-like tyrosine kinase 3 (FLT3) inhibitor. In some embodiments, the FLT3 inhibitor is selected from the group consisting of midostaurin (Rydapt®), gilteritinib (Xospata®), sorafenib, lestaurtinib, quizartinib, and crenolanib. In some embodiments, the additional agent is an isocitrate dehydrogenase 1 (IDH1) or isocitrate dehydrogenase 2 (IDH2) inhibitor. In some embodiments, the IDH1 or IDH2 inhibitor is ivosidenib (Tibsovo®) or enasidenib (Idhifa®). In some embodiments, the additional agent is a B-cell lymphoma 2 (BCL2) inhibitor. In some embodiments, the BCL2 inhibitor is venetoclax (Venclexta®). In some embodiments, the additional agent is a CD33-targeting agent. In some embodiments, the CD33-targeting agent is gemtuzumab ozogamicin (Mylotarg™) or vadastuximab talirine (SGN-CD33A). In some embodiments, the additional agent is a cell cycle checkpoint inhibitor. In some embodiments, the cell cycle checkpoint inhibitor is an Aurora kinase inhibitor, a Polo-like kinase 1 (PLK1) inhibitor, a cyclin dependent kinase (CDK) inhibitor, or a checkpoint kinase 1 (CHK1) inhibitor. In some embodiments, the additional agent is an immune checkpoint inhibitor. In some embodiments, the immune checkpoint inhibitor is an anti-CTLA-4 antibody, an anti-PD-1 antibody, or an anti-PD-L1 antibody.

In yet another aspect, the present disclosure provides a chimeric antigen receptor (CAR) comprising an extracellular antigen-binding domain, a transmembrane domain, a signaling domain, and optionally a costimulatory domain, wherein the extracellular antigen-binding domain specifically binds to a target protein or antigen selected from the group consisting of CD63, CD151, CD72, CD84, CD69, and CD109.

In some embodiments, the extracellular antigen-binding domain comprises a single-chain variable fragment (scFv) of an antibody that specifically binds to the target protein.

In some embodiments, the extracellular antigen-binding domain comprises the ABP described in the present disclosure.

In some embodiments, the signaling domain comprises the intracellular domain of CD3ζ.

In some embodiments, the CAR further comprising a costimulatory domain, wherein the costimulatory domain is a CD28 costimulatory domain, a 4-1BB costimulatory domain, a CD27 costimulatory domain, an OX40 costimulatory domain, or an ICOS costimulatory domain.

In some embodiments, the costimulatory domain is a 4-1BB costimulatory domain. In some embodiments, the 4-1BB costimulatory domain an amino acid sequence having at least 90%, at least 95%, or at least 100% sequence identity to the amino acid sequence of SEQ ID NO: 105.

In some embodiments, the transmembrane domain is a CD28 transmembrane domain.

In some embodiments, the CD28 transmembrane domain an amino acid sequence having at least 90%, at least 95%, or at least 100% sequence identity to the amino acid sequence of: SEQ ID NO: 100.

In some embodiments, the CAR further comprising a hinge region.

In some embodiments, the hinge region is a hinge region derived from a CD28 polypeptide.

In some embodiments, the hinge region comprises an amino acid sequence having at least 90%, at least 95%, or at least 100% sequence identity to the amino acid sequence of: SEQ ID NO: 99.

In some embodiments, the signaling domain is a CD3zeta signaling domain.

In some embodiments, the CD3zeta signaling domain comprises an amino acid sequence having at least 90%, at least 95%, or at least 100% sequence identity to the amino acid sequence of: SEQ ID NO: 101.

In some embodiments, the CAR comprises a polypeptide having at least 80%, at least 85%, at least 90%, at least 95%, at least 97%, at least 98%, at least 99%, or 100% sequence identity to an amino acid sequence selected from SEQ ID NOs.: 35-44.

In some embodiments, the extracellular antigen-binding domain specifically binds to human CD84.

In some embodiments, the extracellular antigen-binding domain specifically binds to human CD69.

In some embodiments, the CAR comprises a polypeptide having at least 80%, at least 85%, at least 90%, at least 95%, at least 97%, at least 98%, at least 99%, or 100% sequence identity to an amino acid selected from: SEQ ID NOs.: 35-42.

In some embodiments, the CAR comprises a polypeptide having at least 80%, at least 85%, at least 90%, at least 95%, at least 97%, at least 98%, at least 99%, or 100% sequence identity to an amino acid of SEQ ID NOs: 43, 44, or 36.

In one aspect, the present disclosure provides a polynucleotide encoding the CAR described in the present disclosure.

In some embodiments, the CAR comprises a nucleotide sequence having at least 75%, at least 80%, at least 85%, at least 90%, at least 95%, at least 97%, or 100% sequence identity to the nucleotide sequence of SEQ ID NOs: 24-34.

In one aspect, the present disclosure provides a vector comprising the CAR polynucleotide in the present disclosure.

In one aspect, the present disclosure provides an immunoresponsive cell expressing the CAR described in the present disclosure.

In one aspect, the present disclosure provides an immunoresponsive cell comprising the CAR polynucleotide described in the present disclosure or the vector described in the present disclosure.

In some embodiments, the immunoresponsive cell is an αβ T cell, a γδ T cell, or a Natural Killer (NK) cell.

In some embodiments, the αβ T cell is a CD4⁺ T cell or a CD8⁺ T cell.

In one aspect, the present disclosure provides a method of preparing the immunoresponsive cell described in the present disclosure, the method comprising transfecting or transducing the CAR polynucleotide or the vector described in the present disclosure into an immune cell.

In some embodiments, the method comprises expanding the immune cell for at least 48 hours.

In some embodiments, the immune cell is transduced at a multiplicity of infection of 10.

In some embodiments, the method further comprises, after transducing, washing the vector of claim 58, and expanding the transduced immune cell for at least 14 days.

In one aspect, the present disclosure provides a method of treating a subject, the method comprising: administering a therapeutically effective amount of the ABP described in the present disclosure, the ABP-drug conjugate described in the present disclosure, the pharmaceutical composition described in the present disclosure, or the immunoresponsive cell described in the present disclosure.

In some embodiments, the subject has a myeloid disorders (MD) or acute leukemia (AL).

In some embodiments, the myeloid disorders (MD) and acute leukemias (AL) are of pediatric or adult onset.

In some embodiments, the extracellular antigen-binding domain comprises a single-chain variable fragment (scFv) of an antibody that specifically binds to the target protein. In some embodiments, the signaling domain comprises the intracellular domain of CD3ζ. In some embodiments, the CAR further comprises a costimulatory domain, wherein the costimulatory domain is a CD28 costimulatory domain, a 4-1BB costimulatory domain, a CD27 costimulatory domain, an OX40 costimulatory domain, or an ICOS costimulatory domain.

In one aspect, the present disclosure provides a polynucleotide encoding the CAR. In another aspect, the present disclosure provides a vector comprising the polynucleotide. In yet another aspect, the present disclosure provides an immunoresponsive cell expressing the CAR described in the present disclosure and an immunoresponsive cell comprising the polynucleotide.

In some embodiments, the immunoresponsive cell is an αβ T cell, a γδ T cell, or a Natural Killer (NK) cell. In some embodiments, the αβ T cell is a CD4+ T cell, a CD3⁺ T cell, or a CD8+ T cell.

In one aspect, the present disclosure provides a method of preparing the immunoresponsive cell, the method comprising transfecting or transducing the polynucleotide or the vector into an immunoresponsive cell.

In another aspect, the present disclosure provides a method of treating a subject with a myeloid disorders (MD) or acute leukemia (AL), the method comprising: administering a therapeutically effective amount of the immunoresponsive cell. In some embodiments, the myeloid disorders (MD) and acute leukemias (AL) are of pediatric or adult onset.

In some embodiments, the immunoresponsive cell is administered in combination with an additional agent. In some embodiments, the additional agent is a chemotherapeutic or biological agent. In some embodiments, the chemotherapeutic agent is selected from the group consisting of cytarabine, daunorubicin, idarubicin, cladribine, mitoxantrone, azacitidine, decitabine, and CPX-351 (Vyxeos®). In some embodiments, the additional agent is a hedgehog pathway inhibitor. In some embodiments, the hedgehog pathway inhibitor is a sonic hedgehog pathway inhibitor. In some embodiments, the sonic hedgehog pathway inhibitor is selected from vismodegib, sonidigib, and arsenic trioxide (ATO). In some embodiments, the hedgehog pathway inhibitor is glasdegib (Daurismo™). In some embodiments, the additional agent is an FMS-like tyrosine kinase 3 (FLT3) inhibitor. In some embodiments, the FLT3 inhibitor is selected from the group consisting of midostaurin (Rydapt®), gilteritinib (Xospata®), sorafenib, lestaurtinib, quizartinib, and crenolanib. In some embodiments, the additional agent is an isocitrate dehydrogenase 1 (IDH1) or isocitrate dehydrogenase 2 (IDH2) inhibitor. In some embodiments, the IDH1 or IDH2 inhibitor is ivosidenib (Tibsovo®) or enasidenib (Idhifa®). In some embodiments, the additional agent is a B-cell lymphoma 2 (BCL2) inhibitor. In some embodiments, the BCL2 inhibitor is venetoclax (Venclexta®). In some embodiments, the additional agent is a CD33-targeting agent. In some embodiments, the CD33-targeting agent is gemtuzumab ozogamicin (Mylotarg™) or vadastuximab talirine (SGN-CD33A). In some embodiments, the additional agent is a cell cycle checkpoint inhibitor. In some embodiments, the cell cycle checkpoint inhibitor is an Aurora kinase inhibitor, a Polo-like kinase 1 (PLK1) inhibitor, a cyclin dependent kinase (CDK) inhibitor, or a checkpoint kinase 1 (CHK1) inhibitor. In some embodiments, the additional agent is an immune checkpoint inhibitor. In some embodiments, the immune checkpoint inhibitor is an anti-CTLA-4 antibody, an anti-PD-1 antibody, or an anti-PD-L1 antibody.

In one aspect, the present disclosure provides a bispecific T-cell engager (BiTE) comprising an antigen-binding domain and a T-cell activating domain, wherein the antigen-binding domain specifically binds to a target protein/antigen selected from the group consisting of CD63, CD151, CD72, CD84, CD69, and CD109.

In some embodiments, the antigen-binding domain comprises a single-chain variable fragment (scFv) of an antibody that specifically binds to the target protein.

In some embodiments, the T-cell activating domain comprises the intracellular domain of CD3ζ.

In some embodiments, the T-cell activating domain comprises a single-chain variable fragment (scFV) of an antibody that specifically binds to CD3.

In another aspect, the present disclosure provides a polynucleotide encoding the BiTE. In another aspect, the present disclosure provides a vector comprising the polynucleotide encoding the BiTE.

In another aspect, the present disclosure provides a method of treating a subject with a myeloid disorders (MD) or acute leukemia (AL), the method comprising: administering a therapeutically effective amount of the BiTE.

In some embodiments, the myeloid disorders (MD) and acute leukemias (AL) are of pediatric or adult onset.

In some embodiments, the BiTE or vector is administered in combination with an additional agent.

In some embodiments, the additional agent is a chemotherapeutic or biological agent. In some embodiments, the chemotherapeutic agent is selected from the group consisting of cytarabine, daunorubicin, idarubicin, cladribine, mitoxantrone, azacitidine, decitabine, and CPX-351 (Vyxeos®). In some embodiments, the additional agent is a hedgehog pathway inhibitor. In some embodiments, the hedgehog pathway inhibitor is a sonic hedgehog pathway inhibitor. In some embodiments, the sonic hedgehog pathway inhibitor is selected from vismodegib, sonidigib, and arsenic trioxide (ATO). In some embodiments, the hedgehog pathway inhibitor is glasdegib (Daurismo™). In some embodiments, the additional agent is an FMS-like tyrosine kinase 3 (FLT3) inhibitor. In some embodiments, the FLT3 inhibitor is selected from the group consisting of midostaurin (Rydapt®), gilteritinib (Xospata®), sorafenib, lestaurtinib, quizartinib, and crenolanib. In some embodiments, the additional agent is an isocitrate dehydrogenase 1 (IDH1) or isocitrate dehydrogenase 2 (IDH2) inhibitor. In some embodiments, the IDH1 or IDH2 inhibitor is ivosidenib (Tibsovo®) or enasidenib (Idhifa®). In some embodiments, the additional agent is a B-cell lymphoma 2 (BCL2) inhibitor. In some embodiments, the BCL2 inhibitor is venetoclax (Venclexta®). In some embodiments, the additional agent is a CD33-targeting agent. In some embodiments, the CD33-targeting agent is gemtuzumab ozogamicin (Mylotarg™) or vadastuximab talirine (SGN-CD33A). In some embodiments, the additional agent is a cell cycle checkpoint inhibitor. In some embodiments, the cell cycle checkpoint inhibitor is a Aurora kinase inhibitor, a Polo-like kinase 1 (PLK1) inhibitor, a cyclin dependent kinase (CDK) inhibitor, or a checkpoint kinase 1 (CHK1) inhibitor. In some embodiments, the additional agent is an immune checkpoint inhibitor. In some embodiments, the immune checkpoint inhibitor is an anti-CTLA-4 antibody, an anti-PD-1 antibody, or an anti-PD-L1 antibody.

### 2. BRIEF DESCRIPTION OF THE SEVERAL VIEWS OF THE DRAWINGS

These and other features, aspects, and advantages of the present disclosure will become better understood with the following description, and accompanying drawings, where:

FIG. 1 is a flowchart illustrating the process of identifying novel AML TSAs described in Example 1. The screening flowchart shows the original 2 lists of genes selected from AML gene expression data at diagnosis along with the prioritization based on specific inclusion and exclusion criteria defining the 26/76 genes that proceeded to flow cytometry expression analysis in AML cell lines (SHI-1, HL-60 MOLM-13, MV4; 11, and Kasumi-1) as well as in healthy control hematopoietic cells (PBMC sorted for CD3⁺, CD19⁺ and CD33⁺ subpopulations and CD34⁺ cells isolated from cord blood). The best candidate TSA localization on AML cell surface was confirmed by immunofluorescence.

FIG. 2A shows data from flow cytometric analysis performed with two different antibodies against CD69 (light-grey histogram) versus an isotype (dark grey histogram) in several AML cell lines (HL-60, SHI-1, KASUM-1, MOLM-13 and MV4-11). Cytometric analysis data for an isotype is provided as a control (dark grey histogram). An isotype control is an antibody that maintains similar properties to the primary antibody but lacks specific target binding. FIG. 2B shows an image of fluorescent immunohistochemistry of the AML cell line (SHI-1) with the two antibodies against CD69 (white dots) together with membrane staining (Membrite: co-localization of CD69 with plasma membrane-specific dye). FIG. 2C shows flow cytometric data from analysis performed with the CD69 antibodies on healthy hematopoietic cells, i.e., healthy CD3, CD19 and CD33 sub-populations derived from PBMCs and CD34 positive cells derived from cord blood.

FIG. 3A shows data from flow cytometric analysis performed with two different antibodies against CD63 (light-grey histogram) on several AML cell lines (HL-60, SHI-1, KASUM-1, MOLM-13 and MV4-11). Cytometric analysis data for an isotype is provided as a control (dark grey histogram). FIG. 3B shows an image of fluorescent immunohistochemistry of the AML cell line (HL-60) with the two antibodies against CD63 (white dots) together with membrane staining (Membrite: co-localization of CD63 with plasma membrane-specific dye). FIG. 3C shows data from flow cytometric analysis performed with the CD63 antibodies on healthy hematopoietic cells, i.e., healthy CD3, CD19 and CD33 sub-populations derived from PBMCs and CD34 positive cells derived from cord blood.

FIG. 4A shows data from flow cytometric analysis performed with two different antibodies against CD151 (light-grey histogram) on several AML cell lines (HL-60, SHI-1, KASUM-1, MOLM-13 and MV4-11). Cytometric analysis data for an isotype is provided as a control (dark grey histogram). FIG. 4B shows an image of fluorescent immunohistochemistry of the AML cell line (HL-60) with the two antibodies against CD151 (white dots) together with membrane staining (Membrite: co-localization of CD151 with plasma membrane-specific dye). FIG. 4C shows data from flow cytometric analysis performed with the CD151 antibodies on healthy hematopoietic cells, i.e., healthy CD3, CD19 and CD33 sub-populations derived from PBMCs and CD34 positive cells derived from cord blood.

FIG. 5A shows data from flow cytometric analysis performed with two different antibodies against CD84 (light-grey histogram) on several AML cell lines (HL-60, SHI-1, KASUM-1, MOLM-13 and MV4-11). Cytometric analysis data for an isotype is provided as a control (dark grey histogram). FIG. 5B shows an image of fluorescent immunohistochemistry of the AML cell line (HL-60) with the two antibodies against CD84 (white dots) together with membrane staining (Membrite: co-localization of CD84 with plasma membrane-specific dye). FIG. 5C shows data from flow cytometric analysis performed with the CD84 antibodies on healthy hematopoietic cells, i.e., healthy CD3, CD19 and CD33 sub-populations derived from PBMCs and CD34 positive cells derived from cord blood.

FIG. 6A shows data from flow cytometric analysis performed with two different antibodies against CD109 (light-grey histogram) on several AML cell lines (HL-60, SHI-1, KASUM-1, MOLM-13 and MV4-11). Cytometric analysis data for an isotype is provided as a control (dark grey histogram). FIG. 6B shows an image of fluorescent immunohistochemistry of the AML cell line (Kasumi-1 or HL-60) with the two antibodies against CD109 (white dots) together with membrane staining (Membrite: co-localization of CD109 with plasma membrane-specific dye). FIG. 6C shows data from flow cytometric analysis performed with the CD109 antibodies on healthy hematopoietic cells, i.e., healthy CD3, CD19 and CD33 sub-populations derived from PBMCs and CD34 positive cells derived from cord blood.

FIG. 7A shows data from flow cytometric analysis performed with two different antibodies against CD72 (light-grey histogram) on several AML cell lines (HL-60, SHI-1, KASUM-1, MOLM-13 and MV4-11). Cytometric analysis data for an isotype is provided as a control (dark-grey histogram). FIG. 7B shows an image of fluorescent immunohistochemistry of the AML cell line (SHI-1) with the two antibodies against CD72 (white dots) together with membrane staining (Membrite: co-localization of CD72 with plasma membrane-specific dye). FIG. 7C shows data from flow cytometric analysis performed with the CD72 antibodies on healthy hematopoietic cells, i.e., healthy CD3, CD19 and CD33 sub-populations derived from PBMCs and CD34 positive cells derived from cord blood.

FIG. 8 shows flow cytometry results on human primary skin fibroblasts for testing specificity of TSA.

FIG. 9 shows flow cytometry results on different cancer cell lines for testing specificity of TSA. TSA expression levels were assessed in cell lines from different cancer types by flow cytometry and are shown as histograms (light grey) versus isotype control (grey).

FIG. 10 shows flow cytometry results on AML cell lines using different commercial antibodies.

FIG. 11 shows TSA mRNA expression in AML samples collected at diagnosis and at remission following therapy. TSA mRNA expression was assessed by quantitative PCR in 14 paired pediatric AML bone marrow samples collected at diagnosis (RQ=1) and at remission following therapy (dots represent mRNA expression values at remission with respect to diagnosis). Patients are stratified by AML genetic risk (SR=standard risk; HR=high risk). Albeit TSA mRNA expression is heterogeneous, a significant (*denotes p<0.05) reduction in CD69, CD63 and CD84 mRNA expression following therapy was detected (RQ<1).

FIGS. 12A-12B show TSA expression by flow cytometry in the clinic. FIG. 12A shows lack of CD69 and CD84 expression in CD34⁺CD38⁻ subpopulation. FIG. 12B shows TSA expression in an AML sample at diagnosis, showing the highest expression of CD69 and CD84 in AML blasts.

FIG. 13. Pipeline of novel AML CAR selection and construction for CD69 and CD84. Ten ScFv sequences were selected from phage display and prioritized based on specificity for being cloned into a CAR cassette in a 3^{rd} generation lentivirus transfer plasmid for expression.

FIG. 14. CAR-T cell manufacturing workflow. Schematic representation of the lentiviral transduction process and CAR-T cell manufacturing. The workflow includes isolation of donor T cells at day 1, efficient activation by TransAct, and gene transfer of the CAR-LV construct at day 3. From day 4 CAR-T cells (generated with the ScFv sequences B8 or F12 for CD84, and G1 or H3 for CD69) underwent expansion in TexMACS medium supplemented with IL-7 and IL-15 for 14 days. At day 17 CAR-T cells underwent immunophenotyping and were grown in coculture with HL-60 or SHI-1 (CD84⁺ CD69⁺) AML cell lines.

FIG. 15. Transduction efficiency was determined at day 17 by measurement of vector copy number (VCN, #of integrated viral copies per cell). VCN was determined by digital droplet PCR (ddPCR) analysis with primers within the lentiviral backbone for all CAR-T cell constructs for every experiment performed. Each symbol represents a different CAR construct (generated with ScFv sequences B8 or F12 for CD84, and G1 or H3 for CD69, and mock transduced [empty CAR]).

FIG. 16. Flow cytometry cell-surface expression of CD69 and CD84 on indicated AML cell lines and isotype control (dark grey peak). Representative histogram of 3 independent experiments.

FIG. 17. Representative fold expansion kinetics of T cells during CAR-T cell manufacturing (from day 0 to day 14). Mock transduced (empty CAR) and T cells were monitored, and cell number was calculated by flow cytometry. Data are represented as mean±SEM (n=2 to 7, P>0.05). Each symbol represents a different CAR construct (generated with ScFv sequences B8 or F12 for CD84, and G1 or H3 for CD69, and mock transduced [empty CAR]).

FIGS. 18A-18B. Frequencies of viable T lymphocytes (FIG. 18A) and percentages of CD4⁺ and CD8⁺ cells (FIG. 18B) within lymphocytes at the end of manufacturing (day 14) measured by flow cytometry. Bars and symbols indicate the different CAR-T cell products (generated with ScFv sequences B8 or F12 for CD84, and G1 or H3 for CD69, and mock transduced [empty CAR]). Data are presented as mean±SEM of 2 to 5 different experiments.

FIGS. 19A-19B. Representative flow cytometry plots (FIG. 19A) and frequencies of T cell subsets (FIG. 19B) from day 1 to the end of CAR-T cell manufacturing (day 14). Immunophenotype was determined by flow cytometry: naïve (Tₙ) and stem cell memory (T_{scm}), CD197(CCR7)⁺CD45RO⁻; central memory (T_{cm}), CD197(CCR7)⁺CD45RO⁺; terminally differentiated (T_{ef}), CD197(CCR7)⁻CD45RO⁻; effector memory (Tₑₘ) and transitional memory (Tₜₘ), CD197(CCR7)⁻CD45RO⁺. Bars indicate the different CAR-T cell products (generated with ScFv sequences B8 or F12 for CD84, and G1 or H3 for CD69, and mock transduced [empty CAR]).

FIGS. 20A-20B. Immunophenotype of CAR-T cells to evaluate their activation (FIG. 20A) and exhaustion (FIG. 20B) by expression of the indicated markers was determined by flow cytometry at day 1 and at day 17. Data represent mean±SEM of 1 to 4 different experiments. Bars indicate the different CAR-T cell products (generated with ScFv sequences B8 or F12 for CD84, and G1 or H3 for CD69, and mock transduced [empty CAR]).

FIG. 21. T cells engineered to express the CD84 (F12, B8) CARs and CD69 (G1, H3) CARs recognize and kill target AML cell lines. Target HL-60 and SHI-1 (CD84⁺ CD69⁺) AML cell lines were grown in co-culture with TexMACS media (--), or effector CAR-T cells in a E:T ratio of 1:1 for 48 hours. Target AML cell lines (monitored by CD33⁺) stained with 7AAD and Annexin-V were analyzed by flow cytometry. SHI-1 and HL-60 cell lines had a higher proportion of cell death when co-cultured with CAR-T cells than that with mock transduced cells (empty CAR) (*P<0.05, **P<0.01 vs. empty CAR). Bars indicate the different CAR-T cell products (generated with ScFv sequences B8 or F12 for CD84, and G1 or H3 for CD69, and empty CAR). Data are represented as mean:SEM of 1 to 4 different experiments.

FIG. 22. Each bar represents the percentage of killing (% of lysis potency) exerted by a different CAR-T product on target AML cell lines. Bars indicate the different CAR-T cell products (generated with ScFv sequences B8 or F12 for CD84, and G1 or H3 for CD69, and mock transduced [empty CAR]). Data are represented as mean±SEM of 1 to 3 different experiments (*P<0.05 vs. empty CAR). CAR-T cell lysis potency is calculated as follows:

$$\% of lysis = 100 - \frac{N of alive AML cells in coculture with CAR T cells}{N of alive AML cells cultured alone} * 100$$

FIG. 23. Percentage of CAR-T cell lysis measured by bioluminescence (BLI) in luciferase (LUC)-transduced target AML cell lines. Each bar represents the percentage of killing exerted by a different CAR-T product on AML-LUC cell lines, normalized respect to BLI values of the relative AML cell line cultured alone. Bars indicate the different CAR-T cell products (generated with ScFv sequences B8 or F12 for CD84, and G1 or H3 for CD69, and mock transduced [empty CAR]). Data are represented as mean±SEM of 2 different experiments (***P<0.001; ****P<0.0001, vs. empty CAR).

FIG. 24. Absolute number of live CAR-T cells cultured alone (-) or with target AML cell lines at an effector:target (E:T) ratio of 1:1 for 48 hours, quantified by flow cytometry. Data show persistence of effector cells and lysis of the target AML cells. The dotted line represents CAR-T cells at day 17 prior to the co-culture. Each symbol indicates the different CAR-T cell products (generated with ScFv sequences B8 or F12 for CD84, and G1 or H3 for CD69, and mock transduced [empty CAR]) and cell lines.

FIGS. 25A-25B. (FIG. 25A) Representative images and (FIG. 25B) absolute number of colony forming units (CFU) generated from CD34⁺ cells cultured alone ( . . . ) or with CAR-T cells at an E:T ratio of 1:1 for 6 hours and then plated in MethoCult for 14 days. Bar and symbols indicate the different CAR-T cell products (generated with ScFv sequences B8 or F12 for CD84, and G1 or H3 for CD69, and mock transduced [empty CAR]) and human CD34⁺ cells. Data are represented as mean±SEM of 2-3 different experiments (n.s. p>0.05 not significant).

FIGS. 26A-26C. Luciferase bioluminescence signals observed in leukemic murine xenografts is provided in FIG. 26A. It shows that CD69 (H3) CAR-T cell effect on a CD69+ AML cell line growth in leukemic murine xenografts. FIG. 26B provides in vivo lysis potency of corresponding AML cell lines by CAR-T cells represented by luciferase signal reduction (represented as total flux) (*P<0.05 vs. mock transduced cells [empty CAR]). FIG. 26C shows CD69 (H3 and G1) and CD84 (B8 and F12) CAR-T cells in vivo lysis potency on CD69+CD84+ AML cell line (*P<0.05 with respect to empty CAR). NSG mice were injected with 0.5×106 SHI-1-LUC cells and, 2 days later, with 1.5×10⁶ CAR-T (1:3 ratio. n=7 animals/group). Data are represented as mean±SEM. AML engraftment and spread were monitored weekly by luciferase bioluminescence.

FIG. 27 provides nucleotide sequences of the CAR-T constructs (SEQ ID NOs.: 24-34) and protein sequences (SEQ ID NOs: 35-44 and 36) of the CAR-T constructs, respectively, in order of appearance.

### 3. DETAILED DESCRIPTION

### 3.1. Definitions

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification, unless otherwise indicated. Enzymatic reactions and purification techniques are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are those well-known and commonly used in the art. Standard techniques can be used for chemical syntheses, chemical analyses, pharmaceutical preparation, formulation, and delivery, and treatment of patients.

The following terms, unless otherwise indicated, shall be understood to have the following meanings.

Unless further modified by the name of a non-human species, the terms “CD63,” “CD63 protein,” and “CD63 antigen” are used interchangeably herein to refer to human CD63, or any variants (e.g., splice variants and allelic variants), isoforms, or species orthologs of human CD63 that are naturally expressed by cells, including neoplastic cells, or that are expressed by cells transfected with a CD63 gene (NCBI Accession No.: NG_008347, gene ID 967).

Unless further modified by the name of a non-human species, the terms “CD151,” “CD151 protein,” and “CD151 antigen” are used interchangeably herein to refer to human CD151, or any variants (e.g., splice variants and allelic variants), isoforms, or species orthologs of human CD151 that are naturally expressed by cells, including neoplastic cells, or that are expressed by cells transfected with a CD151 gene (NCBI Accession No.: NG_007478.1, gene ID 977).

Unless further modified by the name of a non-human species, the terms “CD72,” “CD72 protein,” and “CD72 antigen” are used interchangeably herein to refer to human CD72, or any variants (e.g., splice variants and allelic variants), isoforms, or species orthologs of human CD72 that are naturally expressed by cells, including neoplastic cells, or that are expressed by cells transfected with a CD72 gene (NCBI Accession No.: NC_000009.12, gene ID 971).

Unless further modified by the name of a non-human species, the terms “CD84,” “CD84 protein,” and “CD84 antigen” are used interchangeably herein to refer to human CD84, or any variants (e.g., splice variants and allelic variants), isoforms, or species orthologs of human CD84 that are naturally expressed by cells, including neoplastic cells, or that are expressed by cells transfected with a CD84 gene (NCBI Accession No.: NC_000001.11, gene ID 8832).

Unless further modified by the name of a non-human species, the terms “CD69,” “CD69 protein,” and “CD69 antigen” are used interchangeably herein to refer to human CD69, or any variants (e.g., splice variants and allelic variants), isoforms, or species orthologs of human CD69 that are naturally expressed by cells, including neoplastic cells, or that are expressed by cells transfected with a CD69 gene (NCBI Accession No.: NC_000012.12, gene ID 969).

Unless further modified by the name of a non-human species, the terms “CD109,” “CD109 protein,” and “CD109 antigen” are used interchangeably herein to refer to human CD69, or any variants (e.g., splice variants and allelic variants), isoforms, or species orthologs of human CD109 that are naturally expressed by cells, including neoplastic cells, or that are expressed by cells transfected with a CD109 gene (NCBI Accession No.: NC_000006.12, gene ID 135228).

The term “antigen-binding protein” (ABP) refers to a protein comprising one or more antigen-binding domains that specifically bind to an antigen or epitope. In some embodiments, the antigen-binding domain binds the antigen or epitope with specificity and affinity similar to that of naturally occurring antibodies. In some embodiments, the ABP comprises an antibody. In some embodiments, the ABP consists of an antibody. In some embodiments, the ABP consists essentially of an antibody. In some embodiments, the ABP comprises an alternative scaffold. In some embodiments, the ABP consists of an alternative scaffold. In some embodiments, the ABP consists essentially of an alternative scaffold. In some embodiments, the ABP comprises an antibody fragment. In some embodiments, the ABP consists of an antibody fragment. In some embodiments, the ABP consists essentially of an antibody fragment. For example, A “CD63,” “anti-CD63 ABP,” or “CD63-specific ABP” is an ABP, as provided herein, which specifically binds to the antigen CD63. In some embodiments, the ABP binds the extracellular domain of CD63, CD151, CD72, CD84, CD69, or CD109. In certain embodiments, a CD63, CD151, CD72, CD84, CD69, or CD109 ABP provided herein binds to an epitope of CD151, CD72, CD84, CD69, or CD109, respectively, that is conserved between or among CD151, CD72, CD84, CD69, or CD109 proteins from different species.

The term “antibody” is used herein in its broadest sense and includes certain types of immunoglobulin molecules comprising one or more antigen-binding domains that specifically bind to an antigen or epitope. An antibody specifically includes intact antibodies (e.g., intact immunoglobulins), antibody fragments, and multi-specific antibodies. One example of an antigen-binding domain is an antigen-binding domain formed by a V_{H}-V_{L} dimer. An antibody is one type of ABP.

The term “antigen-binding domain” means the portion of an ABP that is capable of specifically binding to an antigen or epitope.

The terms “full length antibody,” “intact antibody,” and “whole antibody” are used herein interchangeably to refer to an antibody having a structure substantially similar to a naturally occurring antibody structure and having heavy chains that comprise an Fc region.

The term “Fc region” means the C-terminal region of an immunoglobulin heavy chain that, in naturally occurring antibodies, interacts with Fc receptors and certain proteins of the complement system. The structures of the Fc regions of various immunoglobulins, and the glycosylation sites contained therein, are known in the art. See Schroeder and Cavacini, J. Allergy Clin. Immunol., 2010, 125:S41-52, incorporated by reference in its entirety. The Fc region may be a naturally occurring Fc region, or an Fc region modified as described elsewhere in this disclosure.

An “antibody fragment” comprises a portion of an intact antibody, such as the antigen-binding or variable region of an intact antibody. Antibody fragments include, for example, Fv fragments, Fab fragments, F(ab′)2 fragments, Fab′ fragments, scFv (sFv) fragments, and scFv-Fc fragments.

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (C_{H}1) of the heavy chain. Fab fragments may be generated, for example, by recombinant methods or by papain digestion of a full-length antibody.

“F(ab′)2” fragments contain two Fab′ fragments joined, near the hinge region, by disulfide bonds. F(ab′)2 fragments may be generated, for example, by recombinant methods or by pepsin digestion of an intact antibody. The F(ab′) fragments can be dissociated, for example, by treatment with ß-mercaptoethanol.

“Single-chain Fv” or “sFv” or “scFv” antibody fragments comprise a V_{H} domain and a V_{L} domain in a single polypeptide chain. The V_{H} and V_{L} are generally linked by a peptide linker. See Plückthun A. (1994). In some embodiments, the linker is a (GGGGS)n (SEQ ID NO: 1). In some embodiments, n=1, 2, 3, 4, 5, or 6. See Antibodies from Escherichia coli. In Rosenberg M. & Moore G. P. (Eds.), The Pharmacology of Monoclonal Antibodies vol. 113 (pp. 269-315). Springer-Verlag, New York, incorporated by reference in its entirety.

“scFv-Fc” fragments comprise an scFv attached to an Fc domain. For example, an Fe domain may be attached to the C-terminal of the scFv. The Fe domain may follow the V_{H} or V_{L}, depending on the orientation of the variable domains in the scFv (i.e., V_{H}-V_{L} or V_{L}-V_{H}). Any suitable Fe domain known in the art or described herein may be used. In some cases, the Fe domain comprises an IgG4 Fe domain.

The term “single domain antibody” refers to a molecule in which one variable domain of an antibody specifically binds to an antigen without the presence of the other variable domain.

A “monospecific ABP” is an ABP that comprises a binding site that specifically binds to a single epitope. An example of a monospecific ABP is a naturally occurring IgG molecule which, while divalent, recognizes the same epitope at each antigen-binding domain. The binding specificity may be present in any suitable valency.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “chimeric antibody” refers to an antibody in which a portion of the heavy and/or light chain is derived from a particular source or species, while the remainder of the heavy and/or light chain is derived from a different source or species.

“Humanized” forms of non-human antibodies are chimeric antibodies that contain minimal sequence derived from the non-human antibody. A humanized antibody is generally a human antibody (recipient antibody) in which residues from one or more CDRs are replaced by residues from one or more CDRs of a non-human antibody (donor antibody). The donor antibody can be any suitable non-human antibody, such as a mouse, rat, rabbit, chicken, camelid, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

An “isolated ABP” or “isolated nucleic acid” is an ABP or nucleic acid that has been separated and/or recovered from a component of its natural environment. Components of the natural environment may include enzymes, hormones, and other proteinaceous or nonproteinaceous materials. In some embodiments, an isolated ABP is purified to a degree sufficient to obtain at least 15 residues of N-terminal or internal amino acid sequence, for example by use of a spinning cup sequenator. In some embodiments, an isolated ABP is purified to homogeneity by gel electrophoresis (e.g., SDS-PAGE) under reducing or nonreducing conditions, with detection by Coomassie blue or silver stain. An isolated ABP includes an ABP in situ within recombinant cells, since at least one component of the ABP's natural environment is not present. In some embodiments, an isolated ABP or isolated nucleic acid is prepared by at least one purification step. In some embodiments, an isolated ABP or isolated nucleic acid is purified to at least 80%, 85%, 90%, 95%, or 99% by weight. In some embodiments, an isolated ABP or isolated nucleic acid is purified to at least 80%, 85%, 90%, 95%, or 99% by volume. In some embodiments, an isolated ABP or isolated nucleic acid is provided as a solution comprising at least 85%, 90%, 95%, 98%, 99% to 100% ABP or nucleic acid by weight. In some embodiments, an isolated ABP or isolated nucleic acid is provided as a solution comprising at least 85%, 90%, 95%, 98%, 99% to 100% ABP or nucleic acid by volume.

“Affinity” refers to the strength of the sum of non-covalent interactions between a single binding site of a molecule (e.g., an ABP) and its binding partner (e.g., an antigen or epitope). Unless indicated otherwise, as used herein, “affinity” refers to intrinsic binding affinity, which reflects a 1:1 interaction between members of a binding pair (e.g., ABP and antigen or epitope). The affinity of a molecule X for its partner Y can be represented by the dissociation equilibrium constant (K_{D}). The kinetic components that contribute to the dissociation equilibrium constant are described in more detail below. Affinity can be measured by common methods known in the art, including those described herein. Affinity can be determined, for example, using surface plasmon resonance (SPR) technology (e.g., BIACORE®) or biolayer interferometry (e.g., FORTEBIO®), or by monoclonal competitive ELISA tests.

With regard to the binding of an ABP to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the ABP to the target molecule is competitively inhibited by the control molecule. In some embodiments, the affinity of a CD63, CD151, CD72, CD84, CD69, or CD109 ABP for a non-target molecule is less than about 50% of the affinity for CD63, CD151, CD72, CD84, CD69, or CD109, respectively. In some embodiments, the affinity of a CD63, CD151, CD72, CD84, CD69, or CD109 ABP for a non-target molecule is less than about 40% of the affinity for CD63, CD151, CD72, CD84, CD69, or CD109, respectively. In some embodiments, the affinity of a CD63, CD151, CD72, CD84, CD69, or CD109 ABP for a non-target molecule is less than about 30% of the affinity for CD63, CD151, CD72, CD84, CD69, or CD109. In some embodiments, the affinity of a CD63, CD151, CD72, CD84, CD69, or CD109 ABP for a non-target molecule is less than about 20% of the affinity for CD63, CD151, CD72, CD84, CD69, or CD109, respectively. In some embodiments, the affinity of a CD63, CD151, CD72, CD84, CD69, or CD109 ABP for a non-target molecule is less than about 10% of the affinity for CD63, CD151, CD72, CD84, CD69, or CD109, respectively. In some embodiments, the affinity of a CD63, CD151, CD72, CD84, CD69, or CD109 ABP for a non-target molecule is less than about 1% of the affinity for CD63, CD151, CD72, CD84, CD69, or CD109, respectively. In some embodiments, the affinity of a CD63, CD151, CD72, CD84, CD69, or CD109 ABP for a non-target molecule is less than about 0.1% of the affinity for CD63, CD151, CD72, CD84, CD69, or CD109, respectively.

The term “kd” (sec-1), as used herein, refers to the dissociation rate constant of a particular ABP-antigen interaction. This value is also referred to as the koff value.

The term “ka” (M-1×sec-1), as used herein, refers to the association rate constant of a particular ABP-antigen interaction. This value is also referred to as the kon value.

The term “K_{D}” (M), as used herein, refers to the dissociation equilibrium constant of a particular ABP-antigen interaction. K_{D}=kd/ka.

The term “K_{A}” (M-1), as used herein, refers to the association equilibrium constant of a particular ABP-antigen interaction. K_{A}=ka/kd.

An “affinity matured” ABP is one with one or more alterations (e.g., in one or more CDRs or FRs) that result in an improvement in the affinity of the ABP for its antigen, compared to a parent ABP which does not possess the alteration(s). In one embodiment, an affinity matured ABP has nanomolar or picomolar affinity for the target antigen. Affinity matured ABPs may be produced using a variety of methods known in the art. For example, Marks et al. (Bio/Technology, 1992, 10:779-783, incorporated by reference in its entirety) describes affinity maturation by VH and VL domain shuffling. Random mutagenesis of CDR and/or framework residues is described by, for example, Barbas et al. (Proc. Nat. Acad. Sci. U.S.A., 1994, 91:3809-3813); Schier et al., Gene, 1995, 169:147-155; Yelton et al., J. Immunol., 1995, 155:1994-2004; Jackson et al., J. Immunol., 1995, 154:3310-33199; and Hawkins et al, J. Mol. Biol., 1992, 226:889-896; each of which is incorporated by reference in its entirety.

An “immunoconjugate” is an ABP conjugated to one or more heterologous molecule(s).

“Effector functions” refer to those biological activities mediated by the Fc region of an antibody, which activities may vary depending on the antibody isotype. Examples of antibody effector functions include C1q binding to activate complement dependent cytotoxicity (CDC), Fc receptor binding to activate antibody-dependent cellular cytotoxicity (ADCC), and antibody dependent cellular phagocytosis (ADCP).

When used herein in the context of two or more ABPs, the term “competes with” or “cross-competes with” indicates that the two or more ABPs compete for binding to an antigen (e.g., CD63, CD151, CD72, CD84, CD69, or CD109). In one exemplary assay, CD63, CD151, CD72, CD84, CD69, or CD109 is coated on a surface and contacted with a first CD63, CD151, CD72, CD84, CD69, or CD109 ABP, respectively, after which a second CD63, CD151, CD72, CD84, CD69, or CD109 ABP is added. In another exemplary assay, a first CD63, CD151, CD72, CD84, CD69, or CD109 ABP is coated on a surface and contacted with CD63, CD151, CD72, CD84, CD69, or CD109, and then a second CD63, CD151, CD72, CD84, CD69, or CD109 ABP is added. If the presence of the first CD63, CD151, CD72, CD84, CD69, or CD109 ABP reduces binding of the second CD63, CD151, CD72, CD84, CD69, or CD109 ABP, in either assay, then the ABPs compete. The term “competes with” also includes combinations of ABPs where one ABP reduces binding of another ABP, but where no competition is observed when the ABPs are added in the reverse order. However, in some embodiments, the first and second ABPs inhibit binding of each other, regardless of the order in which they are added. In some embodiments, one ABP reduces binding of another ABP to its antigen by at least 25%, at least 50%, at least 60%, at least 70%, at least 80%, at least 85%, at least 90%, or at least 95%. A skilled artisan can select the concentrations of the antibodies used in the competition assays based on the affinities of the ABPs for CD63, CD151, CD72, CD84, CD69, or CD109 and the valency of the ABPs. The assays described in this definition are illustrative, and a skilled artisan can utilize any suitable assay to determine if antibodies compete with each other. Suitable assays are described, for example, in Cox et al., “Immunoassay Methods,” in Assay Guidance Manual [Internet], Updated Dec. 24, 2014 (www.ncbi.nlm.nih.gov/books/NBK92434/; accessed Sep. 29, 2015); Silman et al., Cytometry, 2001, 44:30-37; and Finco et al., J. Pharm. Biomed. Anal., 2011, 54:351-358; each of which is incorporated by reference in its entirety.

The term “epitope” means a portion of an antigen the specifically binds to an ABP. Epitopes frequently consist of surface-accessible amino acid residues and/or sugar side chains and may have specific three-dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. The epitope to which an ABP binds can be determined using known techniques for epitope determination such as, for example, testing for ABP binding to CD63, CD151, CD72, CD84, CD69, or CD109 variants with different point-mutations, or to chimeric CD63, CD151, CD72, CD84, CD69, or CD109 variants.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

The term “treating” (and variations thereof such as “treat” or “treatment”) refers to clinical intervention in an attempt to alter the natural course of a disease or condition in a subject in need thereof. Treatment can be performed both for prophylaxis and during the course of clinical pathology. Desirable effects of treatment include preventing occurrence or recurrence of disease, alleviation of symptoms, diminish of any direct or indirect pathological consequences of the disease, preventing metastasis, decreasing the rate of disease progression, amelioration or palliation of the disease state, and remission or improved prognosis.

As used herein, the term “subject” means a mammalian subject. Exemplary subjects include humans, monkeys, dogs, cats, mice, rats, cows, horses, camels, goats, rabbits, and sheep. In certain embodiments, the subject is a human. In some embodiments the subject has a disease or condition that can be treated with an ABP, ABP-drug conjugated, or immunoresponsive cells comprising a CAR provided herein. In some embodiments, the disease or condition is a cancer.

The term “cytotoxic agent,” as used herein, refers to a substance that inhibits or prevents a cellular function and/or causes cell death or destruction.

The term “myeloid”, as used herein, includes all cells belonging to the granulocyte (i.e., neutrophil, eosinophil, basophil), monocyte/macrophage, erythroid, megakaryocyte, and mast cell lineages. Myeloid malignancies are clonal diseases of hematopoietic stem or progenitor cells. These malignancies can be present in the bone marrow and peripheral blood. They can result from genetic and epigenetic alterations that perturb key processes such as self-renewal, proliferation and impaired differentiation.

### 3.2. Other Interpretational Conventions

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless otherwise indicated, reference to a compound that has one or more stereocenters intends each stereoisomer, and all combinations of stereoisomers, thereof.

### 3.3. Compositions Targeting Tumor Specific Antigen

One aspect of the present disclosure relates to a protein targeting a tumor specific antigen selected from CD63, CD151, CD72, CD84, CD69, and CD109.

In some embodiments, the tumor specific antigen is CD63. In some embodiments, CD63 is a protein encoded by the CD63 gene (12q13.2) (NCBI Accession No.: NG_008347, gene ID 967).

In some embodiments, the tumor specific antigen is CD151. In some embodiments, CD151 is a protein encoded by the CD151 gene (lip15.5) (NCBI Accession No.: NG_007478.1, gene ID 977). Multiple alternatively spliced transcript variants that encode the same protein have been described for this gene. Any of the splice variants can be used in various embodiments.

In some embodiments, the tumor specific antigen is CD72. In some embodiments, CD72 is a protein encoded by the CD72 gene (9p13.3) (NCBI Accession No.: NC_000009.12, gene ID 971).

In some embodiments, the tumor specific antigen is CD84. In some embodiments, CD84 is a protein encoded by the CD84 gene (1q23.3) (NCBI Accession No.: NC_000001.11, gene ID 8832).

In some embodiments, the tumor specific antigen is CD69. In some embodiments, CD69 is a protein encoded by the CD69 gene (12pl3.31) (NCBI Accession No.: NC_000012.12, gene ID 969).

In some embodiments, the tumor specific antigen is CD109. In some embodiments, CD109 is a protein encoded by the CD109 gene (6q13) (NCBI Accession No.: NC_000006.12, gene ID 135228).

### 3.3.1. Antigen-Binding Protein (ABP)

In one aspect, the present disclosure provides an antigen-binding protein (ABP) that specifically binds to CD63, CD151, CD72, CD84, CD69, or CD109.

In certain embodiments, the ABP specifically binds to CD63. In certain embodiments, the ABP specifically binds to CD151. In certain embodiments, the ABP specifically binds to CD72. In certain embodiments, the ABP specifically binds to CD84. In certain embodiments, the ABP specifically binds to CD69. In certain embodiments, the ABP specifically binds to CD109. In some embodiments, the ABP is an isolated antigen binding protein (ABP) that specifically binds human CD63, CD151, CD72, CD84, CD69, or CD109. In certain embodiments, the ABP specifically binds to CD84 or CD69.

In some embodiments, the ABP comprises a human Fc.

In some embodiments, the ABP is human, humanized, or chimeric.

In particular embodiments, the ABP is monoclonal.

In some embodiments, the ABP is capable of inducing antibody-dependent cell-mediated cytotoxicity (ADCC) when administered. In some embodiments, the ABP that binds to cell surface CD63, CD151, CD72, CD84, CD69, or CD109, effects antibody-dependent cell-mediated cytotoxicity (ADCC). In some embodiments, natural killer (NK) cells effect ADCC by binding of the ABP's Fc domain to CD16 on the NK cell surface.

In some embodiments, the ABP comprises an antibody fragment. In some embodiments, the ABP comprises an immunoglobulin constant region. An antibody fragment may also be any synthetic or genetically engineered protein. For example, antibody fragments include isolated fragments consisting of the light chain variable region, “Fv” fragments consisting of the variable regions of the heavy and light chains, recombinant single chain polypeptide molecules in which light and heavy variable regions are connected by a peptide linker (scFv proteins).

Another form of an antibody fragment is a peptide comprising one or more complementarity determining regions (CDRs) of an antibody. CDRs (also termed “minimal recognition units”, or “hypervariable region”) can be incorporated into a molecule either covalently or noncovalently to make it an antigen binding protein. CDRs can be obtained by constructing polynucleotides that encode the CDR of interest. Such polynucleotides are prepared, for example, by using the polymerase chain reaction to synthesize the variable region using mRNA of antibody producing cells as a template (see, for example, Larrick et al., Methods: A Companion to Methods in Enzymology 2:106, 1991; Courtenay Luck, “Genetic Manipulation of Monoclonal Antibodies,” in Monoclonal Antibodies: Production, Engineering and Clinical Application, Ritter et al. (eds.), page 166 (Cambridge University Press 1995); and Ward et al., “Genetic Manipulation and Expression of Antibodies,” in Monoclonal Antibodies: Principles and Applications, Birch et al., (eds.), page 137 (Wiley Liss, Inc. 1995).

Thus, in one embodiment, the antibody fragment comprises at least one CDR as described herein. The binding agent may comprise at least two, three, four, five or six CDR's as described herein. The antibody fragment may further comprise at least one variable region domain of an antibody described herein. The variable region domain may be of any size or amino acid composition and will generally comprise at least one CDR sequence responsible for binding to human CD63, CD151, CD72, CD84, CD69, or CD109, for example HCDR1, HCDR2, HCDR3, LCDR1, LCDR2, and LCDR3, specifically described herein and which is adjacent to or in frame with one or more framework sequences. In general terms, the variable (V) region domain may be any suitable arrangement of immunoglobulin heavy (V_{H}) and/or light (V_{L}) chain variable domains. Thus, for example, the V region domain may be monomeric and be a V_{H} or V_{L} domain, which is capable of independently binding to a target antigen with an affinity ranging from 1 nM to 1 pM. Alternatively, the V region domain may be dimeric and contain V_{H} V_{H}, V_{H} V_{L}, or V_{L} V_{L}, dimers. The V region dimer comprises at least one V_{H} and at least one V_{L} chain that may be non-covalently associated (hereinafter referred to as Fv). If desired, the chains may be covalently coupled either directly, for example via a disulfide bond between the two variable domains, or through a linker, for example a peptide linker, to form a single chain Fv (scFv).

The variable region domain may be any naturally occurring variable domain or an engineered version thereof. By engineered version is meant a variable region domain that has been created using recombinant DNA engineering techniques. Such engineered versions include those created, for example, from a specific antibody variable region by insertions, deletions, or changes in or to the amino acid sequences of the specific antibody. Particular examples include engineered variable region domains containing at least one CDR and optionally one or more framework amino acids from a first antibody and the remainder of the variable region domain from a second antibody.

The variable region domain may be covalently attached at a C terminal amino acid to at least one other antibody domain or a fragment thereof. Thus, for example, a V_{H} domain that is present in the variable region domain may be linked to an immunoglobulin CH1 domain, or a fragment thereof. Similarly, a V_{L} domain may be linked to a CK domain or a fragment thereof. In this way, for example, the antibody may be a Fab fragment wherein the antigen binding domain contains associated V_{H} and V_{L} domains covalently linked at their C termini to a CH1 and CK domain, respectively. The CH1 domain may be extended with further amino acids, for example to provide a hinge region or a portion of a hinge region domain as found in a Fab′ fragment, or to provide further domains, such as antibody CH2 and CH3 domains.

As described herein, antibodies comprise at least one of these CDRs. For example, one or more CDR may be incorporated into known antibody framework regions (IgG1, IgG2, etc.), or conjugated to a suitable vehicle to enhance the half-life thereof. Suitable vehicles include, but are not limited to Fc, polyethylene glycol (PEG), albumin, transferrin, and the like. These and other suitable vehicles are known in the art. Such conjugated CDR peptides may be in monomeric, dimeric, tetrameric, or other form. In one embodiment, one or more water-soluble polymer is bonded at one or more specific position, for example at the amino terminus, of a binding agent.

ABPs of the present disclosure that specifically binds to CD84 or CD69 can be found in Table 10 (V_{L} and V_{H} CDRs) and Table 11 (light chain and heavy chain variable regions.

TABLE 10
V_{L} and V_{H} CDR sequences of ABPs

| Name | Target | LC CDR1 | LC CDR2 | LC CDR3 | HC CDR1 | HC CDR2 | HC CDR3 |
|---|---|---|---|---|---|---|---|
| A1 | | SGSSSNIGSNYVA | DTNKRPS | GTWDSSLSAEV | SNTASWN | KTEYRSGWFFEYAPSVGG | DSYNDYDNDGFDI |
| | | (SEQ ID NO: 45) | (SEQ ID | (SEQ ID NO: 55) | (SEQ ID | (SEQ ID NO: 65) | (SEQ ID NO: 70) |
| | | | NO: 52) | | NO: 62) | | |
| C1 | | SGSSSNIGSNYVA | DTNKRPS | GTWDSSLSAEV | SNSASWN | KTEYRSKWYYEFAPSVKS | DSYNGYDFDGFDI |
| | | (SEQ ID NO: 45) | (SEQ ID | (SEQ ID NO: 55) | (SEQ ID | (SEQ ID NO: 66) | (SEQ ID NO: 71) |
| | | | NO: 52) | | NO: 63) | | |
| F1 | | SGSSSNVGNNYVA | DNYKRPS | GSWDSSLSAEV | SNSASWN | KTEYRSKWFDEYAPSVKG | DSYNGYDFDGFDI |
| | | (SEQ ID NO: 46) | (SEQ ID | (SEQ ID NO: 56) | (SEQ ID | (SEQ ID NO: 67) | (SEQ ID NO: 71) |
| | | | NO: 53) | | NO: 63) | | |
| G1 | CD69 | SGSSSNIGNNYVS | DNNKRPS | GTWDSSLSAEM | STTASWN | KTEYRSKWFDEYAPSVKG | DSYNGYDFDGFDI |
| | | (SEQ ID NO: 47) | (SEQ ID | (SEQ ID NO: 57) | (SEQ ID | (SEQ ID NO: 67) | (SEQ ID NO: 71) |
| | | | NO: 54) | | NO: 64) | | |
| C2 | | SGSSSNIGDNL VS | DNNKRPS | ESWDNNLTAEV | SNSASWN | KVEYRSKWYDEYAPSVKS | DSYNGHAFDGFDV |
| | | (SEQ ID NO: 48) | (SEQ ID | (SEQ ID NO: 58) | (SEQ ID | (SEQ ID NO: 68) | (SEQ ID NO: 72) |
| | | | NO: 54) | | NO: 63) | | |
| F2 | | SGGSSNIEKNYVS | DTNKRPS | GAWDNILNSEM | SNSASWN | KTEYRSKWFDEYAPSVKG | DSYNGYDFDGFDI |
| | | (SEQ ID NO: 49) | (SEQ ID | (SEQ ID NO: 59) | (SEQ ID | (SEQ ID NO: 67) | (SEQ ID NO: 71) |
| | | | NO: 52) | | NO: 63) | | |
| H2 | | SGGSSNIEKNYVS | DTNKRPS | GAWDNILNSEM | SNSASWN | KTEYRSKWFDEYAPSVKG | DSYNGYDFDGFDI |
| | | (SEQ ID NO: 49) | (SEQ ID | (SEQ ID NO: 59) | (SEQ ID | (SEQ ID NO: 67) | (SEQ ID NO: 71) |
| | | | NO: 52) | | NO: 63) | | |
| H3 | CD69 | SGRNSNIANNFVS | DNNKRPS | ESWDSSLTAEV | SNTASWN | KTEYRSQWLHEYAPSVRS | DSYNGYAFDGFDT |
| | | (SEQ ID NO: 50) | (SEQ ID | (SEQ ID NO: 60) | (SEQ ID | (SEQ ID NO: 69) | (SEQ ID NO: 73) |
| | | | NO: 54) | | NO: 62) | | |
| F12 | CD84 | SGSSSNIGNNYVS | DNNKRPS | GTWDSSLSAEV | SNSASWN | KVEYRSKWYDEYAPSVKS | DSYNGHAFDGFDV |
| | | (SEQ ID NO: 47) | (SEQ ID | (SEQ ID NO: 55) | (SEQ ID | (SEQ ID NO: 68) | (SEQ ID NO: 72) |
| | | | NO: 54) | | NO: 63) | | |
| B8 | CD84 | SGTTSNIANNFVS | DNNKRPS | GVWDNSLNAEV | SNSASWN | KVEYRSKWYDEYAPSVKS | DSYNGHAFDGFDV |
| | | (SEQ ID NO: 51) | (SEQ ID | (SEQ ID NO: 61) | (SEQ ID | (SEQ ID NO: 68) | (SEQ ID NO: 72) |
| | | | NO: 54) | | NO: 63) | | |
| B11 | | SGSSSNIGSNYVA | DTNKRPS | GTWDSSLSAEV | SNSASWN | KTEYRSKWYYEFAPSVKS | DSYNGYDFDGFDI |
| | | (SEQ ID NO: 45) | (SEQ ID | (SEQ ID NO: 55) | (SEQ ID | (SEQ ID NO: 66) | (SEQ ID NO: 71) |
| | | | NO: 52) | | NO: 63) | | |

In some embodiments, the ABP comprises: a light chain variable domain (V_{L}) CDR1 comprising an amino acid sequence having at least 90%, at least 95%, at least 97%, at least 98%, at least 99%, or 100% sequence identity to the amino acid sequence selected from: SEQ ID NOs.: 45-51 of Table 10.

In some embodiments, the ABP comprises a light chain variable domain (V_{L}) CDR2 comprising an amino acid sequence having at least 90%, at least 95%, at least 97%, at least 98%, at least 99%, or 100% sequence identity to the amino acid sequence selected from SEQ ID NOs: 52-54 of Table 10.

In some embodiments, the ABP comprises a light chain variable domain (V_{L}) CDR3 comprising an amino acid sequence having at least 90%, at least 95%, at least 97%, at least 98%, at least 99%, or 100% sequence identity to the amino acid sequence selected from: SEQ ID NOs: 55-61 of Table 10.

In some embodiments, the ABP comprises: (a) V_{L} CDR1 comprising an amino acid sequence having at least 90%, at least 95%, at least 97%, at least 98%, at least 99%, or 100% sequence identity to the amino acid sequence selected from: SEQ ID NOs: 45-51; (b) a V_{L} CDR2 comprising an amino acid sequence having at least 90%, at least 95%, at least 97%, at least 98%, at least 99%, or 100% sequence identity to the amino acid sequence selected from: SEQ ID NOs: 52-54; and (c) a V_{L} CDR3 comprising an amino acid sequence having at least 90%, at least 95%, at least 97%, at least 98%, at least 99%, or 100% sequence identity to the amino acid sequence selected from: SEQ ID NO: 55-61 of Table 10.

In some embodiments, the ABP comprises: (a) a light chain variable domain (V_{H}) CDR1 comprising an amino acid sequence having at least 90%, at least 95%, at least 97%, at least 98%, at least 99%, or 100% sequence identity to the amino acid sequence selected from: SEQ ID NOs.: 62-64. In some embodiments, the ABP comprises (b) a V_{H} CDR2 comprising an amino acid sequence having at least 90%, at least 95%, at least 97%, at least 98%, at least 99%, or 100% sequence identity to the amino acid sequence selected from: SEQ ID NOs.: 65-69. In some embodiments, the ABP comprises a V_{H} CDR3 comprising an amino acid sequence having at least 90%, at least 95%, at least 97%, at least 98%, at least 99%, or 100% sequence identity to the amino acid sequence selected from: SEQ ID NOs.: 70-73.

In some embodiments, the ABP comprises: (a) V_{H} CDR1 comprising an amino acid sequence having at least 90%, at least 95%, at least 97%, at least 98%, at least 99%, or 100% sequence identity to the amino acid sequence selected from: SNTASWN (SEQ ID NO: 62); SNSASWN (SEQ ID NO: 63); and STTASWN (SEQ ID NO: 64); (b) a V_{H} CDR2 comprising an amino acid sequence having at least 90%, at least 95%, at least 97%, at least 98%, at least 99%, or 100% sequence identity to the amino acid sequence selected from: SEQ ID NOs: 65-69; and (c) a V_{H} CDR3 comprising an amino acid sequence having at least 90%, at least 95%, at least 97%, at least 98%, at least 99%, or 100% sequence identity to the amino acid sequence selected from: SEQ ID NOs: 70-73.

In some embodiments, the ABP comprises CDR sequences identical to an antibody selected from A1, C1, F1, G1, C2, F2, H2, H3, F12, B8, and B11.

In some embodiments, the ABP comprises V_{L} CDR1 having the sequence of SEQ ID NO: 45, V_{L} CDR2 having the sequence of SEQ ID NO: 52, V_{L} CDR3 having the sequence of SEQ ID NO: 55, V_{H} CDR1 having the sequence of SEQ ID NO: 62, V_{H} CDR2 having the sequence of SEQ ID NO: 65, and V_{H} CDR3 having the sequence of SEQ ID NO: 70.

In some embodiments, the ABP comprises V_{L} CDR1 having the sequence of SEQ ID NO: 45, V_{L} CDR2 having the sequence of SEQ ID NO: 52, V_{L} CDR3 having the sequence of SEQ ID NO: 55, V_{H} CDR1 having the sequence of SEQ ID NO: 63, V_{H} CDR2 having the sequence of SEQ ID NO: 66, and V_{H} CDR3 having the sequence of SEQ ID NO: 71.

In some embodiments, the ABP comprises V_{L} CDR1 having the sequence of SEQ ID NO: 46, V_{L} CDR2 having the sequence of SEQ ID NO: 53, V_{L} CDR3 having the sequence of SEQ ID NO: 56, V_{H} CDR1 having the sequence of SEQ ID NO: 63, V_{H} CDR2 having the sequence of SEQ ID NO: 67, and V_{H} CDR3 having the sequence of SEQ ID NO: 71.

In some embodiments, the ABP comprises V_{L} CDR1 having the sequence of SEQ ID NO: 47, V_{L} CDR2 having the sequence of SEQ ID NO: 54, V_{L} CDR3 having the sequence of SEQ ID NO: 57, V_{H} CDR1 having the sequence of SEQ ID NO: 64, V_{H} CDR2 having the sequence of SEQ ID NO: 67, and V_{H} CDR3 having the sequence of SEQ ID NO: 71.

In some embodiments, the ABP comprises V_{L} CDR1 having the sequence of SEQ ID NO: 48, V_{L} CDR2 having the sequence of SEQ ID NO: 54, V_{L} CDR3 having the sequence of SEQ ID NO: 58, V_{H} CDR1 having the sequence of SEQ ID NO: 63, V_{H} CDR2 having the sequence of SEQ ID NO: 68, and V_{H} CDR3 having the sequence of SEQ ID NO: 72.

In some embodiments, the ABP comprises V_{L} CDR1 having the sequence of SEQ ID NO: 49, V_{L} CDR2 having the sequence of SEQ ID NO: 52, V_{L} CDR3 having the sequence of SEQ ID NO: 59, V_{H} CDR1 having the sequence of SEQ ID NO: 63, V_{H} CDR2 having the sequence of SEQ ID NO: 67, and V_{H} CDR3 having the sequence of SEQ ID NO: 71.

In some embodiments, the ABP comprises V_{L} CDR1 having the sequence of SEQ ID NO: 49, V_{L} CDR2 having the sequence of SEQ ID NO: 52, V_{L} CDR3 having the sequence of SEQ ID NO: 59, V_{H} CDR1 having the sequence of SEQ ID NO: 63, V_{H} CDR2 having the sequence of SEQ ID NO: 67, and V_{H} CDR3 having the sequence of SEQ ID NO: 71.

In some embodiments, the ABP comprises V_{L} CDR1 having the sequence of SEQ ID NO: 50, V_{L} CDR2 having the sequence of SEQ ID NO: 54, V_{L} CDR3 having the sequence of SEQ ID NO: 60, V_{H} CDR1 having the sequence of SEQ ID NO: 62, V_{H} CDR2 having the sequence of SEQ ID NO: 69, and V_{H} CDR3 having the sequence of SEQ ID NO: 73.

In some embodiments, the ABP comprises V_{L} CDR1 having the sequence of SEQ ID NO: 47, V_{L} CDR2 having the sequence of SEQ ID NO: 54, V_{L} CDR3 having the sequence of SEQ ID NO: 55, V_{H} CDR1 having the sequence of SEQ ID NO: 63, V_{H} CDR2 having the sequence of SEQ ID NO: 68, and V_{H} CDR3 having the sequence of SEQ ID NO: 72.

In some embodiments, the ABP comprises V_{L} CDR1 having the sequence of SEQ ID NO: 51, V_{L} CDR2 having the sequence of SEQ ID NO: 54, V_{L} CDR3 having the sequence of SEQ ID NO: 61, V_{H} CDR1 having the sequence of SEQ ID NO: 63, V_{H} CDR2 having the sequence of SEQ ID NO: 68, and V_{H} CDR3 having the sequence of SEQ ID NO: 72.

In some embodiments, the ABP comprises V_{L} CDR1 having the sequence of SEQ ID NO: 45, V_{L} CDR2 having the sequence of SEQ ID NO: 52, V_{L} CDR3 having the sequence of SEQ ID NO: 55, V_{H} CDR1 having the sequence of SEQ ID NO: 63, V_{H} CDR2 having the sequence of SEQ ID NO: 66, and V_{H} CDR3 having the sequence of SEQ ID NO: 71.

In some embodiments, the ABP comprises an antibody. In some embodiments, the ABP is a monoclonal antibody. In some embodiments, the ABP is selected from a human antibody, a humanized antibody, or a chimeric antibody. In some embodiments, the ABP is a single chain variable fragment (scFv). In some embodiments, the ABP comprises an antibody fragment. In some embodiments, the ABP comprises an immunoglobulin constant region.

In some embodiments, the ABP comprises a variable domain comprising an amino acid sequence having at least 80%, at least 85%, at least 90%, at least 95%, at least 97%, at least 99%, or at least 100% sequence identity to the amino acid sequence of: any one of SEQ ID NOs.: 74-88 of Table 11.

| Name | Target | Variable heavy chain sequence | Variable light chain sequence |
|---|---|---|---|
| A1 | | QVQLQQSGPGLVQPSRTLSLTCAISGDSISSNTAS | QSVLTQPPSVSAAPGQKVTISCSGSSSNIGSNYVA |
| | | WNWIRQSPSRGLEWVGKTEYRSGWFFEYAPSVGGR | WYQQLPGTAPKLLIYDTNKRPSGIPDRFSGSKSGT |
| | | VNISPDTSKNQFSLQLNSVTPEDTAVYFCARDSYN | SATLGITGLQTGDEADYYCGTWDSSLSAEVFGTGT |
| | | DYDNDGFDIWGQGTLVTVSS (SEQ ID NO: 82) | KVTVL (SEQ ID NO: 74) |
| C1 | | QVQLQQSGPGLVKPSQTLSLTCAISGDSVSSNSAS | QSVLTQPPSVSAAPGQKVTISCSGSSSNIGSNYVA |
| | | WNWIRQSPSRGLEWLGKTEYRSKWYYEFAPSVKSR | WYQQLPGTAPKLLIYDTNKRPSGIPDRFSGSKSGT |
| | | ITITPDTSKNQFSLQLNSVTPEDTAVYYCVRDSYN | SATLGITGLQTGDEADYYCGTWDSSLSAEVFGTGT |
| | | GYDFDGFDIWGQGTLVTVSS (SEQ ID NO: 83) | KVTVL (SEQ ID NO: 74) |
| F1 | | QVQLQQSGPGLVKPSQTLSLTCAISGDSVFSNSAS | QSVLTQPPSVSAAPGQQVIISCSGSSSNVGNNYVA |
| | | WNWIRQSPSRGLEWLGKTEYRSKWFDEYAPSVKGR | WYQQVPGAAPRLLIYDNYKRPSGIPDRFSGSKSGT |
| | | IAIRPDTSKNQVSLQLNSVTPEDTAVYYCVRDSYN | SATLGITGLQSGDEADYYCGSWDSSLSAEVFGGGT |
| | | GYDFDGFDIWGQGTLVTVSS (SEQ ID NO: 84) | KLTAL (SEQ ID NO: 75) |
| G1 | CD69 | QSVVTQPPSVSAAPGQKVTISCSGSSSNIGNNYVS | QSVVTQPPSVSAAPGQKVTISCSGSSSNIGNNYVS |
| | | WYQQLPGTAPKLLIYDNNKRPSGIPDRFSGSKSGT | WYQQLPGTAPKLLIYDNNKRPSGIPDRFSGSKSGT |
| | | SATLGITGLQTGDEADYYCGTWDSSLSAEMFGGGT | SATLGITGLQTGDEADYYCGTWDSSLSAEMFGGGT |
| | | KVTVL (SEQ ID NO: 85) | KVTV (SEQ ID NO: 76) |
| C2 | | QVQLQQSGPGLVKPSQTLSLTCAISGDSVSSNSAS | QSVLTQPPSVSAAPGQKVTISCSGSSSNIGDNLVS |
| | | WNWIRQSPSRGLEWLGKVEYRSKWYDEYAPSVKSR | WYQQLPGTAPKLLIYDNNKRPSGIPDRFSGSKSGT |
| | | IAIRPDTSKNQVSLHLISVTPDDTAVYYCARDSYN | SATLDITGLQTGDEADYYCESWDNNLTAEVFGTGT |
| | | GHAFDGFDVWGQGTLVTVSS (SEQ ID NO: 86) | KVTVL (SEQ ID NO: 77) |
| F2 | | QVQLQQSGPGLVKPSQTLSLTCAISGDSVFSNSAS | QSVLTQPPSVSAAPGQKVTISCSGSSSNIGDNLVS |
| | | WNWIRQSPSRGLEWLGKTEYRSKWFDEYAPSVKGR | WYQQLPGTAPKLLIYDNNKRPSGIPDRFSGSKSGT |
| | | IAIRPDTSKNQVSLQLNSVTPEDTAVYYCVRDSYN | SATLDITGLQTGDEADYYCESWDNNLTAEVFGTGT |
| | | GYDFDGFDIWGQGTLVTVSS (SEQ ID NO: 84) | KVTVL (SEQ ID NO: 77) |
| H2 | | QVQLQQSGPGLVKPSQTLSLTCAISGDSVFSNSAS | QSVVTQPPSVSAAPGQKVTISCSGSSSNIGNNYVS |
| | | WNWIRQSPSRGLEWLGKTEYRSKWFDEYAPSVKGR | WYQQLPGTAPKLLIYDNNKRPSGIPDRFSGSKSGT |
| | | IAIRPDTSKNQVSLQLNSVTPEDTAVYYCVRDSYN | SATLGITGLQTGDEADYYCGTWDSSLSAEVFGGGT |
| | | GYDFDGFDIWGQGTLVTVSS (SEQ ID NO: 84) | KLTVL (SEQ ID NO: 78) |
| H3 | CD69 | QVQLQQSGPGLVKPSQTLSLTCDISGDSVFSNTAS | QSVVTQPPSVSAAPGQKVTISCSGRNSNIANNFVS |
| | | WNWIRQSPSRGLEWLGKTEYRSQWLHEYAPSVRSR | WYRQLPGTAPKLLIYDNNKRPSGIPDRFSGSKSGT |
| | | IAIRPDTSKNQVSLQLSFVTPEDTAVYYCVRDSYN | SATLAITGLQTGDEADYYCESWDSSLTAEVFGTGT |
| | | GYAFDGFDTWGQGTLVTVSS (SEQ ID NO: 87) | KVTVL (SEQ ID NO: 79) |
| F12 | CD84 | QVQLQQSGPGLVKPSQTLSLTCAISGDSVSSNSAS | QSVVTQPPSVSAAPGQKVTISCSGSSSNIGNNYVS |
| | | WNWIRQSPSRGLEWLGKVEYRSKWYDEYAPSVKSR | WYQQLPGTAPKLLIYDNNKRPSGIPDRFSGSKSGT |
| | | IAIRPDTSKNQVSLHLISVTPDDTAVYYCARDSYN | SATLGITGLQTGDEADYYCGTWDSSLSAEVFGGGT |
| | | GHAFDGFDVWGQGTMVTVSS (SEQ ID NO: 88) | KVTVL (SEQ ID NO: 80) |
| B8 | CD84 | QVQLQQSGPGLVKPSQTLSLTCAISGDSVSSNSAS | QSVLTQPPSLSAAPGQRVTISCSGTTSNIANNFVS |
| | | WNWIRQSPSRGLEWLGKVEYRSKWYDEYAPSVKSR | WYQQLSGSAPKLLINDNNKRPSGIPDRFSGSKSGA |
| | | IAIRPDTSKNQVSLHLISVTPDDTAVYYCARDSYN | SATLAITGLQTEDEADYYCGVWDNSLNAEVFGGGT |
| | | GHAFDGFDVWGQGTMVTVSS (SEQ ID NO: 88) | KVTVL (SEQ ID NO: 81) |
| B11 | | QVQLQQSGPGLVKPSQTLSLTCAISGDSVSSNSAS | QSVLTQPPSVSAAPGQKVTISCSGSSSNIGSNYVA |
| | | WNWIRQSPSRGLEWLGKTEYRSKWYYEFAPSVKSR | WYQQLPGTAPKLLIYDTNKRPSGIPDRFSGSKSGT |
| | | ITITPDTSKNQFSLQLNSVTPEDTAVYYCVRDSYN | SATLGITGLQTGDEADYYCGTWDSSLSAEVFGTGT |
| | | GYDFDGFDIWGQGTLVTVSS (SEQ ID NO: 83) | KVTVL (SEQ ID NO: 74) |

In some embodiments, the ABP comprises a heavy chain variable domain comprising an amino acid sequence having at least 80%, at least 85%, at least 90%, at least 95%, at least 97%, at least 99%, or at least 100% sequence identity to the amino acid sequence of any one of SEQ ID NOs.: 82-88 of Table 11.

In some embodiments, the ABP comprises a light chain variable domain comprising an amino acid sequence having at least 80%, at least 85%, at least 90%, at least 95%, at least 97%, at least 99%, or at least 100% sequence identity to the amino acid sequence of any one of SEQ ID NOs.: 74-81 of Table 11.

In some embodiments, the ABP comprises a heavy chain variable domain and a light chain variable domain of an antibody selected from A1, C1, F1, G1, C2, F2, H2, H3, F12, B8, and B11.

In some embodiments, the ABP comprises a heavy chain variable domain having the sequence of SEQ ID NO: 82 and a light chain variable domain having the sequence of SEQ ID NO: 74.

In some embodiments, the ABP comprises a heavy chain variable domain having the sequence of SEQ ID NO: 83 and a light chain variable domain having the sequence of SEQ ID NO: 74.

In some embodiments, the ABP comprises a heavy chain variable domain having the sequence of SEQ ID NO: 84 and a light chain variable domain having the sequence of SEQ ID NO: 75.

In some embodiments, the ABP comprises a heavy chain variable domain having the sequence of SEQ ID NO: 85 and a light chain variable domain having the sequence of SEQ ID NO: 76.

In some embodiments, the ABP comprises a heavy chain variable domain having the sequence of SEQ ID NO: 86 and a light chain variable domain having the sequence of SEQ ID NO: 77.

In some embodiments, the ABP comprises a heavy chain variable domain having the sequence of SEQ ID NO: 84 and a light chain variable domain having the sequence of SEQ ID NO: 77.

In some embodiments, the ABP comprises a heavy chain variable domain having the sequence of SEQ ID NO: 84 and a light chain variable domain having the sequence of SEQ ID NO: 78.

In some embodiments, the ABP comprises a heavy chain variable domain having the sequence of SEQ ID NO: 87 and a light chain variable domain having the sequence of SEQ ID NO: 79.

In some embodiments, the ABP comprises a heavy chain variable domain having the sequence of SEQ ID NO: 88 and a light chain variable domain having the sequence of SEQ ID NO: 80.

In some embodiments, the ABP comprises a heavy chain variable domain having the sequence of SEQ ID NO: 88 and a light chain variable domain having the sequence of SEQ ID NO: 81.

In some embodiments, the ABP comprises a heavy chain variable domain having the sequence of SEQ ID NO: 83 and a light chain variable domain having the sequence of SEQ ID NO: 74.

In some embodiments, the ABP comprises an scFv. In some embodiments, the ABP is an scFv. In some embodiments, the scFv comprises an amino acid sequence having at least 80%, at least 85%, at least 90%, at least 95%, at least 97%, at least 98%, at least 99%, or 100% sequence identity to the amino acid sequence of any one of SEQ ID NOs.: 89-98 of Table 12.

TABLE 12
scFv amino acid sequences of ABPs

| | | SEQ ID |
|---|---|---|
| scFv | Amino Acid Sequence | NO: |
| A1 | QVQLQQSGPGLVQPSRTLSLTCAISGDSISSNTASWNWIRQSPSRGLEWVGKTEYRSGWFFE | 89 |
| | YAPSVGGRVNISPDTSKNQFSLQLNSVTPEDTAVYFCARDSYNDYDNDGFDIWGQGTLVTVS | |
| | SGGGGSGGGGSGGGGSGGGGSQSVLTQPPSVSAAPGQKVTISCSGSSSNIGSNYVAWYQQLP | |
| | GTAPKLLIYDTNKRPSGIPDRFSGSKSGTSATLGITGLQTGDEADYYCGTWDSSLSAEVFGT | |
| | GTKVTVL | |
| C1 | QVQLQQSGPGLVKPSQTLSLTCAISGDSVSSNSASWNWIRQSPSRGLEWLGKTEYRSKWYYE | 90 |
| | FAPSVKSRITITPDTSKNQFSLQLNSVTPEDTAVYYCVRDSYNGYDFDGFDIWGQGTLVTVS | |
| | SGGGGSGGGGSGGGGSGGGGSQSVLTQPPSVSAAPGQKVTISCSGSSSNIGSNYVAWYQQLP | |
| | GTAPKLLIYDTNKRPSGIPDRFSGSKSGTSATLGITGLQTGDEADYYCGTWDSSLSAEVFGT | |
| | GTKVTVL | |
| F1 | QVQLQQSGPGLVKPSQTLSLTCAISGDSVFSNSASWNWIRQSPSRGLEWLGKTEYRSKWFDE | 91 |
| | YAPSVKGRIAIRPDTSKNQVSLQLNSVTPEDTAVYYCVRDSYNGYDFDGFDIWGQGTLVTVS | |
| | SGGGGSGGGGSGGGGSGGGGSQSVLTQPPSVSAAPGQQVIISCSGSSSNVGNNYVAWYQQVP | |
| | GAAPRLLIYDNYKRPSGIPDRFSGSKSGTSATLGITGLQSGDEADYYCGSWDSSLSAEVFGG | |
| | GTKLTAL | |
| G1 | QVQLQQSGPGLVKPSQTLSLTCAISGDSVFSTTASWNWIRQSPSRGLEWLGKTEYRSKWFDE | 92 |
| | YAPSVKGRIAIRPDTSRNQVSLQLNSMTPEDTAVYFCVRDSYNGYDFDGFDIWGQGTTVTVS | |
| | SGGGGSGGGGSGGGGSGGGGSQSVVTQPPSVSAAPGQKVTISCSGSSSNIGNNYVSWYQQLP | |
| | GTAPKLLIYDNNKRPSGIPDRFSGSKSGTSATLGITGLQTGDEADYYCGTWDSSLSAEMFGG | |
| | GTKVTVL | |
| C2 | QVQLQQSGPGLVKPSQTLSLTCAISGDSVSSNSASWNWIRQSPSRGLEWLGKVEYRSKWYDE | 93 |
| | YAPSVKSRIAIRPDTSKNQVSLHLISVTPDDTAVYYCARDSYNGHAFDGFDVWGQGTLVTVS | |
| | SGGGGSGGGGSGGGGSGGGGSQSVLTQPPSVSAAPGQKVTISCSGSSSNIGDNLVSWYQQLP | |
| | GTAPKLLIYDNNKRPSGIPDRFSGSKSGTSATLDITGLQTGDEADYYCESWDNNLTAEVFGT | |
| | GTKVTVL | |
| F2 | QVQLQQSGPGLVKPSQTLSLTCAISGDSVFSNSASWNWIRQSPSRGLEWLGKTEYRSKWFDE | 94 |
| | YAPSVKGRIAIRPDTSKNQVSLQLNSVTPEDTAVYYCVRDSYNGYDFDGFDIWGQGTLVTVS | |
| | SGGGGSGGGGSGGGGSGGGGSQSVLTQPPSVSAAPGQKVTISCSGGSSNIEKNYVSWYQQLP | |
| | GTAPKLLIYDTNKRPSGIPDRFSGSKSGTSATLGITGLQTGDEADYYCGAWDNILNSEMFGG | |
| | GTKLTVL | |
| H2 | QVQLQQSGPGLVKPSQTLSLTCAISGDSVFSNSASWNWIRQSPSRGLEWLGKTEYRSKWFDE | 95 |
| | YAPSVKGRIAIRPDTSKNQVSLQLNSVTPEDTAVYYCVRDSYNGYDFDGFDIWGQGTLVTVS | |
| | SGGGGSGGGGSGGGGSGGGGSQSVVTQPPSVSAAPGQKVTISCSGSSSNIGNNYVSWYQQLP | |
| | GTAPKLLIYDNNKRPSGIPDRFSGSKSGTSATLGITGLQTGDEADYYCGTWDSSLSAEVFGG | |
| | GTKLTVL | |
| H3 | QVQLQQSGPGLVKPSQTLSLTCDISGDSVFSNTASWNWIRQSPSRGLEWLGKTEYRSQWLHE | 96 |
| | YAPSVRSRIAIRPDTSKNQVSLQLSFVTPEDTAVYYCVRDSYNGYAFDGFDTWGQGTLVTVS | |
| | SGGGGSGGGGSGGGGSGGGGSQSVVTQPPSVSAAPGQKVTISCSGRNSNIANNFVSWYRQLP | |
| | GTAPKLLIYDNNKRPSGIPDRFSGSKSGTSATLAITGLQTGDEADYYCESWDSSLTAEVFGT | |
| | GTKVTVL | |
| F12 | QVQLQQSGPGLVKPSQTLSLTCAISGDSVSSNSASWNWIRQSPSRGLEWLGKVEYRSKWYDE | 97 |
| | YAPSVKSRIAIRPDTSKNQVSLHLISVTPDDTAVYYCARDSYNGHAFDGFDVWGQGTMVTVS | |
| | SGGGGSGGGGSGGGGSGGGGSQSVVTQPPSVSAAPGQKVTISCSGSSSNIGNNYVSWYQQLP | |
| | GTAPKLLIYDNNKRPSGIPDRFSGSKSGTSATLGITGLQTGDEADYYCGTWDSSLSAEVFGG | |
| | GTKVTVL | |
| B8 | QVQLQQSGPGLVKPSQTLSLTCAISGDSVSSNSASWNWIRQSPSRGLEWLGKVEYRSKWYDE | 98 |
| | YAPSVKSRIAIRPDTSKNQVSLHLISVTPDDTAVYYCARDSYNGHAFDGFDVWGQGTMVTVS | |
| | SGGGGSGGGGSGGGGSGGGGSQSVLTQPPSLSAAPGQRVTISCSGTTSNIANNFVSWYQQLS | |
| | GSAPKLLINDNNKRPSGIPDRFSGSKSGASATLAITGLQTEDEADYYCGVWDNSLNAEVFGG | |
| | GTKVTVL | |
| B11 | QVQLQQSGPGLVKPSQTLSLTCAISGDSVSSNSASWNWIRQSPSRGLEWLGKTEYRSKWYYE | 90 |
| | FAPSVKSRITITPDTSKNQFSLQLNSVTPEDTAVYYCVRDSYNGYDFDGFDIWGQGTLVTVS | |
| | SGGGGSGGGGSGGGGSGGGGSQSVLTQPPSVSAAPGQKVTISCSGSSSNIGSNYVAWYQQLP | |
| | GTAPKLLIYDTNKRPSGIPDRFSGSKSGTSATLGITGLQTGDEADYYCGTWDSSLSAEVFGT | |
| | GTKVTVL | |

In certain embodiments, the ABP comprises a Fab, Fab′, F(ab′)₂, Fv, scFv, (scFv)₂, single chain antibody molecule, dual variable domain antibody, single variable domain antibody, linear antibody, V domain antibody, bispecific tandem bivalent scFvs, or bispecific T-cell engager (BiTE).

In particular embodiments, the ABP comprises an Fe, optionally human Fc.

In some embodiments, the ABP comprises a heavy chain constant region of a class selected from IgG, IgA, IgD, IgE, and IgM. In some embodiments, the ABP comprises a heavy chain constant region of the class IgG and a subclass selected from IgG1, IgG2, IgG3, and IgG4. In some embodiments, the ABP comprises a heavy chain constant region of IgG. In certain embodiments, the ABP comprises a heavy chain constant region of IgG1.

In some embodiments, the ABP is bispecific or multispecific. In some embodiments, the ABP is afucosylated.

Also provided are kits comprising one or more of the pharmaceutical compositions comprising the ABPs, and instructions for use of the pharmaceutical composition. Also provided is a pharmaceutical composition comprising an ABP described herein.

Also provided are isolated polynucleotides encoding the ABPs provided herein, or portions thereof. Also provided are vectors comprising such polynucleotides. Also provided are recombinant host cells comprising such polynucleotides and recombinant host cells comprising such vectors.

Also provided are methods of producing the ABP using the polynucleotides, vectors, or host cells provided herein. In some aspects, the present disclosure provides a method of producing an ABP that specifically binds human CD63, CD151, CD72, CD84, CD69, or CD109, comprising: expressing the ABP in the host cell, and isolating the ABP.

### 3.3.2. ABP-Drug Conjugate

Another aspect of the present disclosure provides an antigen-binding protein (ABP)-drug conjugate.

In some embodiments, the ABP-drug conjugate comprises an antigen-binding protein (ABP), a cytotoxic agent linked to the ABP, and optionally a linker that links the cytotoxic agent to the ABP, where the ABP specifically binds to a target protein selected from the group consisting of CD63, CD151, CD72, CD84, CD69, and CD109.

In some embodiments, the ABP-drug conjugate comprises one or more of the antigen-binding proteins (ABPs) described in 3.3.1.

In certain embodiments, the ABP specifically binds to CD63. In certain embodiments, the ABP specifically binds to CD151. In certain embodiments, the ABP specifically binds to CD72. In certain embodiments, the ABP specifically binds to CD84. In certain embodiments, the ABP specifically binds to CD69. In certain embodiments, the ABP specifically binds to CD109.

In some embodiments, the ABP is human, humanized, or chimeric. In some embodiments, the ABP is monoclonal. In some embodiments, the ABP is bispecific or multispecific.

In some embodiments, the ABP comprises a Fab, Fab′, F(ab′)₂, Fv, scFv, (scFv)₂, single chain antibody molecule, dual variable domain antibody, single variable domain antibody, linear antibody, or V domain antibody.

In some embodiments, the ABP comprises an Fc, optionally human Fc.

In some embodiments, the ABP comprises a heavy chain constant region of a class selected from IgG, IgA, IgD, IgE, and IgM. In some embodiments, the ABP comprises a heavy chain constant region of the class IgG and a subclass selected from IgG1, IgG2, IgG3, and IgG4. In some embodiments, the ABP comprises a heavy chain constant region of IgG1. In some embodiments, the ABP comprises a variable heavy chain region. In some embodiments, the ABP comprises a variable light chain region. In some embodiments, the ABP comprises a variable heavy chain region and variable light chain region. In some embodiments, the ABP is an antibody binding fragment. Antibodies and antigen binding fragments are described 3.4.1 “Antigen binding protein (ABP)”.

In some embodiments, the cytotoxic agent comprises an anti-angiogenic agent, a pro-apoptotic agent, an anti-mitotic agent, an anti-kinase agent, an alkylating agent, a hormone, a hormone agonist, a hormone antagonist, a chemokine, a drug, a prodrug, a toxin, an enzyme, an antimetabolite, an antibiotic, an alkaloid, or a radioactive isotope.

In some embodiments, the linker is a cleavable linker. In some embodiments, the linker is a non-cleavable linker.

In some embodiments, the backbone of the linker is 100 atoms or less in length, such as 50 atoms or less, or 20 atoms or less in length. In other embodiments, the backbone of the linker is 100 atoms or greater in length. A linker or linkage may be a covalent bond that connects two groups or a chain of between 1 and 100 atoms in length, such as between 1 and 50 atoms in length or 1 and 20 atoms in length, for example of about 1, 2, 3, 4, 5, 6, 8, 10, 12, 14, 16, 18 or 20 carbon atoms in length, where the linker may be linear, branched, cyclic or a single atom. In certain embodiments, one, two, three, four or five or more carbon atoms of a linker backbone may be optionally substituted with a sulfur, nitrogen or oxygen heteroatom. The bonds between backbone atoms may be saturated or unsaturated, usually not more than one, two, or three unsaturated bonds will be present in a linker backbone. The linker may include one or more substituent groups, for example with an alkyl, aryl or alkenyl group. A linker may include, without limitations, oligo(ethylene glycol); ethers, thioethers, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be peptidic or non-peptidic.

Non-limiting examples of antibody-drug conjugate linkers and chemistries can be found in US Application Publication No.: US20200277306, and US20190328900A1; and U.S. Pat. No. 7,750,116B1, which are hereby incorporated by reference in their entireties. Additional non-limiting examples of ADC chemistries are described in Olivier et al., (2017) “Antibody-Drug Conjugates: Fundamentals, Drug Development, and Clinical Outcomes to Target Cancer” ISBN: 978-1-119-06068-0), which is hereby incorporated by reference in its entirety.

Aspects of the present disclosure include a polynucleotide encoding the ABP-drug conjugate. In some embodiments, the polynucleotide further comprises a sequence homologous to a target genomic region for site-specific integration. In some embodiments, the polynucleotide is designed for gene editing, using endonuclease such as CRISPR-Cas system, zinc-finger nucleases, transcription activator-like effector nucleases (TALENs), and meganucleases.

In some embodiments, the polynucleotide is in a viral or a non-viral vector. The vector can be used to deliver the polynucleotide to a target cell in vitro or in vivo.

In some embodiments, the method comprises administering a non-viral vector comprising the polynucleotide, or pharmaceutical composition thereof. In some embodiments, the non-viral vector or non-viral method is used to deliver the polynucleotide to a target cell in vitro or in vivo.

Non-limiting examples of non-viral delivery methods that can be used in the present methods for delivering the polynucleotide include, but are not limited to: physical methods, needle, micro-projectile gene transfer or gene gun, electroporation, sonoporation, photoporation, magnetofection, hydroporation, mechanical massage, chemical vectors inorganic particles, calcium phosphate particles, magnetic particles, polymer based vectors, or gene delivery agents such as silica, gold, cationic lipids, lipid nano emulsions, solid lipid nanoparticles polyethylenimine (PEI), chitosan, Poly (DL-Lactide) (PLA) and Poly (DL-Lactide-co-glycoside) (PLGA), dendrimers, or polymethacrylate. Such methods can be found in Ramamoorth et al., (Ramamoorth et al., (2015) J. Clin. Diagnostic Res. 9(1): GE01-GE06, and Sung et al., (Sung et al., (2019) Biomaterials Research 23(8), pgs 1-87), which are hereby incorporated by reference in their entireties.

### 3.3.3. Bispecific T-Cell Engager (BiTE)

Aspects of the present disclosure include a bispecific T-cell engager (BiTE) comprising an antigen-binding domain and a T-cell activating domain.

In some embodiments, the antigen-binding domain specifically binds to a target antigen selected from the group consisting of CD63, CD151, CD72, CD84, CD69, and CD109. In some embodiments, the antigen-binding domain is any one of the ABPs described herein.

In certain embodiments, the antigen-binding domain is an extracellular antigen-binding domain. In certain embodiments, the antigen-binding domain specifically binds to CD63. In certain embodiments, the antigen-binding domain specifically binds to CD151. In certain embodiments, the antigen-binding domain specifically binds to CD72. In certain embodiments, the antigen-binding domain specifically binds to CD84. In certain embodiments, the antigen-binding domain specifically binds to CD69. In certain embodiments, the antigen-binding domain specifically binds to CD109.

An example of a BiTE is a fusion of an extracellular recognition domain (e.g., an antigen-binding domain), and one or more T-cell activating domains. Upon antigen engagement, the intracellular signaling portion of the BiTE can initiate an activation-related response in an T cell specific molecule, thereby stimulating T cell activation, tumor killing, and/or cytokine production.

In some embodiments, the BiTE comprises an antigen-binding domain that specifically binds to a target protein selected from the group consisting of CD63, CD151, CD72, CD84, CD69, and CD109.

In some embodiments, a bispecific T-cell engager (BiTE) can contain two scFvs produced as a single polypeptide chain. In certain embodiments, the BiTE comprises two scFVs of an antibody that specifically binds to the target protein (CD63, CD151, CD72, CD84, CD69, or CD109). Methods of making and using BiTE antibodies are described in the art. See, e.g., Cioffi et al., Clin Cancer Res 18: 465, Brischwein et al., Mol Immunol 43:1129-43 (2006); Amann M et al., Cancer Res 68:143-51 (2008); Schlereth et al., Cancer Res 65: 2882-2889 (2005); and Schlereth et al., Cancer Immunol Immunother 55:785-796 (2006); Huehls A., et al., Immunol Cell Biol. 93(3): 290-296 (2014); Wang et al., Antibodies. 8(32): 1-30 (2019), which are hereby incorporated by reference in their entireties.

In some embodiments, the extracellular antigen-binding domain comprises a single-chain variable fragment (scFv) of an antibody that specifically binds to the target protein (CD63, CD151, CD72, CD84, CD69, or CD109). In some embodiments, the T-cell activating domain comprises a single-chain variable fragment (scFV) of an antibody that specifically binds to CD3. In some embodiments, the T-cell activating domain comprises the intracellular domain of CD3ζ. In some embodiments, the T-cell activating domain comprises a ZAP-70 intracellular signaling domain. In some embodiments, the intracellular signaling domain comprises an immunoreceptor tyrosine-based activation motif (ITAM).

In a non-limiting example when the antigen-binding domain and the T-cell activating domain both comprise scFvs, the scFv can be generated by connecting the heavy and light chains of each Fv with a serine-glycine linker sequence.

In certain embodiments, the linker comprises one or more, two or more, three or more, four or more, five or more, or six or more GGGGSrepeats (“GGGGS” disclosed as SEQ ID NO: 2). In certain embodiments, the linker comprises an amino acid sequence of GGGGS (SEQ ID NO: 2).

In certain embodiments, the linker comprises one or more, two or more, three or more, four or more, five or more, six or more, seven or more, eight or more, nine or more, or ten or more SGGGG repeats (“SGGGG” disclosed as SEQ ID NO: 3). In certain embodiments, the linker comprises an amino acid sequence of SGGGG (SEQ ID NO: 3).

In certain embodiments, the linker can make the peptide sufficiently long and flexible to allow the heavy and light chains to associate in a normal conformation.

In some embodiments, the linker is a rigid linker, a cleavable linker, or a flexible linker.

In certain embodiments, the BiTE comprises a linker that connects the antigen binding domain and the T-cell activating domain. As a non-limiting example, when the antigen-binding domain and the T-cell activating domain both comprise scFvs, a GGGGS (SEQ ID NO: 2) repeat linker can connect the two scFvs.

In some embodiments, the linker comprises the amino acid sequence of GGGGS (SEQ ID NO: 2). In certain embodiments, the linker comprises the amino acid sequence motif (G4S)ₙ, wherein n is 1, 2, 3, 4, 5, 6, 7, 8, 9, or 10 (SEQ ID NO: 4). In some embodiments, the linker comprises the amino acid sequence of SGGGG (SEQ ID NO: 3). In certain embodiments, the linker comprises the amino acid sequence motif (SG4)ₙ, wherein n is 1, 2, 3, 4, 5, 6, 7, 8, 9, or 10 (SEQ ID NO: 5). In some embodiments, the linker connects the two scFvs. In some embodiments, the linker comprises one or more, two or more, three or more, four or more, five or more, six or more, seven or more, eight or more, nine or more, or ten or more GGGGS repeats (“GGGGS” disclosed as SEQ ID NO: 2).

In some embodiments, the linker comprises 1-25 amino acids. In some embodiments, the linker comprises 1-20 amino acids. In some embodiments, the linker comprises 1-15 amino acids. In certain embodiments, the linker comprises 1-10 amino acids. In certain embodiments, the linker comprises 1-9 amino acids. In certain embodiments, the linker comprises 1-8 amino acids. In certain embodiments, the linker comprises 1-7 amino acids. In some embodiments, the linker comprises 1-6 amino acids. In certain embodiments, the linker comprises 1-5 amino acids. In certain embodiments, the linker comprises 1-4 amino acids. In some embodiments, the linker comprises 1-2 amino acids. In certain embodiments, the linker comprises 1-10 amino acids. In certain embodiments, the length of the linker can determine the flexibility of movement between the two scFvs and can be adjusted by including more or fewer linker repeats to optimize binding to both target cells. For example, in some embodiments, a short flexible linker connecting two scFvs can provide free rotation of the antigen-binding domain and T-cell activating domain.

In some embodiments, the linker comprises the amino acid sequence GGGGSGGGGSGGGGSGGGGS (SEQ ID NO: 6). In some embodiments, the linker comprises the amino acid sequence GGGGSGGGGSGGGGSGGGGSGGGGS (SEQ ID NO: 7).

Non-limiting examples of linkers can be found in Chen et al., (Chen X., Zaro J. L., Shen W. C. Fusion protein linkers: Property, design and functionality. Adv. Drug Deliv. Rev. 2013; 65:1357-1369), which is hereby incorporated by reference in its entirety.

In certain embodiments, the linker is selected from: (GGGGS)₃ (SEQ ID NO: 8), (G)₈ (SEQ ID NO: 9), (G)₆ (SEQ ID NO: 10), (EAAAK)₃ (SEQ ID NO: 11), (EAAAK)ₙ (SEQ ID NO: 12), wherein n is 1-3, A(EAAAK)₄ALEA(EAAAK)₄A (SEQ ID NO: 13), PAPAP (SEQ ID NO: 14), AEAAAKEAAAKA (SEQ ID NO: 15), (Ala-Pro)ₙ (wherein n is 5-17) (SEQ ID NO: 16), VSQTSKLTR↓AETVFPDV^{b} (SEQ ID NO: 17), PLG ↓ LWA ^{c} (SEQ ID NO: 18), RVL↓AEA (SEQ ID NO: 19); EDVVCC↓SMSY (SEQ ID NO: 20), GGIEGR↓GS^{c} (SEQ ID NO: 21), TRHRQPR↓GWE (SEQ ID NO: 22), and AGNRVRR↓SVG (SEQ ID NO: 23). ^{a}Protease sensitive cleavage sites are indicated with “↓”; ^{b}Factor XIa/FVIIa sensitive cleavage; ^{c}Matrix metalloprotease-1 sensitive cleavage sequences, one example provided here; ^{d}MHV PR (HIV-1 protease); NS3 protease (HCV protease); Factor Xa sensitive cleavage, respectively; ^{e}Furin sensitive cleavage; ^{f}Cathepsin B sensitive cleavage.

Aspects of the present disclosure include a host cell comprising the BiTE.

In some embodiments, the antigen-binding domain binds to an epitope on a CD63, CD151, CD72, CD84, CD69, or CD109. In some embodiments, said antigen-binding domain comprises the CDRs of the CD63, CD151, CD72, CD84, CD69, or CD109 antibody. In some embodiments, said antigen-binding domain comprises the V_{H} and V_{L} domains of the CD63, CD151, CD72, CD84, CD69, or CD109 antibody. In some embodiments, said antigen-binding domain comprises an CD63, CD151, CD72, CD84, CD69, or CD109 single-chain variable fragment (scFv).

Aspects of the present disclosure include a polynucleotide encoding the BiTE. In some embodiments, the polynucleotide further comprises a sequence homologous to a target genomic region for site-specific integration. In some embodiments, the polynucleotide is designed for gene editing, using endonuclease such as CRISPR-Cas system, zinc-finger nucleases, transcription activator-like effector nucleases (TALENs), and meganucleases.

Aspects of the present disclosure include a vector comprising the polynucleotide. Any vectors that may be used for gene delivery may be used. In some variations, a viral vector (such as AAV, adenovirus, lentivirus, a retrovirus) is used. Non-limiting examples of vectors that can be used in the present disclosure include, but are not limited to human immunodeficiency virus; HSV, herpes simplex virus; MMSV, Moloney murine sarcoma virus; MSCV, lentivirus, murine stem cell virus; SFV, Semliki Forest virus; SIN, Sindbis virus; VEE, Venezuelan equine encephalitis virus; VSV, vesicular stomatitis virus; VV, and vaccinia virus.

In some embodiments, the vector is a recombinant AAV vector. In some embodiments, the vector for use in the methods of the disclosure is encapsidated into a virus particle (e.g. AAV virus particle including, but not limited to, AAV1, AAV2, AAV3, AAV4, AAV5, AAV6, AAV7, AAV8, AAV9, AAV10, AAV11, AAV12, AAV13, AAV14, AAV15, and AAV16).

In some embodiments, the method comprises administering a vector comprising a polynucleotide encoding the BiTE, or pharmaceutical composition thereof. For example, T cells can be modified using viral or non-viral vectors to promote specific targeting of blast cells via expression of exogenous BiTE. In some embodiments, the vector is used in vitro to generate immunoresponsive cells expressing BiTE.

In some embodiments, the method comprises administering a vector comprising a polynucleotide encoding the BiTE or a pharmaceutical composition thereof.

In some embodiments, the method comprises administering a non-viral vector comprising the polynucleotide, or pharmaceutical composition thereof. In some embodiments, the non-viral vector or non-viral method is used to deliver the polynucleotide to a target cell in vitro or in vivo.

Non-limiting examples of non-viral delivery methods that can be used in the present methods for delivering the polynucleotide include, but are not limited to: physical methods, needle, micro-projectile gene transfer or gene gun, electroporation, sonoporation, photoporation, magnetofection, hydroporation, mechanical massage, chemical vectors inorganic particles, calcium phosphate particles, magnetic particles, polymer based vectors, or gene delivery agents such as silica, gold, cationic lipids, lipid nano emulsions, solid lipid nanoparticles polyethylenimine (PEI), chitosan, Poly (DL-Lactide) (PLA) and Poly (DL-Lactide-co-glycoside) (PLGA), dendrimers, or polymethacrylate. Such methods can be found in Ramamoorth et al., (Ramamoorth et al., (2015) J. Clin. Diagnostic Res. 9(1): GE01-GE06, and Sung et al., (Sung et al., (2019) Biomaterials Research 23(8), pgs 1-87), which are hereby incorporated by reference in their entireties.

### 3.3.4. Chimeric Antigen Receptor (CAR)

Aspects of the present disclosure include a chimeric antigen receptor (CAR) comprising an extracellular antigen-binding domain, a transmembrane domain, a signaling domain, and optionally, a costimulatory domain,

In some embodiments, the extracellular antigen-binding domain specifically binds to a target antigen selected from the group consisting of CD63, CD151, CD72, CD84, CD69, and CD109. In certain embodiments, the antigen-binding domain specifically binds to CD63. In certain embodiments, the antigen-binding domain specifically binds to CD151. In certain embodiments, the antigen-binding domain specifically binds to CD72. In certain embodiments, the antigen-binding domain specifically binds to CD84. In certain embodiments, the antigen-binding domain specifically binds to CD69. In certain embodiments, the antigen-binding domain specifically binds to CD109.

In some embodiments, the antigen-binding domain is any one of the ABPs described herein.

An example of a CAR is a fusion of an extracellular recognition domain (e.g., an antigen-binding domain), a transmembrane domain, and one or more intracellular signaling domains. Upon antigen engagement, the intracellular signaling portion of the CAR can initiate an activation-related response in an immune cell, such are release of cytolytic molecules to induce tumor cell death, etc.

In some embodiments, the CAR comprises an extracellular antigen-binding domain, a transmembrane domain, a signaling domain, and optionally a costimulatory domain, wherein the extracellular antigen-binding domain specifically binds to a target protein/antigen selected from the group consisting of CD63, CD151, CD72, CD84, CD69, and CD109. In certain embodiments, the CAR further comprises a hinge region of a polypeptide.

In some embodiments, the extracellular antigen-binding domain comprises a single-chain variable fragment (scFv) of an antibody that specifically binds to the target protein (CD63, CD151, CD72, CD84, CD69, or CD109).

In some embodiments, the signaling domain comprises the intracellular domain of CD3ζ. In some embodiments, the signaling domain comprises a ZAP-70 intracellular signaling domain. In some embodiments, the intracellular signaling domain comprises an immunoreceptor tyrosine-based activation motif (ITAM).

In some embodiments, further comprising one or more (e.g., two or more, three or more, four or more, or five or more) costimulatory domains, wherein the costimulatory domain is a CD28 costimulatory domain, a 4-1BB costimulatory domain, a CD27 costimulatory domain, an OX40 costimulatory domain, or an ICOS costimulatory domain. In some embodiments, the costimulatory domain is selected from 4-1BB, CD28, ICOS, OX-40, BTLA, CD27, CD30, GITR, and HVEM.

In some embodiments, the CAR comprises 4-1BB, CD28, or a fragment thereof. In certain embodiments, the CAR comprises CD28 transmembrane domain and 4-1BB. In certain embodiments, the CAR comprises a hinge region, CD28 transmembrane domain, and 4-1BB. In certain embodiments, the hinge region is from a CD28 polypeptide.

In some embodiments, the CAR comprises the hinge region of the CD28 polypeptide, wherein the hinge region comprises at least 80%, at least 85%, at least 90%, at least 95%, at least 97%, at least 98%, at least 99%, or 100% sequence identity to an amino acid sequence of: IEVMYPPPYLDNEKSNGTIIHVKGKHLCPSPLFPGPSKP (SEQ ID NO: 99).

In some embodiments, the CAR comprises the transmembrane domain of the CD28 polypeptide having at least 80%, at least 85%, at least 90%, at least 95%, at least 97%, at least 98%, at least 99%, or 100% sequence identity to an amino acid sequence of:

| (SEQ ID NO: 100) | |
|---|---|
| | FWVLVVVGGVLACYSLLVTVAFIIFWV. |

In some embodiments, the CD28 costimulatory domain has at least 80%, at least 85%, at least 90%, at least 95%, at least 97%, at least 98%, at least 99%, or 100% sequence identity to an amino acid sequence of:

(SEQ ID NO: 102)
IEVMYPPPYLDNEKSNGTIIHVKGKHLCPSPLFPGPSKPFWVLVVVGGVL
ACYSLLVTVAFIIFWV.

In some embodiments, the zeta (CD3ζ) signaling domain has at least 80%, at least 85%, at least 90%, at least 95%, at least 97%, at least 98%, at least 99%, or 100% sequence identity to an amino acid sequence of

(SEQ ID NO: 101)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR.

In some embodiments, the CAR comprises a CD28 transmembrane (TM) and 4-1BB costimulatory domains in combination with the zeta (CD3ζ) signaling domain.

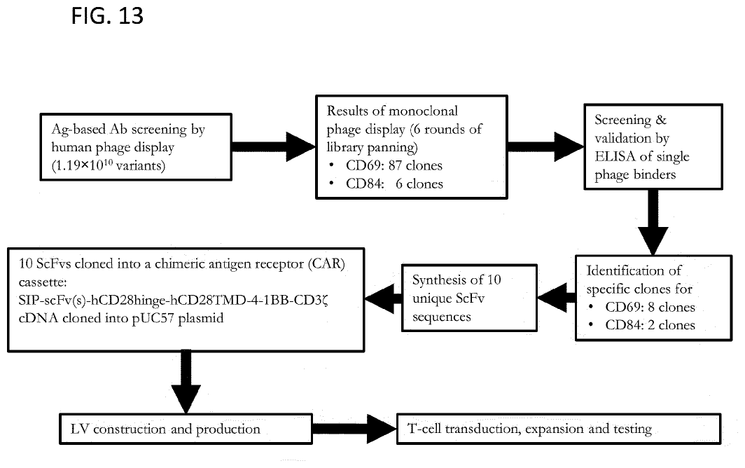

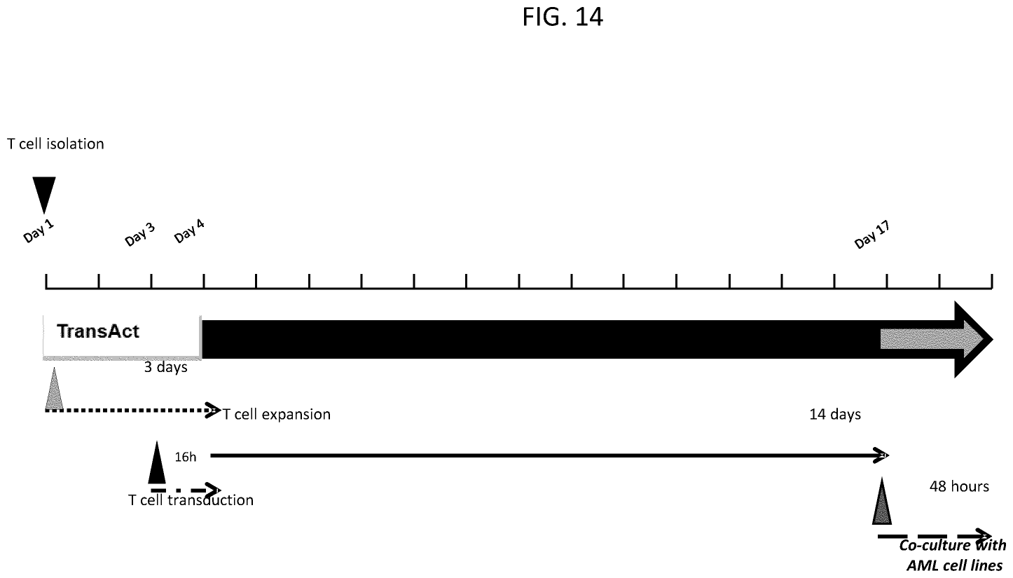

In some embodiments, the CAR construct is placed into a plasmid, such as a pUC57 plasmid. Using restriction enzymes technology, the coding sequence of CAR can be cut and placed into a vector transfer plasmid for high titer viral vector production (see e.g., FIGS. 13 and 14).

In some embodiments, the CAR comprises a signal peptide (e.g., signal interfering peptide (SIP)). In certain embodiments, the signal peptide has at least 80%, at least 85%, at least 90%, at least 95%, at least 97%, at least 98%, at least 99%, or 100% sequence identity to an amino acid sequence of: MKHLWFFLLLVAAPRWVLS (SEQ ID NO: 103). In certain embodiments, the signal peptide is encoded by a nucleic acid comprising a nucleotide sequence having at least 80%, at least 85%, at least 90%, at least 95%, at least 97%, at least 98%, at least 99%, or 100% sequence identity to the nucleotide sequence of:

(SEQ ID NO: 104)
ATGAAACACCTGTGGTTCTTCCTCCTGCTGGTGGCAGCTCCCAGATGGGT
CCTGTCC.

In some embodiments, the extracellular antigen-binding domain is a single-chain variable fragment (scFv). In some embodiments, the antigen-binding domain comprises a scFv of an antibody that specifically binds to the target protein (CD63, CD151, CD72, CD84, CD69, and/or CD109). In certain embodiments, the scFv binds to the target protein CD63. In certain embodiments, the scFv binds to the target protein CD151. In certain embodiments, the scFv binds to the target protein CD72. In certain embodiments, the scFv binds to the target protein CD84. In certain embodiments, the scFv binds to the target protein CD69. In certain embodiments, the scFv binds to the target protein CD109. In some embodiments, the scFv comprises an amino acid sequence having at least 80%, at least 85%, at least 90%, at least 95%, at least 97%, at least 98%, at least 99%, or 100% sequence identity to the amino acid sequence selected from SEQ ID NOs.: 89-98 of Table 12.

In some embodiments, the scFv comprises a polypeptide having at least 80%, at least 85%, at least 90%, at least 95%, at least 97%, at least 98%, at least 99%, or 100% sequence identity to an amino acid sequence of: SEQ ID NO: 89.

In some embodiments, the scFv comprises a nucleotide sequence encoding the scFv region, wherein the nucleotide sequence encodes an scFv with an amino acid sequence having at least 80%, at least 85%, at least 90%, at least 95%, at least 97%, at least 98%, at least 99%, or 100% sequence identity to the amino acid sequence of: SEQ ID NO: 89 as above.

In some embodiments, the scFv comprises a polypeptide having at least 80%, at least 85%, at least 90%, at least 95%, at least 97%, at least 98%, at least 99%, or 100% sequence identity to an amino acid sequence of: SEQ ID NO: 90. In some embodiments, the scFv comprises a nucleotide sequence encoding the scFv region, wherein the nucleotide sequence encodes an scFv with an amino acid sequence having at least 80%, at least 85%, at least 90%, at least 95%, at least 97%, at least 98%, at least 99%, or 100% sequence identity to the amino acid sequence of: SEQ ID NO: 90.

In some embodiments, the scFv comprises a polypeptide having at least 80%, at least 85%, at least 90%, at least 95%, at least 97%, at least 98%, at least 99%, or 100% sequence identity to an amino acid sequence of: SEQ ID NO: 91. In some embodiments, the scFv comprises a nucleotide sequence encoding the scFv region, wherein the nucleotide sequence encodes an scFv with an amino acid sequence having at least 80%, at least 85%, at least 90%, at least 95%, at least 97%, at least 98%, at least 99%, or 100% sequence identity to the amino acid sequence of SEQ ID NO: 91.

In some embodiments, the scFv comprises a polypeptide having at least 80%, at least 85%, at least 90%, at least 95%, at least 97%, at least 98%, at least 99%, or 100% sequence identity to an amino acid sequence of: SEQ ID NO: 92. In some embodiments, the scFv comprises a nucleotide sequence encoding the scFv region, wherein the nucleotide sequence encodes an scFv with an amino acid sequence having at least 80%, at least 85%, at least 90%, at least 95%, at least 97%, at least 98%, at least 99%, or 100% sequence identity to the amino acid sequence of SEQ ID NO: 92.

In some embodiments, the scFv comprises a polypeptide having at least 80%, at least 85%, at least 90%, at least 95%, at least 97%, at least 98%, at least 99%, or 100% sequence identity to an amino acid sequence of: SEQ ID NO: 93. In some embodiments, the scFv comprises a nucleotide sequence encoding the scFv region, wherein the nucleotide sequence encodes an scFv with an amino acid sequence having at least 80%, at least 85%, at least 90%, at least 95%, at least 97%, at least 98%, at least 99%, or 100% sequence identity to the amino acid sequence of SEQ ID NO: 93.

In some embodiments, the scFv comprises a polypeptide having at least 80%, at least 85%, at least 90%, at least 95%, at least 97%, at least 98%, at least 99%, or 100% sequence identity to an amino acid sequence of: SEQ ID NO: 94. In some embodiments, the scFv comprises a nucleotide sequence encoding the scFv region, wherein the nucleotide sequence encodes an scFv with an amino acid sequence having at least 80%, at least 85%, at least 90%, at least 95%, at least 97%, at least 98%, at least 99%, or 100% sequence identity to the amino acid sequence of SEQ ID NO: 94.

In some embodiments, the scFv comprises a polypeptide having at least 80%, at least 85%, at least 90%, at least 95%, at least 97%, at least 98%, at least 99%, or 100% sequence identity to an amino acid sequence of: SEQ ID NO: 95. In some embodiments, the scFv comprises a nucleotide sequence encoding the scFv region, wherein the nucleotide sequence encodes an scFv with an amino acid sequence having at least 80%, at least 85%, at least 90%, at least 95%, at least 97%, at least 98%, at least 99%, or 100% sequence identity to the amino acid sequence of SEQ ID NO: 95.

In some embodiments, the scFv comprises a polypeptide having at least 80%, at least 85%, at least 90%, at least 95%, at least 97%, at least 98%, at least 99%, or 100% sequence identity to an amino acid sequence of: SEQ ID NO: 96. In some embodiments, the scFv comprises a nucleotide sequence encoding the scFv region, wherein the nucleotide sequence encodes an scFv with an amino acid sequence having at least 80%, at least 85%, at least 90%, at least 95%, at least 97%, at least 98%, at least 99%, or 100% sequence identity to the amino acid sequence of SEQ ID NO: 96.

In some embodiments, the scFv comprises a polypeptide having at least 80%, at least 85%, at least 90%, at least 95%, at least 97%, at least 98%, at least 99%, or 100% sequence identity to an amino acid sequence of: SEQ ID NO: 97. In some embodiments, the scFv comprises a nucleotide sequence encoding the scFv region, wherein the nucleotide sequence encodes an scFv with an amino acid sequence having at least 80%, at least 85%, at least 90%, at least 95%, at least 97%, at least 98%, at least 99%, or 100% sequence identity to the amino acid sequence of SEQ ID NO: 97.

In some embodiments, the scFv comprises a polypeptide having at least 80%, at least 85%, at least 90%, at least 95%, at least 97%, at least 98%, at least 99%, or 100% sequence identity to an amino acid sequence of: SEQ ID NO: 98. In some embodiments, the scFv comprises a nucleotide sequence encoding the scFv region, wherein the nucleotide sequence encodes an scFv with an amino acid sequence having at least 80%, at least 85%, at least 90%, at least 95%, at least 97%, at least 98%, at least 99%, or 100% sequence identity to the amino acid sequence of SEQ ID NO: 98.

In some embodiments, the CAR comprises a signal peptide, an scFv region, one or more co-stimulatory domains, and a signaling domain.

In some embodiments, the CAR comprises a polypeptide having at least 80%, at least 85%, at least 90%, at least 95%, at least 97%, at least 98%, at least 99%, or 100% sequence identity to an amino acid sequence selected from SEQ ID NOs.: 35-44.

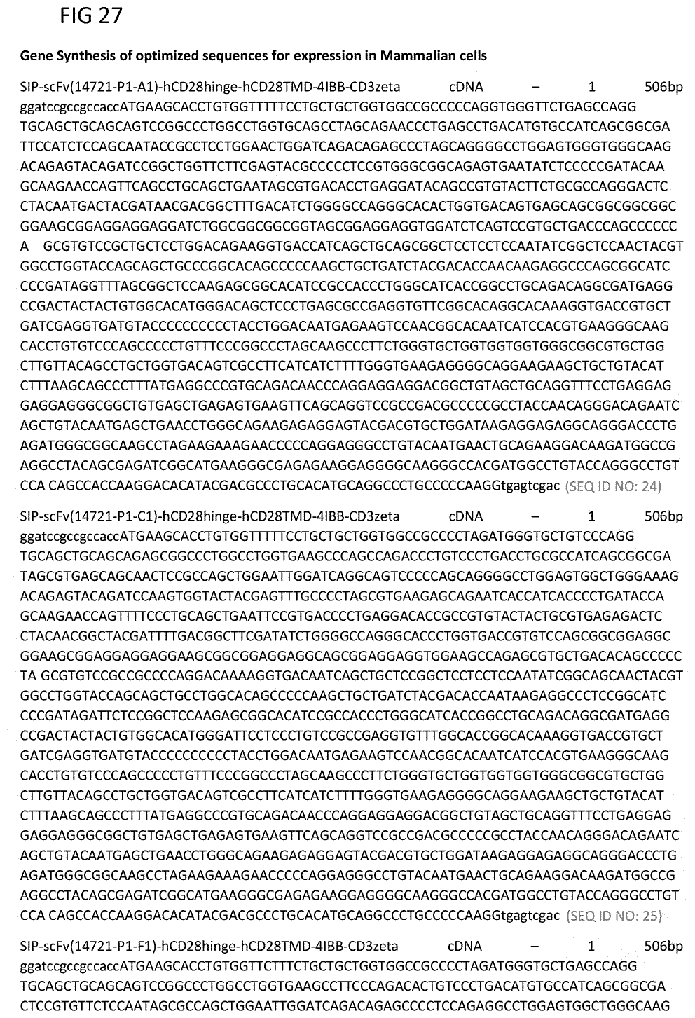

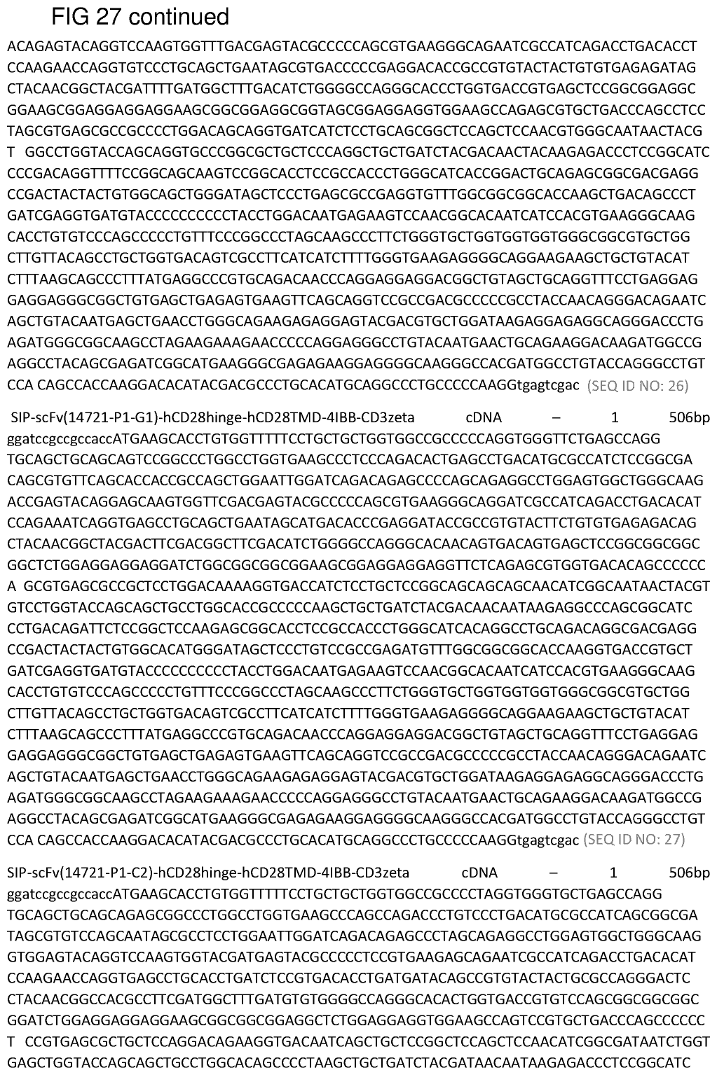

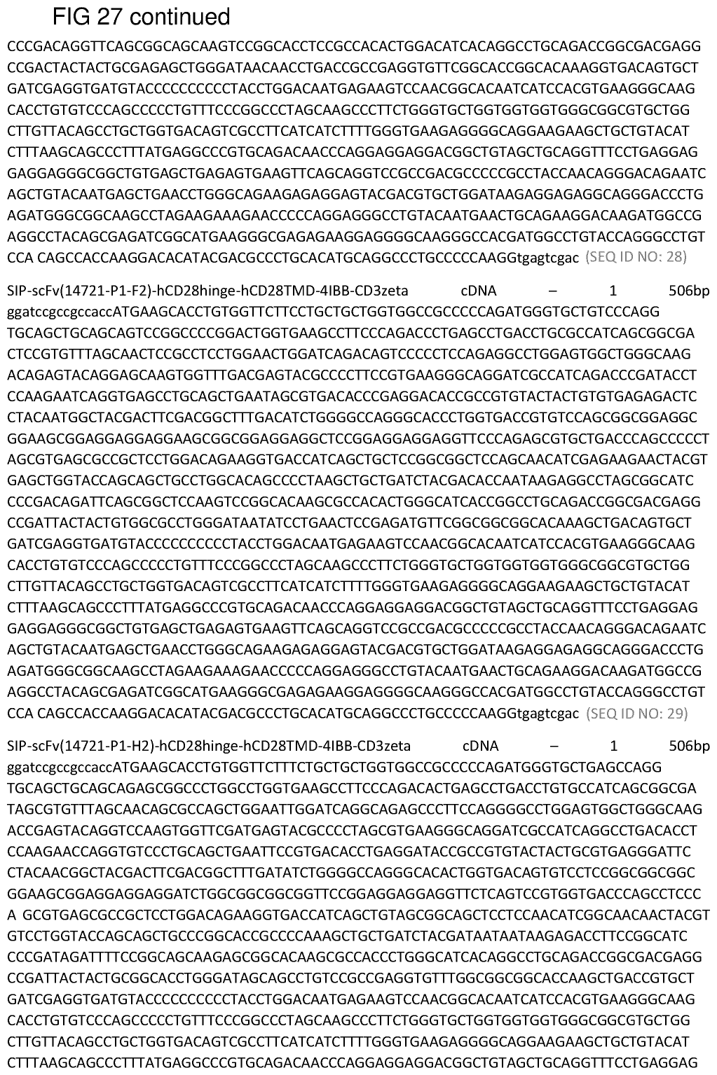

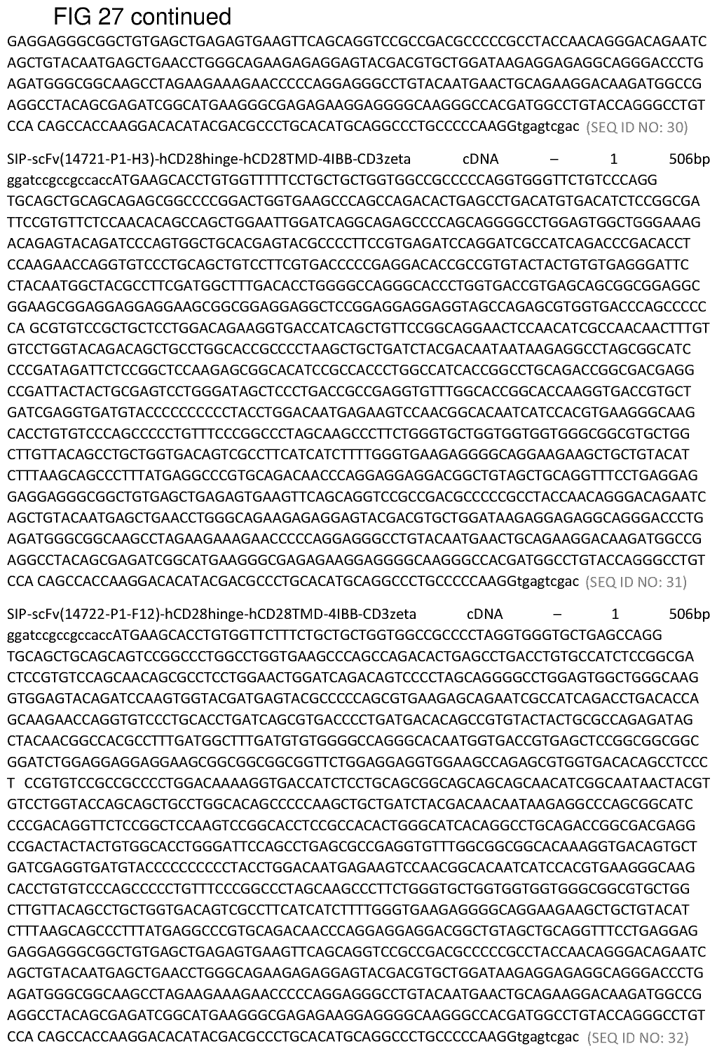

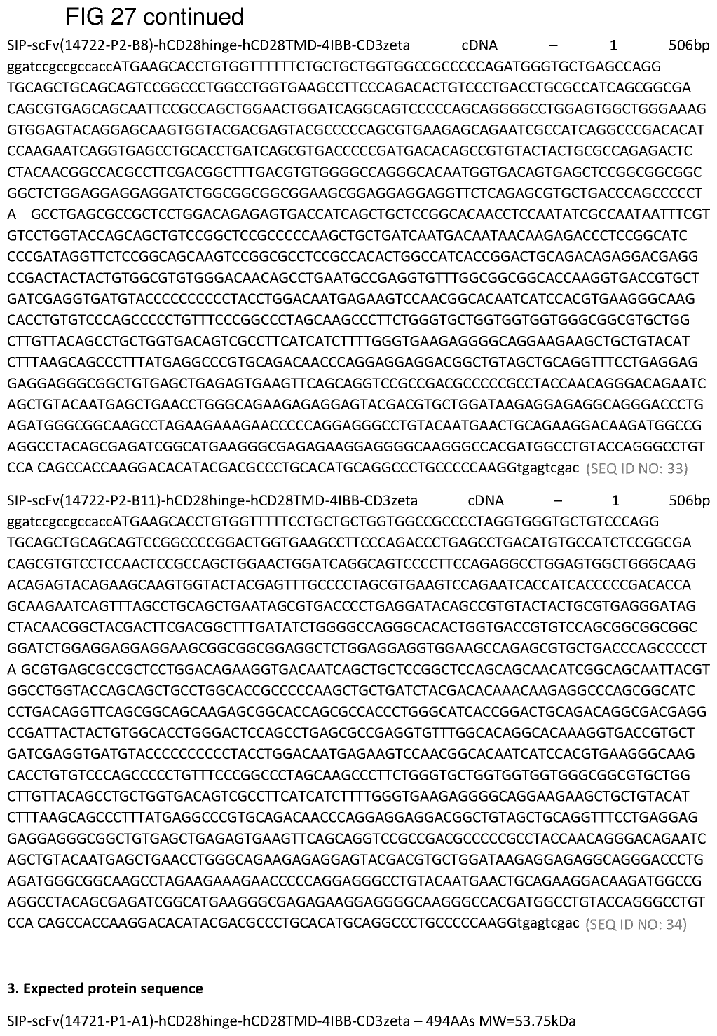

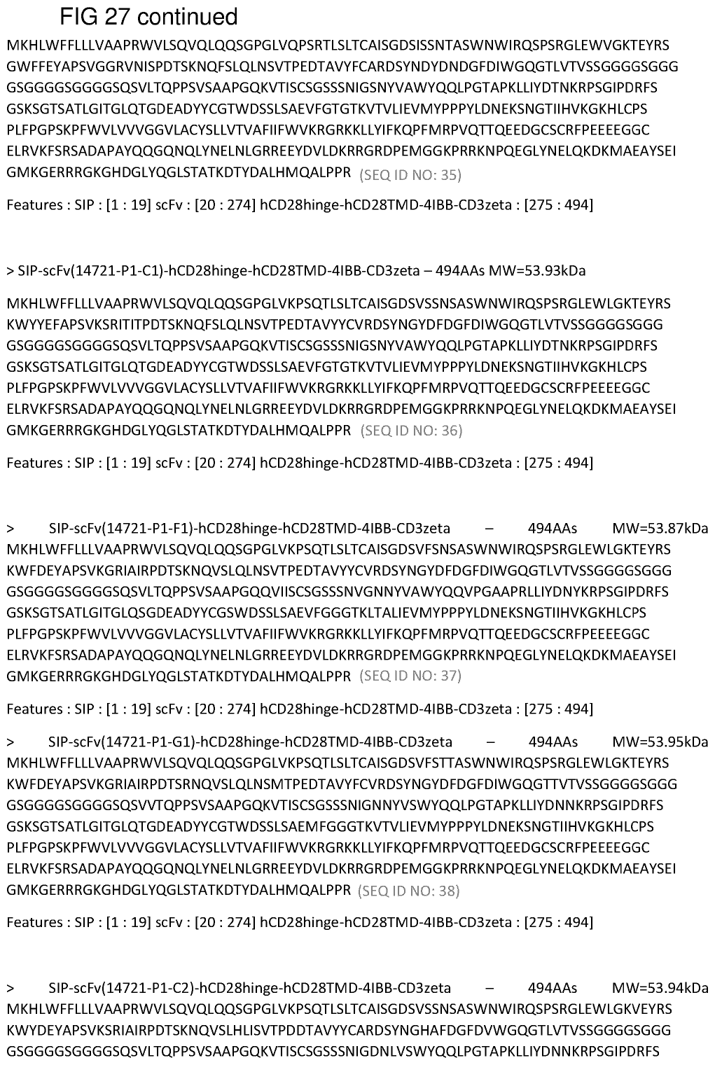

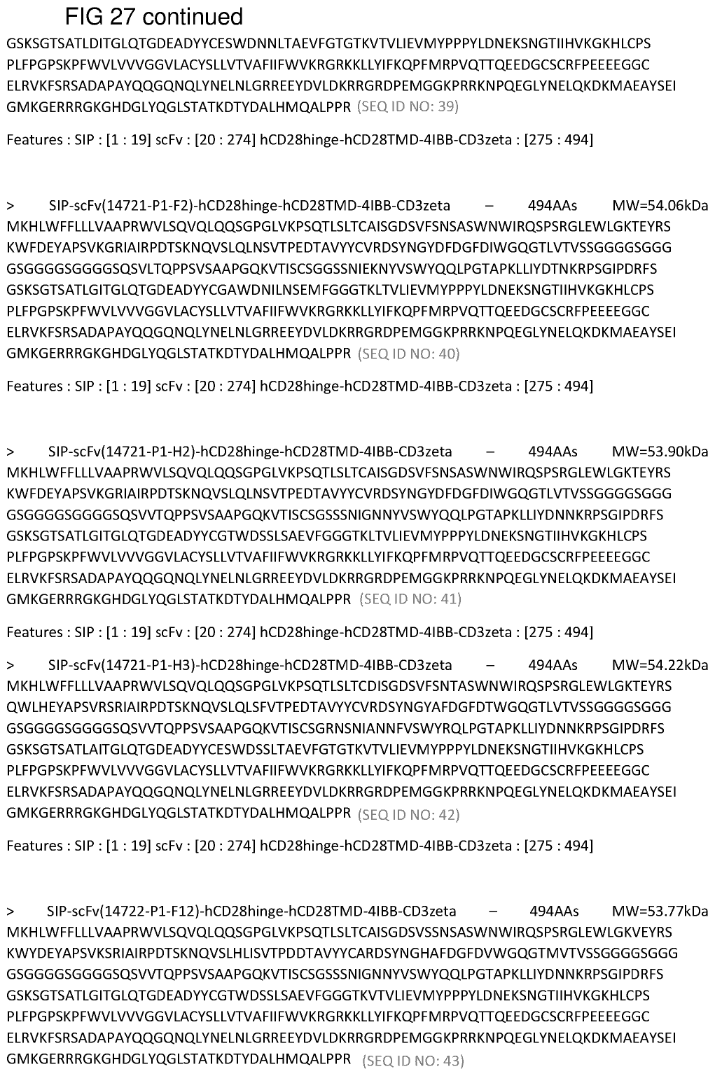

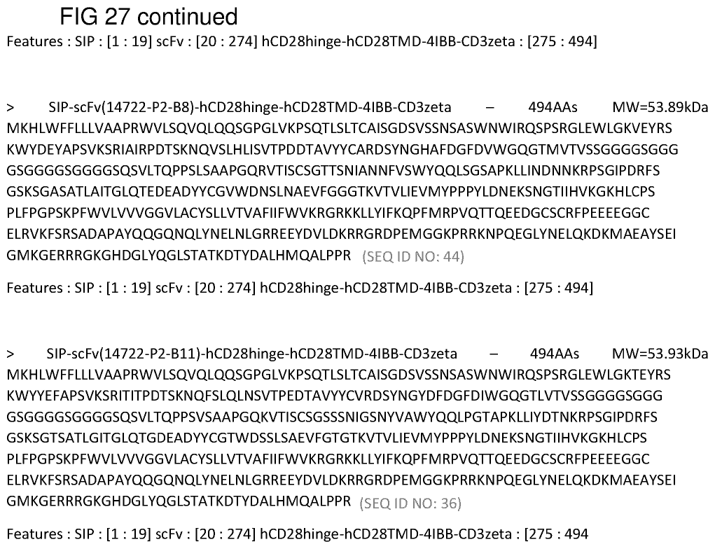

In some embodiments, the CAR comprises a polypeptide having at least 80%, at least 85%, at least 90%, at least 95%, at least 97%, at least 98%, at least 99%, or 100% sequence identity to an amino acid sequence of SEQ ID NO: 35 of FIG. 27. In some embodiments, the CAR comprises a nucleic acid having at least 80%, at least 85%, at least 90%, at least 95%, at least 97%, at least 98%, at least 99%, or 100% sequence identity to the nucleotide sequence of SEQ ID NO: 24 of FIG. 27.

In some embodiments, the CAR comprises a polypeptide having at least 80%, at least 85%, at least 90%, at least 95%, at least 97%, at least 98%, at least 99%, or 100% sequence identity to an amino acid sequence of SEQ ID NO: 36 of FIG. 27. In some embodiments, the CAR comprises a nucleic acid having at least 80%, at least 85%, at least 90%, at least 95%, at least 97%, at least 98%, at least 99%, or 100% sequence identity to the nucleotide sequence of SEQ ID NO: 25 of FIG. 27.

In some embodiments, the CAR comprises a polypeptide having at least 80%, at least 85%, at least 90%, at least 95%, at least 97%, at least 98%, at least 99%, or 100% sequence identity to an amino acid sequence of SEQ ID NO: 37 of FIG. 27. In some embodiments, the CAR comprises a nucleic acid having at least 80%, at least 85%, at least 90%, at least 95%, at least 97%, at least 98%, at least 99%, or 100% sequence identity to the nucleotide sequence of SEQ ID NO: 26 of FIG. 27.

In some embodiments, the CAR comprises a polypeptide having at least 80%, at least 85%, at least 90%, at least 95%, at least 97%, at least 98%, at least 99%, or 100% sequence identity to an amino acid sequence of SEQ ID NO: 38 of FIG. 27. In some embodiments, the CAR comprises a nucleic acid having at least 80%, at least 85%, at least 90%, at least 95%, at least 97%, at least 98%, at least 99%, or 100% sequence identity to the nucleotide sequence of SEQ ID NO: 27 of FIG. 27.

In some embodiments, the CAR comprises a polypeptide having at least 80%, at least 85%, at least 90%, at least 95%, at least 97%, at least 98%, at least 99%, or 100% sequence identity to an amino acid sequence of SEQ ID NO: 39 of FIG. 27. In some embodiments, the CAR comprises a nucleic acid having at least 80%, at least 85%, at least 90%, at least 95%, at least 97%, at least 98%, at least 99%, or 100% sequence identity to the nucleotide sequence of SEQ ID NO: 28 of FIG. 27.

In some embodiments, the CAR comprises a polypeptide having at least 80%, at least 85%, at least 90%, at least 95%, at least 97%, at least 98%, at least 99%, or 100% sequence identity to an amino acid sequence of SEQ ID NO: 40 of FIG. 27. In some embodiments, the CAR comprises a nucleic acid having at least 80%, at least 85%, at least 90%, at least 95%, at least 97%, at least 98%, at least 99%, or 100% sequence identity to the nucleotide sequence of SEQ ID NO: 29 of FIG. 27.

In some embodiments, the CAR comprises a polypeptide having at least 80%, at least 85%, at least 90%, at least 95%, at least 97%, at least 98%, at least 99%, or 100% sequence identity to an amino acid sequence of SEQ ID NO: 41 of FIG. 27. In some embodiments, the CAR comprises a nucleic acid having at least 80%, at least 85%, at least 90%, at least 95%, at least 97%, at least 98%, at least 99%, or 100% sequence identity to the nucleotide sequence of SEQ ID NO: 30 of FIG. 27.

In some embodiments, the CAR comprises a polypeptide having at least 80%, at least 85%, at least 90%, at least 95%, at least 97%, at least 98%, at least 99%, or 100% sequence identity to an amino acid sequence of SEQ ID NO: 42 of FIG. 27. In some embodiments, the CAR comprises a nucleic acid having at least 80%, at least 85%, at least 90%, at least 95%, at least 97%, at least 98%, at least 99%, or 100% sequence identity to the nucleotide sequence of SEQ ID NO: 31 of FIG. 27.

In some embodiments, the CAR comprises a polypeptide having at least 80%, at least 85%, at least 90%, at least 95%, at least 97%, at least 98%, at least 99%, or 100% sequence identity to an amino acid sequence of SEQ ID NO: 43 of FIG. 27. In some embodiments, the CAR comprises a nucleic acid having at least 80%, at least 85%, at least 90%, at least 95%, at least 97%, at least 98%, at least 99%, or 100% sequence identity to the nucleotide sequence of SEQ ID NO: 32 of FIG. 27.

In some embodiments, the CAR comprises a polypeptide having at least 80%, at least 85%, at least 90%, at least 95%, at least 97%, at least 98%, at least 99%, or 100% sequence identity to an amino acid sequence of SEQ ID NO: 44 of FIG. 27. In some embodiments, the CAR comprises a nucleic acid having at least 80%, at least 85%, at least 90%, at least 95%, at least 97%, at least 98%, at least 99%, or 100% sequence identity to the nucleotide sequence of SEQ ID NO: 33 of FIG. 27.

In some embodiments, the CAR comprises a polypeptide having at least 80%, at least 85%, at least 90%, at least 95%, at least 97%, at least 98%, at least 99%, or 100% sequence identity to an amino acid sequence of SEQ ID NO: 36 of FIG. 27. In some embodiments, the CAR comprises a nucleic acid having at least 80%, at least 85%, at least 90%, at least 95%, at least 97%, at least 98%, at least 99%, or 100% sequence identity to the nucleotide sequence of SEQ ID NO: 34 of FIG. 27.

Aspects of the present disclosure include a host cell comprising the CAR. In some embodiments, the host cell is a T-cell. In some embodiments, the host cell is a CD4⁺ and CD8⁺ T-cell. In certain embodiments, the host cell is naïve cell memory (Tₙ⁺) T-cell. In some embodiments, the host cell is stem cell memory (T_{scm}) T cell. In some embodiments, the host cell is a stem cell memory ((T_{cm}) CD197[CCR7⁺]⁺CD45R0⁻) T cell. In some embodiments, the host cell is a central memory (T_{cm}, CD197(CCR7)⁺CD45R0⁺) T cell. CD197 is used interchangeably herein as “CCR7”.

Aspects of the present disclosure include one or more host cells comprising the CAR. In certain embodiments, the one or more host cells comprising the CAR is selected from naïve and stem cell memory (Tₙ⁺ T_{scm}, CD197[CCR7⁺]⁺CD45R0⁻), and central memory (T_{cm}, CD197[CCR7]⁺CD45R0⁺) T cells.

In some embodiments, the CAR comprises: an extracellular antigen-binding domain, a transmembrane domain, and a signaling domain. In some embodiments, the CAR is a second-generation CAR comprising an extracellular antigen-binding domain, a transmembrane domain, a signaling domain, and a costimulatory domain. In some embodiments, the CAR comprises: an extracellular antigen-binding domain, a hinge region of a polypeptide, a transmembrane domain, and a signaling domain.

In some embodiments, the extracellular antigen-binding domain binds to an epitope on a CD63, CD151, CD72, CD84, CD69, or CD109 target antigen. In some embodiments, said extracellular antigen-binding domain comprises the CDRs of the CD63, CD151, CD72, CD84, CD69, or CD109 antibody. In some embodiments, said extracellular antigen-binding domain comprises the V_{H} and V_{L} domains of the CD63, CD151, CD72, CD84, CD69, or CD109 antibody. In some embodiments, said extracellular antigen-binding domain comprises an CD63, CD151, CD72, CD84, CD69, or CD109 single-chain variable fragment (scFv).

Aspects of the present disclosure include a polynucleotide encoding the CAR. In some embodiments, the polynucleotide further comprises a sequence homologous to a target genomic region for site-specific integration. In some embodiments, the polynucleotide is designed for gene editing, using endonuclease such as CRISPR-Cas system, zinc-finger nucleases, transcription activator-like effector nucleases (TALENs), and meganucleases.

Aspects of the present disclosure include a vector comprising the polynucleotide. Any vectors that may be used for gene delivery may be used. In some variations, a viral vector (such as AAV, adenovirus, lentivirus, a retrovirus) is used. Non-limiting examples of vectors that can be used in the present disclosure include, but are not limited to human immunodeficiency virus; HSV, herpes simplex virus; MMSV, Moloney murine sarcoma virus; MSCV, lentivirus, murine stem cell virus; SFV, Semliki Forest virus; SIN, Sindbis virus; VEE, Venezuelan equine encephalitis virus; VSV, vesicular stomatitis virus; VV, and vaccinia virus.

In some embodiments, the vector is a lentiviral vector. Lentiviruses are a subclass of Retroviruses. However, Lentivirus can integrate into the genome of non-dividing cells, while Retroviruses can infect only dividing cells.

Lentiviral vectors are usually produced from packaging cell line, commonly HEK293, transformed with several plasmids. The plasmids include (1) packaging plasmids encoding the virion proteins such as capsid and the reverse transcriptase, (2) a plasmid comprising an exogenous gene to be delivered to the target.

When the virus enters the cell, the viral genome in the form of RNA is reverse-transcribed to produce DNA, which is then inserted into the genome by the viral integrase enzyme. Thus, the exogenous delivered with the Lentiviral vector can remain in the genome and is passed on to the progeny of the cell when it divides.

In some embodiments, the vector is a recombinant AAV vector. In some embodiments, the vector for use in the methods of the disclosure is encapsidated into a virus particle (e.g. AAV virus particle including, but not limited to, AAV1, AAV2, AAV3, AAV4, AAV5, AAV6, AAV7, AAV8, AAV9, AAV10, AAV11, AAV12, AAV13, AAV14, AAV15, and AAV16).

Adeno-associated viruses are capable of infecting non-dividing cells and various types of cells, making them useful in constructing the gene delivery system of this disclosure. The detailed descriptions for use and preparation of AAV vector are found in U.S. Pat. Nos. 5,139,941 and 4,797,368.

Research results for AAV as gene delivery systems are disclosed in LaFace et al, Viology, 162: 483486 (1988), Zhou et al., Exp. Hematol. (NY), 21:928-933(1993), Walsh et al, J. Clin. Invest., 94:1440-1448(1994) and Flotte et al., Gene Therapy, 2:29-37(1995). Typically, a recombinant AAV virus is made by cotransfecting a plasmid containing the gene of interest (i.e., decorin gene and nucleotide sequence of interest to be delivered) flanked by the two AAV terminal repeats (McLaughlin et al., 1988; Samulski et al., 1989) and an expression plasmid containing the wild type AAV coding sequences without the terminal repeats (McCarty et al., J. Viral., 65:2936-2945(1991)).

In some embodiments, the method comprises administering a vector comprising a polynucleotide encoding the CAR, or pharmaceutical composition thereof. For example, T cells can be modified using viral or non-viral vectors to promote specific targeting of blast cells via expression of exogenous CARs. In some embodiments, the vector is used in vitro to generate immunoresponsive cells expressing CAR (e.g., CAR-T cells). Aspects of the present disclosure include an immunoresponsive cell expressing the CAR. Aspects of the present disclosure include an immunoresponsive cell comprising the polynucleotide encoding the CAR or the vector of comprising the polynucleotide.

In some embodiments, the method comprises administering a non-viral vector comprising the polynucleotide, or pharmaceutical composition thereof. In some embodiments, the non-viral vector or non-viral method is used to deliver the polynucleotide to a target cell in vitro or in vivo.

Non-limiting examples of non-viral delivery methods that can be used in the present methods for delivering the polynucleotide include, but are not limited to: physical methods, needle, micro-projectile gene transfer or gene gun, electroporation, sonoporation, photoporation, magnetofection, hydroporation, mechanical massage, chemical vectors inorganic particles, calcium phosphate particles, magnetic particles, polymer based vectors, or gene delivery agents such as silica, gold, cationic lipids, lipid nano emulsions, solid lipid nanoparticles polyethylenimine (PEI), chitosan, Poly (DL-Lactide) (PLA) and Poly (DL-Lactide-co-glycoside) (PLGA), dendrimers, or polymethacrylate. Such methods can be found in Ramamoorth et al., (Ramamoorth et al., (2015) J. Clin. Diagnostic Res. 9(1): GE01-GE06), and Sung et al., (Sung et al., (2019) Biomaterials Research 23(8), pgs 1-87), which are hereby incorporated by reference in their entireties.

In some embodiments, the immunoresponsive cell is a bispecific CAR-T targeting two target proteins selected form the group consisting of CD63, CD151, CD72, CD84, CD69, and CD109. In some embodiments, the immunoresponsive cell is a bispecific CAR-T targeting (i) one target protein selected form the group consisting of CD63, CD151, CD72, CD84, CD69, and CD109; and (ii) CD3. In some embodiments, the immunoresponsive cell is a bispecific CAR-T targeting (i) one target protein selected form the group consisting of CD63, CD151, CD72, CD84, CD69, and CD109; and (ii) CD123.

In some embodiments, the immunoresponsive cell is an αβ T cell, a γδ T cell, or a Natural Killer (NK) cell. In some embodiments, the αβ T cell is a CD3⁺, T cell, or CD4⁺ T cell or a CD8⁺ T cell.

An aspect of the present disclosure comprises a method of preparing an immunoresponsive cell. In some embodiments, the method comprises transfecting or transducing the polynucleotide encoding the CAR or the vector containing the polynucleotide encoding the CAR into an immunoresponsive cell. In some embodiments, the method comprises expanding the immunoresponsive cell for at least 48 hours. In some embodiments, the immunoresponsive cell is transduced at a multiplicity of infection of at least 10.

In some embodiments, the method further comprises, after transducing, washing the vector, and expanding the CAR T-cells for at least 14 days.

Transduction efficiency can be measured by digital droplet PCR (ddPCR), and can be expressed as vector copy number (VCN) per cell. In some embodiments, the immunoresponsive CAR T-cell comprises a mean VCN ranging from 2.5 and 33.5 (e.g., 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31.5, 32, 32.5, 33, 33.5, and the like).

In some embodiments, after transducing the polynucleotide encoding the CAR or the vector containing the polynucleotide encoding the CAR into an immunoresponsive cell, the method comprises measuring an expansion rate of the immunoresponsive cell. In certain embodiments, the expansion rate of the immunoresponsive cell is between 2 and 20 folds (e.g., 2, 4, 6, 8, 10, 12, 14, 16, 18, 20 folds).

In some embodiments, the immunoresponsive cell is naïve and stem cell memory (Tₙ+T_{scm}, CD197[CCR7]⁺CD45R0⁻), and/or central memory (T_{cm}, CD197(CCR7)⁺CD45R0⁺) T cells.

In some embodiments, the immunoresponsive CAR T-cells exhibit both activation and exhaustion at the same extent as empty CAR-T cells. This can be shown by the upregulation of CD25, CD154, CD107a and CD137, as well as of CD366, CD223 and CD279. In certain embodiments, the immunoresponsive cell containing the CAR does not alter T-cell function.

### 3.4. Methods of Diagnosis

In one aspect, the present disclosure provides methods of diagnosing cancer. In particular, the methods can be used to diagnose myeloid disorders or acute leukemias in a subject. The method comprises the step of detecting the presence or absence or level of a tumor specific antigen in a biological sample of the subject, wherein the tumor specific antigen selected from: CD63, CD151, CD72, CD84, CD69, and CD109.

In some embodiments, the step of detecting comprises contacting the biological sample with an ABP, wherein the ABP specifically binds to the tumor specific antigen. In some embodiments, the ABP is an antibody or antigen binding fragment that that binds to the tumor specific antigen.

The ABP that may be used for the step of detecting are described in section 3.3.1 “Antigen binding protein (ABP)”.

In some embodiments, the step of detecting comprises flow cytometry, immunocytochemistry, immunohistochemistry, fluorescence, or enzyme-linked immunosorbent assay (ELISA). In some embodiments, the ABP is labeled. In some embodiments, the ABP is labeled with a fluorophore, or an enzyme.

In some embodiments, the step of detecting comprises measuring mRNA level of the tumor specific antigen in the biological sample. In some embodiments, the mRNA level is measured by in situ hybridization, reverse transcription-polymerase chain reaction (RT-PCR), or by next generation sequencing.

In some embodiments, the method further comprises the step of treatment based on the diagnosis results.

### 3.4.1. Biological Sample

In some embodiments, the biological sample is a blood sample of the subject. In certain embodiments, the blood sample is a peripheral blood sample. In some embodiments, the biological sample is a bone marrow sample. In some embodiments, the biological sample comprises a solid or liquid tumor. In some embodiments, the biological sample is an AML Patient-Derived Xenograft (PDX).

In some embodiments, the biological sample comprises blast cells. In certain embodiments, the blast cells are selected from myeloid blast (e.g., myeloblast), lymphoid blast cells, or a combination of myeloid and lymphoid blast cells. In some embodiments, the blast cells are myeloid blast cells. In some embodiments, the blast cells are lymphoid blast cells. In some embodiments, the blast cells are a combination of myeloid and lymphoid blast cells. In some embodiments, the biological sample is AML blasts.

### 3.4.2. Detection of Presence or Level of TSA

In some embodiments, the method comprises detecting presence/absence or level (amount) of one TSA selected from CD63, CD151, CD72, CD84, CD69, and CD109. In some embodiments, the method comprises detecting presence/absence or level of two TSAs selected from CD63, CD151, CD72, CD84, CD69, and CD109. In some embodiments, the method comprises detecting presence/absence or level of three TSAs selected from CD63, CD151, CD72, CD84, CD69, and CD109. In some embodiments, the method comprises detecting presence/absence or level of four TSAs selected from CD63, CD151, CD72, CD84, CD69, and CD109. In some embodiments, the method comprises detecting presence/absence or level of five TSAs selected from CD63, CD151, CD72, CD84, CD69, and CD109. In some embodiments, the method comprises detecting presence/absence or level of six TSAs selected from CD63, CD151, CD72, CD84, CD69, and CD109.

In some embodiments, the step of detecting comprises contacting the biological sample with an ABP. In some embodiments, the ABP specifically binds to one of the tumor specific antigens. In some embodiments, the ABP is an anti-CD63 antibody. In some embodiments, the ABP is an anti-CD151 antibody. In some embodiments, the ABP is an anti-CD72 antibody. In some embodiments, the ABP is an anti-CD84 antibody. In some embodiments, the ABP is an anti-CD69 antibody. In some embodiments, the ABP is an anti-CD109 antibody.

In some embodiments, the ABP is labeled. In some embodiments, the ABP is labeled with a fluorophore, or an enzyme. In some embodiments, the ABP is labeled with a radioisotope. In some embodiments, the ABP is coupled with alkaline phosphatase, horseradish peroxidase, beta-galactosidase, Tobacco Etch Virus nuclear-inclusion-a endopeptidase (“TEV protease”). In some embodiments, the ABP is coupled with a fluorophore selected from the group consisting of 1,8-ANS, 4-methylumbelliferone, 7-amino-4-methylcoumarin, 7-hydroxy-4-methylcoumarin, Acridine, Alexa Fluor 350™, Alexa Fluor 405™, AMCA, AMCA-X, ATTO Rho6G, ATTO Rho11, ATTO Rho12, ATTO Rho13, ATTO Rho14, ATTO Rho101, Pacific Blue, Alexa Fluor 43Q™ Alexa Fluor480™, Alexa Fluor488™, BODIPY 492/515, Alexa Fluor 532™, Alexa Fluor 546™, Alexa Fluor555™ Alexa Fluor594™ BODIPY 505/515, Cy2, cyQUANT GR, FITC, Fluo-3, Fluo-4, GFP (EGFP), mHoneydew, Oregon Green™ 488, Oregon Green™ 514, EYFP, DsRed, DsRed2, dTomato, Cy3.5, Phycoerythrin (PE), Rhodamine Red, mTangerine, mStrawberry, mOrange, mBanana, Tetramethylrhodamine (TRITC), R-Phycoerythrin, ROX, DyLight 594, Calcium Crimson, Alexa Fluor594™, Alexa Fluor610™, Texas Red, mCherry, mKate, Alexa Fluor660™, Alexa Fluor680™ allophycocyanin, DRAQ-5, carboxynaphthofluorescein, C7, DyLight 750, Cellvue NIR780, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxy coumarin, Naphtho fluorescein, PyMPO, 5-carboxy-4′,5′-dichloro-2′,7′-dimethoxy fluorescein, 5-carboxy-2′,4′,5′,7′-tetrachlorofluorescein, 5-carboxyfluorescein, 5-carboxyrhodamine, 6-carboxyrhodamine, 6-carboxytetramethyl amino, Cascade Blue, Cy2, Cy3, Cy5,6-FAM, dansyl chloride, HEX, 6-JOE, NBD (7-nitrobenz-2-oxa-1,3-diazole), Oregon Green 488, Oregon Green 500, Oregon Green 514, Pacific Blue, phthalic acid, terephthalic acid, isophthalic acid, cresyl fast violet, cresyl blue violet, brilliant cresyl blue, para-aminobenzoic acid, erythrosine, phthalocyanines, azomethines, cyanines, xanthines, succinylfluoresceins, rare earth metal cryptates, europium trisbipyridine diamine, a europium cryptate or chelate, diamine, dicyanins, and La Jolla blue dye. In some embodiments, the ABP is conjugated with quantum dots.

In some embodiments, the step of detecting comprises flow cytometry, immunohistochemistry, immunofluorescence, or enzyme-linked immunosorbent assay (ELISA). In some embodiments, the step of detecting comprises western blotting. In some embodiments, the step of detecting comprises mass spectrometry.

In some embodiments, the step of detecting comprises measuring mRNA level of the tumor specific antigen in the biological sample. In certain embodiments, the method comprises measuring mRNA levels of CD63. In certain embodiments, the method comprises measuring mRNA levels of CD151. In certain embodiments, the method comprises measuring mRNA levels of CD72. In certain embodiments, the method comprises measuring mRNA levels of CD84. In certain embodiments, the method comprises measuring mRNA levels of CD69. In certain embodiments, the method comprises measuring mRNA levels of CD109.

In some embodiments, the mRNA level is measured by in situ hybridization, reverse transcription-polymerase chain reaction (RT-PCR), or by sequencing (e.g., next generation sequencing).

In some embodiments, the method further comprises the step of determining the presence or absence of cancer in the subject based on the presence/absence or level of the TSA in the sample from the subject.

In some embodiments, the subject is diagnosed to have a myeloid disorder (MD) or an acute leukemia (AL) when it shows expression of at least one TSA selected from CD63, CD151, CD72, CD84, CD69, and CD109. In some embodiments, the subject is diagnosed to have a myeloid disorder (MD) or an acute leukemia (AL) when it shows expression of at least two, three, four or five TSAs selected from CD63, CD151, CD72, CD84, CD69, and CD109.

In some embodiments, the subject is diagnosed to have a myeloid disorder (MD) or an acute leukemia (AL) when it shows expression of one TSA selected from CD63, CD151, CD72, CD84, CD69, and CD109. In some embodiments, the subject is diagnosed to have a myeloid disorder (MD) or an acute leukemia (AL) when it shows expression of two, three, four or five TSAs selected from CD63, CD151, CD72, CD84, CD69, and CD109. In some embodiments, the subject is diagnosed to have myeloid disorder (MD) or acute leukemia (AL) when it shows expression of CD63, CD151, CD72, CD84, CD69, and CD109.

In some embodiments, the subject is diagnosed to have a myeloid disorder (MD) or an acute leukemia (AL) when it shows increased expression of at least one TSA selected from CD63, CD151, CD72, CD84, CD69, and CD109 compared to a control sample. In some embodiments, the subject is diagnosed to have a myeloid disorder (MD) or an acute leukemia (AL) when it shows increased expression of one TSA selected from CD63, CD151, CD72, CD84, CD69, and CD109 compared to a control sample. In some embodiments, the subject is diagnosed to have a myeloid disorder (MD) or an acute leukemia (AL) when it shows increased expression of two, three, four or five TSAs selected from CD63, CD151, CD72, CD84, CD69, and CD109 compared to a control sample. In some embodiments, the subject is diagnosed to have a myeloid disorder (MD) or an acute leukemia (AL) when it shows increased expression of CD63, CD151, CD72, CD84, CD69, and CD109 compared to a control sample.

In some embodiments, the subject is diagnosed to have a myeloid disorder (MD) or an acute leukemia (AL) when it shows increased expression of at least one TSA selected from CD63, CD151, CD72, CD84, CD69, CD109, and further at least one, at least two, at least three, at least four, at least five, at least six, at least seven, at least eight, or at least nine TSA selected from CD19, CD34, CD33, CD7, CD38, CD117, CD45, CD3, and HLA-DR, compared to a control sample. In some embodiments, the subject is diagnosed to have a myeloid disorder (MD) or an acute leukemia (AL) when it shows increased expression of one TSA selected from CD63, CD151, CD72, CD84, CD69, CD109, and further at least one, at least two, at least three, at least four, at least five, at least six, at least seven, at least eight, or at least nine TSA selected from CD19, CD34, CD33, CD7, CD38, CD117, CD45, CD3, and HLA-DR, compared to a control sample. In some embodiments, the subject is diagnosed to have a myeloid disorder (MD) or an acute leukemia (AL) when it shows increased expression of two, three, four or five TSAs selected from CD63, CD151, CD72, CD84, CD69, CD109, and further at least one, at least two, at least three, at least four, at least five, at least six, at least seven, at least eight, or at least nine TSA selected from CD19, CD34, CD33, CD7, CD38, CD117, CD45, CD3, and HLA-DR compared to a control sample. In some embodiments, the subject is diagnosed to have a myeloid disorder (MD) or an acute leukemia (AL) when it shows increased expression of CD63, CD151, CD72, CD84, CD69, CD109, and further at least one, at least two, at least three, at least four, at least five, at least six, at least seven, at least eight, or at least nine TSA selected from CD19, CD34, CD33, CD7, CD38, CD117, CD45, CD3, and HLA-DR compared to a control sample.

In one embodiment, a subject is diagnosed to have a MD or an AL when it shows at least 10%, at least 20%, at least 30%, at least 40%, at least 50%, at least 60%, at least 70%, at least 80%, or at least 90% increase in expression of the TSA in comparison to a predetermined reference level of the TSA or in comparison to a sample from a healthy subject. In some embodiments, a subject is diagnosed to have a MD or an AL when it shows at least 2, 3, 4, 5, 10, 20, 50 or 100-fold increase in expression of the TSA in comparison to a predetermined reference level of the TSA or in comparison to a sample from a healthy subject.

In some embodiments, the method further comprises the step of determining the presence or absence of cancer in the subject based on the detection of the presence or level of the tumor specific antigen in the sample from the subject.

### 3.4.3. Myeloid Disorders and Acute Leukemias

In some embodiments the myeloid disorder is a myeloid malignancy. In some embodiments the myeloid disorder is a myeloid leukemia. In certain embodiments, the myeloid leukemia is acute myeloid leukemia (AML).

In some embodiments, the myeloid disorder is a myeloid neoplasm. In some embodiments, the myeloid disorder is selected from myelodysplastic syndrome (MDSs), myeloproliferative neoplasms (MPNs), myelodysplastic/myeloproliferative neoplasms (MDS/MPN), and myeloid malignancies associated with eosinophilia and abnormalities of growth factor receptors derived from platelets or fibroblasts.

In some embodiments, the MDS is selected from refractory cytopenia with unilineage dysplasia (refractory anemia; refractory neutropenia, refractory thrombocytopenia), refractory anemia with ring siderblasts, refractory cytopenia with multilineage dysplasia, refractory anemia with excess blasts-1, refractory anemia with excess blasts-2, myelodysplastic syndrome with isolated del(5q), and myelodysplastic syndrome. In some embodiments, the MPN is selected from chronic myelogenous leukemia, polycythemia vera, essential thombocythemia, primary myelofibrosis, chronic neutrophilic leukemia, chronic eosinophilic leukemia, hypereosinophilic syndrome, and mast cell disease. In some embodiments, the MDS/MPN is selected from chronic myelomonocytic leukemia, juvenile myelomonocytic leukemia, and atypical chronic myeloid leukemia. In some embodiments, the myeloid neoplasm is selected from myeloid neoplasms associated with PDGFRA rearrangement, myeloid neoplasms associated with PDGFRB rearrangement, and myeloid neoplasms associated with FGFR1 rearrangement (e.g., 8p11 myeloproliferative syndrome). In some embodiments, the acute leukemia is selected from acute lymphocytic leukemia (ALL), acute myelogenous leukemia (AML), lymphocytic leukemia (LL), myelogenous leukemia (ML).

In some embodiments, the myeloid disorders and acute leukemias have onset in pediatric or adult age. In some embodiments, the myeloid disorders and acute leukemias is pediatric AML. In certain embodiments, the myeloid disorders and acute leukemias have onset in subjects that are between 1 to 18 years old. In certain embodiments, the myeloid disorders and acute leukemias have onset in subjects that are between 1 day to 18 years old. In certain embodiments, the myeloid disorders and acute leukemias have onset in subjects that are between 1 day to 1 year old.

In some embodiments, the myeloid disorders and acute leukemias is adult AML. In certain embodiments, the myeloid disorders and acute leukemias have onset in adults that are older than 18, older than 20, older than 25, older than 30, older than 35 older than 40, older than 45, older than 50, older than 55, older than 60, or older than 65.

### 3.5. Methods of Treatment

In another aspect, the present disclosure provides a method of treating a subject with hematological malignancies. In certain embodiments, the hematological malignancy is a refractory B-cell malignancy. B-cell malignancies include, but are not limited to: B-cell acute lymphoblastic leukemia, chronic lymphocytic leukemia/small lymphocytic lymphoma, monoclonal B-cell lymphocytosis, B-cell prolymphocytic leukemia, splenic marginal zone lymphoma, hairy cell leukemia, splenic B-cell lymphoma/leukemia, unclassifiable, splenic diffuse red pulp small B-cell lymphoma, hairy cell leukemia-variant, lymphoplasmacytic lymphoma, Waldenstrom macroglobulinemia, monoclonal gammopathy of undetermined significance (MGUS) IgM, m heavy-chain disease, g heavy-chain disease, a heavy-chain disease, MGUS IgG/A, plasma cell myeloma, solitary plasmacytoma of bone, extraosseous plasmacytoma, monoclonal immunoglobulin deposition diseases, extranodal marginal zone lymphoma of mucosa-associated lymphoid tissue (MALT lymphoma), nodal marginal zone lymphoma, pediatric nodal marginal zone lymphoma, follicular lymphoma, in situ follicular neoplasia, duodenal-type follicular lymphoma, pediatric-type follicular lymphoma, large B-cell lymphoma with TRF4 rearrangement, primary cutaneous follicle center lymphoma, mantle cell lymphoma, in situ mantle cell neoplasia, diffuse large B-cell lymphoma (DLBCL) NOS, including germinal center B-cell type, and activated B-cell type; T-cell/histiocyte-rich large B-cell lymphoma, primary DLBCL of the central nervous system, primary cutaneous DLBCL, leg type, EBV+ DLBCL NOS, EBV+ mucocutaneous ulcer, DLBCL associated with chronic inflammation, lymphomatoid granulomatosis, primary mediastinal (thymic) large B-cell lymphoma, intravascular large B-cell lymphoma, ALK+ large B-cell lymphoma, plasmablastic lymphoma, primary effusion lymphoma, HHV8+ DLBCL NOS, Burkitt lymphoma, Burkitt-like lymphoma with 11q aberration, high-grade B-cell lymphoma, with MYC and BCL2 and/or BCL6 rearrangements, high-grade B-cell lymphoma NOS, and B-cell lymphoma, unclassifiable, with features intermediate between DLBCL and classical Hodgkin lymphoma. In some embodiments, a malignancy treated with an anti-CD72 nanobody as described herein is Hodgkin lymphoma, e.g., nodular lymphocyte predominant Hodgkin lymphoma, or classical Hodgkin lymphoma, including nodular sclerosis classical Hodgkin lymphoma, lymphocyte-rich classical Hodgkin lymphoma, mixed cellularity classical Hodgkin lymphoma, and lymphocyte-depleted classical Hodgkin lymphoma. In some embodiments, the subject has a posttransplant lymphoproliferative disorder (PTLD), such as plasmacytic hyperplasia PTLD, infectious mononucleosis PTLD, florid follicular hyperplasia PTLD, polymorphic PTLD, monomorphic PTLD (B- and T-/NK-cell types), or classical Hodgkin lymphoma PTLD.

In one aspect, the present disclosure provides a method of treating a subject with hematological malignancies, such as malignant B cells, or malignancy that comprises malignant myeloid cells. In some embodiments, the hematological malignancy is B-cell leukemia. In some embodiments, the B-cell leukemia is chronic lymphocytic leukemia. In some embodiments, the B-cell leukemia is mixed-lineage leukemia (MILL). In some embodiments, the hematological malignancy is a non-Hodgkin's lymphoma. In some embodiments, the is hematological malignancy is multiple myeloma. In some embodiments, the hematological malignancy comprises myeloid cells that express any one of a target protein selected from: CD63, CD151, CD72, CD84, CD69, and CD109. In some embodiments, the hematological malignancy comprises one or more, two or more, three or more, four or more, or five or more of the target proteins.

In one aspect, the present disclosure provides a method of treating a subject with myeloid disorders (MD) or acute leukemia (AL).

In some embodiments, the subject comprises one or more, two or more, three or more, four or more, or five or more of the target proteins.

In some embodiments, the methods of the present disclosure comprises the step of administering to the subject an effective amount of a therapeutic agent that specifically binds to a target protein selected from the group consisting of CD63, CD151, CD72, CD84, CD69, and CD109. The therapeutic agent can be the antigen-binding protein (ABP), ABP-drug conjugate, bispecific T-cell engager (BiTE), or immunoresponsive cell expressing a chimeric antigen receptor (CAR) specific to the target protein, described herein.

In some embodiments, the therapeutic agent is administered in an amount sufficient to affect survival of the cells expressing CD63, CD151, CD72, CD84, CD69, or CD109. In certain embodiments, the therapeutic agent is administered in an amount sufficient to eliminate the cells expressing CD63, CD151, CD72, CD84, CD69, or CD109. In some embodiments, the therapeutic agent is administered in an amount sufficient to reduce or kill the cells expressing CD63, CD151, CD72, CD84, CD69, or CD109. In some embodiments, the therapeutic agent is administered in an amount sufficient to reduce or kill the cancer cells expressing CD63, CD151, CD72, CD84, CD69, or CD109. In some embodiments, the therapeutic agent is administered in an amount sufficient to inhibit a biological effect associated with CD63, CD151, CD72, CD84, CD69, or CD109. In some embodiments, the therapeutic agent is administered in an amount sufficient to affect survival of cancer cells in the subject.

Aspects of the present disclosure include a polynucleotide encoding the ABP ABP-drug conjugate, bispecific T-cell engager (BiTE), or chimeric antigen receptor (CAR). In some embodiments, the polynucleotide further comprises a sequence homologous to a target genomic region for site-specific integration. In some embodiments, the polynucleotide is designed for gene editing, using an endonuclease such as CRISPR-Cas system, zinc-finger nucleases, transcription activator-like effector nucleases (TALENs), and meganucleases.

Aspects of the present disclosure include a vector comprising the polynucleotide. Any vectors that may be used for gene delivery may be used. In some variations, a viral vector (such as AAV, adenovirus, lentivirus, a retrovirus) is used. Non-limiting examples of vectors that can be used in the present disclosure include, but are not limited to human immunodeficiency virus; HSV, herpes simplex virus; MMSV, Moloney murine sarcoma virus; MSCV, lentivirus, murine stem cell virus; SFV, Semliki Forest virus; SIN, Sindbis virus; VEE, Venezuelan equine encephalitis virus; VSV, vesicular stomatitis virus; VV, and vaccinia virus.

In some embodiments, the vector is a lentiviral vector. Lentiviruses are a subclass of Retroviruses. However, Lentivirus can integrate into the genome of non-dividing cells, while Retroviruses can infect only dividing cells.

In some embodiments, the vector is a recombinant AAV vector. In some embodiments, the vector for use in the methods of the disclosure is encapsidated into a virus particle (e.g. AAV virus particle including, but not limited to, AAV1, AAV2, AAV3, AAV4, AAV5, AAV6, AAV7, AAV8, AAV9, AAV10, AAV11, AAV12, AAV13, AAV14, AAV15, and AAV16).

In some embodiments, the method comprises administering a vector comprising the polynucleotide, or pharmaceutical composition thereof. In some embodiments, the vector is used to deliver the polynucleotide to a target cell in vitro or in vivo.

In some embodiments, the method comprises administering a non-viral vector comprising a polynucleotide encoding the therapeutic agent, or pharmaceutical composition thereof. In some embodiments, the non-viral vector or non-viral method is used to deliver the polynucleotide to a target cell in vitro or in vivo.

Non-limiting examples of non-viral delivery methods that can be used in the present methods for delivering the polynucleotide encoding the ABP include, but are not limited to: physical methods, needle, micro-projectile gene transfer or gene gun, electroporation, sonoporation, photoporation, magnetofection, hydroporation, mechanical massage, chemical vectors inorganic particles, calcium phosphate particles, magnetic particles, polymer based vectors, or gene delivery agents such as silica, gold, cationic lipids, lipid nano emulsions, solid lipid nanoparticles polyethylenimine (PEI), chitosan, Poly (DL-Lactide) (PLA) and Poly (DL-Lactide-co-glycoside) (PLGA), dendrimers, or polymethacrylate. Such methods can be found in Ramamoorth et al., (Ramamoorth et al., (2015) J. Clin. Diagnostic Res. 9(1): GE01-GE06), and Sung et al., (Sung et al., (2019) Biomaterials Research 23(8), pgs 1-87), which are hereby incorporated by reference in their entireties.

In some embodiments, the method further comprises the step of detecting the presence/absence or level of a tumor specific antigen in a biological sample of the subject as described in section 3.4 before or after administration of the therapeutic agent. In some embodiments, the treatment method provided herein is used to treat a subject diagnosed to have MD or AL using the method described in section 3.4. In some embodiments, the diagnostic method described in section 3.4 is used to check efficacy of the treatment method provided herein. In some embodiments, the method comprises the step of determining presence or absence of myeloid disorder (MD) and acute leukemia (AL) blast cells in a sample obtained from the subject.

In some embodiments, the methods of the present disclosure comprise treating a subject with a myeloid disorders (MD) or acute leukemia (AL), comprising administering an effective amount of the ABP, the ABP-drug conjugate, the BiTE, the CAR, or the immunoresponsive cell comprising the CAR or a pharmaceutical composition thereof.

In some embodiments, the ABP, the ABP-drug conjugate, BiTE, or the immunoresponsive cells expressing the CAR is administered in an amount sufficient to affect survival of the cells expressing CD63, CD151, CD72, CD84, CD69, or CD109. In certain embodiments, the ABP, the ABP-drug conjugate, the BiTE, or the immunoresponsive cells expressing the CAR is administered in an amount sufficient to eliminate the cells expressing CD63, CD151, CD72, CD84, CD69, or CD109. In some embodiments, the ABP, the ABP-drug conjugate, the BiTE, or the immunoresponsive cells expressing the CAR is administered in an amount sufficient to reduce or kill the cells expressing CD63, CD151, CD72, CD84, CD69, or CD109. In some embodiments, the ABP, the ABP-drug conjugate, the BiTE, or the immunoresponsive cells expressing the CAR is administered in an amount sufficient to reduce or kill the cancer cells expressing CD63, CD151, CD72, CD84, CD69, or CD109. In some embodiments, the ABP, the ABP-drug conjugate, the BiTE, or the immunoresponsive cells expressing the CAR is administered in an amount sufficient to reduce or kill the cancer cells in the subject.

In some embodiments, the ABP, the ABP-drug conjugate, the BiTE, or the CAR binds to the target protein with a K_{D} of less than or equal to 50 nM, 10 nM, 5 nM, 1 nM, 0.5 nM or 0.1 nM, as measured by surface plasmon resonance (SPR) assay. In some embodiments, the ABP, the ABP-drug conjugate, the BiTE, or the CAR comprises an antigen binding domain that binds to the target protein with a K_{D} of less than or equal to 50 nM, 10 nM, 5 nM, 1 nM, 0.5 nM or 0.1 nM, as measured by surface plasmon resonance (SPR) assay.

In some embodiments, the ABP, the ABP-drug conjugate, the BiTE, or the CAR binds human CD63, CD151, CD72, CD84, CD69, or CD109 with a K_{D} of less than 500 nM, 50 nM, 10 nM, 5 nM, 1 nM, 0.5 nM or 0.1 nM as measure by bio-layer interferometry. In some embodiments, the ABP, the ABP-drug conjugate, the BiTE, or the CAR comprises an antigen binding domain that binds human CD63, CD151, CD72, CD84, CD69, or CD109 with a K_{D} of less than 500 nM, 50 nM, 10 nM, 5 nM, 1 nM, 0.5 nM or 0.1 nM as measure by bio-layer interferometry.

In some embodiments, the ABP, the ABP-drug conjugate, the BiTE, or the CAR comprises an antigen binding domain of a commercial antibody or its modification obtained by affinity maturation. In some embodiments, the commercially available antibody is an antibody against CD69 (e.g., Invitrogen 14-0699-82, ab201570, R&D MAB2359). In some embodiments, the commercially available antibody is an antibody against CD63 (e.g., ab59479, BD556019). In some embodiments, the commercially available antibody is an antibody against CD151 (e.g., Invitrogen MA5-16443, BD 556056). In some embodiments, the commercially available antibody is an antibody against CD84 (e.g., Biolegend 326002, NOVUS NBP2-44345). In some embodiments, the commercially available antibody is an antibody against CD109 (e.g., R&D MAB4385, BD56019). In some embodiments, the commercially available antibody is an antibody against CD72 (e.g., Biolegend 316202, BD 555917, MAB 5405, Invitrogen PAS-97567).

In some embodiments, the ABP, the ABP-drug conjugate, the BiTE, or the CAR comprises an antigen binding domain of A1, C1, F1, G1, C2, F2, H2, H3, F12, B8, and B11, or a modification thereof.

### 3.5.1. Additional Agents

In some embodiments, the methods of treatment provided herein further comprise administering an additional agent in combination with the ABP or pharmaceutical composition thereof, ABP-drug conjugate or pharmaceutical composition thereof, a BiTE or pharmaceutical composition thereof, or an immunoresponsive cell expressing a CAR or pharmaceutical composition thereof. In some embodiments, the additional agent is administered separately, sequentially or together with the ABP or pharmaceutical composition thereof, ABP-drug conjugate or pharmaceutical composition thereof, a BiTE or pharmaceutical composition thereof or an immunoresponsive cell expressing a CAR or pharmaceutical composition thereof.

In some embodiments, the ABP or the pharmaceutical composition thereof is administered in combination with an additional agent. In some embodiments, the ABP-drug conjugate or the pharmaceutical composition thereof is administered in combination with an additional agent. In some embodiments, the immunoresponsive cell expressing a CAR or the pharmaceutical composition thereof is administered in combination with an additional agent.

In some embodiments, the additional agent is a chemotherapeutic or biological agent.

In certain embodiments, the additional agent is a chemotherapeutic agent. In particular embodiments, the chemotherapeutic agent is selected from the group consisting of cytarabine, daunorubicin, idarubicin, cladribine, mitoxantrone, azacitidine, decitabine, and CPX-351 (Vyxeos®).

In particular embodiments, the chemotherapeutic agent is Fludarabine. In other particular embodiments, the chemotherapeutic agent is Cyclophosphamide.

In certain embodiments, the additional agent is a biological agent. In some embodiments, the biological agent is an antibody against a target protein other than CD63, CD151, CD72, CD84, CD69, or CD109.

In some embodiments, the additional agent is a hedgehog pathway inhibitor. In some embodiments, the hedgehog pathway inhibitor is a sonic hedgehog pathway inhibitor. In some embodiments, the sonic hedgehog pathway inhibitor is selected from vismodegib, sonidigib, and arsenic trioxide (ATO). In certain embodiments, the hedgehog pathway inhibitor is glasdegib (Daurismo™).

In some embodiments, the additional agent is an FMS-like tyrosine kinase 3 (FLT3) inhibitor. In certain embodiments, the FLT3 inhibitor is selected from the group consisting of midostaurin (Rydapt®), gilteritinib (Xospata®), sorafenib, lestaurtinib, quizartinib, and crenolanib.

In some embodiments, the additional agent is an isocitrate dehydrogenase 1 (IDH1) or isocitrate dehydrogenase 2 (IDH2) inhibitor. In certain embodiments, the IDH1 or IDH2 inhibitor is ivosidenib (Tibsovo®) or enasidenib (Idhifa®).

In some embodiments, the additional agent is a B-cell lymphoma 2 (BCL2) inhibitor. In certain embodiments, the BCL2 inhibitor is venetoclax (Venclexta®).

In some embodiments, the additional agent is a CD33-targeting agent. In certain embodiments, the CD33-targeting agent is gemtuzumab ozogamicin (Mylotarg™) or vadastuximab talirine (SGN-CD33A).

In some embodiments, the additional agent is a cell cycle checkpoint inhibitor. In some embodiments, the cell cycle checkpoint inhibitor is an Aurora kinase inhibitor, a Polo-like kinase 1 (PLK1) inhibitor, a cyclin dependent kinase (CDK) inhibitor, or a checkpoint kinase 1 (CHK1) inhibitor.

In some embodiments, the additional agent is an immune checkpoint inhibitor. In certain embodiments, the immune checkpoint inhibitor is an anti-CTLA-4 antibody, an anti-PD-1 antibody, or an anti-PD-L1 antibody.

### 3.6. Pharmaceutical Compositions

In yet another aspect, the present disclosure provides pharmaceutical compositions containing the antigen-binding protein (ABP), the ABP-drug conjugate, the BiTE, or the immunoresponsive cell expressing a chimeric antigen receptor (CAR) described herein. Such compositions comprise an effective amount of an antigen-binding protein (ABP), an ABP-drug conjugate, a BiTE, or an immunoresponsive cell expressing a chimeric antigen receptor (CAR) in a mixture with a pharmaceutically acceptable excipient.

In one aspect, the present disclosure includes a pharmaceutical composition comprising the ABP and a pharmaceutically acceptable excipient.

In another aspect, the present disclosure includes a pharmaceutical composition comprising the ABP-drug conjugate and a pharmaceutically acceptable excipient.

In another aspect, the present disclosure includes a pharmaceutical composition comprising the BiTE and a pharmaceutically acceptable excipient.

In another aspect, the present disclosure includes a pharmaceutical composition comprising the immunoresponsive cell expressing a chimeric antigen receptor (CAR) and a pharmaceutically acceptable excipient.

The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition.

Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, other organic acids); bulking agents (such as mannitol or glycine), chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants. Neutral buffered saline or saline mixed with conspecific serum albumin are examples of appropriate diluents. In accordance with appropriate industry standards, preservatives such as benzyl alcohol may also be added. The composition may be formulated as a lyophilizate using appropriate excipient solutions (e.g., sucrose) as diluents. Suitable components are nontoxic to recipients at the dosages and concentrations employed.

Optionally, the composition additionally comprises one or more additional agents, for example, physiologically active agents, such as, an anti-angiogenic substance, a chemotherapeutic substance (such as capecitabine, 5-fluorouracil, or doxorubicin), an analgesic substance, etc., non-exclusive examples of which are provided herein.

In another embodiment of the disclosure, the compositions disclosed herein may be formulated in a neutral or salt form. Illustrative pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective.

The carriers can further comprise any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a human.

The optimal pharmaceutical composition will be determined by one skilled in the art depending upon, for example, the intended route of administration, delivery format, and desired dosage. See for example, Remington's Pharmaceutical Sciences, supra. Such compositions may influence the physical state, stability, rate of in vivo release, and rate of in vivo clearance of the polypeptide. For example, suitable compositions may be water for injection, physiological saline solution for parenteral administration.

In some embodiments, the active ingredient (e.g., ABP, ABP-drug conjugate) is present in the pharmaceutical composition at a concentration of at least 0.01 mg/ml, at least 0.1 mg/ml, at least 0.5 mg/ml, or at least 1 mg/ml. In certain embodiments, the active ingredient is present in the pharmaceutical composition at a concentration of at least 1 mg/ml, 2 mg/ml, 3 mg/ml, 4 mg/ml, 5 mg/ml, 10 mg/ml, 15 mg/ml, 20 mg/ml, or 25 mg/ml. In certain embodiments, the active ingredient is present in the pharmaceutical composition at a concentration of at least 30 mg/ml, 35 mg/ml, 40 mg/ml, 45 mg/ml or 50 mg/ml.

In some embodiments, the pharmaceutical composition further comprises one or more additional active ingredients in addition to the proteins or polypeptides of the present disclosure. The additional active ingredient is one or more of the additional agents described in 3.5.1.

The pharmaceutical composition can be formulated for administration by any route of administration appropriate for human or veterinary medicine. In some embodiments, the pharmaceutical composition is adapted for injection. In some embodiments, the pharmaceutical composition is formulated for intravenous, intramuscular, intraperitoneal or subcutaneous administration. In some embodiments, the pharmaceutical composition is adapted for intravenous infusion. In some embodiments, the pharmaceutical composition is formulated for intrathecal or intracerebroventricular administration.

In various embodiments, the unit dosage form is a vial, ampule, bottle, or pre-filled syringe. In some embodiments, the unit dosage form contains at least 0.01 mg, 0.1 mg, 0.5 mg, 1 mg, 2.5 mg, 5 mg, 10 mg, 12.5 mg, 25 mg, 50 mg, 75 mg, or 100 mg of the ABP or ABP-drug conjugate. In some embodiments, the unit dosage form contains at least 125 mg, 150 mg, 175 mg, or 200 mg of the ABP or ABP-drug conjugate. In some embodiments, the unit dosage form contains at least 250 mg of the ABP or ABP-drug conjugate. In some embodiments, the unit dosage form contains at least 1×10⁴, 1×10⁵, 1×10⁶, 1.5×10⁶, 1×10⁷, 2×10⁶, 2.5×10⁶, 2.5×10⁷, 3×10⁶, 3.5×10⁶, 4×10⁶, 4.5×10⁶, 5×10⁶, 5×10⁷, 1×10⁸, 2.5×10⁸, 5×10⁸, 7.5×10⁸, 1×10⁹, 2.5×10⁹, 5×10⁹, 1×10¹⁰, 2.5×10¹⁰, 5×10¹⁰, or 1×10⁹ immunoresponsive cells expressing a CAR. In some embodiments, the unit dosage form contains at least 1.5×10⁴, 1.5×10⁵, 1.5×10⁶, 1.5×10⁷, 2.5×10⁷, 5×10⁷, 1×10⁸, 2.5×10⁸, 5×10⁸, 7.5×10⁸, 1×10⁹, 2.5×10⁹, 5×10⁹, 1×10¹⁰, 2.5×10¹⁰, 5×10¹⁰, or 1×10⁹ immunoresponsive cells expressing a CAR.

In typical embodiments, the pharmaceutical composition in the unit dosage form is in liquid form. In various embodiments, the unit dosage form contains between 0.1 mL and 50 ml of the pharmaceutical composition. In some embodiments, the unit dosage form contains 1 ml, 2.5 ml, 5 ml, 7.5 ml, 10 ml, 25 ml, or 50 ml of pharmaceutical composition.

In particular embodiments, the unit dosage form is a vial containing 1 ml of the pharmaceutical composition at a concentration of 0.01 mg/ml, 0.1 mg/ml, 0.5 mg/ml, or 1 mg/ml. In some embodiments, the unit dosage form is a vial containing 2 ml of the pharmaceutical composition at a concentration of 0.01 mg/ml, 0.1 mg/ml, 0.5 mg/ml, or 1 mg/ml.

In some embodiments, the pharmaceutical composition in the unit dosage form is in solid form, such as a lyophilate, suitable for solubilization.

The amount of active ingredient which can be combined with a carrier material to produce a single dosage form will generally be that amount of the compound which produces a therapeutic effect.

### 4. EXAMPLES

### 4.1. Example 1: Identification of Tumor-Specific Antigens

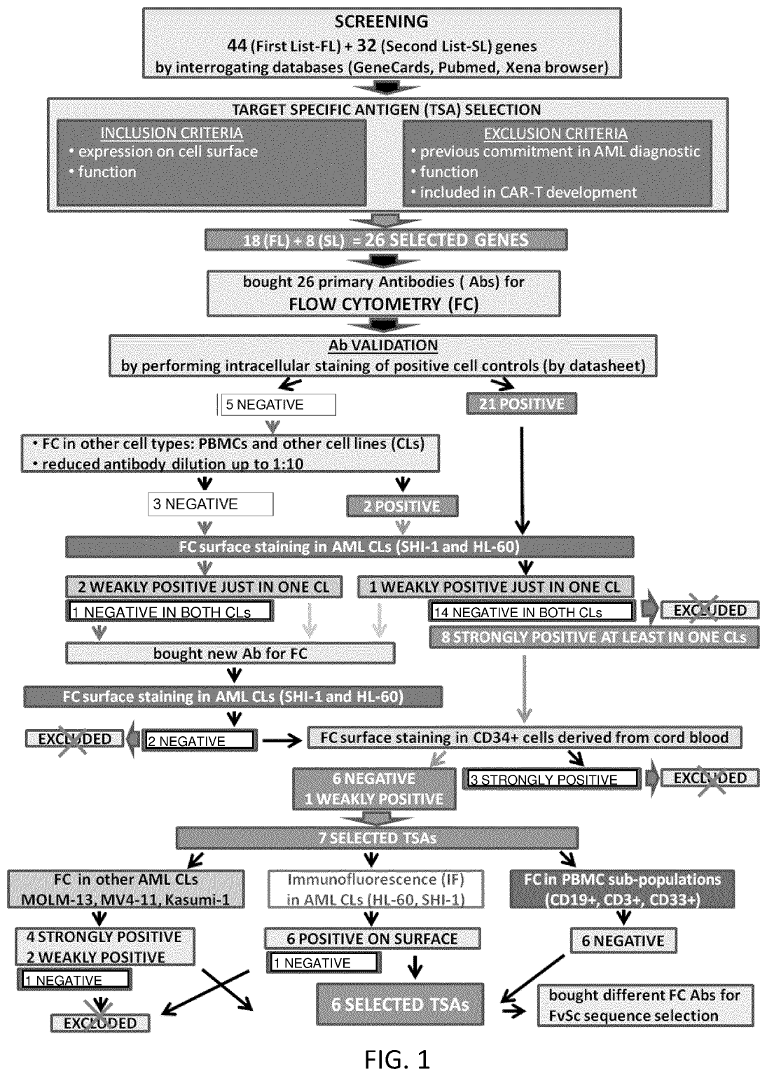

Tumor-specific antigens (TSA) were identified from AMIL patient samples as summarized in FIG. 1.

Briefly, candidate genes for tumor-specific antigens were obtained from in silico target antigen discovery analysis by applying a refined selection process. The genes were selected for being hyper-expressed by AMIL blasts according to gene expression profiles previously generated for risk stratification from 85 pediatric AMIL samples (GSE75461) by the HTA 2.0 (Affymetrix) platform. The HTA 2.0 (Affymetrix) platform maximizes gathering of valuable information by minimizing the conserved sequence synthesized on an array. This high-resolution array design contained an unprecedented >6.0 million probes covering coding transcripts and non-coding transcripts. 70% of the probes on this array covered exons for coding transcripts, and the remaining 30% of probes on the array covered exon-exon splice junctions and noncoding transcripts. The unparalleled coverage of this array provided the insight into all coding and non-coding transcripts available.

The data were used to identify hyper-expressed genes, namely genes that had expression above the upper inflection point of the distribution (>3.4) by all 85 AML samples and not by bone marrow healthy control samples (over-expressed genes by limma contrast of the AML patient cohort and 3 HTA-arrays obtained from three bone marrow samples of pediatric healthy donors after Benjamini-Hochberg multiple correction).

Among the genes, a subset was selected when they were cellular surface protein antigens (CSPAs) according a validated surfaceome protein data set http://wlab.ethz.ch/cspa/. Candidates from this process were then validated using a public dataset, the TARGET database, from which the gene expression matrixes of 262 AML pediatric samples were downloaded. The TARGET data was RMA normalized; the 85 patient cohort of Italian AMLs and the 262 TAGET AML samples were z-score normalized, so it was possible to compare relative expression of selected genes via box plot between the two cohorts. This allowed identification of 44 genes (first list—FL) as the best representative surface antigens of pediatric AML at the onset of disease, which are not expressed in healthy donor bone marrow.

The 44 candidate genes of this first list-FL were then analyzed by interrogating public databases for broad coverage and specificity, aiming at selecting the most promising targets to enter the following in vitro test of protein expression. The GeneCards software was used to look at TSA functional description, gene chromosomal localization, association with known pathologies/cancer, mRNA expression in normal human tissues, estimated protein expression, subcellular locations, and involved pathways; and Pubmed was used to interrogate TSA previous involvement in cancer including AML, and to exclude their involvement in previous CAR-T development projects, and XenaBrowser was used to predict the expression selectivity of candidate TSA in AML. This is an online platform to explore public and private, multi-omic and clinical/phenotypes including 1500+ datasets and 50+ cancer types. This tool provides an interactive online visualization of seminal cancer genomics datasets, including data from The Cancer Genome Atlas (TCGA), International Cancer Genome Consortium (ICGC), Genomic Data Commons (GDC), and UCSC RNA-seq compendium. Xena supports virtually any functional genomic data, including SNVs, INDELs, large structural variants, copy number variation, gene, transcript, exon, miRNA, LncRNA, protein-expressions, DNA methylation, ATAC-seq signals, phenotypic annotations, and higher-level derived genomic parameters. This process of refinement applied as inclusion criteria gene function (associated to cancer) and localization on cell surface, and as exclusion criteria i) function considering enzymes and ribosomal components, ii) localization other than the cell surface, including nuclear membranes; iii) previous commitment in AML diagnostic panels or CAR-T development, iv) being previously part of a patent looking at Espacenet, Trade Mark information, patentscope, and v) a high documented expression in multiple human tissues. At the end of this refinement process, 26 out of the 44 genes of the FL were discarded, providing 18 candidate TSAs.

The Applicant then produced a second list—SL by interrogating the same in silico data set and selecting those genes whose expression was below the inflection point of the distribution (>2.4 and <3.4). This new list included additional 32 candidate TSAs that underwent the same analytical process described above for TSAs refinement. 24 out of the 32 TSAs identified in the SL were discarded due to exclusion criteria (i-v), providing 8 additional candidate TSAs.

These 26 candidate TSAs (18 candidate TSAs from FL and 8 additional candidate TSAs from SL) were then characterized for protein expression and localization by flow cytometry (FC). Primary commercial antibodies recognizing the candidate TSAs were first tested with positive control cells. Once the antibodies were demonstrated to provide staining on controls, cell surface staining for the TSAs was performed with a minimum of 2 AML cell lines (SHI-1 and HL-60). Eleven out of the 26 candidate TSAs were confirmed to be expressed on the AML cell surface (strongly or weakly expressed as indicated in FIG. 1) by flow cytometry.

Six AML-TSAs (CD63, CD151, CD72, CD84, CD69 and CD109) were finally selected based on strong positive signals and specificity detected by flow cytometry with the AML cell lines SHI-1 and HL-60 (FIG. 1). For further validation, their expression on additional AML cell lines including KASUMI-1, MOLM-13, and MV4-11 was tested by immunofluorescence employing the same antibodies used for flow cytometry.

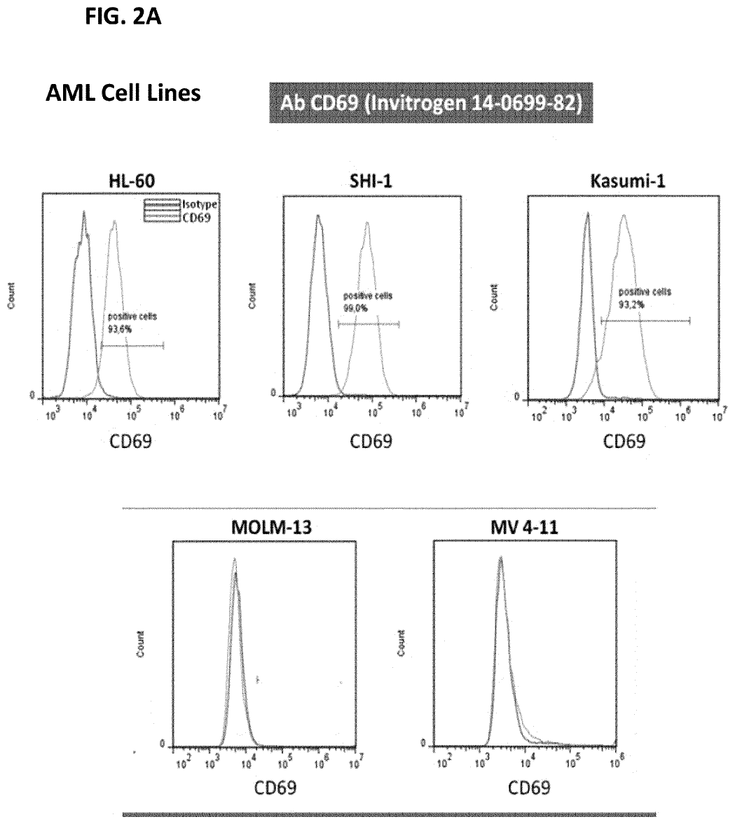

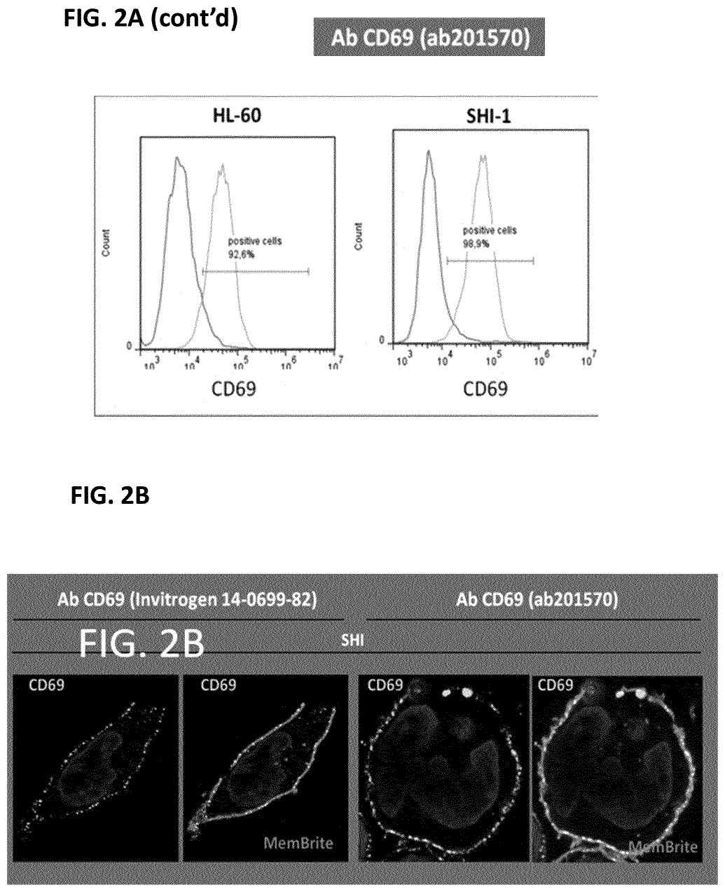

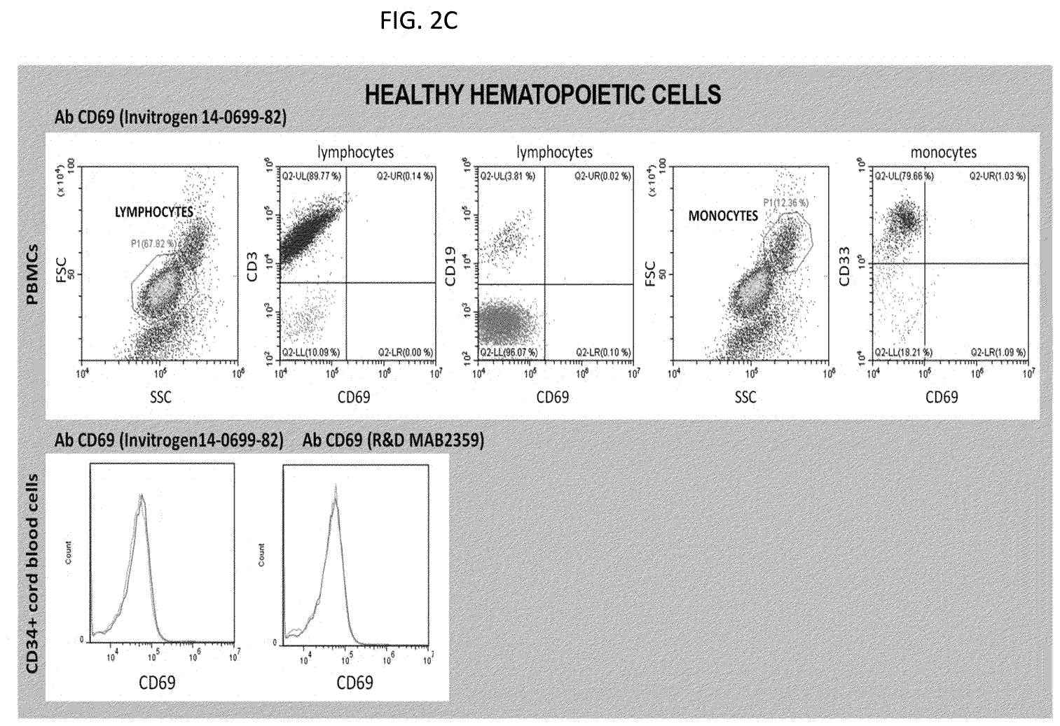

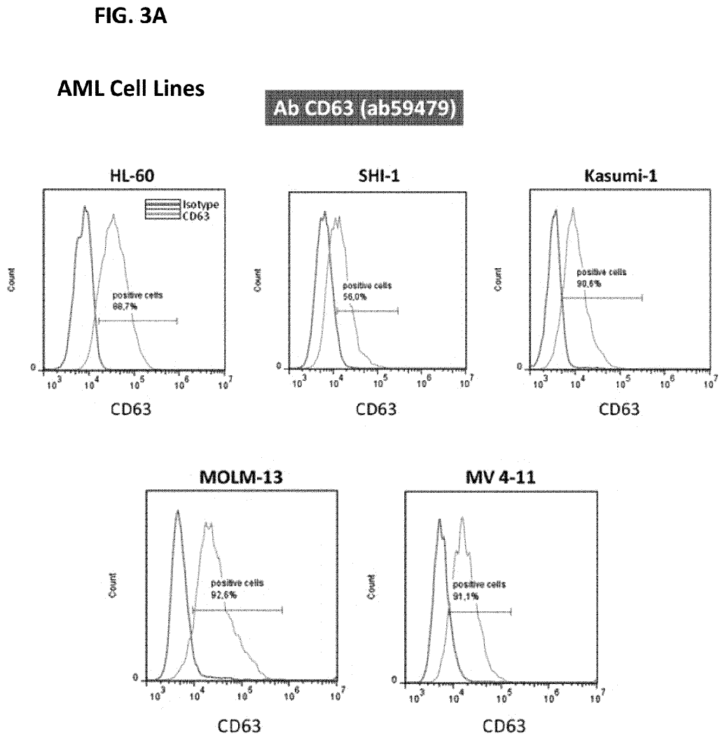

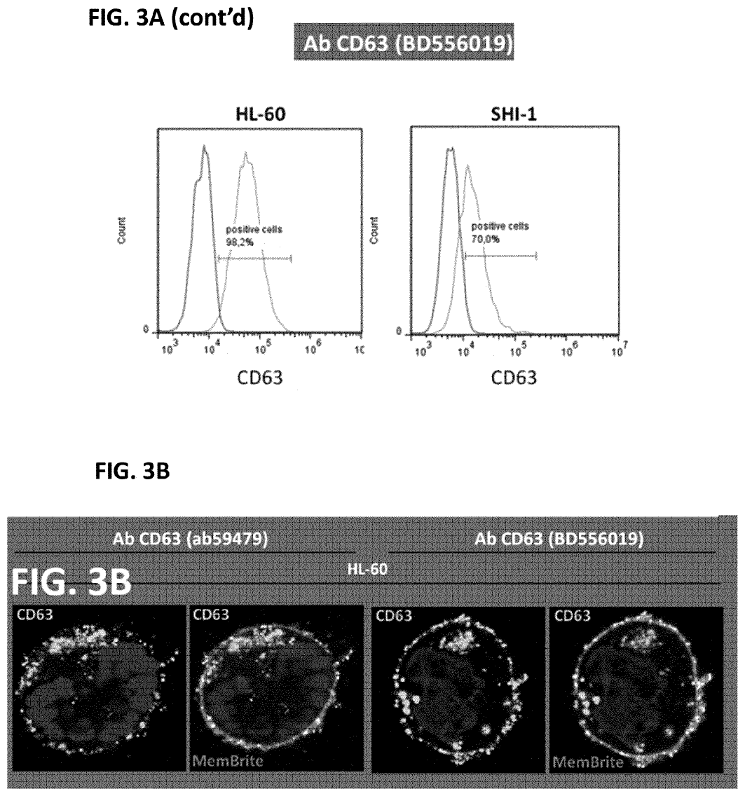

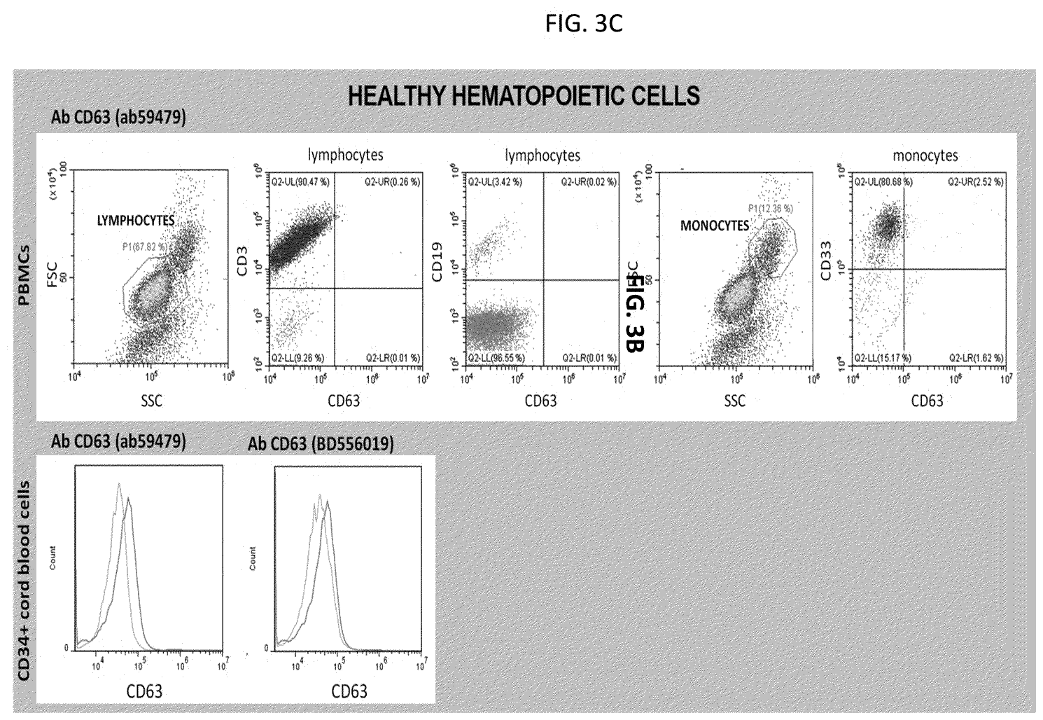

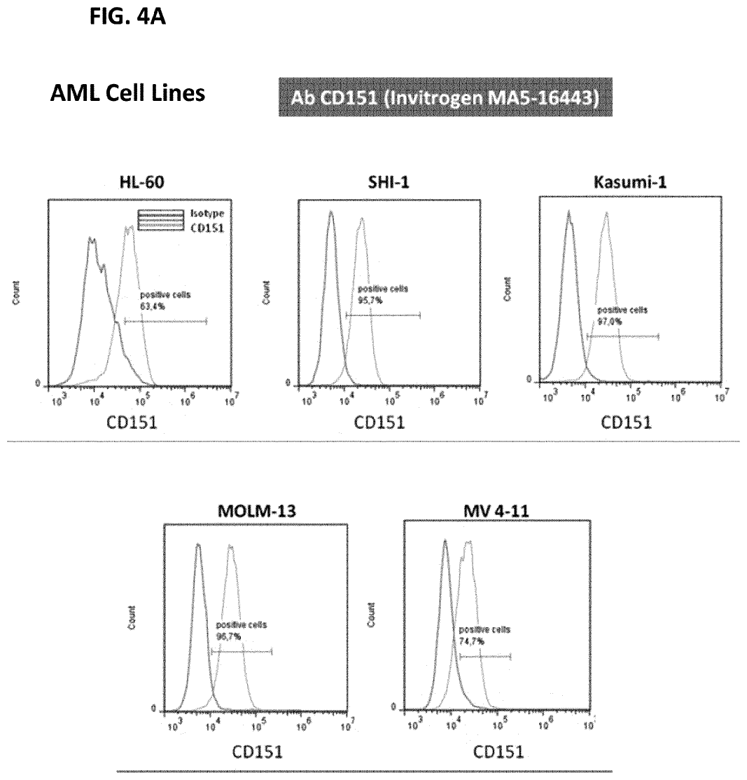

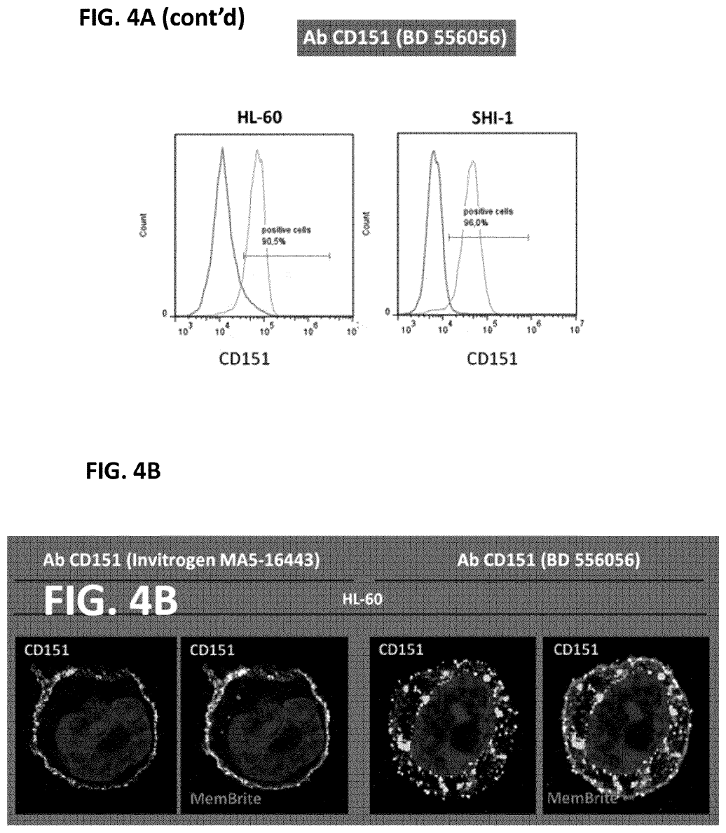

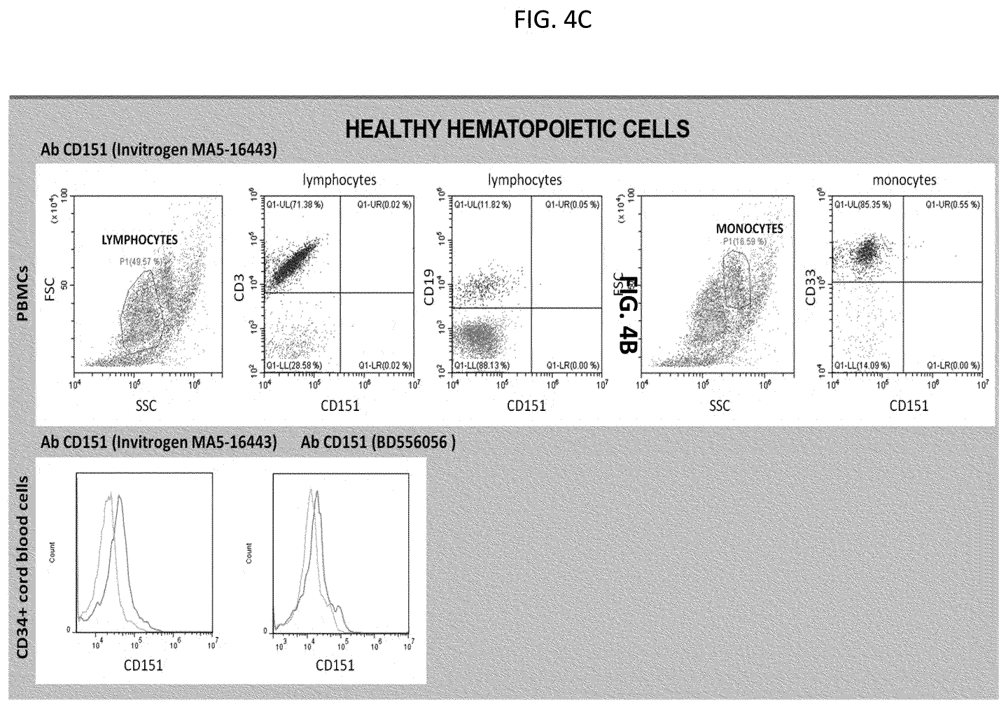

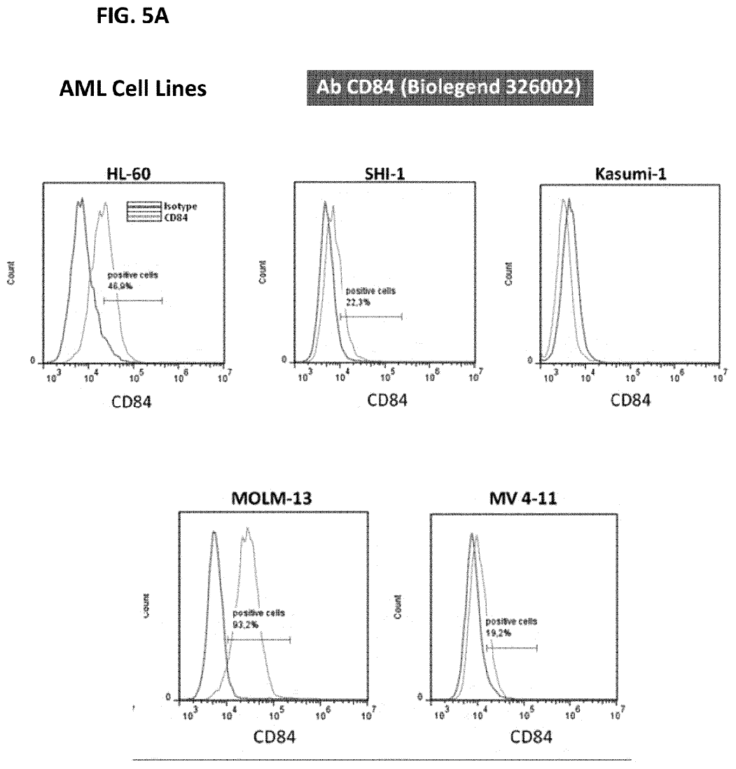

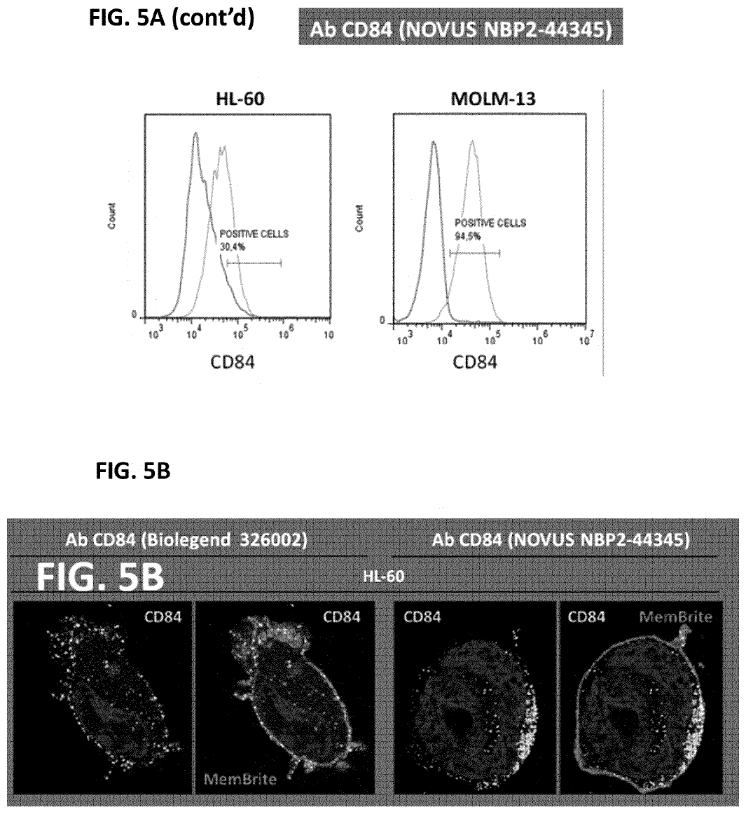

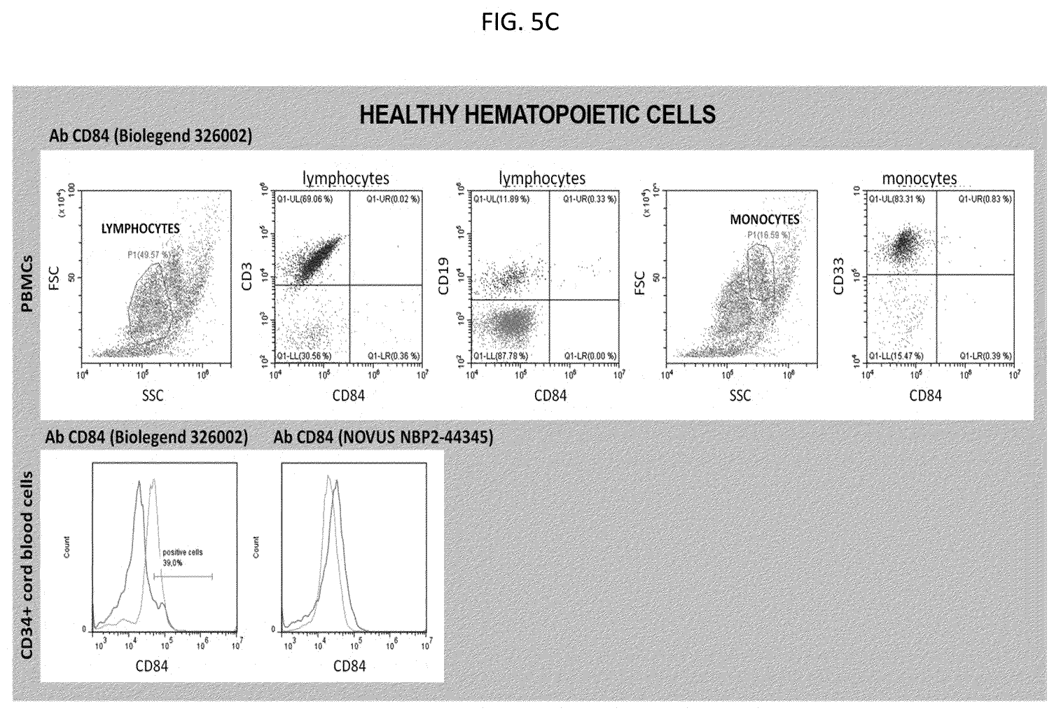

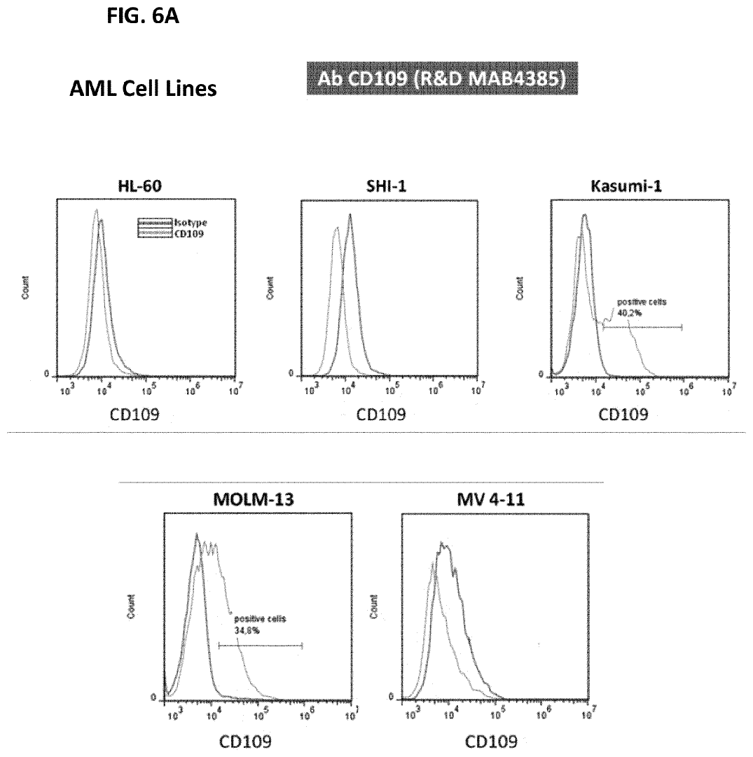

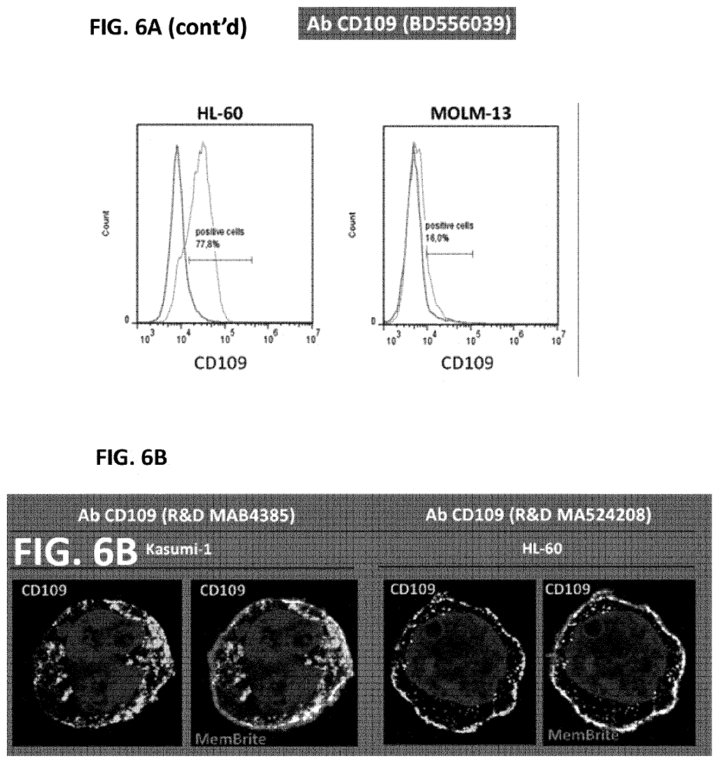

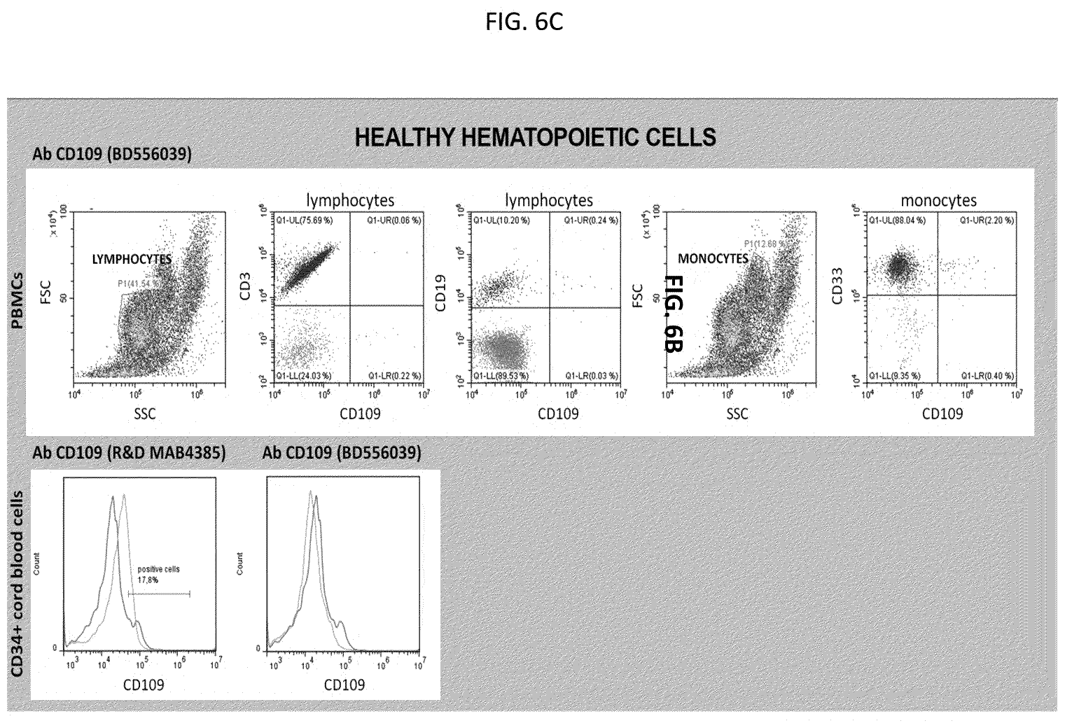

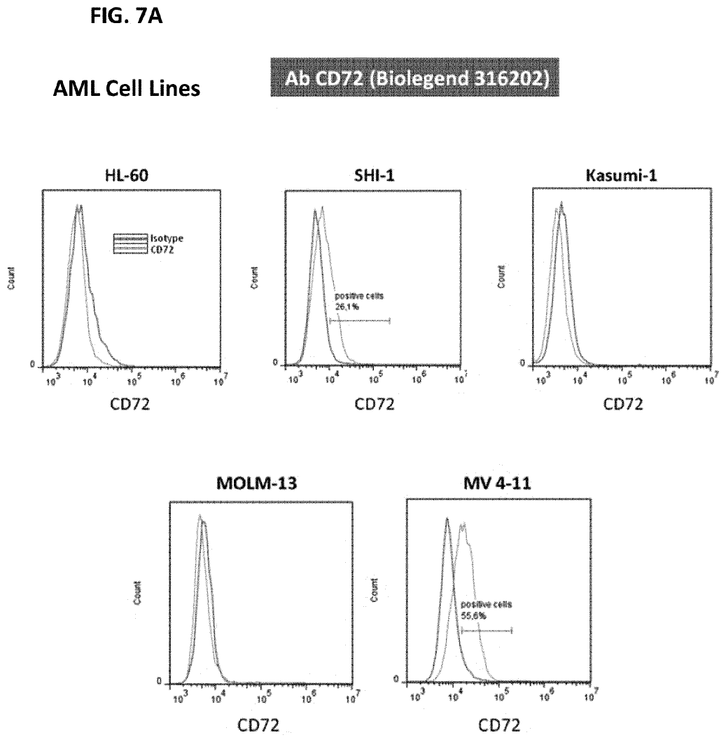

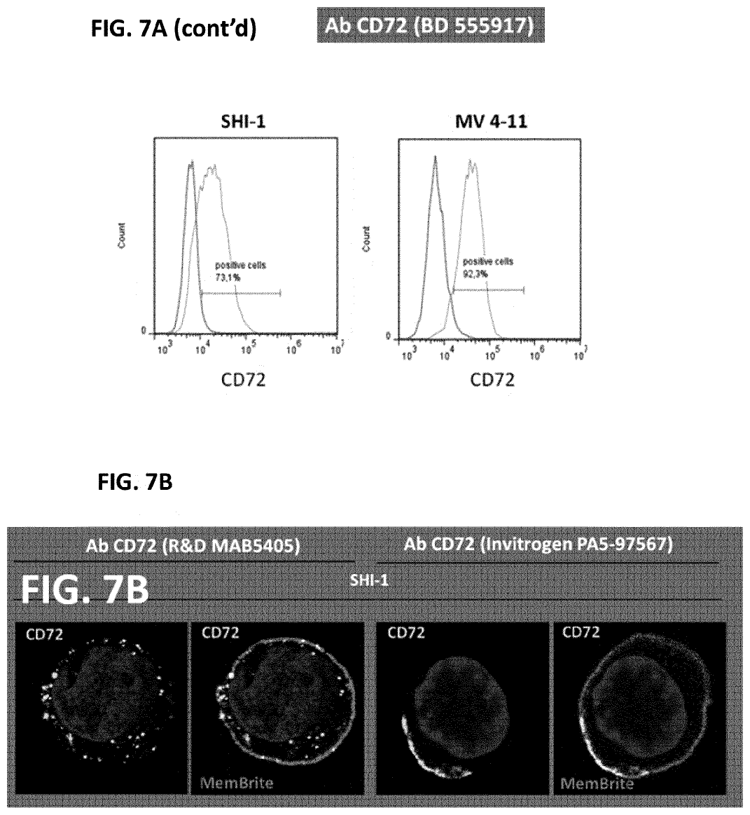

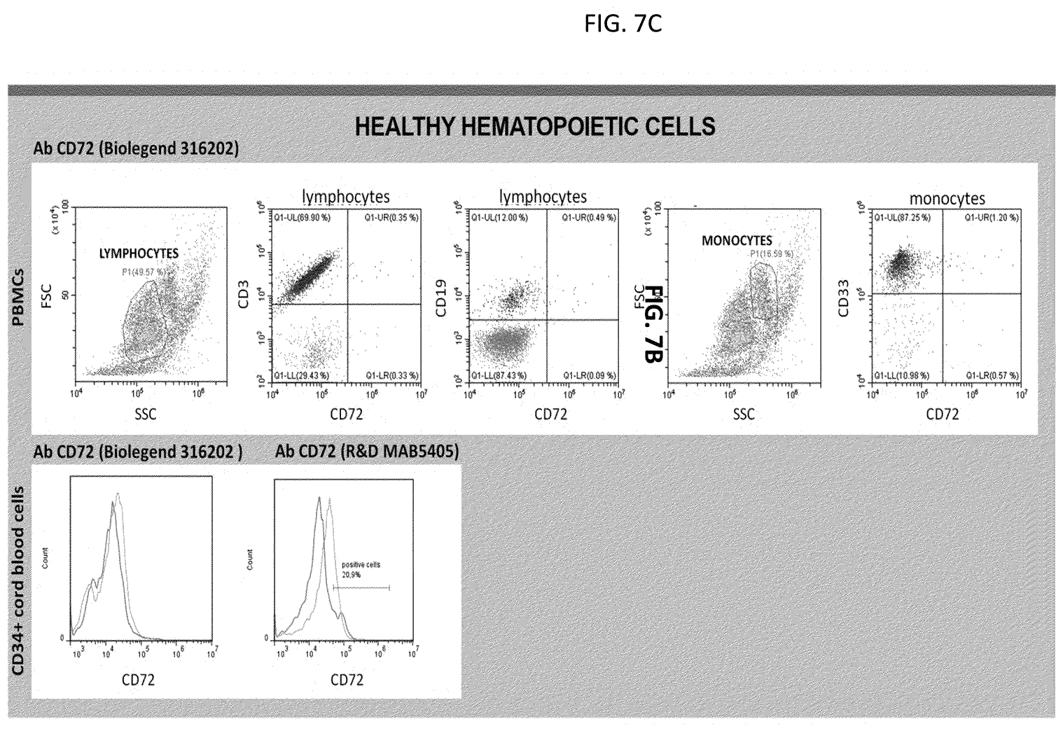

The flow cytometry and fluorescent immunohistochemistry data are provided in FIGS. 2A and 2B (CD69), FIGS. 3A and 3B (CD63), FIGS. 4A and 4B (CD151), FIGS. 5A and 5B (CD84), FIGS. 6A and 6B (CD109), and FIGS. 7A and 7B (CD72).

Expression of the antigen was specific to AML cells—much lower or no expression was detected by flow cytometry of healthy hematopoietic cells—fractionated subpopulations obtained from healthy donor peripheral blood mononuclear cells (PBMCs), including CD19+ B-lymphocytes, CD3+ T-lymphocytes, CD33+ myeloid precursors cells, and on CD34+ cells extracted from cord blood samples, as provided in FIG. 2C (CD69), FIG. 3C (CD63), FIG. 4C (CD151), FIG. 5C (CD84), FIG. 6C (CD109), and FIG. 7C (CD72). Expression of TSAs in healthy BM sub-populations, in normal regenerating bone marrow, in CD34⁺ and CD34⁺CD38⁻ subpopulations in normal regenerating bone marrow, in gated blasts of acute lymphoblastic leukemia patients, in gated blasts of AML patients at diagnosis, in gated blasts of AML patients at relapse, in gated blasts of AML patients with residual blasts, and in gated blasts of other malignancy patients such as patients with myeloid neoplasms is also summarized below in Tables 1-8, respectively.

TABLE 1
Expression of selected TSAs in healthy
blood mononuclear cell sub-populations

| | CD19 | CD3 | | |
|---|---|---|---|---|
| | B- Lym- | T -Lym- | CD33 | CD33 |
| | phocytes | phocytes | Monocytes | Granulocytes |
| CD69 | 1/6 POS D | 3/9 POS D | 1/8 POS D | 3/9 POS M |
| | 5/6 NEG | 6/9 NEG | 7/8 NEG | 6/9 NEG |
| CD63 | 6/6 NEG | 9/9 NEG | 1/8 POS B | 1/9 POS B |
| | | | 2/8 POS M | 3/9 POS D |
| | | | 5/8 NEG | 5/9 NEG |
| CD151 | 6/6 NEG | 5/9 POS D | 3/8 POS M | 3/9 POS D |
| | | 4/9 NEG | 1/8 POS D | 6/9 NEG |
| | | | 4/8 NEG | |
| CD84 | 6/6 NEG | 6/9 POS D | 3/8 POS M | 9/9 NEG |
| | | 3/9 NEG | 5/8 NEG | |
| CD109 | 2/6 POS D | 1/8 POS M | 8/8 NEG | 9/9 NEG |
| | 4/6 NEG | 6/8 POS D | | |
| | | 1/8 NEG | | |
| CD72 | 1/6 POS D | 2/9 POS D | 8/8 NEG | 9/9 NEG |
| | 5/6 POS M | 7/9 NEG | | |

STRONG POSITIVE (WHO classification)
POS B: positive bright
POS M: positive medium
POS H: positive heterogeneous
PP2: partial positive 2 (AIEOP-BFM Classification)
WEAK POSITIVE (WHO classification)
POS D: positive dim
PP1: partial positive 1 (AIEOP-BFM Classification)

Table 1 provides the expression of TSA in healthy blood samples and showed heterogeneous dim expression of TSA in peripheral blood mononuclear cell subpopulations.

TABLE 2
Expression of selected TSAs subpopulations
of normal regenerating bone marrow

| | B- Lym- | T -Lym- | | |
|---|---|---|---|---|
| | phocytes | phocytes | Monocytes | Myelocytes |
| CD69 | NEG | POS B* | NEG | NEG |
| | | (STRONG) | | |
| CD63 | NEG | NEG | POS M | POS H |
| | | | (STRONG) | (STRONG) |
| CD151 | NEG | NEG | POS M | NEG |
| | | | (STRONG) | |
| CD84 | NEG | NEG | POS B | POS H |
| | | | (STRONG) | (STRONG) |
| CD109 | NEG | NEG | NEG | NEG |
| CD72 | POS B | NEG | NEG | NEG |
| | (STRONG) | | | |

STRONG POSITIVE (WHO classification)
POS B: positive bright
POS M: positive medium
POS H: positive heterogeneous
PP2: partial positive 2 (AIEOP-BFM Classification)
WEAK POSITIVE (WHO classification)
POS D: positive dim
PP1: partial positive 1 (AIEOP-BFM Classification)

Table 2 provides TSA expression in healthy subpopulations from regenerating bone marrow collected at end of therapy and showed a low/dim expression in the subpopulation of the myeloid lineage in 3/6 TSAs.

TABLE 3
Expression of selected TSAs in CD34+ and CD34+ CD38−
subpopulations in normal regenerating bone marrow

| | CD34+ | CD34+ CD38− |
|---|---|---|
| CD69 | NEG | NEG |
| CD63 | 6/11 WEAK POS (D) | 6/11 WEAK POS (D) |
| | 5/11 NEG | 5/11 NEG |
| CD151 | 9/12 WEAK POS (PP1) | 7/12 WEAK POS (D) |
| | 1/12 STRONG POS (PP2) | 3/12 STRONG POS (M) |
| | 2/12 NEG | 2/12 NEG |
| CD84 | 11/12 WEAK POS (PP1) | 12/12 WEAK POS (D) |
| | 1/12 STRONG POS (PP2) | |
| CD109 | STRONG POS (H) | STRONG POS (B) |
| CD72 | STRONG POS (PP2) | NEG |

STRONG POSITIVE (WHO classification)
POS B: positive bright
POS M: positive medium
POS H: positive heterogeneous
PP2: partial positive 2 (AIEOP-BFM Classification)
WEAK POSITIVE (WHO classification)
POS D: positive dim
PP1: partial positive 1 (AIEOP-BFM Classification)

Table 3 provides TSA expression in the stem-precursors CD34⁺CD38⁻ subpopulation from regenerating bone marrow collected at end of therapy and showed a weak expression for some of the TSA.

TABLE 4
Expression of TSA in B or T acute lymphoblastic
leukemias at diagnosis

| | B-blasts | T-blasts |
|---|---|---|
| CD69 | 3/6 POS H (STRONG POS) | POS D (WEAK POS) |
| | 3/6 POS D (WEAK POS) | |
| CD63 | 1/6 POS H | \|NEG |
| | 5/6 NEG | |
| CD151 | 6/6 NEG | NEG |
| CD84 | 6/6 NEG | POS M (STRONG POS) |
| CD109 | 5/6 POS D (WEAK POS) | \|NEG |
| | 1/6 NEG | |
| CD72 | 4/6 POS B (STRONG POS) | \|NEG |
| | 2/6 POS H (STRONG POS) | |

STRONG POSITIVE (WHO classification)
POS B: positive bright
POS M: positive medium
POS H: positive heterogeneous (AIEOP-BFM Classification)
PP2: partial positive 2
WEAK POSITIVE (WHO classification)
POS D: positive dim
PP1: partial positive 1 (AIEOP-BFM Classification)

Table 4 provides TSA expression in acute lymphoblastic leukemia samples at diagnosis and showed all TSA suitable to detect B-lymphoblasts, whereas T-lymphoblasts were exclusively expressing CD69 and CD84.

TABLE 5
Expression of TSA in AML at diagnosis

| | Blasts | Blasts |
|---|---|---|
| | (WHO Classification) | (AIEOP-BFM Classification) |
| CD69 | 1/10 POS B | 4/10 STRONG POS |
| | 3/10 POS M | 4/30 WEAK POS |
| | 1/10 POS H | 2/30 NEG |
| | 3/10 POS D | |
| | 2/30 NEG | |
| CD63 | 1/10 POS B | 5/10 STRONG POS |
| | 3/10 POS H | 3/10 WEAK POS |
| | 1/10 POS M | 2/10 NEG |
| | 1/10 PP2 D | |
| | 2/10 POS D | |
| | 2/10 NEG | |
| CD151 | 1/10 POS B | 3/10 STRONG POS |
| | 2/10 POS M | 5/10 WEAK POS |
| | 2/10 POS H | 2/10 NEG |
| | 3/10 POS D | |
| | 2/10 NEG | |
| CD84 | 5/10 POS B | 8/10 STRONG POS |
| | 3/10 POS M | 2/10 WEAK POS |
| | 1/10 POS D | |
| | 1/10 PP1 | |
| CD109 | 2/5 POS H | 2/5 STRONG POS |
| | 3/5 NEG | 3/5 NEG |
| CD72 | 1/3 POS D | 1/3 WEAK POS |
| | 2/3 NEG | 2/3 NEG |

STRONG POSITIVE (WHO classification)
POS B: positive bright
POS M: positive medium
POS H: positive heterogeneous
PP2: partial positive 2 (AIEOP-BFM Classification)
WEAK POSITIVE (WHO classification)
POS D: positive dim
PP1: partial positive 1 (AIEOP-BFM Classification)

Table 5 provides TSA expression in acute myeloid leukemias samples at diagnosis and showed all TSA suitable to detect myeloid blasts, with CD69 and CD84 being the most specific.

TABLE 6
Expression of TSA in AML at relapse

| | Blasts | Blasts |
|---|---|---|
| | (WHO Classification) | (AIEOP-BFM Classification) |
| CD69 | 2/3 POS B | 2/3 STRONG POS |
| | 1/3 DIM | 1/3 WEAK POS |
| CD63 | 1/3 POS H | 1/3 STRONG POS |
| | 1/3 POS D | 1/3 WEAK POS |
| | 1/3 NEG | 1/3 NEG |
| CD151 | 1/3 POS B | 2/3 STRONG POS |
| | 1/3 POS M | |
| | 1/3 POS D | 1/3 WEAK POS |
| CD84 | 3/3 POS B | 3/3 STRONG POS |
| CD109 | 1/3 POS B | 2/3 STRONG POS |
| | 1/3 POS H | 1/3 NEG |
| | 1/3 NEG | |
| CD72 | 1/3 POS M | 1/3 STRONG POS |
| | 2/3 NEG | 2/3 NEG |

STRONG POSITIVE (WHO classification)
POS B: positive bright
POS M: positive medium
POS H: positive heterogeneous
PP2: partial positive 2 (AIEOP-BFM Classification)
WEAK POSITIVE (WHO classification)
POS D: positive dim
PP1: partial positive 1 (AIEOP-BFM Classification)

Table 6 provides TSA expression in acute myeloid leukemia samples at diagnosis shows all TSA suitable to detect blasts at disease relapse, with CD69 and CD84 being the more specific.

TABLE 7
Expression of TSA in treated AML with residual blasts (<5%)

| | Blasts | Blasts |
|---|---|---|
| | (WHO Classification) | (AIEOP-BFM Classification) |
| CD69 | 2/7 POS M | 2/7 STRONG POS |
| | 1/7 PP1 | 3/7 WEAK POS |
| | 2/7 POS D | 2/7 NEG |
| | 2/7 NEG | |
| CD63 | 2/6 POS B | 2/6 STRONG POS |
| | 3/6 POS D | 3/6 WEAK POS |
| | 1/6 NEG | 1/6 NEG |
| CD151 | 1/3 POS M | 1/3 STRONG POS |
| | 2/3 POS D | 2/3 WEAK POS |
| CD84 | 6/7 POS M | 6/7 STRONG POS |
| | 1/7 NEG | 1/7 NEG |
| CD109 | 1/1 NEG | 1/1 NEG |
| CD72 | 1/1 NEG | 1/1 NEG |

STRONG POSITIVE (WHO classification)
POS B: positive bright
POS M: positive medium
POS H: positive heterogeneous
PP2: partial positive 2 (AIEOP-BFM Classification)
WEAK POSITIVE (WHO classification)
POS D: positive dim
PP1: partial positive 1 (AIEOP-BFM Classification)

Table 7 provides TSA expression in bone marrow samples collected during therapy with myeloid residual disease ranging between 0 and 5% of blasts, with CD69 and CD84 being the most suitable to detect a low disease level.

TABLE 8
Expression of TSA in myeloid neoplasms

| | Blasts | Blasts |
|---|---|---|
| | (WHO Classification) | (AIEOP-BFM Classification) |
| CD69 | 1/4 POS B | 1/4 STRONG POS |
| | 2/4 POS D | 2/4 WEAK POS |
| | 1/4 NEG | 1/4 NEG |
| CD63 | 2/3 POS D | 5/7 STRONG POS |
| | 1/3 NEG | 2/7 NEG |
| CD151 | 1/2 POS B | 2/2 STRONG POS |
| | 1/2 POS M | |
| CD84 | 2/4 POS B | 3/4 STRONG POS |
| | 1/4 POS M | 1/4 WEAK POS |
| | 1/4 POS D | |

STRONG POSITIVE (WHO classification)
POS B: positive bright
POS M: positive medium
POS H: positive heterogeneous
PP2: partial positive 2 (AIEOP-BFM Classification)
WEAK POSITIVE (WHO classification)
POS D: positive dim
PP1: partial positive 1 (AIEOP-BFM Classification)

Table 8 provides TSA expression in bone marrow samples of myeloid neoplasms at diagnosis and showed the ability of CD69, CD84 and CD72 to reveal the dysplastic cells.

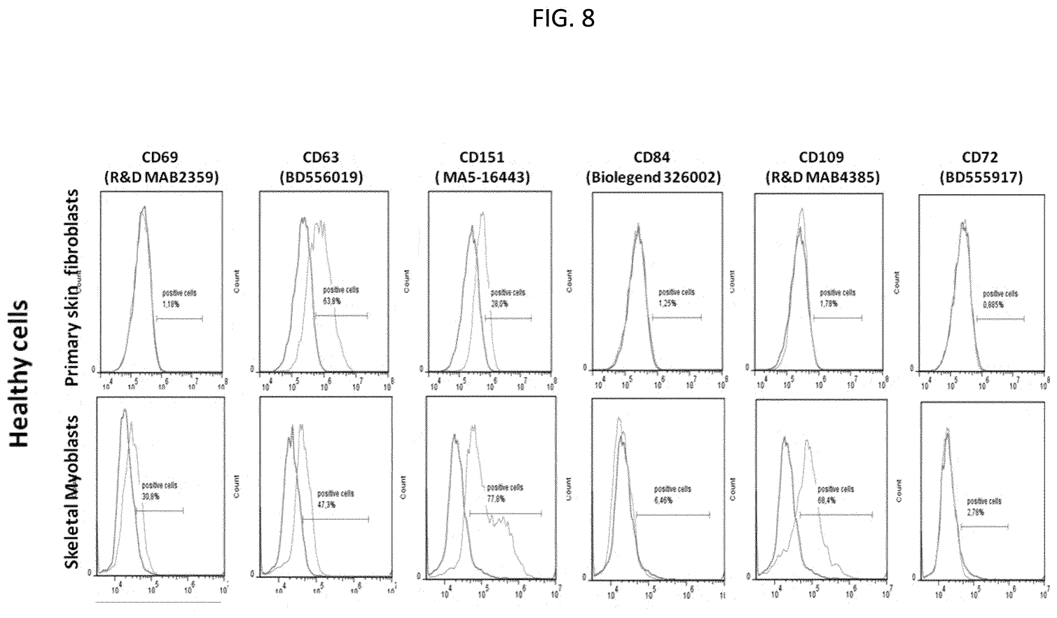

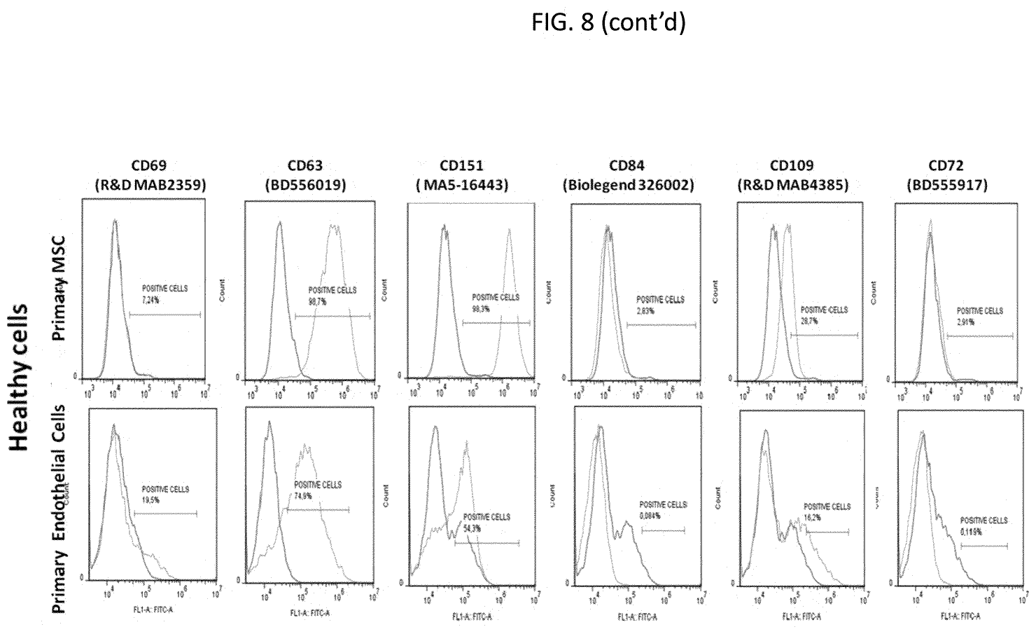

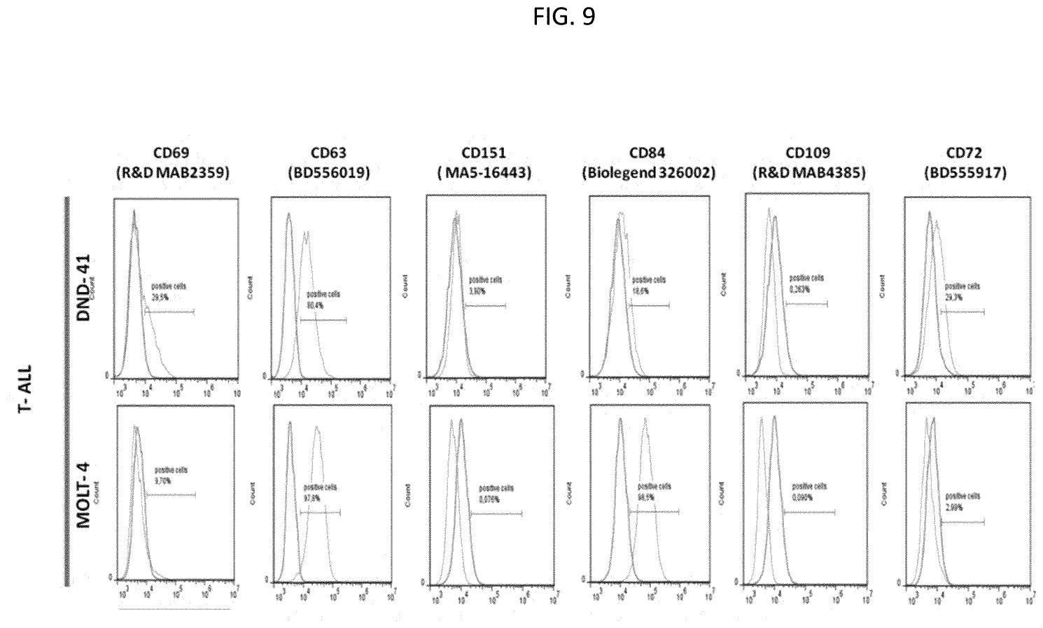

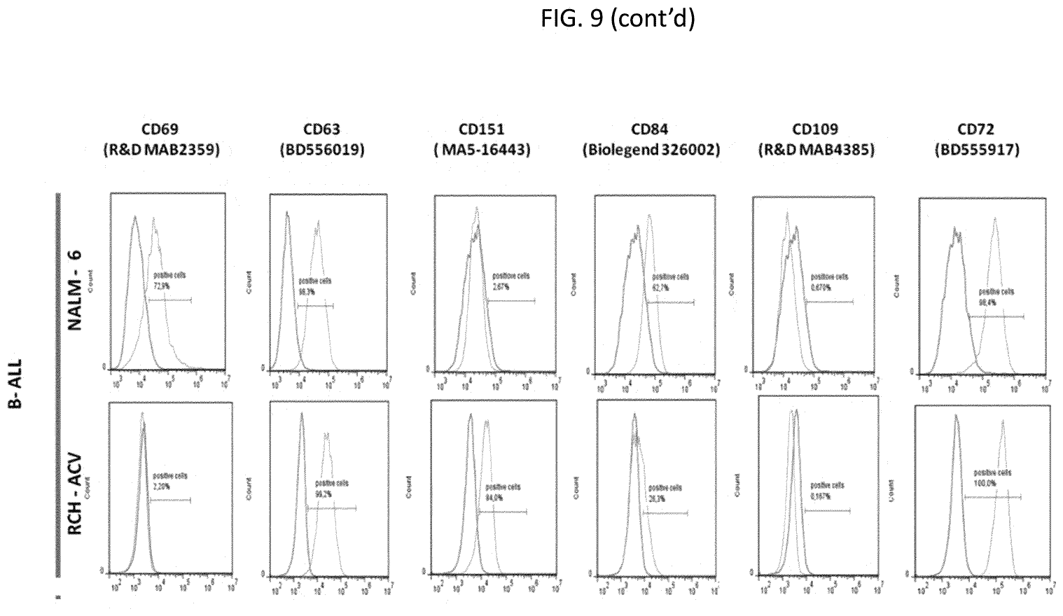

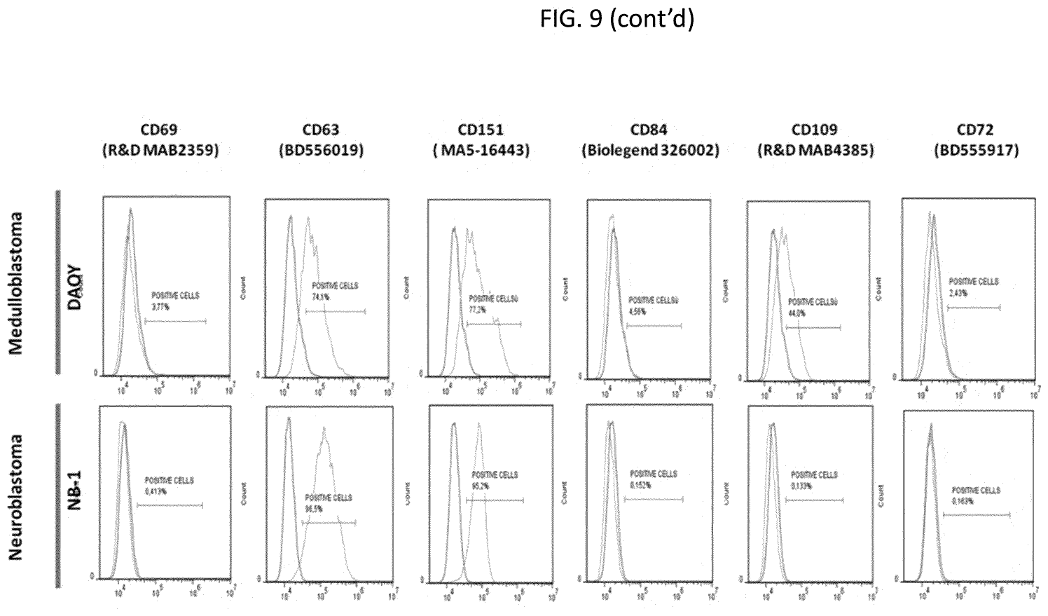

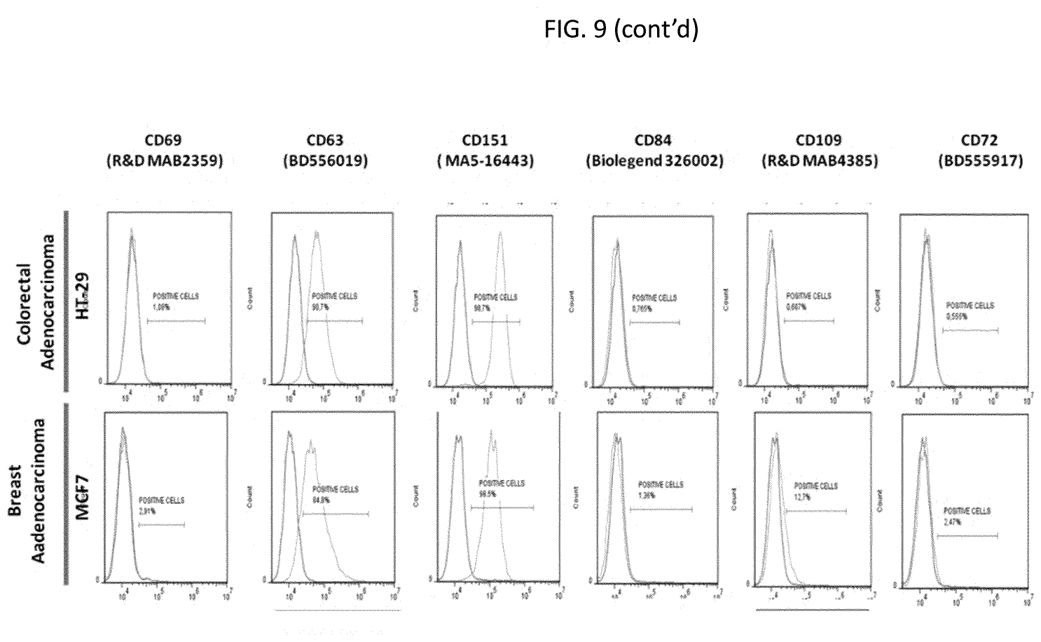

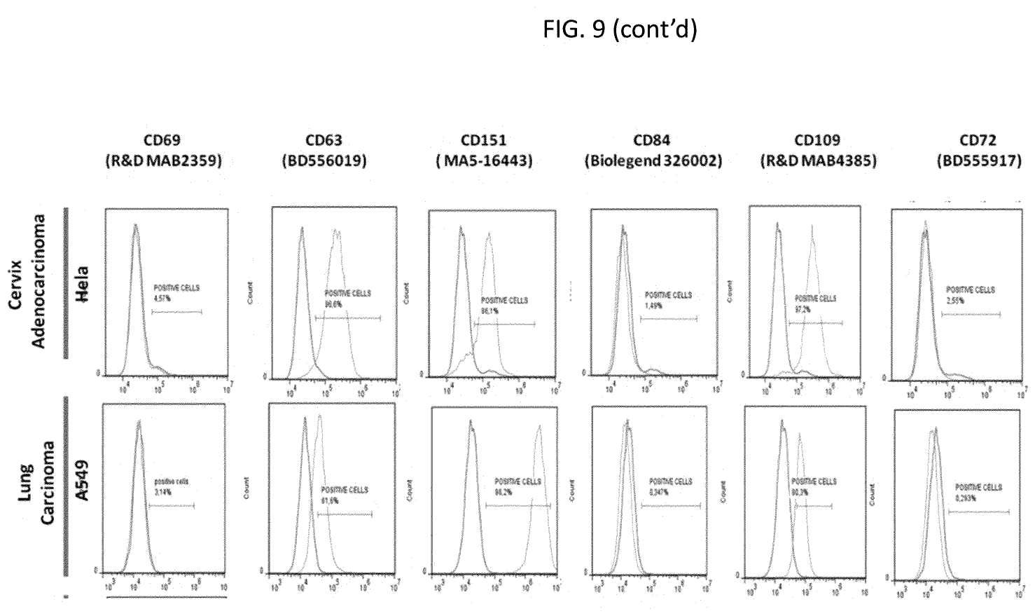

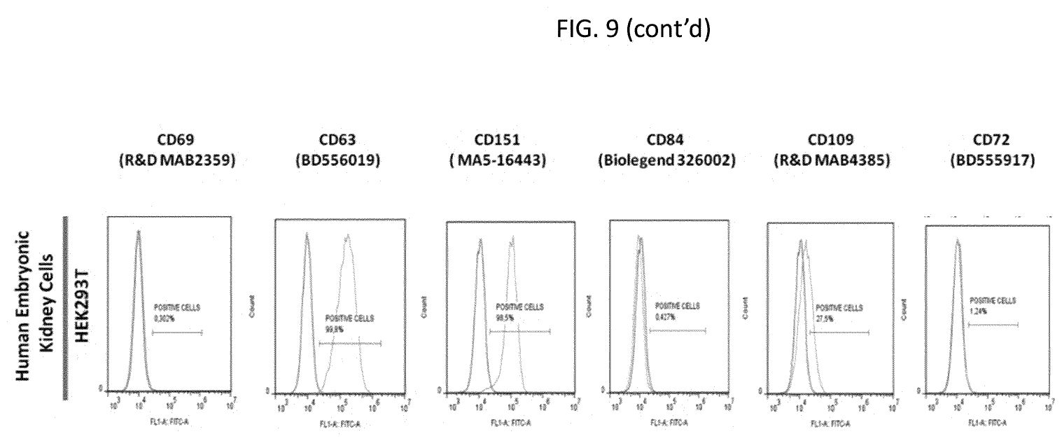

The specific expression was further confirmed by observing no or lower expression of the TSAs on healthy donor primary fibroblasts (FIG. 8).

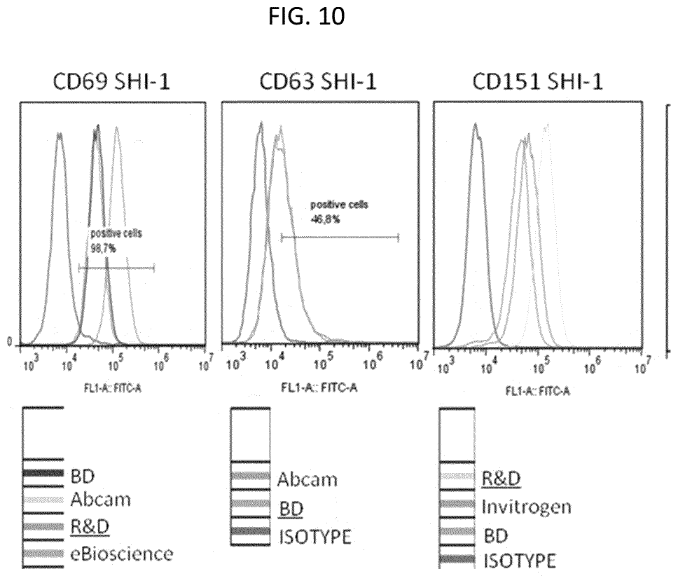

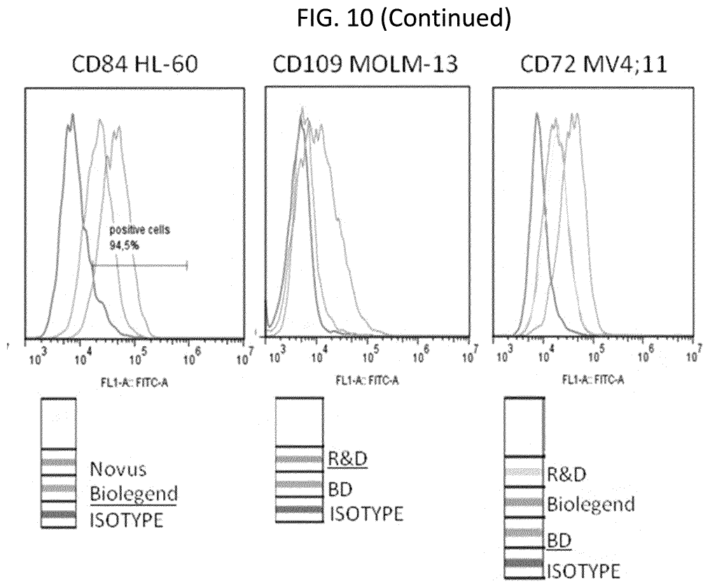

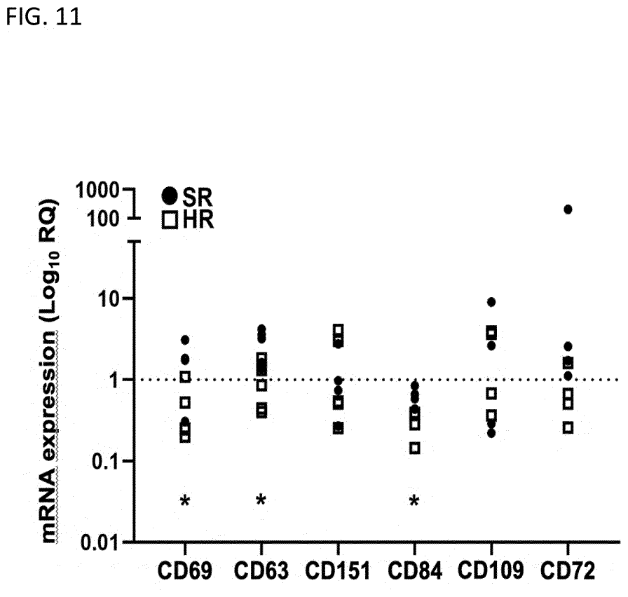

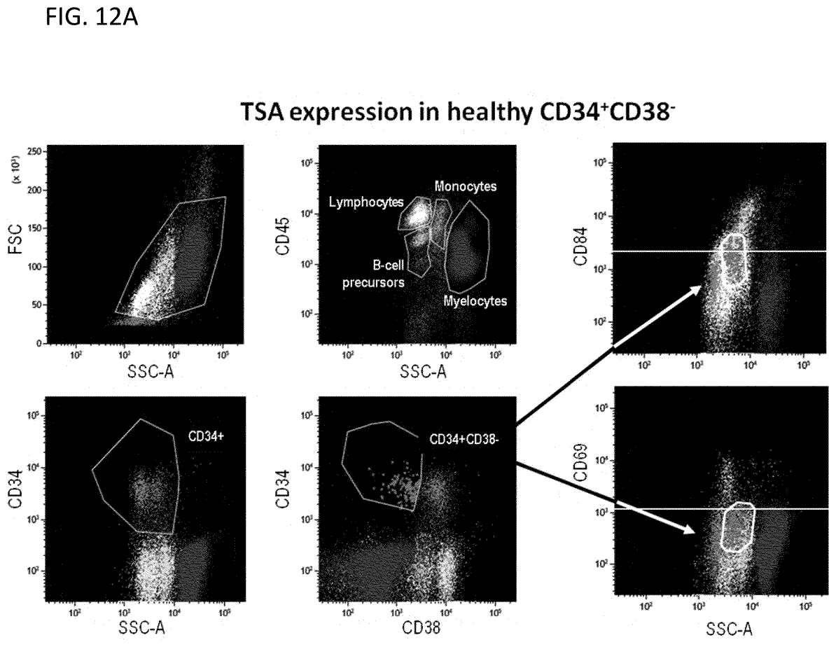

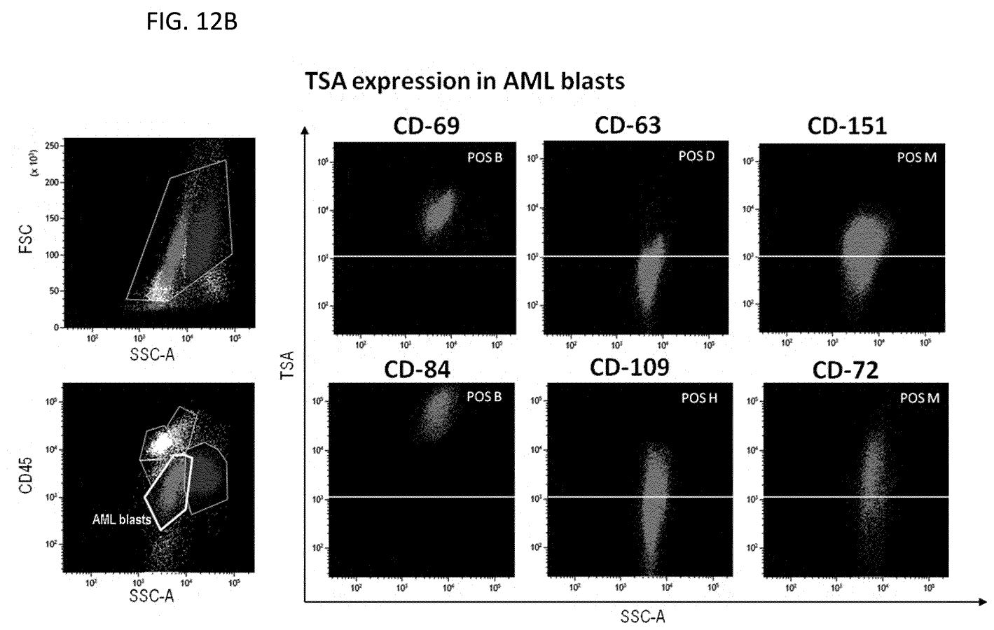

Expression of the TSAs was also tested in other cancer cell lines representing solid and liquid tumors different from AML (FIG. 10). The six TSAs were expressed in various levels, but overall expression levels were low in these other cancer cells.

These studies show that six selected TSAs are robustly and selectively expressed in the majority if not all the tested AML cell lines. Hyper-expression of the six TSAs was also assessed and confirmed with two AML patient-derived explants (PDXs) (generated from primary pediatric AML samples) available in the laboratory, by interrogating their gene expression profiles.

TSA stability and predictive value was investigated by evaluating their mRNA expression in pediatric AML samples obtained for diagnosis or for testing remission after therapy, according to the genetic risk stratification. TSAs hyper-expression was confirmed during the active stage of the disease (at diagnosis) with reduced expression in the samples collected at the end of therapy.

### Methods

Patients' samples and cell lines. Bone marrow (BM) samples were collected, processed and analyzed according to standardized operating procedures. 42 samples from patients affected by AML de novo—30 at diagnosis and 12 AML at relapse. 4 samples from myeloid neoplasms, 7 samples of AML collected during therapy with residual blasts (<5%), 6 samples of B-ALL and 1 of T-ALL at diagnosis, and 13 BM samples, collected during follow up when in disease remission and with regenerating characteristics as previously defined (Buldini B. et al., BJH 2018).

All AML cell lines were purchased by DSMZ. HL-60, Kasumi-1 and MOLM-13 were cultured in RPMI (GIBCO, Life Technologies, Paisley, UK) supplemented with 1% Penicillin-Streptomycin (GIBCO, Life Technologies, Grand Island, NY, USA), 1% L-Glutamine (GIBCO) and 10% Fetal Bovine Serum (GIBCO). MV4-11 and SHI-1 were cultured in Dulbecco's Modified Medium (GIBCO) supplemented with 1% Penicillin-Streptomycin, 1% L-Glutamine and 10% Fetal Bovine Serum.

Peripheral blood mononuclear cells (PBMCs) were obtained from buffy coats of healthy donors with informed consent by Ficoll density separation. CD34 positive cells were obtained from cord blood of healthy donors with informed consent by Ficoll density separation followed by a magnetic isolation in columns from CD34 MicroBead Kit (Miltenyi Biotech GmbH, Bergish Gladbach Germany) according to the protocol. Primary cells from whole blood (maximum 100 μL per tube) was stained with conjugated antibodies.

### Flow Cytometric Analyses.

BM samples were stained with primary conjugated antibody for 15 minutes in the dark, then they were hemolysed and centrifuged. Cells were re-suspended in PBS and analyzed by BD FACS CANTO II acquiring a minimum of 30,000 events.

Primary antibodies used for the clinical multi-parametric analyses (Table 4) were CD69 A488 (R&D Systems, catalog number FAB23591G), CD63 PE (BD Pharmingen, catalog number 556020), CD151 PE (R&D Systems, catalog number FAB1884P), CD84 APC (Biolegend, catalog number 326010), CD109 A488 (R&D Systems, catalog number FAB4385G), CD72 BV421 (BD Pharmingen, catalog number 743794) together with the following anti-human antibodies: CD45 V500 (BD Pharmingen, catalog number 560777), CD45 APC-Cy7 (BD Pharmingen, catalog number 348815), CD34 PC5 (Beckman Coulter, catalog number A07777), CD38 PE-Cy7 (BD Pharmingen, catalog number 335825), CD33 APC (BD Pharmingen, catalog number 551378), CD7 V450 (BD Pharmingen, catalog number 642916), HLA-DR V500 (BD Horizon, catalog number 561224), CD33 PC5 (Beckman Coulter, catalog number I112647), CD117 PC7 (Beckman Coulter, catalog number IM3698), CD34 A750 (Beckman Coulter, catalog number B92463), CD38 V450 (BD Pharmingen, catalog number 561378) CD19 PE (exbio, catalog number 1P-305-T100), CD7 PC5 (Beckman Coulter, catalog number I113613), CD34 APC (BD Pharmingen, catalog number 345804), CD3 APC-Cy7 (BD Pharmingen, catalog number 341110).

TABLE 9
Panels for detecting TSA expression by multi-parametric flow cytometry analysis

| | FITC | PE | PC5 | PC7 | APC | APCCY7 | V450 | V500 |
|---|---|---|---|---|---|---|---|---|
| 1 | CD69 | CD63 | CD34 | CD38 | CD33 | CD45 | CD7 | HLA-DR |
| 2 | CD109 | CD151 | CD33 | CD117 | CD84 | CD34 | CD38 | CD45 |
| 3 | CD69 | CD19 | CD7 | CD38 | CD34 | CD3 | CD72 | CD45 |
| 4 | CD69 | (CD4) | CD7 | CD34 | CD84 | CD45 | CD38 | CD8 |

Table 9 shows an antibody diagnostic panel for determining the immunophenotype at diagnosis by flow cytometry of acute leukemia or myeloid neoplasm.

### Intracellular Staining for the Validation of the Antibodies.

All the primary antibodies listed above were firstly validated by performing an intracellular staining of PBMCs as suggested by guidelines.

Briefly, cells (6×10⁵) were fixed with 4% paraformaldehyde in Phophatase Buffered saline (PBS) for 15 minutes at room temperature and then rinsed with PBS three times. Next, they were permeabilized with 0.1% TWEEN20 in PBS for 20 minutes and blocked with 3% bovine serum albumin (BSA) in PBS for 30 minutes. Permeabilized cells were stained with the primary antibodies together with Fc Receptor Blocking (Miltenyi) for 30 minutes and then incubated with secondary antibodies for 20 minutes.

### Cell Surface Staining.

Primary antibodies were tested for their binding on the surface of AML and other cancer cell lines, PBMC, and CD34 positive cells derived from cord blood. Cells (3×10⁵) were blocked at 3% BSA in PBS and stained with the primary antibodies together with Fc Receptor Blocking for 20 minutes. After washing with PBS, cells were stained with the secondary antibodies for 20 minutes.

For PBMCs subpopulations, an antibody against TSAs was used together with the following anti-human antibodies: CD3-APC (Beckam Coulter, Marseille, France), CD19-PE (Beckam Coulter), CD33-PC5 (Beckam Coulter) for 20 minutes. Next, cells were stained with the secondary antibody for 20 minutes. Cells were analyzed using CytoFLEX (Beckam Coulter) and Flow Jo Software (version 9.7, TreeStar Inc.).

### Immunofluorescence.

Primary antibodies used for these analyses are from eBioscience, Abcam, and BD; secondary antibody goat-anti mouse IgG Alexa488 antibody (Life Technologies). Cells (3×10⁵) were seeded to the bottom of culture chamber slides (FALCON, Big Flats, NY, USA) pre-coated with fibronectin (40 ug/ml) (Corning) for 2 hours at 37° C. After this incubation time, cells were stained with MemBrite Fix dye (Biotium), a specific membrane dye, for 5 minutes at 37° C. according to guidelines. Cells were fixed with 4% formaldehyde in PBS for 15 minutes and, after washing, blocked with 3% BSA in PBS for 30 minutes. After saturation, cells were stained with primary antibodies together with Fc Receptor Blocking overnight at 4° C., and then secondary antibody for 1 hour at room temperature. Cells were imaged with a Zeiss 2.6 laser scanning microscope. Images were analyzed using Image J win 32 software.

### 4.2. Example 2: Method of Treatment Targeting an AML-TSA

The six AML-TSAs (i.e., CD63, CD151, CD72, CD84, CD69, and CD109) have been tested as described in Example 1 and selected based on i) robust and broad expression in primary myeloid neoplasm, acute leukemia and acute myeloid leukemia, ii) lack/low expression in CD34+×CD38+ HSPCs, iii) links to tumorigenesis, and iv) expression in AML cell lines.

For development of a therapy targeting one of the TSAs, two to four different commercial antibodies against each target were tested to design the best single-chain variable fragment (ScFv) against each TSA. For each of the antibodies, flow cytometry results on AML cell lines are provided in FIG. 8.

Additionally, novel antibodies against the TSAs are generated using methods known in the art, for example using procedures involving immunogen preparation, immunization, hybridoma production, screening and purification or procedures for panning of phage-displayed naïve or immunized antibody libraries. The novel antibodies are also tested for their binding affinity and specificity to the TSA target.

An antibody selected for its binding affinity and specificity is used for development of an ABP-drug conjugate and CAR-T cell. The ABP-drug conjugate is generated by conjugating the selected antibody to a cytotoxic agent. The CAR is generated by using the antigen-binding domain of the selected antibody as the extracellular domain. A polynucleotide encoding a CAR comprising the antigen-binding domain, a transmembrane domain, a signaling domain and optionally at least one costimulatory domain is generated. The polynucleotide is transfected to a T cell or an NK cell to generate a CAR-T cell or CAR-NK cell.

Each of the ABP, ABP-drug conjugate and the CAR-T cells is tested in vitro using AML cell lines as well as primary pediatric AML samples. When the ABP, ABP-drug conjugate or the CAR-T cells is applied to a cell culture of AML cell line or primary AML cells, it induces cytotoxic effects specifically against cancer cells.

The ABP-drug conjugate and the CAR-T cells are also tested in vivo using an NSG (NOD/scid IL-2RgnuII) mice injected intravenously with AML cell lines (Isogenic human disease model) and by using patients' derived xenograft (PDX) animal model. Cancer cells in the animal model decrease after administration of the ABP, ABP-drug conjugate or the CAR-T cells in peripheral blood, bone marrow or spleen.

### 4.3. Example 3: Method of Treatment Targeting an AML-TSA Using CAR-T Cells

Validation of the use of the selected TSAs (CD63, CD151, CD72, CD84, CD69, and CD109) as targets for T cell-immunotherapy approaches based on the use of the generated CAR constructs.

Six tumor associated antigens, namely CD69, CD63, CD151, CD84, CD109 and CD72, were identified as uniquely associated to pediatric acute leukemia and myeloid disorders. The CD69 and CD84 antigens were characterized in vitro and in vivo and demonstrated to be highly specific for acute myeloid leukemia (AML) primary samples collected at diagnosis and at relapse.

Based on this data, 11 novel single-chain variable fragment (ScFv) sequences (A1, C1, F1, G1, C2, F2, H2, H3, F12, B8, and B11) were developed by phage display for generating chimeric antigen receptors (CAR) recognizing CD69 or CD84 on AML blasts. The CD72 antigen was shown to be highly specific for acute lymphoblastic leukemia (ALL), with great potential for the diagnosis of ALL patients treated with CD19-directed immunotherapy who frequently lose the CD19 marker.

### Design and Manufacturing of Novel CD84 and CD69 CAR-T Cells

Two novel antigens, CD84 and CD69, were prioritized as potential targets for AML immunotherapy. Phage display was used to find high-affinity antigen binders for both antigens from large combinatorial libraries containing up to billions of antibody targets. Method considered six rounds of panning as the iterative process for enriching phage within a phage population that possess high affinity binding to our targets compared to others, by exposing the library to our antigens after having enriched the population of phages with high-affinity, and then eluting and amplifying only those with the highest binding affinity. This qualitative selection process identified 87 clones for CD69 and 6 clones for CD84. The validation using the ELISA technique, which allows a more precise assessing of antibody-antigen interactions by quantitative immunoassay, confirmed 8 and 2 ScFv sequences being good candidates for CD69 and CD84, respectively. Gene synthesis produced 10 novel ScFv genes that were then cloned into a 3^{rd} generation chimeric antigen construct (CAR) containing the CD28 H/TM and 4-1BB costimulatory domains in combination with the zeta (CD3ζ) signaling domain, into a pUC57 plasmid. By restriction enzymes technology, CARs were cut and pasted into 3^{rd} generation lentiviral transfer plasmids for high titer lentiviral vector (LV) production (FIG. 13). Then, the workflow included the isolation of healthy donor T cells, followed by efficient activation, gene transfer of the CAR construct into the activated T cells, CAR-T cell expansion, phenotyping and analysis of the final cell product alone or after co-culture with target AML cell lines.

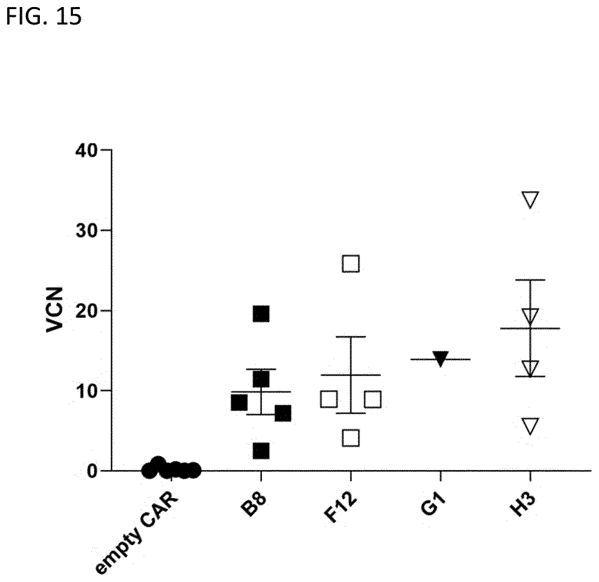

CAR-T cells were manufactured with four different specific ScFvs identified within a human naïve antigen-binding fragment (Fab) phage library, namely 14722-P1-F12 and 14722-P1-B8 recognizing CD84, and 14721-P1-H3 and 14721-P1-G1 recognizing CD69. The nucleotide (SEQ ID NOs: 24-34) and amino acid sequences (SEQ ID NOs: 35-44) of the four CAR constructs is provided in FIG. 27. Prior to viral transduction, T cells were isolated from peripheral blood mononuclear cells (PBMCs) collected from healthy donors by magnetic enrichment using CD4 and CD8 MicroBeads, and subsequently activated with the T Cell TransAct™ medium. Then, after 48 hours of expansion in TexMACS™, T cells were transduced with the CAR encoding LVs at a multiplicity of infection (MOI) of 10 in an IL-7 and IL-15 supplemented medium. After 24 hours of transduction, the vector was washed out and the CAR-T cells underwent expansion for 14 days (FIG. 14). Transduction efficiency was measured by digital droplet polymerase chain reaction (ddPCR) and expressed as vector copy number (VCN) per cell; the mean VCN ranged between 2.5 and 33.7 in all manufactured CAR-T cells, with no significant differences in VCN between the different CAR-T products (FIG. 15). Mock-transduced T cells (empty CAR) were used as control.

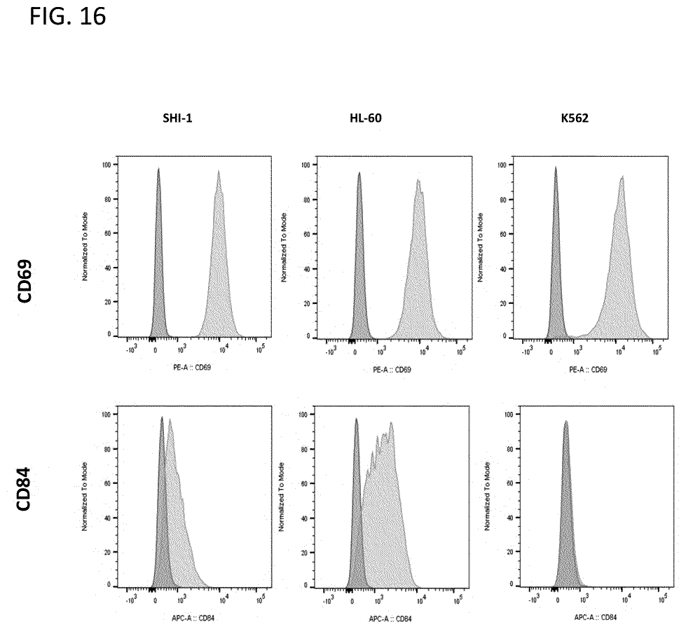

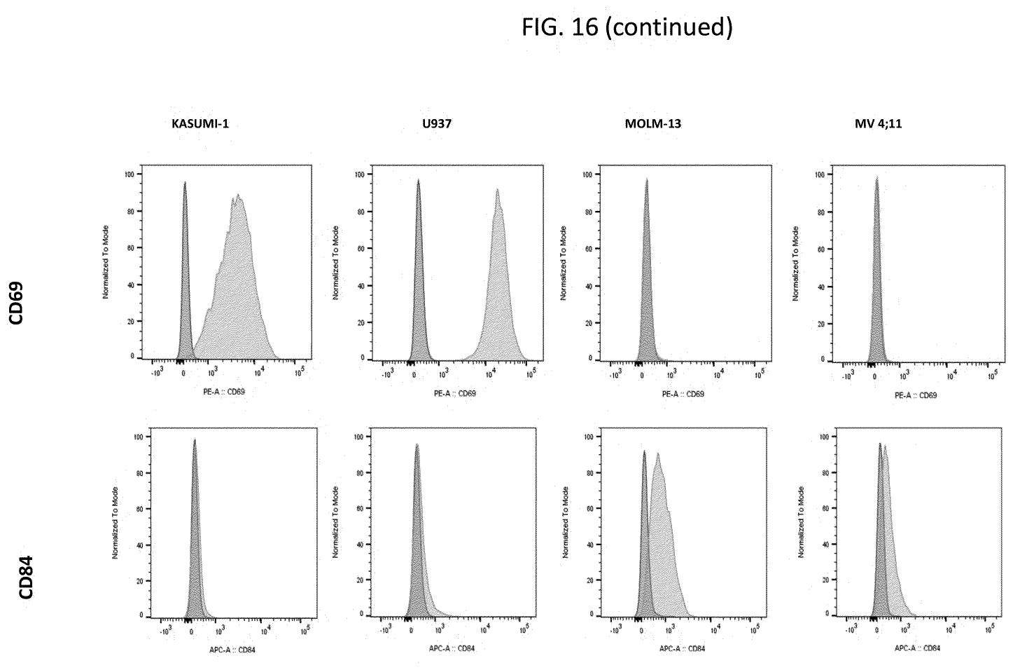

To properly test the potency and specificity of the newly manufactured CD84 and CD69 CAR-T cells in vitro and in vivo, AML cell lines were first screened for CD84 and CD69 cell membrane expression by flow cytometry. HL-60 and SHI-1 expressed both antigens at high levels being target cells (FIG. 16); MV4-11 and MOLM-13 expressed only CD84, whereas Kasumi-1, U937 and K562 cells expressed only CD69 (FIG. 16), being suitable as control cell lines for CD69 or CD84, respectively.

CAR Design does not Affect Expansion, Viability, and Phenotype of CD84 and CD69 CAR-T Cells

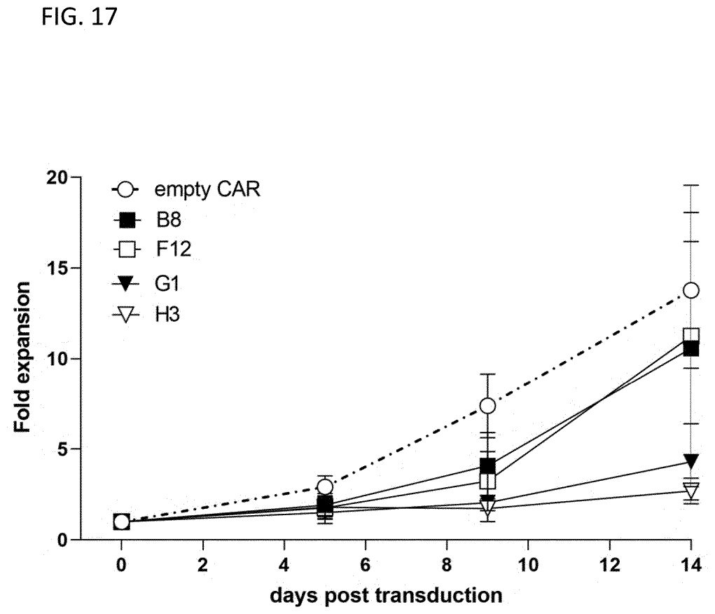

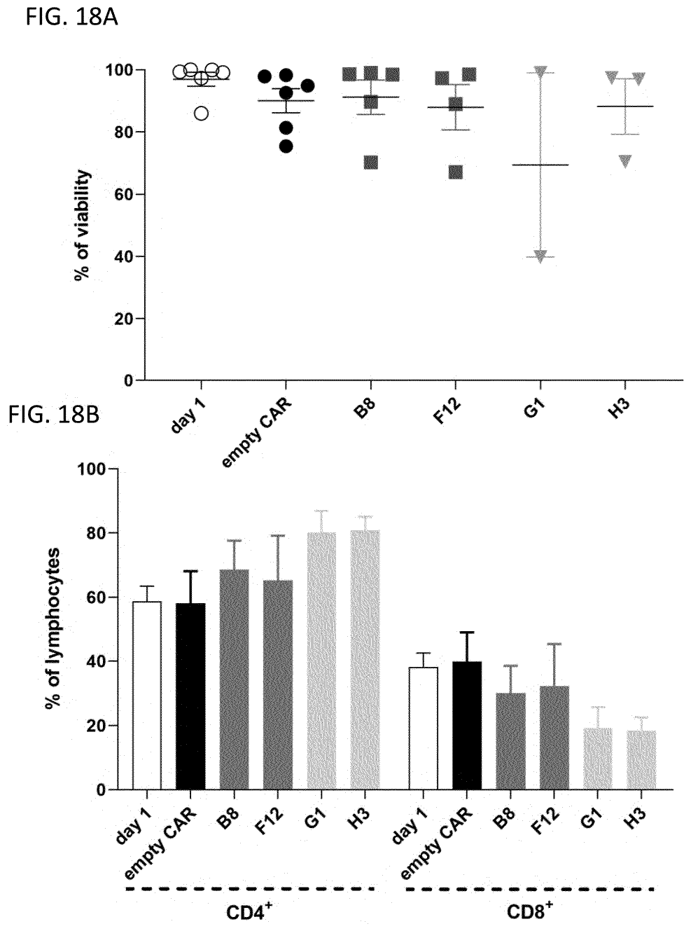

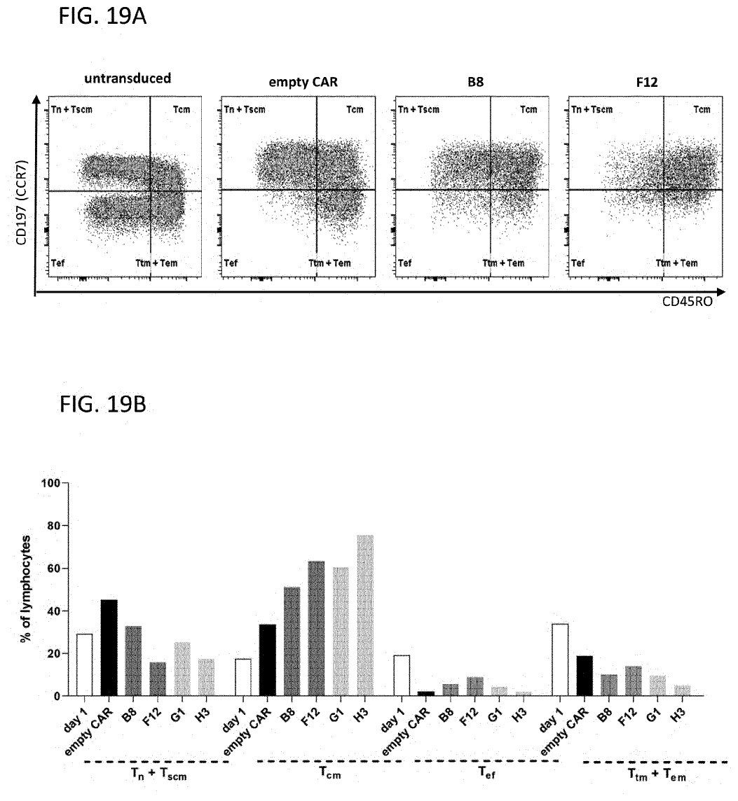

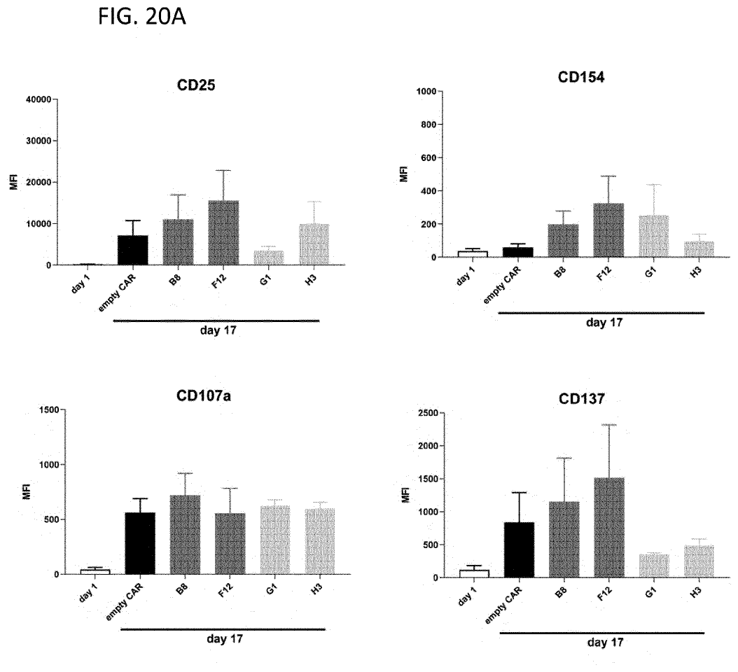

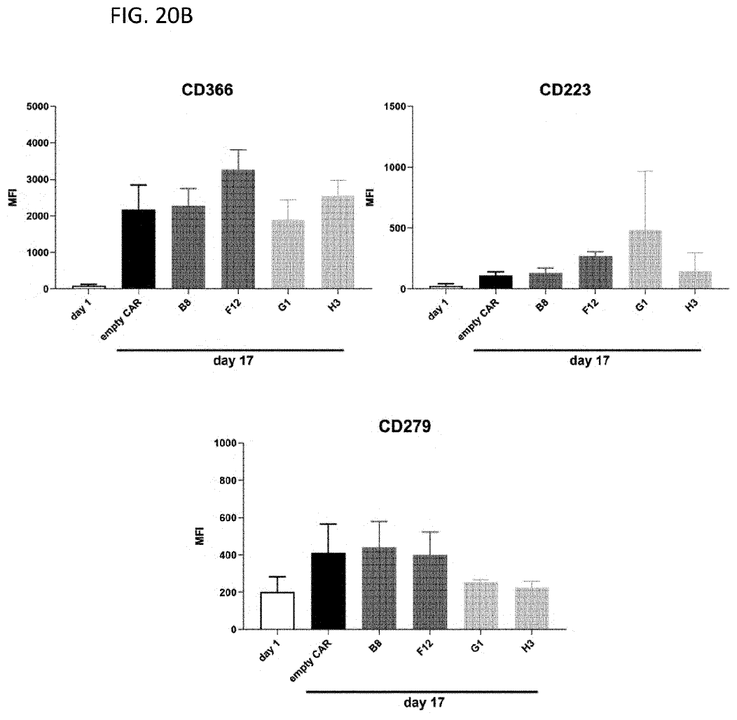

Manufactured CAR-T cells at end of 14 days of expansion (corresponding to day 17 of CAR-T cell manufacturing process) were counted and showed an expansion rate ranging between 2 and 20 folds (FIG. 17), with great variability mainly due to the starting donor PBMCs (FIG. 18A). The T cells were viable, enriched in CD4⁺ over CD8⁺ cells (FIG. 18B), and mostly comprised naïve and stem cell memory (Tₙ+T_{scm}, CD197[CCR7]⁺CD45R0⁻), and central memory (T_{cm}, CD197(CCR7)⁺CD45R0⁺) T cells, as expected (FIGS. 19A and 19B). Upon expansion, the CD84 and CD69 CAR-T cells exhibited both activation and exhaustion at the same extent as empty CAR-T cells, as shown by the upregulation of CD25, CD154, CD107a and CD137, as well as of CD366, CD223 and CD279, in response to culture conditions. It is worth noting that the same results were observed on control T cells (empty CAR), confirming that the CAR cassette was not altering T cell function (FIGS. 20A and 20B).

CD84 and CD69 CAR-T Cells In Vitro have Effective Antitumor Lytic Activity

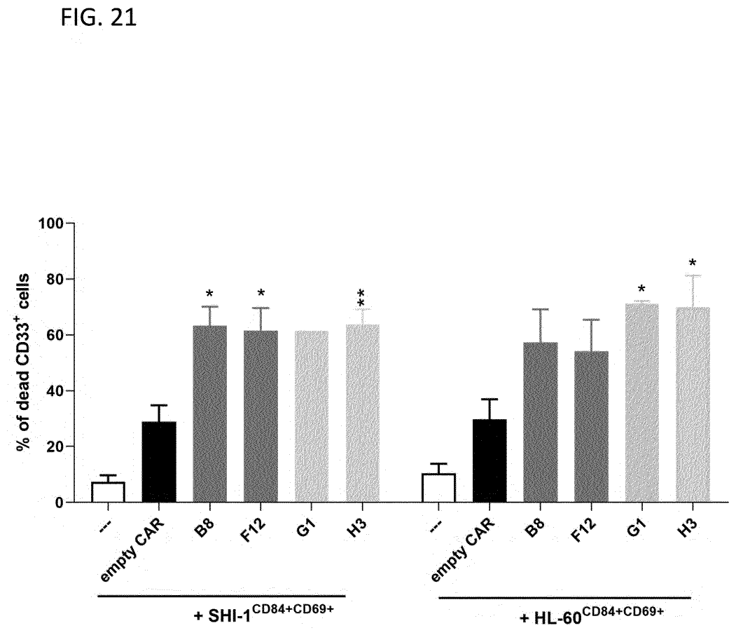

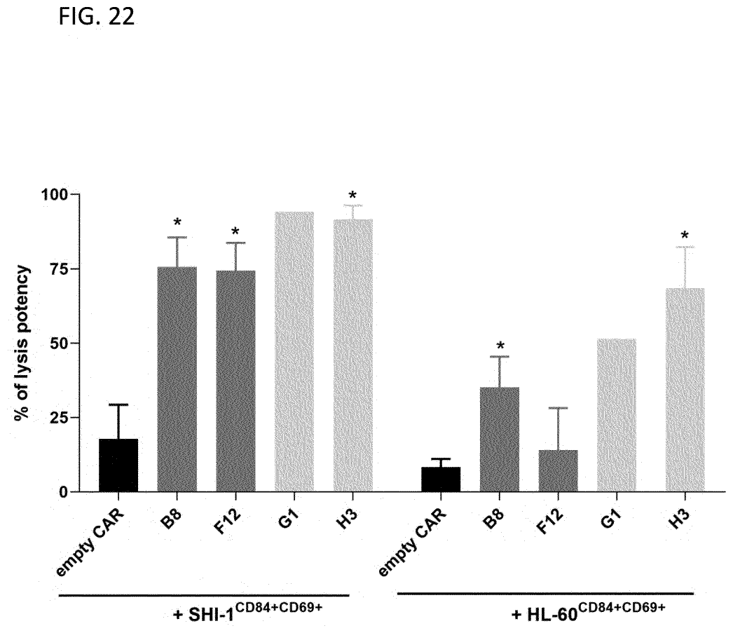

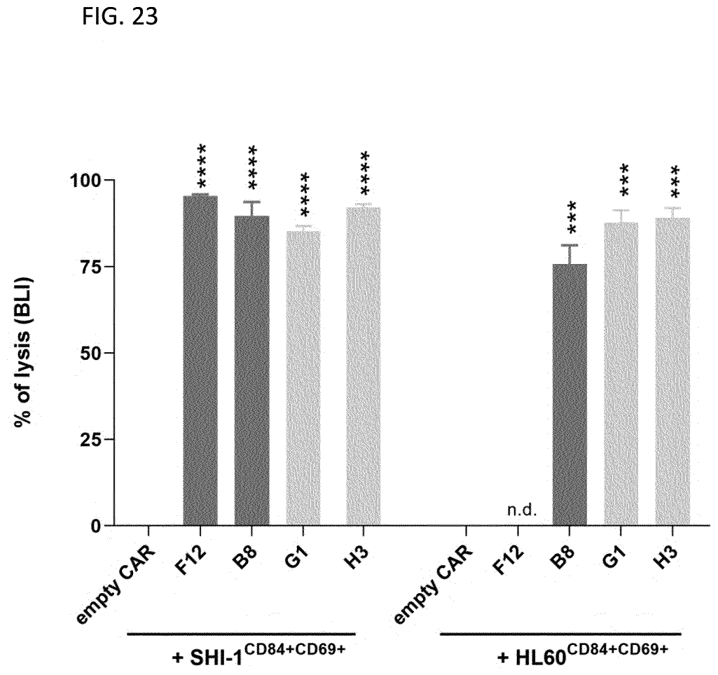

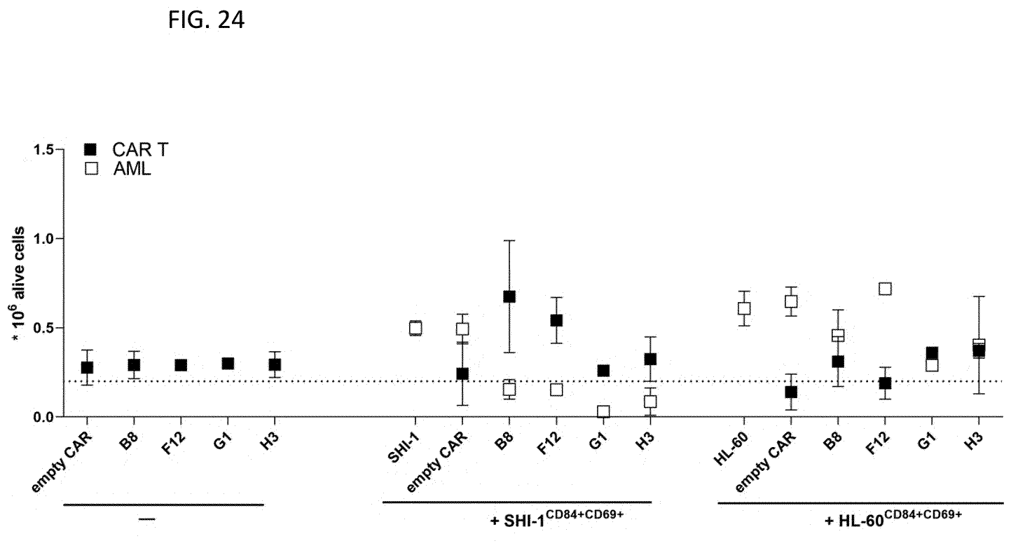

To test the activity of the newly generated CD84 and CD69 CAR-T cells, the CAR-T cells were maintained in media alone or in co-culture with target AML cell lines expressing the antigens. A significant in vitro antitumor activity was measured against the target cell lines by the four ScFv sequences tested, ranging between 10 and 80% on HL-60 and between 50 and 90% on SHI-2 cells. These results are further confirmed by evaluating 1) the frequency of CD33⁺ dead cells (AnnexinV⁺ and/or 7AAD⁺, FIG. 21), 2) the lysis potency (FIG. 22) and 3) the bioluminescence signal in luciferase-transduced AML cells (FIG. 23). The persistence of the activated CAR-T cells was also monitored by cell count up to 48 hours in vitro. The activity of CAR-T cells is characterized by an increased proliferation of T lymphocytes (black squares) with a concomitant reduction of target AML cell number (white squares, FIG. 24).

### CD84 and CD69 CAR-T Cells Display No Toxicity Towards CD34⁺ Hematopoietic Stem and Progenitor Cells (HSPCs)

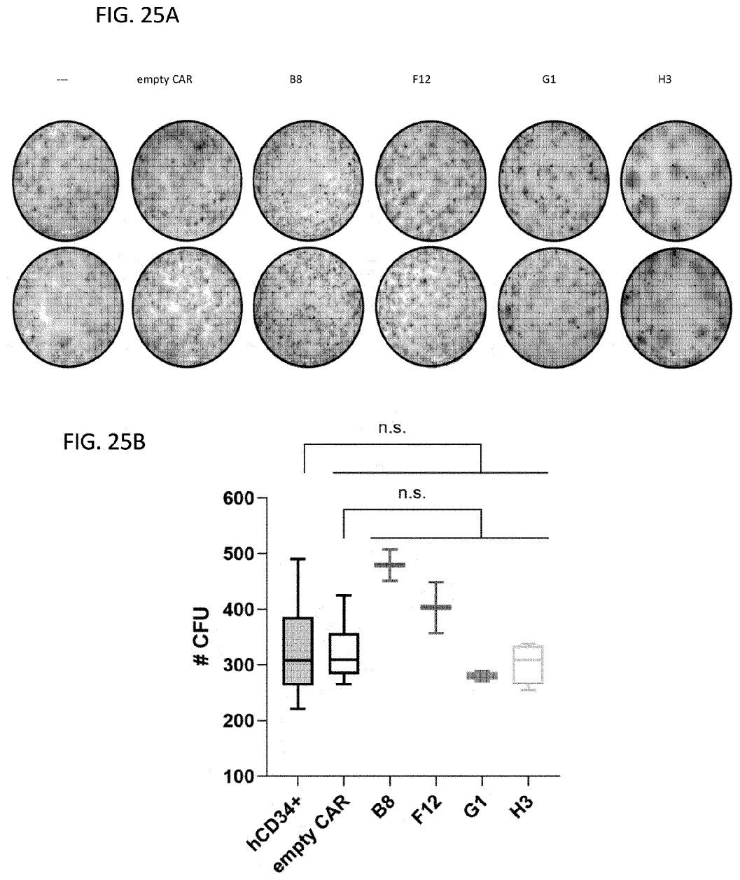

The potential off-target activity of CD84 and CD69 CAR-T cells against CD34⁺ hematopoietic stem and progenitor cells (HSPCs) was interrogated in a standard colony-forming unit (CFU) assay at 1:1 effector:target ratio (E:T). Both CD84 and CD69 CAR-T constructs did not produce significant effects on CD34⁺ cells, as they induced neither a significant reduction in the number of CFUs formed by CD34⁺ HSPCs nor a reduction in CD34⁺ viability (FIG. 25A and FIG. 25B).

### T Cells Expressing CD84 and CD69 CAR Recognize and Kill AML Cell Targets In Vivo

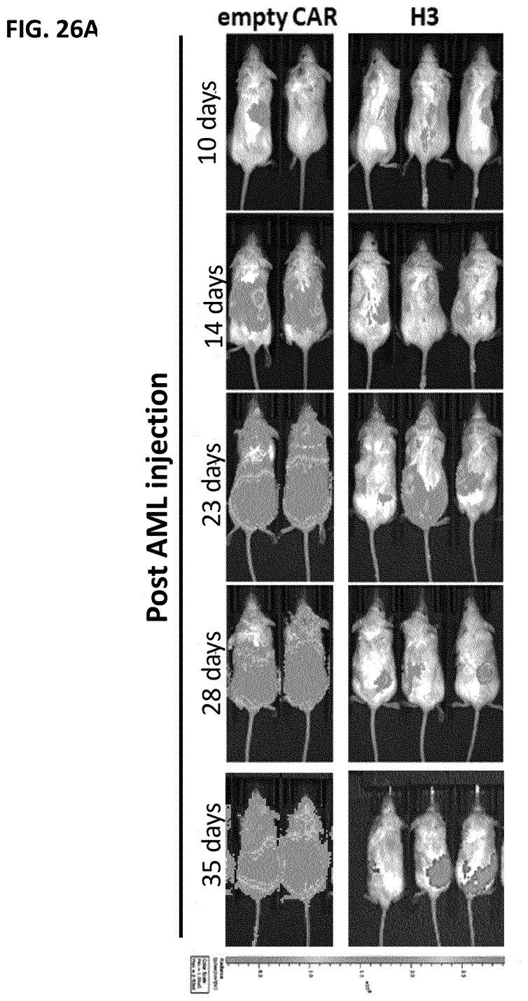

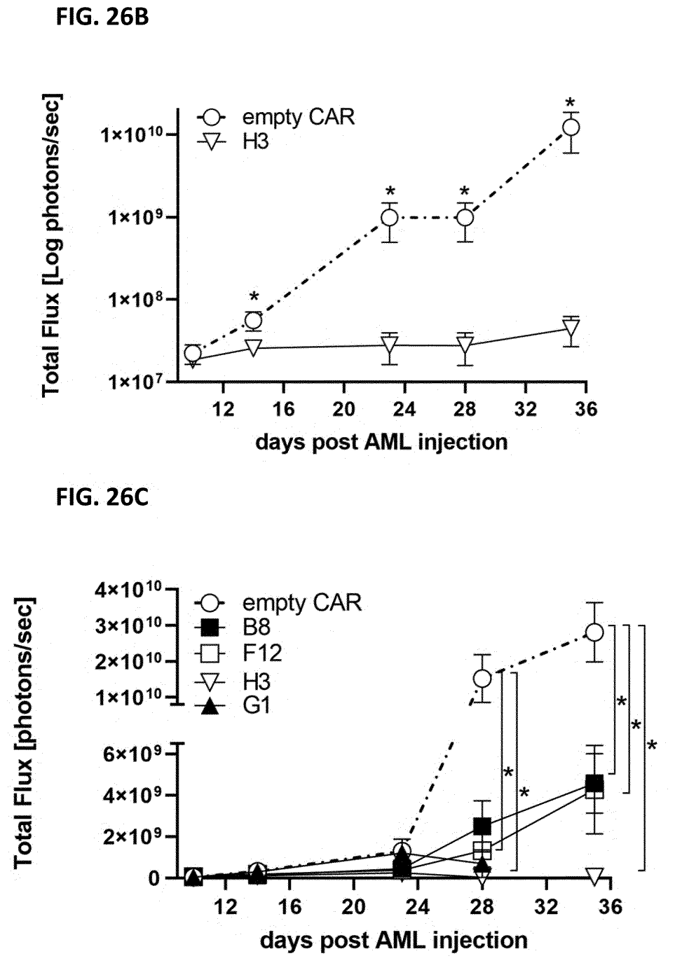

To test CAR-T cell activity in vivo, CAR-T cells targeting CD84 and CD69 were produced and infused them in NOD/SCID gamma (NSG) mice engrafted with the target AML cell lines. AML cell lines were engineered to express luciferase for non-invasive bioluminescence imaging (BLI). Briefly, mice were tail-vein injected with 0.5×10⁶ AML cells/mouse (day 1) and then (day 3) with 1.5×10⁶ CAR-T cells or mock transduced T cells (MOI 10 empty CAR/mouse; n=6-8 per condition tested). Mice treated with B8 and F12 (CD84) and G1 and H3 (CD69) CAR-T cells experienced a statistically significant reduction in leukemic burden, assessed by BLI, already at day 14 following AML challenge, becoming even more evident over time (at day 23 and 35) (FIG. 26A, 26B, 26C).

### Material and Methods

### Human Cell Lines

HL-60 and SHI-1 AML cell lines were used as target cells for TSA screening. These cell lines were grown in RPMI (Life Technologies; 11875093) supplemented with 10% fetal bovine serum (FBS; Life Technologies; 10270106), 1% Penicillin-Streptomycin (P/S, 10000 U/mL, Life Technologies; 15140148) and 1% L-Glutamine (200 mM, ThermoFisher; 25030024) except for SHI-1 cell line that was cultured in DMEM (Life Technologies; 41965039) supplemented with 10% FBS and 1% P/S. HEK293T were grown in IMDM (Euroclone; ECB2072L) supplemented with 10% FBS, 1% P/S and 1% L-glutamine. All cells were cultured at 37° C. in a humidified incubator with 5% CO₂.

### Cloning

The CAR cassettes (1506 bp) were cloned from pUC57 vectors (synthetized by ProteoGenix SA) into a 3^{rd} generation LV transfer plasmid under the control of the human phosphoglycerate kinase promoter (hPGK). One Shot TOP10 chemically competent E. coli (ThermoFisher, Walthan, MA, USA) were transformed, and DNA was extracted with Plasmid DNA Maxiprep Kit (Termo Fisher Scientific; K210017). The procedure was controlled by digestion with BamHI and SalI enzymes and agarose gel electrophoresis. Sanger Sequencing of the plasmids was performed to verify the correct insertion of the cassette in the backbone.

CD4⁺ and CD8⁺ Isolation from PBMCs and T Cell Expansion

PBMCs were isolated from healthy donors by density gradient centrifugation using Lymphoprep (StemCell technologies; 07861). From PBMCs, CD4⁺ and CD8⁺ T cells were magnetically isolated using autoMACS instrument (Miltenyi Biotec; Bergisch Gladsbach, Germany) with CD4 and CD8 MicroBeads (Miltenyi Biotec; 130-045-101 and 130-045-201, respectively) according to manufacturer's instructions. Isolated T cells were then activated at day 1 for in vitro expansion using TransAct (Miltenyi Biotec; 130-111-160) in TexMACS Medium (Miltenyi Biotec; 130-097-196) with 1% P/S and supplemented with recombinant human IL-7 (500 IU/ml) and of IL-15 (84 IU/ml) (Miltenyi Biotec; 130-095-362 and 130-095-764 respectively). At day 4 cells were washed from TransAct and lentivirus and then maintained in TexMACS with IL-7 and IL-15 at 1-2×10⁶ cells per mL.

### Lentiviral Vector Production and Transduction of Human T Cells

LVs were produced via transient transfection of HEK293T packaging cell line as previously described (Langford-Smith et al., 2012). Briefly, 70% confluent cells were co-transfected with Gag/Pol (III gen), Env and Rev packaging plasmids, pAdvantage plasmid (pADV) and the transfer vector plasmid with the cassette. After 48 hours from the transfection, lentiviral supernatant was collected, ultracentrifuged, aliquoted and stocked at −<65° C.

T cells were transduced with LVs on day 3 at a MOI of 10 with 0.01 mg/mL Vectofusin-1 (Miltenyi Biotec; 130-111-163). After 16 hours from the transduction, cells were washed from LV and TransACT. To evaluate transduction efficiency, the VCN per cell were measured. Total genomic DNA from transduced T cells was extracted after 14 days from the transduction with the Dneasy Blood & Tissue Kit (Qiagen; 69504). To determine the VCN per cell, ddPCR was used with the reaction mixture containing ddPCR Supermix for Probes without dUTP (Bio-Rad; 1863024) and the primer-probe sets for target and reference genome (ddPCR™ CNV Assay [FAM)] 10031277; ddPCR™ CNV Assay [HEX] 10031244). The droplets were made by Automated Droplet Generator (Bio-Rad) and read with a QX200 droplet reader (Bio-Rad). The VCN was analyzed with QuantaSoft droplet reader software and determined by the ratio of the target-gene concentration over the reference-gene concentration, multiplied by number of copies of reference gene in the reference genome.

### Activation, Exhaustion and Differentiation Markers by Flow Cytometry

Cells were stained with fluorochrome-conjugated primary antibodies and isotype control for 15 minutes at room temperature. Stained cells were washed and immediately analyzed with FACSCelesta™ Cell Analyzer (BD Biosciences). The following antibodies were used: CD3 (PE-Vio615, Miltenyi Biotec; 130-114-520), CD4 (VioGreen, Miltenyi Biotec; 130-113-230), CD8 (APC, Miltenyi Biotec; 130-110-681), CD45 (VioBlue, Miltenyi Biotec; 130-110-637), CD34 (PE, Miltenyi Biotec; 130-124-456), CD38 (APC, Miltenyi Biotec; 130-123-852), CD197 (CCR7), (VioBlue, Miltenyi Biotec; 130-117-353), CD45RO (APC, Miltenyi Biotec; 130-113-556), CD223 (VioBlue, Miltenyi Biotec; 130-118-549), CD279 (PE, Miltenyi Biotec; 130-120-385), CD366 (APC, Miltenyi Biotec; 130-119-781), CD154 (VioBlue, Miltenyi Biotec; 130-116-615), CD25 (PE, Miltenyi Biotec; 130-114-541), CD137 (APC, Miltenyi Biotec; 130-110-764), CD69 (PE, Miltenyi Biotec; 130-112-613), CD84 (APC, Biolegend; 326010) CD107a (PE, Miltenyi Biotec; 130-111-621), and CD33 (FITC, Miltenyi Biotec; 130-111-018). Analyses were performed by using FlowJo software (BD Biosciences, v10).

### In Vitro Cytotoxicity Assay

Cytotoxic assay was conducted by co-culturing AML target cell lines (positive for antigens) with CAR-T cells or non-transduced T cells for 48 hours at an E:T ratio of 1:1 at the end of T cell expansion (day 17). Co-cultured cells were stained with Annexin V-PE (Miltenyi Biotec; 130-118-363) and 7-AAD Staining Solution (Miltenyi Biotec; 130-11-568) and analyzed by flow cytometry with FACSCelesta™ Cell Analyzer and FlowJo software. Percentage of cells lysis was calculated using the following formula:

$$\% of lysis = 100 - \frac{N of alive AML cells in coculture with CAR T cells}{N of alive AML cells cultured alone} * 100$$

Alternatively, bioluminescence of luciferase-transduced AML cells was analyzed after 48 hours from co-culture with CAR-T cells. Briefly, cells were centrifuged and resuspended in 50 μl of PBS 1× and then incubated with XenoLight D-luciferin firefly (15 mg/mL in PBS; Perkin Elmer, Waltham, MA) for 10 min. Luciferase activity was measured by a Spark Tecan multi-well plate reader (Tecan Group Ltd., Mannedorf, Switzerland) and data signal reduction is reported as percentage of AML cell lysis assessed by bioluminescence imaging (BLI).

### Colonies Forming Assay

After 6 hours of co-culture in a ratio of 1:1, a total of 2×10³ primary CD34⁺ cells (isolated from healthy bone marrow or cord blood) were seeded into 500 μl of MethoCult™ (H4534, Stemcell Technologies, Meda MB, Italy), in 24-well plates and incubated at 37° C. For each co-culture four replicates were plated. Fourteen days after seeding, an adequate volume of a 1:6 solution of 3-[4,5-dimethylthiazol-2-yl]-2,5-diphenyltetrazolium bromide (MTT, Sigma-Aldrich Merck) in Hanks' was added to semisolid medium. Images were acquired by optical microscope with camera, and colonies were counted using ImageJ software.

### In Vivo Experiments in NSG Mice

Procedures involving animals and their care were carried out in accordance with institutional guidelines that comply with national and international laws and policies (EEC Council Directive 86/609, OJ L 358, 12 Dec. 1987) and with “ARRIVE” guidelines (Animals in Research Reporting In Vivo Experiments). Ministry authorization approved: 131/2022-PR. NSG mice (NOD.Cg-PrkdcscidII2rgtm1Wjl/SzJ, female of 4-5 weeks old, 20-25 g/mouse, maximum 5 animals/cage) were injected intravenously (tail vein) with 0.75×10⁶ SHI-1-LUC (transduced with luciferase gene) cells. After 2 days from AML injection, mice were treated by intravenous injection of 1.5×10⁶ CAR-T or untransduced/mock transduced T cells as control at a target:effector ratio of 1:3. Bioluminescence was monitored to verify tumor growth, by intra-peritoneal injections with XenoLight D-luciferin firefly (15 mg/ml in PBS; Perkin Elmer) 10 min prior to measurement (Xenogen IVIS Spectrum bioluminescence/optical imaging system, Xenogen Corporation, Alameda, CA).

### Statistical Analyses

Significant differences in means were tested by either Student's t-test or Wilcoxon non-parametric test, according to the mean distribution. One-way multiple comparisons ANOVA with Tukey's multiple comparison test was used when more than 2 groups/conditions were compared. Graphs and associated statistical analyses were generated using GraphPad Prism 8. All data are presented as mean±standard error of the mean (SEM). *P<0.05, **P<0.01, ***P<0.001, and ****P<0.0001 values are statistically significant.

### Results

The data here presented herein confirm that:
the high expression of all six antigens in a large number of primary cases collected at diagnosis and at relapse of myeloid and lymphoid acute leukemia, associated to very low/null expression on healthy regenerating bone marrow cells and hematopoietic stem cells; and
a specific lytic activity of AML blasts by the CAR-T cells created with the novel ScFv(s) recognizing CD69 and CD84 both in in vitro experiments and in murine xenografts engrafted with AML cell lines.

This data supports the development of immunotherapy and diagnostic approaches based on CD63, CD151, CD72, CD84, CD69, or CD109 for myeloid and lymphoid leukemias.

### Validation of CAR-T Binding and Blast Lysis in Zebrafish

The efficacy of the scFvs binding to the TSA is tested. GFP+ AML cell lines expressing high levels of the TSA (as well as GFP+ cell lines not expressing the TSA as control) are injected in a mixed solution with mCherry+ CAR-T or control T cells in a 1:1 ratio (here after named “mixed cells”) into zebrafish embryos at 48 hours post-fertilization.

On the transplanted animals, the number of circulating fluorescent GFP/mCherry positive cells are evaluated as well as the yellow-merged complex that represents the binding complex at 1, 5, 24 and 72 hours post-injection by fluorescence microscopy and ImageJ software, in all cohorts of animals (approximately 30 embryos each) to determine the binding between AML and CAR T cell.

Next, the scFvs specificity and killing potency is evaluated. The proliferation/death of the xenografted CAR-T cells and the reduction of AML blasts in embryos are evaluated.

25 embryos per group are sacrificed to count GFP+ and mCherry+ cells by flow cytometry at 5 and 24 hours post-injection. The comparison between reporter expression specific TSA-CAR T cells and control vector indicates the lysis efficacy occurring after binding.

### CAR T Validation Efficacy in NSG Mice

### CAR T Binding and Lysis Efficacy

At least three different AML cell lines which express high levels of the TSA, and control TSA negative cell lines, are transduced at high efficiency with the Luc-expressing control LV to constitutively express luciferase and injected in NOD-SCID interleukin-2 receptor gamma null (NSG) mice.

After 3 days mice are treated intravenously with 1-5×10⁶ antigen specific TSA expressing CAR T cells (n=8 animals) or control cells (n=3 to 5 animals). AML cell lines growth in NSG mice post-transplant and CAR-T cell infusion are monitored by imaging luciferase in vivo expression following an intraperitoneal injection of D-Luciferin substrate solution (15 mg/mL).

Winn assay is performed by injecting mice with AML cells mixed with CAR-T cells simultaneously.

In vivo monitoring is performed at 7, 14, 21 and 28 days post-transplant. CAR-T cell persistence is evaluated every two weeks.

AML-patient-derived xenograft (PDX)s are generated using blasts derived from the bone marrow of pediatric patients at diagnosis of de novo AML. The most promising CAR candidates from the previous experiments are tested on AML-PDXs.

### Results

The CAR-T constructs generated for each TSA efficiently and effectively binds to the target protein CD63, CD151, CD72, CD84, CD69, or CD109.

Cancer cells in the animal model decrease after administration of the CAR-T cells specific to the TSA in peripheral blood, bone marrow or spleen.

### 5. EQUIVALENTS AND INCORPORATION BY REFERENCE

While the disclosure has been particularly shown and described with reference to a preferred embodiment and various alternate embodiments, it will be understood by persons skilled in the relevant art that various changes in form and details can be made therein without departing from the spirit and scope of the disclosure.

All references, issued patents and patent applications cited within the body of the instant specification are hereby incorporated by reference in their entirety, for all purposes.

What is claimed is:

1. An antigen-binding protein (ABP) that specifically binds a target protein selected from CD63, CD151, CD72, CD84, CD69 and CD109.

2. The ABP of claim 1, that specifically binds human CD84.

3. The ABP of claim 2, wherein the ABP comprises an amino acid sequence having at least 80%, at least 85%, at least 90%, at least 95%, at least 97%, at least 99%, or 100% sequence identity to the amino acid sequence of SEQ ID NO: 97, SEQ ID NO: 98, or SEQ ID NO: 90.

4. The ABP of claim 3, wherein the ABP comprises:
a. V_{L} CDR1 having a sequence of SEQ ID NO: 51, V_{L} CDR2 having a sequence of SEQ ID NO: 54, V_{L} CDR3 having a sequence of SEQ ID NO: 61, V_{H} CDR1 having a sequence of SEQ ID NO: 63, V_{H} CDR2 having a sequence of SEQ ID NO: 68, and V_{H} CDR3 having a sequence of SEQ ID NO: 72;
b. V_{L} CDR1 having a sequence of SEQ ID NO: 47, V_{L} CDR2 having a sequence of SEQ ID NO: 54, V_{L} CDR3 having a sequence of SEQ ID NO: 55, V_{H} CDR1 having a sequence of SEQ ID NO: 63, V_{H} CDR2 having a sequence of SEQ ID NO: 68, and V_{H} CDR3 having a sequence of SEQ ID NO: 72; or
c. V_{L} CDR1 having a sequence of SEQ ID NO: 45, V_{L} CDR2 having a sequence of SEQ ID NO: 52, V_{L} CDR3 having a sequence of SEQ ID NO: 55, V_{H} CDR1 having a sequence of SEQ ID NO: 63, V_{H} CDR2 having a sequence of SEQ ID NO: 66, and V_{H} CDR3 having a sequence of SEQ ID NO: 71;

5. The ABP of any one of claims 1 to 4, comprising:
a. a light chain variable domain having at least 80%, at least 85%, at least 90%, at least 95%, at least 95%, at least 97% or 100% sequence identity to the amino acid sequence of SEQ ID NO: 80 and a heavy chain variable domain having at least 80%, at least 85%, at least 90%, at least 95%, at least 95%, at least 97% or 100% sequence identity to the amino acid sequence of SEQ ID NO: 88;
b. a light chain variable domain having at least 80%, at least 85%, at least 90%, at least 95%, at least 95%, at least 97% or 100% sequence identity to the amino acid sequence of SEQ ID NO: 81 and a heavy chain variable domain having at least 80%, at least 85%, at least 90%, at least 95%, at least 95%, at least 97% or 100% sequence identity to the amino acid sequence of SEQ ID NO: 88; or
c. a light chain variable domain having at least 80%, at least 85%, at least 90%, at least 95%, at least 95%, at least 97% or 100% sequence identity to the amino acid sequence of SEQ ID NO: 74 and a heavy chain variable domain having at least 80%, at least 85%, at least 90%, at least 95%, at least 95%, at least 97% or 100% sequence identity to the amino acid sequence of SEQ ID NO: 83.

6. The ABP of claim 1, specifically binds human CD69.

7. The ABP of claim 6, wherein the ABP comprises an amino acid sequence having at least 80%, at least 85%, at least 90%, at least 95%, at least 97%, at least 99%, or 100% sequence identity to the amino acid sequence selected from: SEQ ID NOs: 89-96.

8. The ABP of any one of claims 6 to 7, wherein the ABP comprises:
a. V_{L} CDR1 having a sequence of SEQ ID NO: 47, V_{L} CDR2 having a sequence of SEQ ID NO: 54, V_{L} CDR3 having a sequence of SEQ ID NO: 57, V_{H} CDR1 having a sequence of SEQ ID NO: 64, V_{H} CDR2 having a sequence of SEQ ID NO: 67, and V_{H} CDR3 having a sequence of SEQ ID NO: 71;
b. V_{L} CDR1 having a sequence of SEQ ID NO: 50, V_{L} CDR2 having a sequence of SEQ ID NO: 54, V_{L} CDR3 having a sequence of SEQ ID NO: 60, V_{H} CDR1 having a sequence of SEQ ID NO: 62, V_{H} CDR2 having a sequence of SEQ ID NO: 69, and V_{H} CDR3 having a sequence of SEQ ID NO: 73;
c. V_{L} CDR1 having a sequence of SEQ ID NO: 45, V_{L} CDR2 having a sequence of SEQ ID NO: 52, V_{L} CDR3 having a sequence of SEQ ID NO: 55, V_{H} CDR1 having a sequence of SEQ ID NO: 62, V_{H} CDR2 having a sequence of SEQ ID NO: 65, and V_{H} CDR3 having a sequence of SEQ ID NO: 70;
d. V_{L} CDR1 having a sequence of SEQ ID NO: 46, V_{L} CDR2 having a sequence of SEQ ID NO: 53, V_{L} CDR3 having a sequence of SEQ ID NO: 56, V_{H} CDR1 having a sequence of SEQ ID NO: 63, V_{H} CDR2 having a sequence of SEQ ID NO: 67, and V_{H} CDR3 having a sequence of SEQ ID NO: 71;
e. V_{L} CDR1 having a sequence of SEQ ID NO: 48, V_{L} CDR2 having a sequence of SEQ ID NO: 54, V_{L} CDR3 having a sequence of SEQ ID NO: 58, V_{H} CDR1 having a sequence of SEQ ID NO: 63, V_{H} CDR2 having a sequence of SEQ ID NO: 68, and V_{H} CDR3 having a sequence of SEQ ID NO: 72;
f. V_{L} CDR1 having a sequence of SEQ ID NO: 49, V_{L} CDR2 having a sequence of SEQ ID NO: 52, V_{L} CDR3 having a sequence of SEQ ID NO: 59, V_{H} CDR1 having a sequence of SEQ ID NO: 63, V_{H} CDR2 having a sequence of SEQ ID NO: 67, and V_{H} CDR3 having a sequence of SEQ ID NO: 71;
g. V_{L} CDR1 having a sequence of SEQ ID NO: 49, V_{L} CDR2 having a sequence of SEQ ID NO: 52, V_{L} CDR3 having a sequence of SEQ ID NO: 59, V_{H} CDR1 having a sequence of SEQ ID NO: 63, V_{H} CDR2 having a sequence of SEQ ID NO: 67, and V_{H} CDR3 having a sequence of SEQ ID NO: 71; or
h. V_{L} CDR1 having a sequence of SEQ ID NO: 45, V_{L} CDR2 having a sequence of SEQ ID NO: 52, V_{L} CDR3 having a sequence of SEQ ID NO: 55, V_{H} CDR1 having a sequence of SEQ ID NO: 63, V_{H} CDR2 having a sequence of SEQ ID NO: 66, and V_{H} CDR3 having a sequence of SEQ ID NO: 71.

9. The ABP of any one of claims 6 to 8, comprising:
a. a light chain variable domain having at least 80%, at least 85%, at least 90%, at least 95%, at least 95%, at least 97% or 100% sequence identity to the amino acid sequence of SEQ ID NO: 76 and a heavy chain variable domain having at least 80%, at least 85%, at least 90%, at least 95%, at least 95%, at least 97% or 100% sequence identity to the amino acid sequence of SEQ ID NO: 85;
b. a light chain variable domain having at least 80%, at least 85%, at least 90%, at least 95%, at least 95%, at least 97% or 100% sequence identity to the amino acid sequence of SEQ ID NO: 79 and a heavy chain variable domain having at least 80%, at least 85%, at least 90%, at least 95%, at least 95%, at least 97% or 100% sequence identity to the amino acid sequence of SEQ ID NO: 87;
c. a light chain variable domain having at least 80%, at least 85%, at least 90%, at least 95%, at least 95%, at least 97% or 100% sequence identity to the amino acid sequence of SEQ ID NO: 74 and a heavy chain variable domain having at least 80%, at least 85%, at least 90%, at least 95%, at least 95%, at least 97% or 100% sequence identity to the amino acid sequence of SEQ ID NO: 83;
d. a light chain variable domain having at least 80%, at least 85%, at least 90%, at least 95%, at least 95%, at least 97% or 100% sequence identity to the amino acid sequence of SEQ ID NO: 74 and a heavy chain variable domain having at least 80%, at least 85%, at least 90%, at least 95%, at least 95%, at least 97% or 100% sequence identity to the amino acid sequence of SEQ ID NO: 82;
e. a light chain variable domain having at least 80%, at least 85%, at least 90%, at least 95%, at least 95%, at least 97% or 100% sequence identity to the amino acid sequence of SEQ ID NO: 75 and a heavy chain variable domain having at least 80%, at least 85%, at least 90%, at least 95%, at least 95%, at least 97% or 100% sequence identity to the amino acid sequence of SEQ ID NO: 84;
f. a light chain variable domain having at least 80%, at least 85%, at least 90%, at least 95%, at least 95%, at least 97% or 100% sequence identity to the amino acid sequence of SEQ ID NO: 77 and a heavy chain variable domain having at least 80%, at least 85%, at least 90%, at least 95%, at least 95%, at least 97% or 100% sequence identity to the amino acid sequence of SEQ ID NO: 86;
g. a light chain variable domain having at least 80%, at least 85%, at least 90%, at least 95%, at least 95%, at least 97% or 100% sequence identity to the amino acid sequence of SEQ ID NO: 77 and a heavy chain variable domain having at least 80%, at least 85%, at least 90%, at least 95%, at least 95%, at least 97% or 100% sequence identity to the amino acid sequence of SEQ ID NO: 84;
h. a light chain variable domain having at least 80%, at least 85%, at least 90%, at least 95%, at least 95%, at least 97% or 100% sequence identity to the amino acid sequence of SEQ ID NO: 78 and a heavy chain variable domain having at least 80%, at least 85%, at least 90%, at least 95%, at least 95%, at least 97% or 100% sequence identity to the amino acid sequence of SEQ ID NO: 84; or
i. a light chain variable domain having at least 80%, at least 85%, at least 90%, at least 95%, at least 95%, at least 97% or 100% sequence identity to the amino acid sequence of SEQ ID NO: 74 and a heavy chain variable domain having at least 80%, at least 85%, at least 90%, at least 95%, at least 95%, at least 97% or 100% sequence identity to the amino acid sequence of SEQ ID NO: 83.

10. The ABP of claim 1, specifically binds human CD69 and CD84.

11. The ABP of claim 10, wherein the ABP comprises an amino acid sequence having at least 80%, at least 85%, at least 90%, at least 95%, at least 97%, at least 99%, or 100% sequence identity to the amino acid sequence of SEQ ID NO: 36.

12. The ABP of any one of claims 10 to 11, wherein the ABP comprises:
V_{L} CDR1 having a sequence of SEQ ID NO: 45, V_{L} CDR2 having a sequence of SEQ ID NO: 52, V_{L} CDR3 having a sequence of SEQ ID NO: 55, V_{H} CDR1 having a sequence of SEQ ID NO: 63, V_{H} CDR2 having a sequence of SEQ ID NO: 66, and V_{H} CDR3 having a sequence of SEQ ID NO: 71.

13. The ABP of claim 10, wherein the ABP comprises a light chain variable domain having at least 80%, at least 85%, at least 90%, at least 95%, at least 95%, at least 97% or 100% sequence identity to the amino acid sequence of SEQ ID NO: 74 and a heavy chain variable domain having at least 80%, at least 85%, at least 90%, at least 95%, at least 95%, at least 97% or 100% sequence identity to the amino acid sequence of SEQ ID NO: 83.

14. The ABP of claim 1, wherein the ABP comprises an amino acid sequence selected from SEQ ID NOs: 89-98.

15. The ABP of any one of claims 1 to 10, wherein the ABP comprises a Fab, Fab′, F(ab′)₂, Fv, scFv, (scFv)₂, single chain antibody molecule, dual variable domain antibody, single variable domain antibody, linear antibody, or V domain antibody.

16. The ABP of claim 15, wherein the ABP is a Fab, Fab′, F(ab′)₂, Fv, scFv, (scFv)₂, single chain antibody molecule, dual variable domain antibody, single variable domain antibody, linear antibody, or V domain antibody.

17. The ABP of any one of claims 1 to 16, wherein the ABP is a monoclonal antibody.

18. The ABP of any one of claims 1 to 17, wherein the ABP is selected from an IgG, IgM, IgA, IgD, and IgE antibody.

19. The ABP of any one of claims 1-18, comprising a heavy chain constant region of the class IgG and a subclass selected from IgG1, IgG2, IgG3, and IgG4.

20. The ABP of any one of claims 1-19, wherein the ABP is conjugated to a drug.

21. The ABP of any one of claims 1-20, capable of inducing antibody-dependent cell-mediated cytotoxicity (ADCC),
wherein the ABP specifically binds to a target protein selected from the group consisting of CD63, CD151, CD72, CD84, CD69, and CD109,
and wherein the ABP comprises a human Fc.

22. The ABP of claim 21, wherein the ABP is human, humanized or chimeric.

23. The ABP of claim 21 or 22, wherein the ABP is monoclonal.

24. The ABP of any one of claims 1-23, wherein the ABP is bispecific or multispecific.

25. The ABP of any one of claims 1-24, wherein in the ABP comprises a heavy chain constant region of IgG.

26. The ABP of any one of claims 1-25, wherein the ABP is afucosylated.

27. The ABP of any one of claims 1-26, wherein the ABP binds to the target protein with a K_{D} of less than or equal to 50 nM, 10 nM, 5 nM, 1 nM, 0.5 nM or 0.1 nM, as measured by surface plasmon resonance (SPR) assay.

28. An ABP-drug conjugate, comprising: the ABP of any one of claims 1-27, a cytotoxic agent linked to the ABP, and optionally a linker that links the cytotoxic agent to the ABP.

29. The ABP-drug conjugate of claim 28, wherein the cytotoxic agent comprises an anti-angiogenic agent, a pro-apoptotic agent, an anti-mitotic agent, an anti-kinase agent, an alkylating agent, a hormone, a hormone agonist, a hormone antagonist, a chemokine, a drug, a prodrug, a toxin, an enzyme, an antimetabolite, an antibiotic, an alkaloid, or a radioactive isotope.

30. The ABP-drug conjugate of claim 28 or 29, wherein the linker is a cleavable linker or a non-cleavable linked.

31. A bispecific T-cell engager (BiTE) comprising: the ABP of any one of claims 1-27, and a T-cell activating domain.

32. A pharmaceutical composition comprising the ABP of any one of claims 1 to 27, the ABP-drug conjugate of any one of claims 28 to 30, or the BiTE of claim 30, and an excipient.

33. An isolated polynucleotide or set of isolated polynucleotides encoding the ABP of any one of claims 1 to 27 or the BiTE of claim Error! Reference source not found.

34. A vector or set of vectors comprising the isolated polynucleotide of claim 33.

35. A host cell comprising the isolated polynucleotide of claim 33 or the vector of claim 34.

36. A method of producing an isolated antigen binding protein (ABP), comprising expressing the ABP in the host cell of claim 35, and isolating the ABP.

37. A chimeric antigen receptor (CAR) comprising:
a. an extracellular antigen-binding domain,
b. a transmembrane domain,
c. a signaling domain, and
d. optionally at least one costimulatory domain,
wherein the extracellular antigen-binding domain specifically binds to a target protein selected from the group consisting of CD63, CD151, CD72, CD84, CD69, and CD109.

38. The CAR of claim 37, wherein the extracellular antigen-binding domain comprises a single-chain variable fragment (scFv) of an antibody that specifically binds to the target protein.

39. The CAR of claim 37, wherein the extracellular antigen-binding domain comprises the ABP of any one of claims 1-27.

40. The CAR of any one of claims 37-39, wherein the signaling domain comprises the intracellular domain of CD3ζ.

41. The CAR of any one of claims 37 to 39, further comprising a costimulatory domain, wherein the costimulatory domain is a CD28 costimulatory domain, a 4-1BB costimulatory domain, a CD27 costimulatory domain, an OX40 costimulatory domain, or an ICOS costimulatory domain.

42. The CAR of claim 41, wherein the costimulatory domain is a 4-1BB costimulatory domain.

43. The CAR of any one of claims 41 to 42, wherein the 4-1BB costimulatory domain an amino acid sequence having at least 90%, at least 95%, or at least 100% sequence identity to the amino acid sequence of SEQ ID NO: 105.

44. The CAR of any one of claims 37 to 39, wherein the transmembrane domain is a CD28 transmembrane domain.

45. The CAR of claim 44, wherein the CD28 transmembrane domain an amino acid sequence having at least 90%, at least 95%, or at least 100% sequence identity to the amino acid sequence of: SEQ ID NO: 100.

46. The CAR of any one of claims 37 to 45, further comprising a hinge region.

47. The CAR of claim 46, wherein the hinge region is a hinge region derived from a CD28 polypeptide.

48. The CAR of claim 47, wherein the hinge region comprises an amino acid sequence having at least 90%, at least 95%, or at least 100% sequence identity to the amino acid sequence of: SEQ ID NO: 99.

49. The CAR of any one of claims 37 to 48, wherein the signaling domain is a CD3zeta signaling domain.

50. The CAR of claim 49, wherein the CD3zeta signaling domain comprises an amino acid sequence having at least 90%, at least 95%, or at least 100% sequence identity to the amino acid sequence of: SEQ ID NO: 101.

51. The CAR of any one of claims 37 to 50, wherein the CAR comprises a polypeptide having at least 80%, at least 85%, at least 90%, at least 95%, at least 97%, at least 98%, at least 99%, or 100% sequence identity to an amino acid sequence selected from SEQ ID NOs.: 35-44.

52. The CAR of any one of claims 37 to 51, wherein the extracellular antigen-binding domain specifically binds to human CD84.

53. The CAR of any one of claims 37 to 51, wherein the extracellular antigen-binding domain specifically binds to human CD69.

54. The CAR of any one of claims 37 to 53, wherein the CAR comprises a polypeptide having at least 80%, at least 85%, at least 90%, at least 95%, at least 97%, at least 98%, at least 99%, or 100% sequence identity to an amino acid selected from: SEQ ID NOs.: 35-42.

55. The CAR of any one of claims 37 to 53, wherein the CAR comprises a polypeptide having at least 80%, at least 85%, at least 90%, at least 95%, at least 97%, at least 98%, at least 99%, or 100% sequence identity to an amino acid of SEQ ID NOs: 43, 44, or 36.

56. A polynucleotide encoding the CAR of any one of claims 37 to 55.

57. The polynucleotide of claim 56, comprising a nucleotide sequence having at least 75%, at least 80%, at least 85%, at least 90%, at least 95%, at least 97%, or 100% sequence identity to the nucleotide sequence of SEQ ID NOs: 24-34.

58. A vector comprising the polynucleotide of any one of claims 56 to 57.

59. An immunoresponsive cell expressing the CAR of any one of claims 37 to 55.

60. An immunoresponsive cell comprising the polynucleotide of claim 56 or the vector of claim 58.

61. The immunoresponsive cell of any one of claims 59 to 60, wherein the immunoresponsive cell is an αβ T cell, a γδ T cell, or a Natural Killer (NK) cell.

62. The immunoresponsive cell of claim 61, wherein the αβ T cell is a CD4⁺ T cell or a CD8⁺ T cell.

63. A method of preparing the immunoresponsive cell of any one of claims 59 to 62, the method comprising transfecting or transducing the polynucleotide of claim 56 or the vector of claim 58 into an immune cell.

64. The method of claim 63, wherein the method comprises expanding the immune cell for at least 48 hours.

65. The method of claim 64, wherein the immune cell is transduced at a multiplicity of infection of 10.

66. The method of claim 65, wherein the method further comprises, after transducing, washing the vector of claim 58, and expanding the transduced immune cell for at least 14 days.

67. A method of treating a subject, the method comprising: administering a therapeutically effective amount of the ABP of any one of claims 1 to 27, the ABP-drug conjugate of any one of claims 28-30, the pharmaceutical composition of claim 32, or the immunoresponsive cell of any one of claims 59 to 62.

68. The method of claim 67, wherein the subject has a myeloid disorders (MD) or acute leukemia (AL).

69. The method of claim 67, wherein the myeloid disorders (MD) and acute leukemias (AL) are of pediatric or adult onset.

70. The method of claims 59 to 62, wherein the ABP, the pharmaceutical composition or the immunoresponsive cell is administered in combination with an additional agent.

71. The method of claim 70, wherein the additional agent is a chemotherapeutic or biological agent.

72. The method of claim 71, wherein the chemotherapeutic agent is selected from the group consisting of cytarabine, daunorubicin, idarubicin, cladribine, mitoxantrone, azacitidine, decitabine, and CPX-351 (Vyxeos®).

73. The method of claim 70, wherein the additional agent is a hedgehog pathway inhibitor.

74. The method of claim 73, wherein the hedgehog pathway inhibitor is a sonic hedgehog pathway inhibitor.

75. The method of claim 74, wherein the sonic hedgehog pathway inhibitor is selected from vismodegib, sonidigib, and arsenic trioxide (ATO).

76. The method of claim 73, wherein the hedgehog pathway inhibitor is glasdegib (Daurismo™).

77. The method of claim 71, wherein the additional agent is an FMS-like tyrosine kinase 3 (FLT3) inhibitor.

78. The method of claim 77, wherein the FLT3 inhibitor is selected from the group consisting of midostaurin (Rydapt®), gilteritinib (Xospata®), sorafenib, lestaurtinib, quizartinib, and crenolanib.

79. The method of claim 73, wherein the additional agent is an isocitrate dehydrogenase 1 (IDH1) or isocitrate dehydrogenase 2 (IDH2) inhibitor.

80. The method of claim 79, wherein the IDH1 or IDH2 inhibitor is ivosidenib (Tibsovo®) or enasidenib (Idhifa®).

81. The method of claim 73, wherein the additional agent is a B-cell lymphoma 2 (BCL2) inhibitor.

82. The method of claim 81, wherein the BCL2 inhibitor is venetoclax (Venclexta®).

83. The method of claim 73, wherein the additional agent is a CD33-targeting agent.

84. The method of claim 83, wherein the CD33-targeting agent is gemtuzumab ozogamicin (Mylotarg™) or vadastuximab talirine (SGN-CD33A).

85. The method of claim 73, wherein the additional agent is a cell cycle checkpoint inhibitor.

86. The method of claim 85, wherein the cell cycle checkpoint inhibitor is an Aurora kinase inhibitor, a Polo-like kinase 1 (PLK1) inhibitor, a cyclin dependent kinase (CDK) inhibitor, or a checkpoint kinase 1 (CHK1) inhibitor.

87. The method of claim 73, wherein the additional agent is an immune checkpoint inhibitor.

88. The method of claim 73, wherein the immune checkpoint inhibitor is an anti-CTLA-4 antibody, an anti-PD-1 antibody, or an anti-PD-L1 antibody.

89. A method of diagnosing myeloid disorders (MD) and acute leukemias (AL) in a subject, comprising the step of: detecting the presence or level of a tumor specific antigen in a biological sample of the subject, wherein the tumor specific antigen is selected from the group consisting of CD63, CD151, CD72, CD84, CD69, and CD109.

90. The method of claim 89, wherein the biological sample is a blood sample or a bone marrow sample.

91. The method of claim 89 or 90, wherein the biological sample comprises blast cells.

92. The method of claim 91, wherein the blast cells are selected from myeloid blast cells, lymphoid blast cells, or a combination of myeloid and lymphoid blast cells.

93. The method of any one of claims 89 to 92, wherein the step of detecting comprises contacting the biological sample with a target binding protein,
that specifically binds to the tumor specific antigen.

94. The method of claim 93, wherein the target binding protein is an antibody.

95. The method of claim 93, wherein the target binding protein is the ABP of any one of claims 1 to 27.

96. The method of any one of claims 89 to 95, wherein the step of detecting comprises flow cytometry, immunocytochemistry, immunohistochemistry, fluorescence, or enzyme-linked immunosorbent assay (ELISA).

97. The method of any one of claims 93 to 96, wherein the target binding protein is labeled.

98. The method of claim 97, wherein the target binding protein is labeled with a fluorophore, or an enzyme.

99. The method of any one of claims 89 to 92, wherein the step of detecting comprises measuring mRNA level of the tumor specific antigen in the biological sample.

100. The method of claim 99, wherein the mRNA level is measured by in situ hybridization, reverse transcription-polymerase chain reaction (RT-PCR), or by sequencing.

101. The method of any one of claims 89 to 100, wherein the myeloid disorders (MD) and acute leukemias (AL) have onset in pediatric or adult age.

102. The method of any one of claims 89 to 101, further comprising the step of determining the presence or absence of cancer cells in the subject based on the detection of the presence or level of the tumor specific antigen in the biological sample from the subject.

103. The method of any one of claims 89 to 101, further comprising the step of
administering to the subject a therapeutically effective amount of a therapeutic agent that specifically binds to the tumor specific antigen selected from the group consisting of CD63, CD151, CD72, CD84, CD69, and CD109.

104. The method of claim 103, wherein the therapeutic agent is an antigen-binding protein (ABP), an ABP-drug conjugate, an immunoresponsive cell expressing a chimeric antigen receptor (CAR), or a bispecific T-cell engager (BiTE), wherein the ABP, the ABP-drug conjugate, the CAR, or the BiTE specifically binds to the tumor specific antigen selected from the group consisting of CD63, CD151, CD72, CD84, CD69, and CD109.

105. The method of claim 104, wherein the therapeutic agent is the ABP of any one of claims 1-27, the ABP-drug conjugate of any one of claims 28-30, the BiTE of claim Error! Reference source not found., the CAR of any one of claims 37-55, or the immunoresponsive cell of any one of claims 59-62.


SEQUENCE LISTING (105 sequences)

SEQ ID NO: 1 - protein, 5 aa, Artificial Sequence
  source: /note=“Description of Artificial Sequence Synthetic polypeptide”
  source: /note=“See specification as filed for detailed description of substitutions and preferred embodiments”
GGGGS

SEQ ID NO: 2 - protein, 5 aa, Artificial Sequence
  source: /note=“Description of Artificial Sequence Synthetic peptide”
GGGGS

SEQ ID NO: 3 - protein, 5 aa, Artificial Sequence
  source: /note=“Description of Artificial Sequence Synthetic peptide”
SGGGG

SEQ ID NO: 4 - protein, 50 aa, Artificial Sequence
  source: /note=“Description of Artificial Sequence Synthetic polypeptide”
  SITE (1)..(50): /note=“This sequence may encompass 1-10 ′Gly Gly Gly Gly Ser′ repeating units”
GGGGSGGGGSGGGGSGGGGSGGGGSGGGGSGGGGSGGGGSGGGGSGGGGS

SEQ ID NO: 5 - protein, 50 aa, Artificial Sequence
  source: /note=“Description of Artificial Sequence Synthetic polypeptide”
  SITE (1)..(50): /note=“This sequence may encompass 1-10 ′Ser Gly Gly Gly Gly′ repeating units”
SGGGGSGGGGSGGGGSGGGGSGGGGSGGGGSGGGGSGGGGSGGGGSGGGG

SEQ ID NO: 6 - protein, 20 aa, Artificial Sequence
  source: /note=“Description of Artificial Sequence Synthetic peptide”
GGGGSGGGGSGGGGSGGGGS

SEQ ID NO: 7 - protein, 25 aa, Artificial Sequence
  source: /note=“Description of Artificial Sequence Synthetic peptide”
GGGGSGGGGSGGGGSGGGGSGGGGS

SEQ ID NO: 8 - protein, 15 aa, Artificial Sequence
  source: /note=“Description of Artificial Sequence Synthetic peptide”
GGGGSGGGGSGGGGS

SEQ ID NO: 9 - protein, 8 aa, Artificial Sequence
  source: /note=“Description of Artificial Sequence Synthetic peptide”
GGGGGGGG

SEQ ID NO: 10 - protein, 6 aa, Artificial Sequence
  source: /note=“Description of Artificial Sequence Synthetic peptide”
GGGGGG

SEQ ID NO: 11 - protein, 15 aa, Artificial Sequence
  source: /note=“Description of Artificial Sequence Synthetic peptide”
EAAAKEAAAKEAAAK

SEQ ID NO: 12 - protein, 15 aa, Artificial Sequence
  source: /note=“Description of Artificial Sequence Synthetic peptide”
  SITE (1)..(15): /note=“This sequence may encompass 1-3 ′Glu Ala Ala Ala Lys′ repeating units”
EAAAKEAAAKEAAAK

SEQ ID NO: 13 - protein, 46 aa, Artificial Sequence
  source: /note=“Description of Artificial Sequence Synthetic polypeptide”
AEAAAKEAAAKEAAAKEAAAKALEAEAAAKEAAAKEAAAKEAAAKA

SEQ ID NO: 14 - protein, 5 aa, Artificial Sequence
  source: /note=“Description of Artificial Sequence Synthetic peptide”
PAPAP

SEQ ID NO: 15 - protein, 12 aa, Artificial Sequence
  source: /note=“Description of Artificial Sequence Synthetic peptide”
AEAAAKEAAAKA

SEQ ID NO: 16 - protein, 34 aa, Artificial Sequence
  source: /note=“Description of Artificial Sequence Synthetic polypeptide”
  SITE (1)..(34): /note=“This sequence may encompass 5-17 ′Ala Pro′ repeating units”
APAPAPAPAPAPAPAPAPAPAPAPAPAPAPAPAP

SEQ ID NO: 17 - protein, 17 aa, Artificial Sequence
  source: /note=“Description of Artificial Sequence Synthetic peptide”
VSQTSKLTRAETVFPDV

SEQ ID NO: 18 - protein, 6 aa, Artificial Sequence
  source: /note=“Description of Artificial Sequence Synthetic peptide”
PLGLWA

SEQ ID NO: 19 - protein, 6 aa, Artificial Sequence
  source: /note=“Description of Artificial Sequence Synthetic peptide”
RVLAEA

SEQ ID NO: 20 - protein, 10 aa, Artificial Sequence
  source: /note=“Description of Artificial Sequence Synthetic peptide”
EDVVCCSMSY

SEQ ID NO: 21 - protein, 8 aa, Artificial Sequence
  source: /note=“Description of Artificial Sequence Synthetic peptide”
GGIEGRGS

SEQ ID NO: 22 - protein, 10 aa, Artificial Sequence
  source: /note=“Description of Artificial Sequence Synthetic peptide”
TRHRQPRGWE

SEQ ID NO: 23 - protein, 10 aa, Artificial Sequence
  source: /note=“Description of Artificial Sequence Synthetic peptide”
AGNRVRRSVG

SEQ ID NO: 24 - DNA, 1506 bp, Artificial Sequence
  source: /note=“Description of Artificial Sequence Synthetic polynucleotide”
ggatccgccgccaccatgaagcacctgtggtttttcctgctgctggtggccgcccccaggtgggttctgagccaggtgcagctgcagcagtccggccctggcctggtgcagcctagcagaaccctgagcctgacatgtgccatcagcggcgattccatctccagcaataccgcctcctggaactggatcagacagagccctagcaggggcctggagtgggtgggcaagacagagtacagatccggctggttcttcgagtacgccccctccgtgggcggcagagtgaatatctcccccgatacaagcaagaaccagttcagcctgcagctgaatagcgtgacacctgaggatacagccgtgtacttctgcgccagggactcctacaatgactacgataacgacggctttgacatctggggccagggcacactggtgacagtgagcagcggcggcggcggaagcggaggaggaggatctggcggcggcggtagcggaggaggtggatctcagtccgtgctgacccagccccccagcgtgtccgctgctcctggacagaaggtgaccatcagctgcagcggctcctcctccaatatcggctccaactacgtggcctggtaccagcagctgcccggcacagcccccaagctgctgatctacgacaccaacaagaggcccagcggcatccccgataggtttagcggctccaagagcggcacatccgccaccctgggcatcaccggcctgcagacaggcgatgaggccgactactactgtggcacatgggacagctccctgagcgccgaggtgttcggcacaggcacaaaggtgaccgtgctgatcgaggtgatgtacccccccccctacctggacaatgagaagtccaacggcacaatcatccacgtgaagggcaagcacctgtgtcccagccccctgtttcccggccctagcaagcccttctgggtgctggtggtggtgggcggcgtgctggcttgttacagcctgctggtgacagtcgccttcatcatcttttgggtgaagaggggcaggaagaagctgctgtacatctttaagcagccctttatgaggcccgtgcagacaacccaggaggaggacggctgtagctgcaggtttcctgaggaggaggagggcggctgtgagctgagagtgaagttcagcaggtccgccgacgcccccgcctaccaacagggacagaatcagctgtacaatgagctgaacctgggcagaagagaggagtacgacgtgctggataagaggagaggcagggaccctgagatgggcggcaagcctagaagaaagaacccccaggagggcctgtacaatgaactgcagaaggacaagatggccgaggcctacagcgagatcggcatgaagggcgagagaaggaggggcaagggccacgatggcctgtaccagggcctgtccacagccaccaaggacacatacgacgccctgcacatgcaggccctgcccccaaggtgagtcgac

SEQ ID NO: 25 - DNA, 1506 bp, Artificial Sequence
  source: /note=“Description of Artificial Sequence Synthetic polynucleotide”
ggatccgccgccaccatgaagcacctgtggtttttcctgctgctggtggccgcccctagatgggtgctgtcccaggtgcagctgcagcagagcggccctggcctggtgaagcccagccagaccctgtccctgacctgcgccatcagcggcgatagcgtgagcagcaactccgccagctggaattggatcaggcagtcccccagcaggggcctggagtggctgggaaagacagagtacagatccaagtggtactacgagtttgcccctagcgtgaagagcagaatcaccatcacccctgataccagcaagaaccagttttccctgcagctgaattccgtgacccctgaggacaccgccgtgtactactgcgtgagagactcctacaacggctacgattttgacggcttcgatatctggggccagggcaccctggtgaccgtgtccagcggcggaggcggaagcggaggaggaggaagcggcggaggaggcagcggaggaggtggaagccagagcgtgctgacacagccccctagcgtgtccgccgccccaggacaaaaggtgacaatcagctgctccggctcctcctccaatatcggcagcaactacgtggcctggtaccagcagctgcctggcacagcccccaagctgctgatctacgacaccaataagaggccctccggcatccccgatagattctccggctccaagagcggcacatccgccaccctgggcatcaccggcctgcagacaggcgatgaggccgactactactgtggcacatgggattcctccctgtccgccgaggtgtttggcaccggcacaaaggtgaccgtgctgatcgaggtgatgtacccccccccctacctggacaatgagaagtccaacggcacaatcatccacgtgaagggcaagcacctgtgtcccagccccctgtttcccggccctagcaagcccttctgggtgctggtggtggtgggcggcgtgctggcttgttacagcctgctggtgacagtcgccttcatcatcttttgggtgaagaggggcaggaagaagctgctgtacatctttaagcagccctttatgaggcccgtgcagacaacccaggaggaggacggctgtagctgcaggtttcctgaggaggaggagggcggctgtgagctgagagtgaagttcagcaggtccgccgacgcccccgcctaccaacagggacagaatcagctgtacaatgagctgaacctgggcagaagagaggagtacgacgtgctggataagaggagaggcagggaccctgagatgggcggcaagcctagaagaaagaacccccaggagggcctgtacaatgaactgcagaaggacaagatggccgaggcctacagcgagatcggcatgaagggcgagagaaggaggggcaagggccacgatggcctgtaccagggcctgtccacagccaccaaggacacatacgacgccctgcacatgcaggccctgcccccaaggtgagtcgac

SEQ ID NO: 26 - DNA, 1506 bp, Artificial Sequence
  source: /note=“Description of Artificial Sequence Synthetic polynucleotide”
ggatccgccgccaccatgaagcacctgtggttctttctgctgctggtggccgcccctagatgggtgctgagccaggtgcagctgcagcagtccggccctggcctggtgaagccttcccagacactgtccctgacatgtgccatcagcggcgactccgtgttctccaatagcgccagctggaattggatcagacagagcccctccagaggcctggagtggctgggcaagacagagtacaggtccaagtggtttgacgagtacgcccccagcgtgaagggcagaatcgccatcagacctgacacctccaagaaccaggtgtccctgcagctgaatagcgtgacccccgaggacaccgccgtgtactactgtgtgagagatagctacaacggctacgattttgatggctttgacatctggggccagggcaccctggtgaccgtgagctccggcggaggcggaagcggaggaggaggaagcggcggaggcggtagcggaggaggtggaagccagagcgtgctgacccagcctcctagcgtgagcgccgcccctggacagcaggtgatcatctcctgcagcggctccagctccaacgtgggcaataactacgtggcctggtaccagcaggtgcccggcgctgctcccaggctgctgatctacgacaactacaagagaccctccggcatccccgacaggttttccggcagcaagtccggcacctccgccaccctgggcatcaccggactgcagagcggcgacgaggccgactactactgtggcagctgggatagctccctgagcgccgaggtgtttggcggcggcaccaagctgacagccctgatcgaggtgatgtacccccccccctacctggacaatgagaagtccaacggcacaatcatccacgtgaagggcaagcacctgtgtcccagccccctgtttcccggccctagcaagcccttctgggtgctggtggtggtgggcggcgtgctggcttgttacagcctgctggtgacagtcgccttcatcatcttttgggtgaagaggggcaggaagaagctgctgtacatctttaagcagccctttatgaggcccgtgcagacaacccaggaggaggacggctgtagctgcaggtttcctgaggaggaggagggcggctgtgagctgagagtgaagttcagcaggtccgccgacgcccccgcctaccaacagggacagaatcagctgtacaatgagctgaacctgggcagaagagaggagtacgacgtgctggataagaggagaggcagggaccctgagatgggcggcaagcctagaagaaagaacccccaggagggcctgtacaatgaactgcagaaggacaagatggccgaggcctacagcgagatcggcatgaagggcgagagaaggaggggcaagggccacgatggcctgtaccagggcctgtccacagccaccaaggacacatacgacgccctgcacatgcaggccctgcccccaaggtgagtcgac

SEQ ID NO: 27 - DNA, 1506 bp, Artificial Sequence
  source: /note=“Description of Artificial Sequence Synthetic polynucleotide”
ggatccgccgccaccatgaagcacctgtggtttttcctgctgctggtggccgcccccaggtgggttctgagccaggtgcagctgcagcagtccggccctggcctggtgaagccctcccagacactgagcctgacatgcgccatctccggcgacagcgtgttcagcaccaccgccagctggaattggatcagacagagccccagcagaggcctggagtggctgggcaagaccgagtacaggagcaagtggttcgacgagtacgcccccagcgtgaagggcaggatcgccatcagacctgacacatccagaaatcaggtgagcctgcagctgaatagcatgacacccgaggataccgccgtgtacttctgtgtgagagacagctacaacggctacgacttcgacggcttcgacatctggggccagggcacaacagtgacagtgagctccggcggcggcggctctggaggaggaggatctggcggcggcggaagcggaggaggaggttctcagagcgtggtgacacagccccccagcgtgagcgccgctcctggacaaaaggtgaccatctcctgctccggcagcagcagcaacatcggcaataactacgtgtcctggtaccagcagctgcctggcaccgcccccaagctgctgatctacgacaacaataagaggcccagcggcatccctgacagattctccggctccaagagcggcacctccgccaccctgggcatcacaggcctgcagacaggcgacgaggccgactactactgtggcacatgggatagctccctgtccgccgagatgtttggcggcggcaccaaggtgaccgtgctgatcgaggtgatgtacccccccccctacctggacaatgagaagtccaacggcacaatcatccacgtgaagggcaagcacctgtgtcccagccccctgtttcccggccctagcaagcccttctgggtgctggtggtggtgggcggcgtgctggcttgttacagcctgctggtgacagtcgccttcatcatcttttgggtgaagaggggcaggaagaagctgctgtacatctttaagcagccctttatgaggcccgtgcagacaacccaggaggaggacggctgtagctgcaggtttcctgaggaggaggagggcggctgtgagctgagagtgaagttcagcaggtccgccgacgcccccgcctaccaacagggacagaatcagctgtacaatgagctgaacctgggcagaagagaggagtacgacgtgctggataagaggagaggcagggaccctgagatgggcggcaagcctagaagaaagaacccccaggagggcctgtacaatgaactgcagaaggacaagatggccgaggcctacagcgagatcggcatgaagggcgagagaaggaggggcaagggccacgatggcctgtaccagggcctgtccacagccaccaaggacacatacgacgccctgcacatgcaggccctgcccccaaggtgagtcgac

SEQ ID NO: 28 - DNA, 1506 bp, Artificial Sequence
  source: /note=“Description of Artificial Sequence Synthetic polynucleotide”
ggatccgccgccaccatgaagcacctgtggtttttcctgctgctggtggccgcccctaggtgggtgctgagccaggtgcagctgcagcagagcggccctggcctggtgaagcccagccagaccctgtccctgacatgcgccatcagcggcgatagcgtgtccagcaatagcgcctcctggaattggatcagacagagccctagcagaggcctggagtggctgggcaaggtggagtacaggtccaagtggtacgatgagtacgccccctccgtgaagagcagaatcgccatcagacctgacacatccaagaaccaggtgagcctgcacctgatctccgtgacacctgatgatacagccgtgtactactgcgccagggactcctacaacggccacgccttcgatggctttgatgtgtggggccagggcacactggtgaccgtgtccagcggcggcggcggatctggaggaggaggaagcggcggcggaggctctggaggaggtggaagccagtccgtgctgacccagcccccctccgtgagcgctgctccaggacagaaggtgacaatcagctgctccggctccagctccaacatcggcgataatctggtgagctggtaccagcagctgcctggcacagcccctaagctgctgatctacgataacaataagagaccctccggcatccccgacaggttcagcggcagcaagtccggcacctccgccacactggacatcacaggcctgcagaccggcgacgaggccgactactactgcgagagctgggataacaacctgaccgccgaggtgttcggcaccggcacaaaggtgacagtgctgatcgaggtgatgtacccccccccctacctggacaatgagaagtccaacggcacaatcatccacgtgaagggcaagcacctgtgtcccagccccctgtttcccggccctagcaagcccttctgggtgctggtggtggtgggcggcgtgctggcttgttacagcctgctggtgacagtcgccttcatcatcttttgggtgaagaggggcaggaagaagctgctgtacatctttaagcagccctttatgaggcccgtgcagacaacccaggaggaggacggctgtagctgcaggtttcctgaggaggaggagggcggctgtgagctgagagtgaagttcagcaggtccgccgacgcccccgcctaccaacagggacagaatcagctgtacaatgagctgaacctgggcagaagagaggagtacgacgtgctggataagaggagaggcagggaccctgagatgggcggcaagcctagaagaaagaacccccaggagggcctgtacaatgaactgcagaaggacaagatggccgaggcctacagcgagatcggcatgaagggcgagagaaggaggggcaagggccacgatggcctgtaccagggcctgtccacagccaccaaggacacatacgacgccctgcacatgcaggccctgcccccaaggtgagtcgac

SEQ ID NO: 29 - DNA, 1506 bp, Artificial Sequence
  source: /note=“Description of Artificial Sequence Synthetic polynucleotide”
ggatccgccgccaccatgaagcacctgtggttcttcctgctgctggtggccgcccccagatgggtgctgtcccaggtgcagctgcagcagtccggccccggactggtgaagccttcccagaccctgagcctgacctgcgccatcagcggcgactccgtgtttagcaactccgcctcctggaactggatcagacagtccccctccagaggcctggagtggctgggcaagacagagtacaggagcaagtggtttgacgagtacgccccttccgtgaagggcaggatcgccatcagacccgatacctccaagaatcaggtgagcctgcagctgaatagcgtgacacccgaggacaccgccgtgtactactgtgtgagagactcctacaatggctacgacttcgacggctttgacatctggggccagggcaccctggtgaccgtgtccagcggcggaggcggaagcggaggaggaggaagcggcggaggaggctccggaggaggaggttcccagagcgtgctgacccagccccctagcgtgagcgccgctcctggacagaaggtgaccatcagctgctccggcggctccagcaacatcgagaagaactacgtgagctggtaccagcagctgcctggcacagcccctaagctgctgatctacgacaccaataagaggcctagcggcatccccgacagattcagcggctccaagtccggcacaagcgccacactgggcatcaccggcctgcagaccggcgacgaggccgattactactgtggcgcctgggataatatcctgaactccgagatgttcggcggcggcacaaagctgacagtgctgatcgaggtgatgtacccccccccctacctggacaatgagaagtccaacggcacaatcatccacgtgaagggcaagcacctgtgtcccagccccctgtttcccggccctagcaagcccttctgggtgctggtggtggtgggcggcgtgctggcttgttacagcctgctggtgacagtcgccttcatcatcttttgggtgaagaggggcaggaagaagctgctgtacatctttaagcagccctttatgaggcccgtgcagacaacccaggaggaggacggctgtagctgcaggtttcctgaggaggaggagggcggctgtgagctgagagtgaagttcagcaggtccgccgacgcccccgcctaccaacagggacagaatcagctgtacaatgagctgaacctgggcagaagagaggagtacgacgtgctggataagaggagaggcagggaccctgagatgggcggcaagcctagaagaaagaacccccaggagggcctgtacaatgaactgcagaaggacaagatggccgaggcctacagcgagatcggcatgaagggcgagagaaggaggggcaagggccacgatggcctgtaccagggcctgtccacagccaccaaggacacatacgacgccctgcacatgcaggccctgcccccaaggtgagtcgac

SEQ ID NO: 30 - DNA, 1506 bp, Artificial Sequence
  source: /note=“Description of Artificial Sequence Synthetic polynucleotide”
ggatccgccgccaccatgaagcacctgtggttctttctgctgctggtggccgcccccagatgggtgctgagccaggtgcagctgcagcagagcggccctggcctggtgaagccttcccagacactgagcctgacctgtgccatcagcggcgatagcgtgtttagcaacagcgccagctggaattggatcaggcagagcccttccaggggcctggagtggctgggcaagaccgagtacaggtccaagtggttcgatgagtacgcccctagcgtgaagggcaggatcgccatcaggcctgacacctccaagaaccaggtgtccctgcagctgaattccgtgacacctgaggataccgccgtgtactactgcgtgagggattcctacaacggctacgacttcgacggctttgatatctggggccagggcacactggtgacagtgtcctccggcggcggcggaagcggaggaggaggatctggcggcggcggttccggaggaggaggttctcagtccgtggtgacccagcctcccagcgtgagcgccgctcctggacagaaggtgaccatcagctgtagcggcagctcctccaacatcggcaacaactacgtgtcctggtaccagcagctgcccggcaccgccccaaagctgctgatctacgataataataagagaccttccggcatccccgatagattttccggcagcaagagcggcacaagcgccaccctgggcatcacaggcctgcagaccggcgacgaggccgattactactgcggcacctgggatagcagcctgtccgccgaggtgtttggcggcggcaccaagctgaccgtgctgatcgaggtgatgtacccccccccctacctggacaatgagaagtccaacggcacaatcatccacgtgaagggcaagcacctgtgtcccagccccctgtttcccggccctagcaagcccttctgggtgctggtggtggtgggcggcgtgctggcttgttacagcctgctggtgacagtcgccttcatcatcttttgggtgaagaggggcaggaagaagctgctgtacatctttaagcagccctttatgaggcccgtgcagacaacccaggaggaggacggctgtagctgcaggtttcctgaggaggaggagggcggctgtgagctgagagtgaagttcagcaggtccgccgacgcccccgcctaccaacagggacagaatcagctgtacaatgagctgaacctgggcagaagagaggagtacgacgtgctggataagaggagaggcagggaccctgagatgggcggcaagcctagaagaaagaacccccaggagggcctgtacaatgaactgcagaaggacaagatggccgaggcctacagcgagatcggcatgaagggcgagagaaggaggggcaagggccacgatggcctgtaccagggcctgtccacagccaccaaggacacatacgacgccctgcacatgcaggccctgcccccaaggtgagtcgac

SEQ ID NO: 31 - DNA, 1506 bp, Artificial Sequence
  source: /note=“Description of Artificial Sequence Synthetic polynucleotide”
ggatccgccgccaccatgaagcacctgtggtttttcctgctgctggtggccgcccccaggtgggttctgtcccaggtgcagctgcagcagagcggccccggactggtgaagcccagccagacactgagcctgacatgtgacatctccggcgattccgtgttctccaacacagccagctggaattggatcaggcagagccccagcaggggcctggagtggctgggaaagacagagtacagatcccagtggctgcacgagtacgccccttccgtgagatccaggatcgccatcagacccgacacctccaagaaccaggtgtccctgcagctgtccttcgtgacccccgaggacaccgccgtgtactactgtgtgagggattcctacaatggctacgccttcgatggctttgacacctggggccagggcaccctggtgaccgtgagcagcggcggaggcggaagcggaggaggaggaagcggcggaggaggctccggaggaggaggtagccagagcgtggtgacccagccccccagcgtgtccgctgctcctggacagaaggtgaccatcagctgttccggcaggaactccaacatcgccaacaactttgtgtcctggtacagacagctgcctggcaccgcccctaagctgctgatctacgacaataataagaggcctagcggcatccccgatagattctccggctccaagagcggcacatccgccaccctggccatcaccggcctgcagaccggcgacgaggccgattactactgcgagtcctgggatagctccctgaccgccgaggtgtttggcaccggcaccaaggtgaccgtgctgatcgaggtgatgtacccccccccctacctggacaatgagaagtccaacggcacaatcatccacgtgaagggcaagcacctgtgtcccagccccctgtttcccggccctagcaagcccttctgggtgctggtggtggtgggcggcgtgctggcttgttacagcctgctggtgacagtcgccttcatcatcttttgggtgaagaggggcaggaagaagctgctgtacatctttaagcagccctttatgaggcccgtgcagacaacccaggaggaggacggctgtagctgcaggtttcctgaggaggaggagggcggctgtgagctgagagtgaagttcagcaggtccgccgacgcccccgcctaccaacagggacagaatcagctgtacaatgagctgaacctgggcagaagagaggagtacgacgtgctggataagaggagaggcagggaccctgagatgggcggcaagcctagaagaaagaacccccaggagggcctgtacaatgaactgcagaaggacaagatggccgaggcctacagcgagatcggcatgaagggcgagagaaggaggggcaagggccacgatggcctgtaccagggcctgtccacagccaccaaggacacatacgacgccctgcacatgcaggccctgcccccaaggtgagtcgac

SEQ ID NO: 32 - DNA, 1506 bp, Artificial Sequence
  source: /note=“Description of Artificial Sequence Synthetic polynucleotide”
ggatccgccgccaccatgaagcacctgtggttctttctgctgctggtggccgcccctaggtgggtgctgagccaggtgcagctgcagcagtccggccctggcctggtgaagcccagccagacactgagcctgacctgtgccatctccggcgactccgtgtccagcaacagcgcctcctggaactggatcagacagtcccctagcaggggcctggagtggctgggcaaggtggagtacagatccaagtggtacgatgagtacgcccccagcgtgaagagcagaatcgccatcagacctgacaccagcaagaaccaggtgtccctgcacctgatcagcgtgacccctgatgacacagccgtgtactactgcgccagagatagctacaacggccacgcctttgatggctttgatgtgtggggccagggcacaatggtgaccgtgagctccggcggcggcggatctggaggaggaggaagcggcggcggcggttctggaggaggtggaagccagagcgtggtgacacagcctccctccgtgtccgccgcccctggacaaaaggtgaccatctcctgcagcggcagcagcagcaacatcggcaataactacgtgtcctggtaccagcagctgcctggcacagcccccaagctgctgatctacgacaacaataagaggcccagcggcatccccgacaggttctccggctccaagtccggcacctccgccacactgggcatcacaggcctgcagaccggcgacgaggccgactactactgtggcacctgggattccagcctgagcgccgaggtgtttggcggcggcacaaaggtgacagtgctgatcgaggtgatgtacccccccccctacctggacaatgagaagtccaacggcacaatcatccacgtgaagggcaagcacctgtgtcccagccccctgtttcccggccctagcaagcccttctgggtgctggtggtggtgggcggcgtgctggcttgttacagcctgctggtgacagtcgccttcatcatcttttgggtgaagaggggcaggaagaagctgctgtacatctttaagcagccctttatgaggcccgtgcagacaacccaggaggaggacggctgtagctgcaggtttcctgaggaggaggagggcggctgtgagctgagagtgaagttcagcaggtccgccgacgcccccgcctaccaacagggacagaatcagctgtacaatgagctgaacctgggcagaagagaggagtacgacgtgctggataagaggagaggcagggaccctgagatgggcggcaagcctagaagaaagaacccccaggagggcctgtacaatgaactgcagaaggacaagatggccgaggcctacagcgagatcggcatgaagggcgagagaaggaggggcaagggccacgatggcctgtaccagggcctgtccacagccaccaaggacacatacgacgccctgcacatgcaggccctgcccccaaggtgagtcgac

SEQ ID NO: 33 - DNA, 1506 bp, Artificial Sequence
  source: /note=“Description of Artificial Sequence Synthetic polynucleotide”
ggatccgccgccaccatgaagcacctgtggttttttctgctgctggtggccgcccccagatgggtgctgagccaggtgcagctgcagcagtccggccctggcctggtgaagccttcccagacactgtccctgacctgcgccatcagcggcgacagcgtgagcagcaattccgccagctggaactggatcaggcagtcccccagcaggggcctggagtggctgggaaaggtggagtacaggagcaagtggtacgacgagtacgcccccagcgtgaagagcagaatcgccatcaggcccgacacatccaagaatcaggtgagcctgcacctgatcagcgtgacccccgatgacacagccgtgtactactgcgccagagactcctacaacggccacgccttcgacggctttgacgtgtggggccagggcacaatggtgacagtgagctccggcggcggcggctctggaggaggaggatctggcggcggcggaagcggaggaggaggttctcagagcgtgctgacccagccccctagcctgagcgccgctcctggacagagagtgaccatcagctgctccggcacaacctccaatatcgccaataatttcgtgtcctggtaccagcagctgtccggctccgcccccaagctgctgatcaatgacaataacaagagaccctccggcatccccgataggttctccggcagcaagtccggcgcctccgccacactggccatcaccggactgcagacagaggacgaggccgactactactgtggcgtgtgggacaacagcctgaatgccgaggtgtttggcggcggcaccaaggtgaccgtgctgatcgaggtgatgtacccccccccctacctggacaatgagaagtccaacggcacaatcatccacgtgaagggcaagcacctgtgtcccagccccctgtttcccggccctagcaagcccttctgggtgctggtggtggtgggcggcgtgctggcttgttacagcctgctggtgacagtcgccttcatcatcttttgggtgaagaggggcaggaagaagctgctgtacatctttaagcagccctttatgaggcccgtgcagacaacccaggaggaggacggctgtagctgcaggtttcctgaggaggaggagggcggctgtgagctgagagtgaagttcagcaggtccgccgacgcccccgcctaccaacagggacagaatcagctgtacaatgagctgaacctgggcagaagagaggagtacgacgtgctggataagaggagaggcagggaccctgagatgggcggcaagcctagaagaaagaacccccaggagggcctgtacaatgaactgcagaaggacaagatggccgaggcctacagcgagatcggcatgaagggcgagagaaggaggggcaagggccacgatggcctgtaccagggcctgtccacagccaccaaggacacatacgacgccctgcacatgcaggccctgcccccaaggtgagtcgac

SEQ ID NO: 34 - DNA, 1506 bp, Artificial Sequence
  source: /note=“Description of Artificial Sequence Synthetic polynucleotide”
ggatccgccgccaccatgaagcacctgtggtttttcctgctgctggtggccgcccctaggtgggtgctgtcccaggtgcagctgcagcagtccggccccggactggtgaagccttcccagaccctgagcctgacatgtgccatctccggcgacagcgtgtcctccaactccgccagctggaactggatcaggcagtccccttccagaggcctggagtggctgggcaagacagagtacagaagcaagtggtactacgagtttgcccctagcgtgaagtccagaatcaccatcacccccgacaccagcaagaatcagtttagcctgcagctgaatagcgtgacccctgaggatacagccgtgtactactgcgtgagggatagctacaacggctacgacttcgacggctttgatatctggggccagggcacactggtgaccgtgtccagcggcggcggcggatctggaggaggaggaagcggcggcggaggctctggaggaggtggaagccagagcgtgctgacccagccccctagcgtgagcgccgctcctggacagaaggtgacaatcagctgctccggctccagcagcaacatcggcagcaattacgtggcctggtaccagcagctgcctggcaccgcccccaagctgctgatctacgacacaaacaagaggcccagcggcatccctgacaggttcagcggcagcaagagcggcaccagcgccaccctgggcatcaccggactgcagacaggcgacgaggccgattactactgtggcacctgggactccagcctgagcgccgaggtgtttggcacaggcacaaaggtgaccgtgctgatcgaggtgatgtacccccccccctacctggacaatgagaagtccaacggcacaatcatccacgtgaagggcaagcacctgtgtcccagccccctgtttcccggccctagcaagcccttctgggtgctggtggtggtgggcggcgtgctggcttgttacagcctgctggtgacagtcgccttcatcatcttttgggtgaagaggggcaggaagaagctgctgtacatctttaagcagccctttatgaggcccgtgcagacaacccaggaggaggacggctgtagctgcaggtttcctgaggaggaggagggcggctgtgagctgagagtgaagttcagcaggtccgccgacgcccccgcctaccaacagggacagaatcagctgtacaatgagctgaacctgggcagaagagaggagtacgacgtgctggataagaggagaggcagggaccctgagatgggcggcaagcctagaagaaagaacccccaggagggcctgtacaatgaactgcagaaggacaagatggccgaggcctacagcgagatcggcatgaagggcgagagaaggaggggcaagggccacgatggcctgtaccagggcctgtccacagccaccaaggacacatacgacgccctgcacatgcaggccctgcccccaaggtgagtcgac

SEQ ID NO: 35 - protein, 494 aa, Artificial Sequence
  source: /note=“Description of Artificial Sequence Synthetic polypeptide”
MKHLWFFLLLVAAPRWVLSQVQLQQSGPGLVQPSRTLSLTCAISGDSISSNTASWNWIRQSPSRGLEWVGKTEYRSGWFFEYAPSVGGRVNISPDTSKNQFSLQLNSVTPEDTAVYFCARDSYNDYDNDGFDIWGQGTLVTVSSGGGGSGGGGSGGGGSGGGGSQSVLTQPPSVSAAPGQKVTISCSGSSSNIGSNYVAWYQQLPGTAPKLLIYDTNKRPSGIPDRFSGSKSGTSATLGITGLQTGDEADYYCGTWDSSLSAEVFGTGTKVTVLIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 494 aa, Artificial Sequence
  source: /note=“Description of Artificial Sequence Synthetic polypeptide”
MKHLWFFLLLVAAPRWVLSQVQLQQSGPGLVKPSQTLSLTCAISGDSVSSNSASWNWIRQSPSRGLEWLGKTEYRSKWYYEFAPSVKSRITITPDTSKNQFSLQLNSVTPEDTAVYYCVRDSYNGYDFDGFDIWGQGTLVTVSSGGGGSGGGGSGGGGSGGGGSQSVLTQPPSVSAAPGQKVTISCSGSSSNIGSNYVAWYQQLPGTAPKLLIYDTNKRPSGIPDRFSGSKSGTSATLGITGLQTGDEADYYCGTWDSSLSAEVFGTGTKVTVLIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 494 aa, Artificial Sequence
  source: /note=“Description of Artificial Sequence Synthetic polypeptide”
MKHLWFFLLLVAAPRWVLSQVQLQQSGPGLVKPSQTLSLTCAISGDSVFSNSASWNWIRQSPSRGLEWLGKTEYRSKWFDEYAPSVKGRIAIRPDTSKNQVSLQLNSVTPEDTAVYYCVRDSYNGYDFDGFDIWGQGTLVTVSSGGGGSGGGGSGGGGSGGGGSQSVLTQPPSVSAAPGQQVIISCSGSSSNVGNNYVAWYQQVPGAAPRLLIYDNYKRPSGIPDRFSGSKSGTSATLGITGLQSGDEADYYCGSWDSSLSAEVFGGGTKLTALIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 494 aa, Artificial Sequence
  source: /note=“Description of Artificial Sequence Synthetic polypeptide”
MKHLWFFLLLVAAPRWVLSQVQLQQSGPGLVKPSQTLSLTCAISGDSVFSTTASWNWIRQSPSRGLEWLGKTEYRSKWFDEYAPSVKGRIAIRPDTSRNQVSLQLNSMTPEDTAVYFCVRDSYNGYDFDGFDIWGQGTTVTVSSGGGGSGGGGSGGGGSGGGGSQSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAEMFGGGTKVTVLIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 494 aa, Artificial Sequence
  source: /note=“Description of Artificial Sequence Synthetic polypeptide”
MKHLWFFLLLVAAPRWVLSQVQLQQSGPGLVKPSQTLSLTCAISGDSVSSNSASWNWIRQSPSRGLEWLGKVEYRSKWYDEYAPSVKSRIAIRPDTSKNQVSLHLISVTPDDTAVYYCARDSYNGHAFDGFDVWGQGTLVTVSSGGGGSGGGGSGGGGSGGGGSQSVLTQPPSVSAAPGQKVTISCSGSSSNIGDNLVSWYQQLPGTAPKLLIYDNNKRPSGIPDRFSGSKSGTSATLDITGLQTGDEADYYCESWDNNLTAEVFGTGTKVTVLIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 494 aa, Artificial Sequence
  source: /note=“Description of Artificial Sequence Synthetic polypeptide”
MKHLWFFLLLVAAPRWVLSQVQLQQSGPGLVKPSQTLSLTCAISGDSVFSNSASWNWIRQSPSRGLEWLGKTEYRSKWFDEYAPSVKGRIAIRPDTSKNQVSLQLNSVTPEDTAVYYCVRDSYNGYDFDGFDIWGQGTLVTVSSGGGGSGGGGSGGGGSGGGGSQSVLTQPPSVSAAPGQKVTISCSGGSSNIEKNYVSWYQQLPGTAPKLLIYDTNKRPSGIPDRFSGSKSGTSATLGITGLQTGDEADYYCGAWDNILNSEMFGGGTKLTVLIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 494 aa, Artificial Sequence
  source: /note=“Description of Artificial Sequence Synthetic polypeptide”
MKHLWFFLLLVAAPRWVLSQVQLQQSGPGLVKPSQTLSLTCAISGDSVFSNSASWNWIRQSPSRGLEWLGKTEYRSKWFDEYAPSVKGRIAIRPDTSKNQVSLQLNSVTPEDTAVYYCVRDSYNGYDFDGFDIWGQGTLVTVSSGGGGSGGGGSGGGGSGGGGSQSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAEVFGGGTKLTVLIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 494 aa, Artificial Sequence
  source: /note=“Description of Artificial Sequence Synthetic polypeptide”
MKHLWFFLLLVAAPRWVLSQVQLQQSGPGLVKPSQTLSLTCDISGDSVFSNTASWNWIRQSPSRGLEWLGKTEYRSQWLHEYAPSVRSRIAIRPDTSKNQVSLQLSFVTPEDTAVYYCVRDSYNGYAFDGFDTWGQGTLVTVSSGGGGSGGGGSGGGGSGGGGSQSVVTQPPSVSAAPGQKVTISCSGRNSNIANNFVSWYRQLPGTAPKLLIYDNNKRPSGIPDRFSGSKSGTSATLAITGLQTGDEADYYCESWDSSLTAEVFGTGTKVTVLIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 494 aa, Artificial Sequence
  source: /note=“Description of Artificial Sequence Synthetic polypeptide”
MKHLWFFLLLVAAPRWVLSQVQLQQSGPGLVKPSQTLSLTCAISGDSVSSNSASWNWIRQSPSRGLEWLGKVEYRSKWYDEYAPSVKSRIAIRPDTSKNQVSLHLISVTPDDTAVYYCARDSYNGHAFDGFDVWGQGTMVTVSSGGGGSGGGGSGGGGSGGGGSQSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAEVFGGGTKVTVLIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 494 aa, Artificial Sequence
  source: /note=“Description of Artificial Sequence Synthetic polypeptide”
MKHLWFFLLLVAAPRWVLSQVQLQQSGPGLVKPSQTLSLTCAISGDSVSSNSASWNWIRQSPSRGLEWLGKVEYRSKWYDEYAPSVKSRIAIRPDTSKNQVSLHLISVTPDDTAVYYCARDSYNGHAFDGFDVWGQGTMVTVSSGGGGSGGGGSGGGGSGGGGSQSVLTQPPSLSAAPGQRVTISCSGTTSNIANNFVSWYQQLSGSAPKLLINDNNKRPSGIPDRFSGSKSGASATLAITGLQTEDEADYYCGVWDNSLNAEVFGGGTKVTVLIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 13 aa, Artificial Sequence
  source: /note=“Description of Artificial Sequence Synthetic peptide”
SGSSSNIGSNYVA

SEQ ID NO: 46 - protein, 13 aa, Artificial Sequence
  source: /note=“Description of Artificial Sequence Synthetic peptide”
SGSSSNVGNNYVA

SEQ ID NO: 47 - protein, 13 aa, Artificial Sequence
  source: /note=“Description of Artificial Sequence Synthetic peptide”
SGSSSNIGNNYVS

SEQ ID NO: 48 - protein, 13 aa, Artificial Sequence
  source: /note=“Description of Artificial Sequence Synthetic peptide”
SGSSSNIGDNLVS

SEQ ID NO: 49 - protein, 13 aa, Artificial Sequence
  source: /note=“Description of Artificial Sequence Synthetic peptide”
SGGSSNIEKNYVS

SEQ ID NO: 50 - protein, 13 aa, Artificial Sequence
  source: /note=“Description of Artificial Sequence Synthetic peptide”
SGRNSNIANNFVS

SEQ ID NO: 51 - protein, 13 aa, Artificial Sequence
  source: /note=“Description of Artificial Sequence Synthetic peptide”
SGTTSNIANNFVS

SEQ ID NO: 52 - protein, 7 aa, Artificial Sequence
  source: /note=“Description of Artificial Sequence Synthetic peptide”
DTNKRPS

SEQ ID NO: 53 - protein, 7 aa, Artificial Sequence
  source: /note=“Description of Artificial Sequence Synthetic peptide”
DNYKRPS

SEQ ID NO: 54 - protein, 7 aa, Artificial Sequence
  source: /note=“Description of Artificial Sequence Synthetic peptide”
DNNKRPS

SEQ ID NO: 55 - protein, 11 aa, Artificial Sequence
  source: /note=“Description of Artificial Sequence Synthetic peptide”
GTWDSSLSAEV

SEQ ID NO: 56 - protein, 11 aa, Artificial Sequence
  source: /note=“Description of Artificial Sequence Synthetic peptide”
GSWDSSLSAEV

SEQ ID NO: 57 - protein, 11 aa, Artificial Sequence
  source: /note=“Description of Artificial Sequence Synthetic peptide”
GTWDSSLSAEM

SEQ ID NO: 58 - protein, 11 aa, Artificial Sequence
  source: /note=“Description of Artificial Sequence Synthetic peptide”
ESWDNNLTAEV

SEQ ID NO: 59 - protein, 11 aa, Artificial Sequence
  source: /note=“Description of Artificial Sequence Synthetic peptide”
GAWDNILNSEM

SEQ ID NO: 60 - protein, 11 aa, Artificial Sequence
  source: /note=“Description of Artificial Sequence Synthetic peptide”
ESWDSSLTAEV

SEQ ID NO: 61 - protein, 11 aa, Artificial Sequence
  source: /note=“Description of Artificial Sequence Synthetic peptide”
GVWDNSLNAEV

SEQ ID NO: 62 - protein, 7 aa, Artificial Sequence
  source: /note=“Description of Artificial Sequence Synthetic peptide”
SNTASWN

SEQ ID NO: 63 - protein, 7 aa, Artificial Sequence
  source: /note=“Description of Artificial Sequence Synthetic peptide”
SNSASWN

SEQ ID NO: 64 - protein, 7 aa, Artificial Sequence
  source: /note=“Description of Artificial Sequence Synthetic peptide”
STTASWN

SEQ ID NO: 65 - protein, 18 aa, Artificial Sequence
  source: /note=“Description of Artificial Sequence Synthetic peptide”
KTEYRSGWFFEYAPSVGG

SEQ ID NO: 66 - protein, 18 aa, Artificial Sequence
  source: /note=“Description of Artificial Sequence Synthetic peptide”
KTEYRSKWYYEFAPSVKS

SEQ ID NO: 67 - protein, 18 aa, Artificial Sequence
  source: /note=“Description of Artificial Sequence Synthetic peptide”
KTEYRSKWFDEYAPSVKG

SEQ ID NO: 68 - protein, 18 aa, Artificial Sequence
  source: /note=“Description of Artificial Sequence Synthetic peptide”
KVEYRSKWYDEYAPSVKS

SEQ ID NO: 69 - protein, 18 aa, Artificial Sequence
  source: /note=“Description of Artificial Sequence Synthetic peptide”
KTEYRSQWLHEYAPSVRS

SEQ ID NO: 70 - protein, 13 aa, Artificial Sequence
  source: /note=“Description of Artificial Sequence Synthetic peptide”
DSYNDYDNDGFDI

SEQ ID NO: 71 - protein, 13 aa, Artificial Sequence
  source: /note=“Description of Artificial Sequence Synthetic peptide”
DSYNGYDFDGFDI

SEQ ID NO: 72 - protein, 13 aa, Artificial Sequence
  source: /note=“Description of Artificial Sequence Synthetic peptide”
DSYNGHAFDGFDV

SEQ ID NO: 73 - protein, 13 aa, Artificial Sequence
  source: /note=“Description of Artificial Sequence Synthetic peptide”
DSYNGYAFDGFDT

SEQ ID NO: 74 - protein, 110 aa, Artificial Sequence
  source: /note=“Description of Artificial Sequence Synthetic polypeptide”
QSVLTQPPSVSAAPGQKVTISCSGSSSNIGSNYVAWYQQLPGTAPKLLIYDTNKRPSGIPDRFSGSKSGTSATLGITGLQTGDEADYYCGTWDSSLSAEVFGTGTKVTVL

SEQ ID NO: 75 - protein, 110 aa, Artificial Sequence
  source: /note=“Description of Artificial Sequence Synthetic polypeptide”
QSVLTQPPSVSAAPGQQVIISCSGSSSNVGNNYVAWYQQVPGAAPRLLIYDNYKRPSGIPDRFSGSKSGTSATLGITGLQSGDEADYYCGSWDSSLSAEVFGGGTKLTAL

SEQ ID NO: 76 - protein, 109 aa, Artificial Sequence
  source: /note=“Description of Artificial Sequence Synthetic polypeptide”
QSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAEMFGGGTKVTV

SEQ ID NO: 77 - protein, 110 aa, Artificial Sequence
  source: /note=“Description of Artificial Sequence Synthetic polypeptide”
QSVLTQPPSVSAAPGQKVTISCSGSSSNIGDNLVSWYQQLPGTAPKLLIYDNNKRPSGIPDRFSGSKSGTSATLDITGLQTGDEADYYCESWDNNLTAEVFGTGTKVTVL

SEQ ID NO: 78 - protein, 110 aa, Artificial Sequence
  source: /note=“Description of Artificial Sequence Synthetic polypeptide”
QSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAEVFGGGTKLTVL

SEQ ID NO: 79 - protein, 110 aa, Artificial Sequence
  source: /note=“Description of Artificial Sequence Synthetic polypeptide”
QSVVTQPPSVSAAPGQKVTISCSGRNSNIANNFVSWYRQLPGTAPKLLIYDNNKRPSGIPDRFSGSKSGTSATLAITGLQTGDEADYYCESWDSSLTAEVFGTGTKVTVL

SEQ ID NO: 80 - protein, 110 aa, Artificial Sequence
  source: /note=“Description of Artificial Sequence Synthetic polypeptide”
QSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAEVFGGGTKVTVL

SEQ ID NO: 81 - protein, 110 aa, Artificial Sequence
  source: /note=“Description of Artificial Sequence Synthetic polypeptide”
QSVLTQPPSLSAAPGQRVTISCSGTTSNIANNFVSWYQQLSGSAPKLLINDNNKRPSGIPDRFSGSKSGASATLAITGLQTEDEADYYCGVWDNSLNAEVFGGGTKVTVL

SEQ ID NO: 82 - protein, 125 aa, Artificial Sequence
  source: /note=“Description of Artificial Sequence Synthetic polypeptide”
QVQLQQSGPGLVQPSRTLSLTCAISGDSISSNTASWNWIRQSPSRGLEWVGKTEYRSGWFFEYAPSVGGRVNISPDTSKNQFSLQLNSVTPEDTAVYFCARDSYNDYDNDGFDIWGQGTLVTVSS

SEQ ID NO: 83 - protein, 125 aa, Artificial Sequence
  source: /note=“Description of Artificial Sequence Synthetic polypeptide”
QVQLQQSGPGLVKPSQTLSLTCAISGDSVSSNSASWNWIRQSPSRGLEWLGKTEYRSKWYYEFAPSVKSRITITPDTSKNQFSLQLNSVTPEDTAVYYCVRDSYNGYDFDGFDIWGQGTLVTVSS

SEQ ID NO: 84 - protein, 125 aa, Artificial Sequence
  source: /note=“Description of Artificial Sequence Synthetic polypeptide”
QVQLQQSGPGLVKPSQTLSLTCAISGDSVFSNSASWNWIRQSPSRGLEWLGKTEYRSKWFDEYAPSVKGRIAIRPDTSKNQVSLQLNSVTPEDTAVYYCVRDSYNGYDFDGFDIWGQGTLVTVSS

SEQ ID NO: 85 - protein, 110 aa, Artificial Sequence
  source: /note=“Description of Artificial Sequence Synthetic polypeptide”
QSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAEMFGGGTKVTVL

SEQ ID NO: 86 - protein, 125 aa, Artificial Sequence
  source: /note=“Description of Artificial Sequence Synthetic polypeptide”
QVQLQQSGPGLVKPSQTLSLTCAISGDSVSSNSASWNWIRQSPSRGLEWLGKVEYRSKWYDEYAPSVKSRIAIRPDTSKNQVSLHLISVTPDDTAVYYCARDSYNGHAFDGFDVWGQGTLVTVSS

SEQ ID NO: 87 - protein, 125 aa, Artificial Sequence
  source: /note=“Description of Artificial Sequence Synthetic polypeptide”
QVQLQQSGPGLVKPSQTLSLTCDISGDSVFSNTASWNWIRQSPSRGLEWLGKTEYRSQWLHEYAPSVRSRIAIRPDTSKNQVSLQLSFVTPEDTAVYYCVRDSYNGYAFDGFDTWGQGTLVTVSS

SEQ ID NO: 88 - protein, 125 aa, Artificial Sequence
  source: /note=“Description of Artificial Sequence Synthetic polypeptide”
QVQLQQSGPGLVKPSQTLSLTCAISGDSVSSNSASWNWIRQSPSRGLEWLGKVEYRSKWYDEYAPSVKSRIAIRPDTSKNQVSLHLISVTPDDTAVYYCARDSYNGHAFDGFDVWGQGTMVTVSS

SEQ ID NO: 89 - protein, 255 aa, Artificial Sequence
  source: /note=“Description of Artificial Sequence Synthetic polypeptide”
QVQLQQSGPGLVQPSRTLSLTCAISGDSISSNTASWNWIRQSPSRGLEWVGKTEYRSGWFFEYAPSVGGRVNISPDTSKNQFSLQLNSVTPEDTAVYFCARDSYNDYDNDGFDIWGQGTLVTVSSGGGGSGGGGSGGGGSGGGGSQSVLTQPPSVSAAPGQKVTISCSGSSSNIGSNYVAWYQQLPGTAPKLLIYDTNKRPSGIPDRFSGSKSGTSATLGITGLQTGDEADYYCGTWDSSLSAEVFGTGTKVTVL

SEQ ID NO: 90 - protein, 255 aa, Artificial Sequence
  source: /note=“Description of Artificial Sequence Synthetic polypeptide”
QVQLQQSGPGLVKPSQTLSLTCAISGDSVSSNSASWNWIRQSPSRGLEWLGKTEYRSKWYYEFAPSVKSRITITPDTSKNQFSLQLNSVTPEDTAVYYCVRDSYNGYDFDGFDIWGQGTLVTVSSGGGGSGGGGSGGGGSGGGGSQSVLTQPPSVSAAPGQKVTISCSGSSSNIGSNYVAWYQQLPGTAPKLLIYDTNKRPSGIPDRFSGSKSGTSATLGITGLQTGDEADYYCGTWDSSLSAEVFGTGTKVTVL

SEQ ID NO: 91 - protein, 255 aa, Artificial Sequence
  source: /note=“Description of Artificial Sequence Synthetic polypeptide”
QVQLQQSGPGLVKPSQTLSLTCAISGDSVFSNSASWNWIRQSPSRGLEWLGKTEYRSKWFDEYAPSVKGRIAIRPDTSKNQVSLQLNSVTPEDTAVYYCVRDSYNGYDFDGFDIWGQGTLVTVSSGGGGSGGGGSGGGGSGGGGSQSVLTQPPSVSAAPGQQVIISCSGSSSNVGNNYVAWYQQVPGAAPRLLIYDNYKRPSGIPDRFSGSKSGTSATLGITGLQSGDEADYYCGSWDSSLSAEVFGGGTKLTAL

SEQ ID NO: 92 - protein, 255 aa, Artificial Sequence
  source: /note=“Description of Artificial Sequence Synthetic polypeptide”
QVQLQQSGPGLVKPSQTLSLTCAISGDSVFSTTASWNWIRQSPSRGLEWLGKTEYRSKWFDEYAPSVKGRIAIRPDTSRNQVSLQLNSMTPEDTAVYFCVRDSYNGYDFDGFDIWGQGTTVTVSSGGGGSGGGGSGGGGSGGGGSQSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAEMFGGGTKVTVL

SEQ ID NO: 93 - protein, 255 aa, Artificial Sequence
  source: /note=“Description of Artificial Sequence Synthetic polypeptide”
QVQLQQSGPGLVKPSQTLSLTCAISGDSVSSNSASWNWIRQSPSRGLEWLGKVEYRSKWYDEYAPSVKSRIAIRPDTSKNQVSLHLISVTPDDTAVYYCARDSYNGHAFDGFDVWGQGTLVTVSSGGGGSGGGGSGGGGSGGGGSQSVLTQPPSVSAAPGQKVTISCSGSSSNIGDNLVSWYQQLPGTAPKLLIYDNNKRPSGIPDRFSGSKSGTSATLDITGLQTGDEADYYCESWDNNLTAEVFGTGTKVTVL

SEQ ID NO: 94 - protein, 255 aa, Artificial Sequence
  source: /note=“Description of Artificial Sequence Synthetic polypeptide”
QVQLQQSGPGLVKPSQTLSLTCAISGDSVFSNSASWNWIRQSPSRGLEWLGKTEYRSKWFDEYAPSVKGRIAIRPDTSKNQVSLQLNSVTPEDTAVYYCVRDSYNGYDFDGFDIWGQGTLVTVSSGGGGSGGGGSGGGGSGGGGSQSVLTQPPSVSAAPGQKVTISCSGGSSNIEKNYVSWYQQLPGTAPKLLIYDTNKRPSGIPDRFSGSKSGTSATLGITGLQTGDEADYYCGAWDNILNSEMFGGGTKLTVL

SEQ ID NO: 95 - protein, 255 aa, Artificial Sequence
  source: /note=“Description of Artificial Sequence Synthetic polypeptide”
QVQLQQSGPGLVKPSQTLSLTCAISGDSVFSNSASWNWIRQSPSRGLEWLGKTEYRSKWFDEYAPSVKGRIAIRPDTSKNQVSLQLNSVTPEDTAVYYCVRDSYNGYDFDGFDIWGQGTLVTVSSGGGGSGGGGSGGGGSGGGGSQSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAEVFGGGTKLTVL

SEQ ID NO: 96 - protein, 255 aa, Artificial Sequence
  source: /note=“Description of Artificial Sequence Synthetic polypeptide”
QVQLQQSGPGLVKPSQTLSLTCDISGDSVFSNTASWNWIRQSPSRGLEWLGKTEYRSQWLHEYAPSVRSRIAIRPDTSKNQVSLQLSFVTPEDTAVYYCVRDSYNGYAFDGFDTWGQGTLVTVSSGGGGSGGGGSGGGGSGGGGSQSVVTQPPSVSAAPGQKVTISCSGRNSNIANNFVSWYRQLPGTAPKLLIYDNNKRPSGIPDRFSGSKSGTSATLAITGLQTGDEADYYCESWDSSLTAEVFGTGTKVTVL

SEQ ID NO: 97 - protein, 255 aa, Artificial Sequence
  source: /note=“Description of Artificial Sequence Synthetic polypeptide”
QVQLQQSGPGLVKPSQTLSLTCAISGDSVSSNSASWNWIRQSPSRGLEWLGKVEYRSKWYDEYAPSVKSRIAIRPDTSKNQVSLHLISVTPDDTAVYYCARDSYNGHAFDGFDVWGQGTMVTVSSGGGGSGGGGSGGGGSGGGGSQSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAEVFGGGTKVTVL

SEQ ID NO: 98 - protein, 255 aa, Artificial Sequence
  source: /note=“Description of Artificial Sequence Synthetic polypeptide”
QVQLQQSGPGLVKPSQTLSLTCAISGDSVSSNSASWNWIRQSPSRGLEWLGKVEYRSKWYDEYAPSVKSRIAIRPDTSKNQVSLHLISVTPDDTAVYYCARDSYNGHAFDGFDVWGQGTMVTVSSGGGGSGGGGSGGGGSGGGGSQSVLTQPPSLSAAPGQRVTISCSGTTSNIANNFVSWYQQLSGSAPKLLINDNNKRPSGIPDRFSGSKSGASATLAITGLQTEDEADYYCGVWDNSLNAEVFGGGTKVTVL

SEQ ID NO: 99 - protein, 39 aa, Artificial Sequence
  source: /note=“Description of Artificial Sequence Synthetic polypeptide”
IEVMYPPPYLDNEKSNGTIIHVKGKHLCPSPLFPGPSKP

SEQ ID NO: 100 - protein, 27 aa, Artificial Sequence
  source: /note=“Description of Artificial Sequence Synthetic peptide”
FWVLVVVGGVLACYSLLVTVAFIIFWV

SEQ ID NO: 101 - protein, 112 aa, Artificial Sequence
  source: /note=“Description of Artificial Sequence Synthetic polypeptide”
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - protein, 66 aa, Artificial Sequence
  source: /note=“Description of Artificial Sequence Synthetic polypeptide”
IEVMYPPPYLDNEKSNGTIIHVKGKHLCPSPLFPGPSKPFWVLVVVGGVLACYSLLVTVAFIIFWV

SEQ ID NO: 103 - protein, 19 aa, Artificial Sequence
  source: /note=“Description of Artificial Sequence Synthetic peptide”
MKHLWFFLLLVAAPRWVLS

SEQ ID NO: 104 - DNA, 57 bp, Artificial Sequence
  source: /note=“Description of Artificial Sequence Synthetic oligonucleotide”
atgaaacacctgtggttcttcctcctgctggtggcagctcccagatgggtcctgtcc

SEQ ID NO: 105 - protein, 42 aa, Artificial Sequence
  source: /note=“Description of Artificial Sequence Synthetic polypeptide”
KRGRKKLLYIFKQPFMRPVQTTQEEDGCSCRFPEEEEGGCEL